(19) United States
(12) Patent Application Publication
(10) Pub. No.: US 2024/0067657 A1
(43) Pub. Date: Feb. 29, 2024

(54) AMIDO-SUBSTITUTED PYRIDYL COMPOUNDS AND METHODS OF USE THEREOF FOR THE TREATMENT OF HERPESVIRUSES

(71) Applicants: Kira A. ARMACOST, Doylestown, PA (US); Richard Thaddeus BERGER, West Point, PA (US); Andrew J. COOKE, Doylestown, PA (US); Christopher Douglas COX, North Wales, PA (US); Brendan M. CROWLEY, West Point, PA (US); Marc LABROLI, West Point, PA (US); Michael Aaron PLOTKIN, West Point, PA (US); Izzat Tiedje RAHEEM, West Point, PA (US); Anthony W. SHAW, West Point, PA (US); Kelly-Ann S. SCHLEGEL, West Point, PA (US); Jason W. SKUDLAREK, West Point, PA (US); Ling TONG, Kenilworth, NJ (US); Merck Sharp & Dohme LLC, Rahway, NJ (US)

(72) Inventors: Kira A. Armacost, Doylestown, PA (US); Richard Thaddeus Berger, Jr., Harleysville, PA (US); Andrew J. Cooke, Doylestown, PA (US); Christopher Douglas Cox, Harleysville, PA (US); Brendan M. Crowley, Collegeville, PA (US); Marc Labroli, Moorestown, NJ (US); Michael Aaron Plotkin, Frenchtown, NJ (US); Izzat Tiedje Raheem, Doylestown, PA (US); Anthony W. Shaw, Harleysville, PA (US); Kelly-Ann S. Schlegel, Fleetwood, PA (US); Jason W. Skudlarek, Audubon, PA (US); Ling Tong, Warren, NJ (US)

(73) Assignee: Merck Sharp & Dohme LLC, Rahway, NJ (US)

(21) Appl. No.: 18/258,903

(22) PCT Filed: Dec. 21, 2021
(86) PCT No.: PCT/US2021/064478

§ 371 Date: Jun. 22, 2023

Related U.S. Application Data
(60) Provisional application No. 63/131,459, filed on Dec. 29, 2020.

(51) Int. Cl. C07D 498/04; A61K 31/517; C07D 401/12; C07D 401/14; C07D 413/14; C07D 417/14; C07D 471/04; C07D 519/00

(52) U.S. Cl. CPC C07D 498/04; A61K 31/517; C07D 401/12; C07D 401/14; C07D 413/14; C07D 417/14; C07D 471/04; C07D 519/00

(57) ABSTRACT

The present invention relates to novel Amido-Substituted Heterocycle Compounds of Formula (I): and pharmaceutically acceptable salts thereof, wherein R¹, R², R³, R⁴, R⁷, and R⁸ are as defined herein. The present invention also relates to compositions comprising at least one Amido-Substituted Heterocycle Compound, and methods of using the Amido-Substituted Heterocycle Compounds for treating or preventing a herpesvirus infection in a patient.

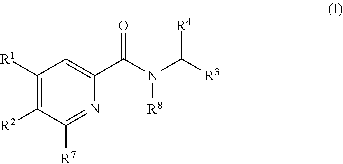

### FIELD OF THE INVENTION

The present invention relates to novel Amido-Substituted Pyridyl Compounds, compositions comprising at least one Amido-Substituted Pyridyl Compound, and methods of using the Amido-Substituted Pyridyl Compounds for treating or preventing herpesvirus infection in a patient.

### BACKGROUND OF THE INVENTION

Human herpes viruses (Herpesviridae) are responsible for causing a wide variety of diseases in humans. Infection with herpes viruses can occur early in life and by adulthood over 95% of the population is infected by at least one herpes virus. These viruses establish a persistent life-long infection through viral latency in neuronal, lymphoid, or myeloid cells. Recurrent episodes of herpes virus disease can be triggered by numerous stimuli, including concurrent viral infections, stress, fatigue, allergies, pregnancy, sunlight or fever. Herpes virus infection in immune competent individuals generally causes mild self-limiting disease, such as: oral (HSV-1), and genital (HSV-2) ulcers, chicken pox (VZV), flu-like syndrome (CMV), and mononucleosis (EBV). In immunocompromised individuals however, primary infection with, or reactivation of an existing herpes virus infection is a major cause of disease and death. Key at-risk immunocompromised populations include patients undergoing solid organ or stem cell transplants, individuals with HIV/AIDS, and ICU patients.

Herpesviridae comprise a diverse family of double-stranded DNA viruses that are classified into three subfamilies (i.e., α, β, and γ) based upon biological characteristics such as cell tropism, diseases caused, viral life-cycle, and site of viral persistence and latency. The family consists of eight members: Herpes Simplex Virus type 1 and 2 (HSV-1, HSV-2), Varicella Zoster Virus (VZV), Epstein-Barr virus (EBV), Cytomegalovirus (CMV), and human herpes viruses 6-8 (HHV6-8).

α-herpes viruses include herpes simplex virus types 1 and 2 (HSV1 and HSV2), and varicella-zoster virus (VZV). HSV1 causes orofacial lesions, commonly known as fever blisters or cold sores. Approximately 30% of the United States population suffers from recurrent episodes of HSV1. HSV2, which is less common than HSV1, causes genital lesions. Primary infection with VZV causes varicella, commonly known as chicken pox. Reactivation of latent VZV manifests as herpes zoster or shingles. Cytomegalovirus (CMV) is a prototypical R herpes virus. Seroprevalance to CMV in the adult population is ˜60%, but certain endemic areas of the world have rates closer to 100%. CMV represents the leading viral cause of morbidity and mortality in at-risk immunocompromised patients. EBV, a 7 herpes virus, causes infectious mononucleosis and is responsible for lymphoid cancers such as Burkitt's and Hodgkin's lymphoma.

Presently, there is no cure for herpes. Medicines have been developed that can prevent or shorten outbreaks, but there is a need for improved therapies for treating herpes virus infection and inhibiting viral replication. The current standard of care for immunocompromised patients at risk for herpes virus disease is pre-emptive treatment with high-dose nucleoside/nucleotide analog drugs such as acyclovir, (val)ganciclovir, and cidofovir, all of which target the viral DNA polymerase. In general, current treatments are virus specific (not broad spectrum), and in the case of (val)ganciclovir and cidofovir cannot be administered prophylactically due to dose-related toxicities including bone marrow suppression and renal toxicity. Although efficacious in many settings, the current nucleos(t)ide drugs are also limited by drug-resistant viral variants and existing cross-resistant variants which may lead to treatment failure. Therefore, there is an urgent medical need for improved, well-tolerated anti-herpes agents.

### SUMMARY OF THE INVENTION

In one aspect, the present invention provides Compounds of Formula (I):

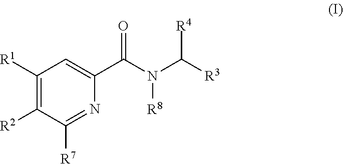

or a pharmaceutically acceptable salt thereof,
wherein:
R¹ is selected from —O—(C₁-C₆ alkyl), 5 to 7-membered monocyclic heterocycloalkyl, —O-(5 to 7-membered monocyclic heterocycloalkyl), 6 to 10-membered bicyclic heterocycloalkyl —O-(6 to 10-membered bicyclic heterocycloalkyl), wherein said 5 to 7-membered monocyclic heterocycloalkyl group, and said 6 to 10-membered bicyclic heterocycloalkyl group, may each be optionally substituted with one or more R^{A} groups, which can be the same or different;
R² is selected from H, —(C₁-C₆ alkylene)ₘ-(8-10 membered bicyclic heteroaryl), C₁-C₆ alkyl, —OH, halo, —O—(C₁-C₆ alkyl), —(C₁-C₆ alkylene)-O—P(O)(OH)₂, —P(O)(OH)₂, —SO₂—(C₁-C₆ alkyl), C₁-C₆ hydroxyalkyl, C₁-C₆ hydroxyalkenyl, —(C₁-C₆ alkenylene)-R⁶, —(C₁-C₆ alkynylene)-R⁶, —CN, —O—(C₁-C₆ haloalkyl), —(C₁-C₆ alkylene)-N(R⁵)₂, —(C₁-C₆ alkylene)-O—(C₁-C₆ alkyl), C₃-C₆ cycloalkyl, 6 to 10-membered bicyclic heterocycloalkyl, —(C₁-C₆ alkylene)ₘ-(3 to 7-membered monocyclic heterocycloalkyl), —(C₁-C₆ alkylene)ₘ-(5 or 6-membered monocyclic heteroaryl), and 9 or 10-membered bicyclic heteroaryl, wherein said C₃-C₆ cycloalkyl group, said 6 to 10-membered bicyclic heterocycloalkyl group, said 3 to 7-membered monocyclic heterocycloalkyl group, said 5 or 6-membered monocyclic heteroaryl group, and said 9 or 10-membered bicyclic heteroaryl group, may each be optionally substituted with one or more R^{B} groups, which can be the same or different;
R³ is phenyl or 5 or 6-membered monocyclic heteroaryl, each of which may each be optionally substituted with one or more R^{C} groups, which can be the same or different, or R³ and R⁸, together with the common nitrogen atom to which they are attached, can combine to form a pyrrolidine ring, which can be optionally substituted with C₁-C₆ alkyl, phenyl or 5 or 6-membered monocyclic heteroaryl, wherein said phenyl group or said 5 or 6-membered monocyclic heteroaryl group, may each be optionally substituted with one or more R^{C} groups, which can be the same or different;
R⁴ is H or C₁-C₆ alkyl;
each occurrence of R⁵ is independently selected from H, C₁-C₆ alkyl, C₃-C₆ cycloalkyl, 3 to 6-membered monocyclic heterocycloalkyl, and C₁-C₆ hydroxyalkyl;
R⁶ is selected from H, 5 or 6-membered monocyclic heteroaryl, C₁-C₆ hydroxyalkyl, and —(C₁-C₆ alkylene)-O—(C₁-C₆ alkyl), wherein said 5 or 6-membered monocyclic heteroaryl group may be optionally substituted with one or more R^{C} groups, which can be the same or different;
R⁷ is selected from H, halo, C₁-C₆ alkyl, C₁-C₆ hydroxyalkyl, —CN, and 5 or 6-membered monocyclic heteroaryl, wherein said 5 or 6-membered monocyclic heteroaryl group may be optionally substituted with one or more R^{C} groups, which can be the same or different;
R⁸ is selected from H, —C(O)O-(5 or 6-membered monocyclic heterocycloalkyl), —C(O)O—(C₁-C₆ alkylene)-O—(C₁-C₆ alkyl), C₁-C₆ hydroxyalkyl, and —(C₁-C₆ alkylene)ₘ-OC(O)—R⁹;
R⁹ is selected from C₃-C₆ cycloalkyl, 5 or 6-membered monocyclic heterocycloalkyl, —(C₁-C₆ alkylene)-OC(═O)—(C₁-C₆ alkyl), C₁-C₆ aminoalkyl, and —(C₁-C₆ alkylene)-O—P(O)(OH)₂, —(C₁-C₆ alkylene)-P(O)(OH)₂, wherein said C₃-C₆ cycloalkyl group, and said 5 or 6-membered monocyclic heterocycloalkyl group may be optionally substituted with one substituent, selected from C₁-C₆ alkyl, —OH, 5 or 6-membered monocyclic heterocycloalkyl, and —O—P(O)(OH)₂, and wherein said 5 or 6-membered monocyclic heterocycloalkyl substituent group may be further optionally substituted with C₁-C₆ alkyl;
each occurrence of R^{A} is independently selected from C₁-C₆ alkyl, —C(O)—C₁-C₆ alkyl, C₁-C₆ haloalkyl, —S(O)₂—(C₁-C₆ alkyl), oxo, and —CN;
each occurrence of R^{B} is independently selected from —OH, halo C₁-C₆ alkyl, —O—(C₁-C₆ alkyl), C₁-C₆ haloalkyl, C₁-C₆ hydroxyalkyl, C₃-C₆ cycloalkyl, 3 to 6-membered monocyclic heterocycloalkyl, —(C₁-C₆ alkylene)-N(R⁴)₂, —(C₁-C₆ alkylene)-O—(C₁-C₆ alkyl), and —(C₁-C₆ alkylene)-SO₂—(C₁-C₆ alkyl);
each occurrence of R^{C} is independently selected from C₁-C₆ alkyl, C₁-C₆ haloalkyl, C₁-C₆ hydroxyalkyl, halo, —CN, C₁-C₆ alkynyl, and NO₂; and each occurrence of m is independently 0 or 1;
   wherein the pyridyl ring nitrogen atom present in formula(I) may be in the form of its N-oxide derivative.

The Compounds of Formula (I) (also referred to herein as the “Amido-Substituted Pyridyl Compounds”), and pharmaceutically acceptable salts thereof can be useful, for example, for inhibiting herpesvirus viral replication or activity, and for treating or preventing herpesvirus infection in a patient. Without being bound by any specific theory, it is believed that the Amido-Substituted Pyridyl Compounds inhibit herpesvirus viral replication by inhibiting herpesvirus polymerase.

Accordingly, the present invention provides methods for treating or preventing herpesvirus infection in a patient, comprising administering to the patient an effective amount of at least one Amido-Substituted Pyridyl Compound.

The details of the invention are set forth in the accompanying detailed description below.

Although any methods and materials similar to those described herein can be used in the practice or testing of the present invention, illustrative methods and materials are now described. Other embodiments, aspects and features of the present invention are either further described in or will be apparent from the ensuing description, examples and appended claims.

### DETAILED DESCRIPTION OF THE INVENTION

The present invention relates to novel Amido-Substituted Pyridyl Compounds, compositions comprising at least one Amido-Substituted Pyridyl Compound, and methods of using the Amido-Substituted Pyridyl Compounds for treating or preventing herpesvirus infection in a patient.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc. . . .

As used herein, and throughout this disclosure, the following terms, unless otherwise indicated, shall be understood to have the following meanings:

A “patient” is a human or non-human mammal. In one embodiment, a patient is a human.

The term “effective amount” as used herein, refers to an amount of Amido-Substituted Pyridyl Compound and/or an additional therapeutic agent, or a composition thereof that is effective in producing the desired therapeutic, ameliorative, inhibitory or preventative effect when administered to a patient suffering from a viral infection or virus-related disorder. In the combination therapies of the present invention, an effective amount can refer to each individual agent or to the combination as a whole, wherein the amounts of all agents administered are together effective, but wherein the component agent of the combination may not be present individually in an effective amount.

The term “preventing,” as used herein with respect to an herpesvirus viral infection or herpesvirus-virus related disorder, refers to reducing the likelihood of herpesvirus infection.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, NH(cycloalkyl), —O—C(O)-alkyl, —O—C(O)-aryl, —O—C(O)— cycloalkyl, —C(O)OH and —C(O)O-alkyl. In one embodiment, an alkyl group is linear. In another embodiment, an alkyl group is branched. Unless otherwise indicated, an alkyl group is unsubstituted.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₆ alkenyl” refers to an alkenyl group having from 2 to 6 carbon atoms. Unless otherwise indicated, an alkenyl group is unsubstituted.

The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₆ alkynyl” refers to an alkynyl group having from 2 to 6 carbon atoms. Unless otherwise indicated, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon atoms. In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms.

The term “alkenylene,” as used herein, refers to an alkenyl group, as defined above, wherein one of the alkenyl group's hydrogen atoms has been replaced with a bond. Non-limiting examples of alkylene groups include —CH═CH—, —CH═CHCH₂—, —CH₂CH₂CH═CH—, and —CH₂(CH₃)C═CH—. In one embodiment, an alkenylene group has from 1 to about 6 carbon atoms.

In another embodiment, an alkenylene group is branched. In another embodiment, an alkenylene group is linear. The term “C₁-C₆ alkenylene” refers to an alkenylene group having from 1 to 6 carbon atoms.

The term “alkynylene,” as used herein, refers to an alkynyl group, as defined above, wherein one of the alkynyl group's hydrogen atoms has been replaced with a bond. Non-limiting examples of alkylene groups include —C≡C—, —C≡CCH₂—, —CH₂CH₂C≡C—, and —CH(CH₃)C≡C—. In one embodiment, an alkynylene group has from 1 to about 6 carbon atoms.

In another embodiment, an alkynylene group is branched. In another embodiment, an alkynylene group is linear. The term “C₁-C₆ alkynylene” refers to an alkynylene group having from 1 to 6 carbon atoms.

The term “aminoalkyl,” as used herein, refers to an alkyl group as defined above, wherein one of the alkyl group's hydrogen atoms has been replaced with —NH₂, —NH(C₁-C₆ alkyl), or —N(C₁-C₆ alkyl)₂. In one embodiment, an aminoalkyl group has from 1 to 6 carbon atoms. Non-limiting examples of aminoalkyl groups include —CH₂NH₂, —CH₂N(CH₃)₂, —CH₂NH₂, and —CH₂NH(CH)₃. The term “C₁-C₆ aminoalkyl” refers to an aminoalkyl group having from 1 to 6 carbon atoms.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. In one embodiment, an aryl group can be optionally fused to a cycloalkyl or cycloalkanoyl group. Non-limiting examples of aryl groups include phenyl and naphthyl. In one embodiment, an aryl group is phenyl. In another embodiment, an aryl group is napthalene. Unless otherwise indicated, an alkyl group is unsubstituted.

The term “cycloalkyl,” as used herein, refers to a non-aromatic mono- or multicyclic ring system comprising from about 3 to about 10 ring carbon atoms. In one embodiment, a cycloalkyl contains from about 5 to about 10 ring carbon atoms. In another embodiment, a cycloalkyl contains from about 3 to about 6 ring atoms. In another embodiment, a cycloalkyl contains from about 5 to about 6 ring atoms. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. Unless otherwise indicated, cycloalkyl group is unsubstituted. In one embodiment, a cycloalkyl group is unsubstituted. The term “3 to 6-membered cycloalkyl” refers to a cycloalkyl group having from 3 to 6 ring carbon atoms. The term “3 to 7-membered cycloalkyl” refers to a cycloalkyl group having from 3 to 7 ring carbon atoms. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

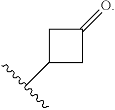 [SMILES: *C1CC(=O)C1]

The term “cycloalkenyl,” as used herein, refers to a non-aromatic mono- or multicyclic ring system comprising from about 4 to about 10 ring carbon atoms and containing at least one endocyclic double bond. In one embodiment, a cycloalkenyl contains from about 4 to about 7 ring carbon atoms. In another embodiment, a cycloalkenyl contains 5 or 6 ring atoms. Non-limiting examples of monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. A ring carbon atom of a cycloalkyl group may be functionalized as a carbonyl group. In one embodiment, a cycloalkenyl group is cyclopentenyl. In another embodiment, a cycloalkenyl group is cyclohexenyl. The term “4 to 6-membered cycloalkenyl” refers to a cycloalkenyl group having from 4 to 6 ring carbon atoms.

The term “halo,” as used herein, means —F, —Cl, —Br or —I.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3F. atoms. Non-limiting examples of haloalkyl groups include —CH₂F, —CHF₂, —CH₂CH₂F—CF₃, —CH₂C₁ and —CCl₃. Consider adding CH₂CHF₂ which is a frequent substituent in the compounds of the invention.

The term “C₁-C₆ haloalkyl” refers to a haloalkyl group having from 1 to 6 carbon atoms.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₆ hydroxyalkyl” refers to a hydroxyalkyl group having from 1 to 6 carbon atoms.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms (“5 to 6-membered monocyclic heteroaryl”). In another embodiment, a heteroaryl group is bicyclic and had 9 or 10 ring atoms (“9 or 10-membered bicyclic heteroaryl”). A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is a 5-membered heteroaryl. In another embodiment, a heteroaryl group is a 6-membered heteroaryl, such as pyridyl. In another embodiment, a “9- or 10-membered bicyclic heteroaryl” group comprises a 5- to 6-membered heterocycloalkyl group fused to a benzene ring, such as:

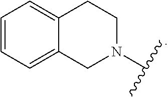 [SMILES: *N1CCc2ccccc2C1]

In still another embodiment, a “9- or 10-membered bicyclic heteroaryl” group comprises a 5- to 6-membered heteroaryl group fused to a cycloalkyl ring or a heterocycloalkyl ring, such as:

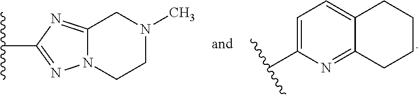 [SMILES: *c1nc2n(n1)CCN(C)C2 ; *c1ccc2c(n1)CCCC2]

The term “heteroarylene,” as used herein, refers to a bivalent group derived from an heteroaryl group, as defined above, by removal of a hydrogen atom from a ring carbon or ring heteroatom of a heteroaryl group. A heteroarylene group can be derived from a monocyclic or multicyclic ring system comprising about 5 to about 14 ring atoms, wherein from 1 to 4 of the ring atoms are each independently O, N or S and the remaining ring atoms are carbon atoms. A heteroarylene group can be optionally substituted by one or more “ring system substituents” which may be the same or different, and are as defined herein below. A heteroarylene group is joined via a ring carbon atom or by a nitrogen atom with an open valence, and any nitrogen atom of a heteroarylene can be optionally oxidized to the corresponding N-oxide. The term “heteroarylene” also encompasses a heteroarylene group, as defined above, which is fused to a benzene ring. Non-limiting examples of heteroarylenes include pyridylene, pyrazinylene, furanylene, thienylene, pyrimidinylene, pyridonylene (including those derived from N-substituted pyridonyls), isoxazolylene, isothiazolylene, oxazolylene, oxadiazolylene, thiazolylene, pyrazolylene, thiophenylene, furazanylene, pyrrolylene, triazolylene, 1,2,4-thiadiazolylene, pyrazinylene, pyridazinylene, quinoxalinylene, phthalazinylene, oxindolylene, imidazo[1,2-a]pyridinylene, imidazo[2,1-b]thiazolylene, benzofurazanylene, indolylene, azaindolylene, benzimidazolylene, benzothienylene, quinolinylene, imidazolylene, benzimidazolylene, thienopyridylene, quinazolinylene, thienopyrimidylene, pyrrolopyridylene, imidazopyridylene, isoquinolinylene, benzoazaindolylene, 1,2,4-triazinylene, benzothiazolylene and the like, and all isomeric forms thereof. The term “heteroarylene” also refers to partially saturated heteroarylene moieties such as, for example, tetrahydroisoquinolylene, tetrahydroquinolylene, and the like. A heteroarylene group is divalent and unless specified otherwise, either available bond on a heteroarylene ring can connect to either group flanking the heteroarylene group. For example, the group “A-heteroarylene-B,” wherein the heteroarylene group is:

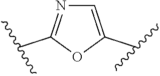 [SMILES: *c1cnc(*)o1]

is understood to represent both.

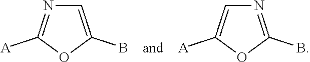

In one embodiment, a heteroarylene group is a monocyclic heteroarylene group or a bicyclic heteroarylene group. In another embodiment, a heteroarylene group is a monocyclic heteroarylene group. In another embodiment, a heteroarylene group is a bicyclic heteroarylene group. In still another embodiment, a heteroarylene group has from about 5 to about 10 ring atoms. In another embodiment, a heteroarylene group is monocyclic and has 5 or 6 ring atoms. In another embodiment, a heteroarylene group is bicyclic and has 9 or 10 ring atoms. In another embodiment, a heteroarylene group is a 5-membered monocyclic heteroarylene. In another embodiment, a heteroarylene group is a 6-membered monocyclic heteroarylene. In another embodiment, a bicyclic heteroarylene group comprises a 5 or 6-membered monocyclic heteroarylene group fused to a benzene ring. In still another embodiment, a heteroaryl group comprises a 5- to 6-membered monocyclic heteroarylene group fused to a cycloalkyl ring or a heterocycloalkyl ring.

The term “heterocycloalkyl,” as used herein, refers to a non-aromatic saturated monocyclic or multicyclic ring system comprising 3 to about 11 ring atoms, wherein from 1 to 4 of the ring atoms are independently O, S, N or Si, and the remainder of the ring atoms are carbon atoms. A heterocycloalkyl group can be joined via a ring carbon, ring silicon atom or ring nitrogen atom. In one embodiment, a heterocycloalkyl group is monocyclic. In one embodiment, a heterocycloalkyl group is monocyclic and has from about 3 to about 7 ring atoms (“3 to 7-membered bicyclic heterocycloalkyl”). In another embodiment, a heterocycloalkyl group is monocyclic has from about 4 to about 7 ring atoms (“4 to 7-membered bicyclic heterocycloalkyl”). In still another embodiment, a heterocycloalkyl group is monocyclic and has 5 or 6 ring atoms (“5 or 6-membered monocyclic heterocycloalkyl”). In one embodiment, a heterocycloalkyl group is bicyclic. In another embodiment, a heterocycloalkyl group is bicyclic and has from about 6 to about 10 ring atoms (“6 to 10-membered bicyclic heterocycloalkyl”). There are no adjacent oxygen and/or sulfur atoms present in the ring system. Any —NH group in a heterocycloalkyl ring may exist protected such as, for example, as an —N(BOC), —N(CBz), —N(Tos) group and the like; such protected heterocycloalkyl groups are considered part of this invention. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone, silacyclopentane, silapyrrolidine and the like, and all isomers thereof. Non-limiting illustrative examples of a silyl-containing heterocycloalkyl group include:

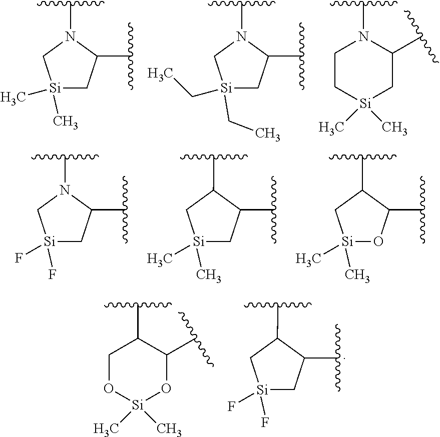

A ring carbon atom of a heterocycloalkyl group may be functionalized as a carbonyl group. Illustrative examples of such a heterocycloalkyl group include, but are not limited to:

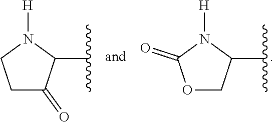 [SMILES: *C1NCCC1=O ; *C1COC(=O)N1]

A ring sulfur atom of a heterocycloalkyl group may also be functionalized as a sulfonyl group. An example of such a heterocycloalkyl group is:

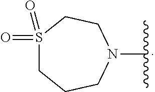 [SMILES: *N1CCCS(=O)(=O)CC1]

In one embodiment, a heterocycloalkyl group is a 5-membered monocyclic heterocycloalkyl. In another embodiment, a heterocycloalkyl group is a 6-membered monocyclic heterocycloalkyl.

A multicyclic heterocycloalkyl group may have rings that are fused, rings that are joined in a spirocyclic manner, and rings that are bridged. In one embodiment, a heterocycloalkyl group can be a bicyclic spirocyclic heteroaryl group having from 7 to 9 ring atoms. Illustrative examples of such a bicyclic heterocycloalkyl group include:

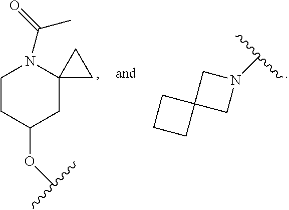 [SMILES: *OC1CCN(C(C)=O)C2(CC2)C1 ; *N1CC2(CCC2)C1]

In another embodiment, a heterocycloalkyl group can be a bicyclic fused heterocycloalkyl group having from 6 to 10 ring atoms. Illustrative examples of such a fused bicyclic heterocycloalkyl group include:

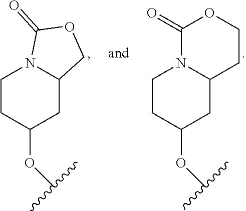 [SMILES: *OC1CCN2C(=O)OCC2C1 ; *OC1CCN2C(=O)OCCC2C1]

In another embodiment, a heterocycloalkyl group can be a bridged heterocycloalkyl group having from 6 to 10 ring atoms. Illustrative examples of such a bridged bicyclic heterocycloalkyl group include:

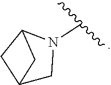 [SMILES: *N1CC2CC1C2]

The term “heterocycloalkenyl,” as used herein, refers to a heterocycloalkyl group, as defined above, wherein the heterocycloalkyl group contains from 4 to 10 ring atoms, and at least one endocyclic carbon-carbon or carbon-nitrogen double bond. A heterocycloalkenyl group can be joined via a ring carbon or ring nitrogen atom. In one embodiment, a heterocycloalkenyl group has from 4 to 6 ring atoms. In another embodiment, a heterocycloalkenyl group is monocyclic and has 5 or 6 ring atoms. In another embodiment, a heterocycloalkenyl group is bicyclic. A heterocycloalkenyl group can optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. A ring carbon atom of a heterocycloalkenyl group may be functionalized as a carbonyl group. Non-limiting examples of heterocycloalkenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluoro-substituted dihydrofuranyl, 7-oxabicyclo[2.2.1]heptenyl, dihydrothiophenyl, dihydrothiopyranyl, and the like and the like. In one embodiment, a heterocycloalkenyl group is a 5-membered heterocycloalkenyl. In another embodiment, a heterocycloalkenyl group is a 6-membered heterocycloalkenyl. The term “4 to 6-membered heterocycloalkenyl” refers to a heterocycloalkenyl group having from 4 to 6 ring atoms.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

Examples of “ring system substituents” include, but are not limited to, alkyl, alkenyl, alkynyl, aryl, heteroaryl, -alkylene-aryl, -arylene-alkyl, -alkylene-heteroaryl,-alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)_{Z}-heteroaryl, —S-alkyl, —S-aryl, —S-heteroaryl, —S-alkylene-aryl, —S-alkyleneheteroaryl, —S(O)₂-alkylene-aryl, —S(O)₂-alkylene-heteroaryl, —Si(alkyl)₂, —Si(aryl)₂, Si(heteroaryl)₂-Si(alkyl)(aryl), —Si(alkyl)(cycloalkyl), —Si(alkyl)(heteroaryl), cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y¹)(Y²), -alkylene-N(Y¹)(Y²), —C(O)N(Y¹)(Y²), and —S(O)₂N(Y¹)(Y²), wherein Y¹ and Y² can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

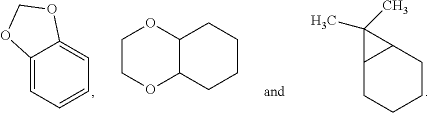

When any substituent or variable (e.g., R¹, m, etc.) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide an Amido-Substituted Pyridyl Compound or a pharmaceutically acceptable salt or solvate of the compound. The transformation may occur by various mechanisms (e.g., by metabolic or chemical processes), such as, for example, through hydrolysis in blood.

For example, if an Amido-Substituted Pyridyl Compound or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₅)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 6 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if an Amido-Substituted Pyridyl Compound contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, —P(O)(OH)₂, —P(O)(O(C₁-C₆)alkyl)₂ or glycosyl (the radical resulting from the removal of a hydroxyl group of the hemiacetal form of a carbohydrate), and the like.

If an Amido-Substituted Pyridyl Compound incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N— or di-N,N—(C₁-C₆)alkylaminoalkyl; —C(Y⁴)Y⁵ wherein Y⁴ is H or methyl and Y⁵ is mono-N— or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Amido-Substituted Pyridyl Compounds can form salts which are also within the scope of this invention. The term “salt(s)”, as employed herein, denotes acidic salts formed with inorganic and/or organic acids, as well as basic salts formed with inorganic and/or organic bases. In addition, when an Amido-Substituted Pyridyl Compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting an Amido-Substituted Pyridyl Compound with an amount of acid or base, such as an equivalent amount, in a medium such as one in which the salt precipitates or in an aqueous medium followed by lyophilization.

Exemplary acid addition salts include acetates, ammonium, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (also known as mesylates), naphthalenesulfonates, nitrates, oxalates, phosphates, propionates, salicylates, succinates, sulfates, tartarates, thiocyanates, toluenesulfonates (also known as tosylates), and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto. In one embodiment, an acid salt is an ammonium salt or a di-ammonium salt.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Amido-Substituted Pyridyl Compounds may be atropisomers (e.g., substituted biaryls), and are considered as part of this invention. Enantiomers can also be directly separated using chiral chromatographic techniques.

It is also possible that the Amido-Substituted Pyridyl Compounds may exist in different tautomeric forms, and all such forms are embraced within the scope of the invention. For example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If an Amido-Substituted Pyridyl Compound incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (H), and deuterium (2H). Protium is the predominant hydrogen isotope found in nature. Enriching for deuterium may provide certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

Polymorphic forms of the Amido-Substituted Pyridyl Compounds, and of the salts, solvates, hydrates, esters and prodrugs of the Amido-Substituted Pyridyl Compounds, are intended to be included in the present invention.

The following abbreviations are used below and have the following meanings: AcOH is acetic acid; n-Ad₂(n-Bu)P palladacycle G2 is di(1-adamantyl)-n-butylphosphine palladacycle, 2^{nd} generation; ATCC is American Type Culture Collection; sec-BuLi is secondary butyllithium; CDI is N,N-carbonyldiimidazole; Celite is diatomaceous earth; DAST is diethylaminosulfur trifluoride; DEAD is diethyl azodicarboxylate; DMF is N,N-dimethylformamide; DMSO is dimethylsulfoxide; EDTA is ethylenediaminetetraacetic acid; ESI is electrospray ionization; EtOAc is ethyl acetate; G2 XPhos is chloro(2-dicyclohexylphosphino-2′,4′,6′-tri-1-propyl-1,1′-biphenyl)(2′-amino-1, F-biphenyl-2-yl) palladium(II); Grubb's catalyst 2^{nd} generation is dichloro[1,3-bis(2,4,6-trimethylphenyl)-2-imidazolidinylidene](benzylidene) (tricyclohexylphosphine) ruthenium(II); HEPES is 2-[4-(2-hydroxyethyl)piperazin-1-yl]ethanesulfonic acid; HPLC is high performance liquid chromatography; Hunig's base is N,N-diisopropylethylamine; iPrOH is isopropanol; (Ir[DF(CF₃)PPY]₂(DTBPY))PF₆ or Ir[dF(CF₃)ppy]₂(dtbbpy)PF₆ is [4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate; LCMS is liquid chromatography/mass spectrometry; LED is light-emitting diode; m-CPBA is meta-chloroperoxybenzoic acid; Me is methyl; MeOH is methanol; MS is mass spectrometry; NCS is N-chlorosuccinimide; NMP is N-methylpyrrolidone; PPIA is peptidylprolyl isomerase A; PSI is pounds/square inch; ROCKPhos palladacycle G2 is [(2-Di-tert-butylphosphino-3-methoxy-6-methyl-2′,4′,6′-triisopropyl-1,1′-biphenyl)-2-(2-aminobiphenyl)]palladium(II) methanesulfonate, 3^{rd} generation; SFC is supercritical fluid chromatography; SPE is solid phase extraction; tert-butyl is tertiary butyl; TEA is triethylamine; TFA is trifluoroacetic acid; THF is tetrahydrofuran; TMEDA is N-tetramethylethylenediamine; TLC is thin-layer chromatography; turbogrignard is isopropyl magnesium chloride complex with lithium chloride; and Xphos is [2-Dicyclohexylphosphino-2′,4′,6′-triisopropylbiphenyl].

### The Compounds of Formula (I)

The present invention provides Amido-Substituted Pyridyl Compounds of Formula (I):

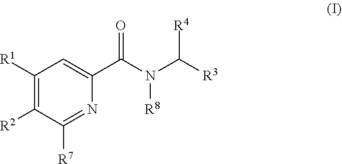

and pharmaceutically acceptable salts thereof, wherein R¹, R², R³, R⁴, R⁷, and R⁸ are as defined above for the Compounds of Formula (I).

In one embodiment, R¹ is selected from:

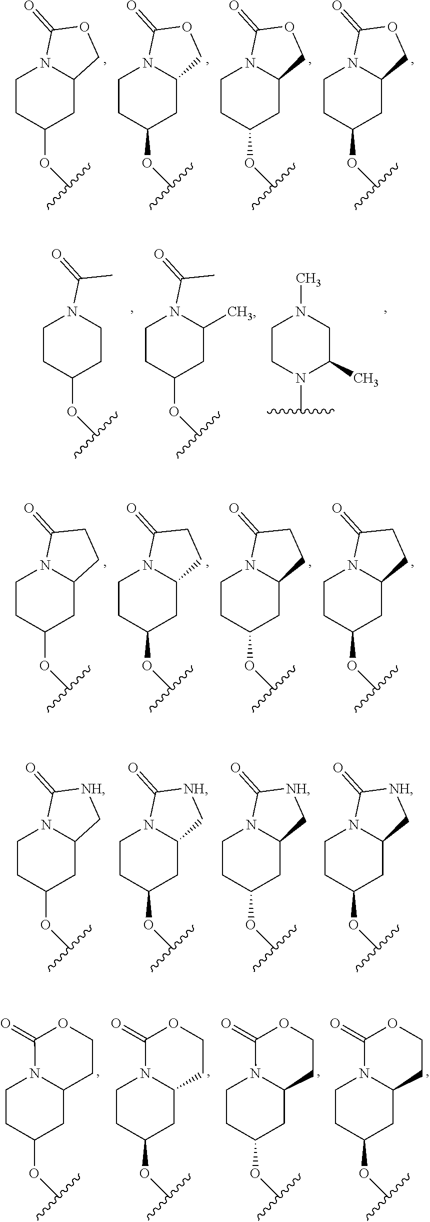

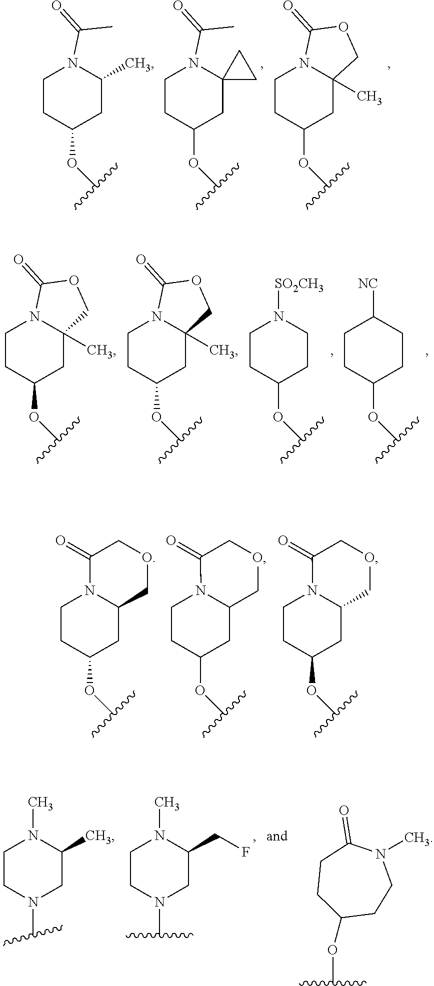

In another embodiment, R¹ is selected from:

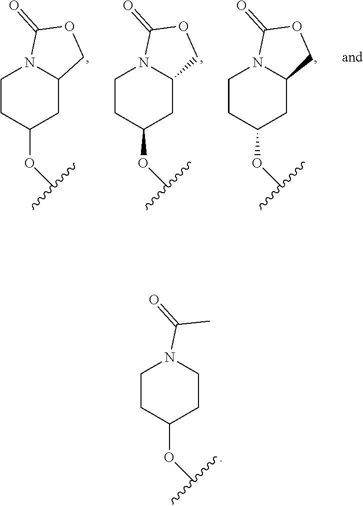

In one embodiment, R² is selected from H, C₁-C₆ alkyl, halo, —CN, —O—(C₁-C₆ alkyl), —CN, —O—(C₁-C₆ haloalkyl), —(C₁-C₆ alkylene)-O—P(O)(OH)₂, and —(C₁-C₆ alkylene)ₘ-(5 or 6-membered monocyclic heteroaryl), wherein said 5 or 6-membered monocyclic heteroaryl group can be optionally substituted with one or more R^{B} groups, which can be the same or different.

In another embodiment, R² is selected from methyl, methoxy, Cl—CN, —CH₂OH, —OH, —SO₂CH₃, —CH₂CH₂OH, —CH₂C(CH₃)₂—OH, —CH═CH—CH₂—OH, —CH₂C(CH₃)₂—OCH₃, —CH₂N(CH₃)—CH₂CH₂OH, —CH₂NH-cyclopropyl, —CH₂N(CH₃)—CH₂CH₂CH₂OH, cyclopropyl, cyclobutyl, —CH₂—O—P(O)(OH)₂, —P(O)(O⁻NH₄⁺)₂, —P(O)(OH)₂, —OCH₂CF₃, tetrahydroypyranyl, —CH₂-tetrahydropyranyl, piperadinyl, —CH₂-piperadinyl, morpholinyl, —CH₂-morpholinyl, azetidinyl, —CH-azetidinyl, oxetanyl, spiro[3,3]heptanyl, 2λ²-azaspiro[3,3]heptanyl, pyrazolyl, triazolyl, isoxazolyl, thiazolyl, isothiazolyl, oxazolyl, 1,2,4-oxadiazolyl, 1,3,4-oxadiazolyl, 1,2,4-thiadiazolyl, and 1,3,4-thiadiazolyl, wherein said cyclopropyl, cyclobutyl, tetrahydropyranyl, piperadinyl, morpholinyl, azetidinyl, oxetanyl, spiro[3,3]heptanyl, 22-azaspiro[3,3]heptanyl, pyrazolyl, triazolyl, isoxazolyl, thiazolyl, isothiazolyl, oxazolyl, 1,3,4-oxadiazolyl, 1,2,4-thiadiazolyl, and 1,3,4-thiadiazolyl groups can be optionally substituted with one or more groups, which can be the same or different, and are selected from —CHF₂, F, —OH, methyl, ethyl, cyclopropyl, n-propyl, isopropyl, t-butyl, isobutyl, —CH₂CF₃, —CH₂C(OH)(CH₃)₂, —CH₂N(CH₃)₂, —CH₂CH₂N(CH₃)₂, —CH₂OH, —CH₂CH₂OH, —CH₂CH₂OCH₃, —CH₂CHF₂, —CH₂CH₂CH(CH₃)₂, oxetane, methoxy, —C(OH)(CH₃)₂, and —CH₂S(O)₂CH₃.

In still another embodiment, R² is selected from:

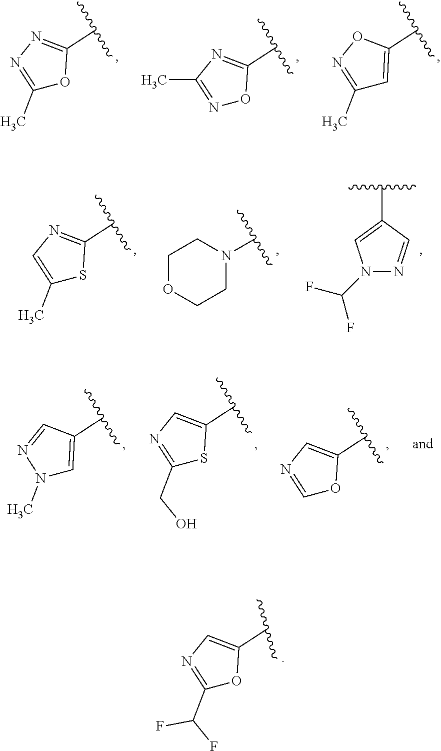

In another embodiment, R² is selected from:

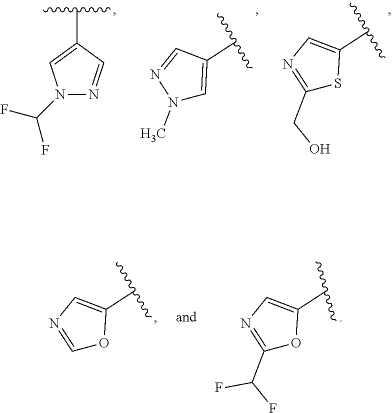

In one embodiment, R³ is phenyl or pyridyl, which can be optionally substituted with up to three R^{C} groups, which can be the same or different, and are each individually selected from F, Cl, Br, I, —CN, —C≡CH, and NO₂.

In another embodiment, R³ is selected from:

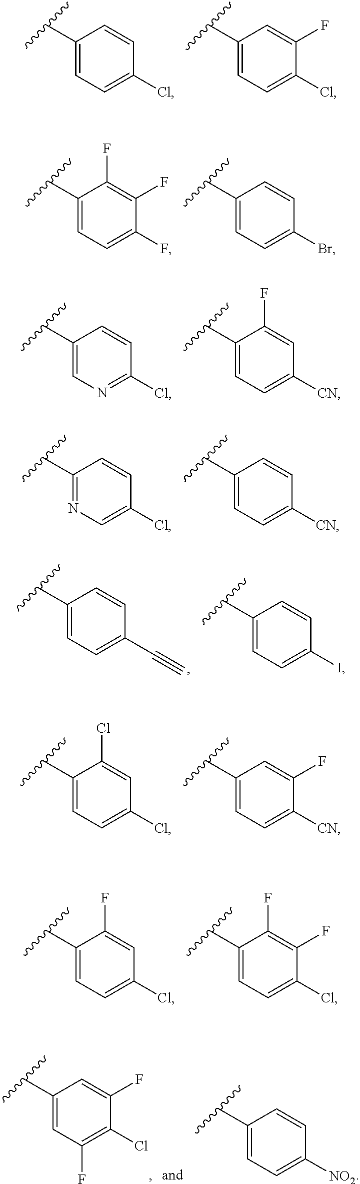

In still another embodiment, R³ is:

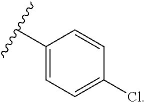 [SMILES: *c1ccc(Cl)cc1]

In one embodiment, R⁴ is H or methyl.

In one embodiment, R⁷ is selected from H, methyl, Cl, —CH₂OH, —CN,

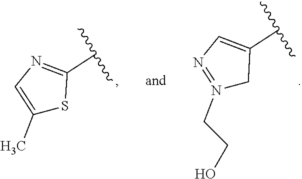

In one embodiment, R⁷ is H.

In one embodiment, R⁸ is selected from H, —CH₂OH, —CH₂SCH₃, —C(O)CH₂CH₂OCH₃,

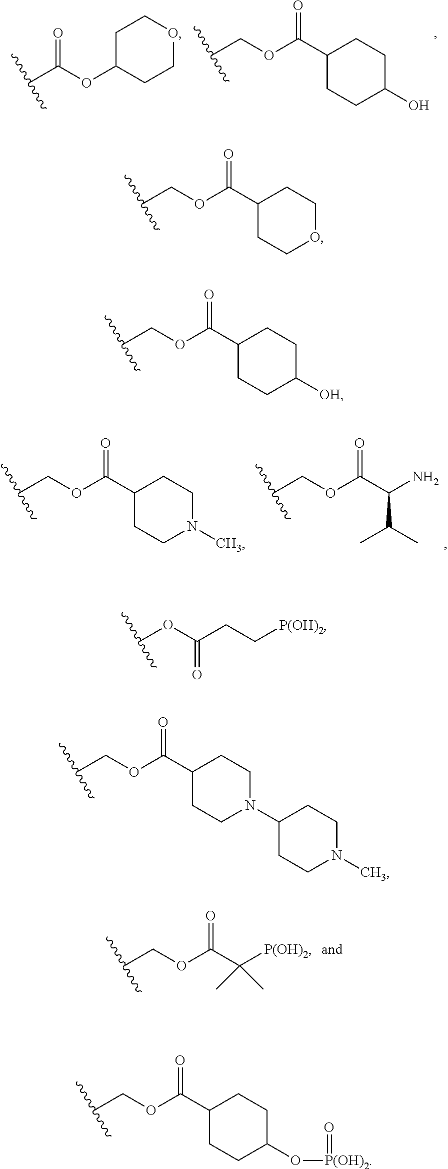

In another embodiment, R⁸ is H.

In another embodiment, R⁷ and R⁸ are each H.

In one embodiment, the compounds of formula (I) have the formula (Ia):

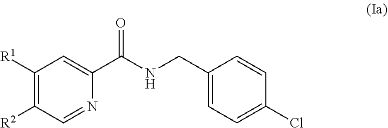

or a pharmaceutically acceptable salt thereof,
wherein:
R¹ is —O-(5 or 6-membered monocyclic heterocycloalkyl) or —O-(9 or 10-membered bicyclic heterocycloalkyl), wherein said 5 or 6-membered monocyclic heteroaryl group, and said 9 or 10-membered bicyclic heterocycloalkyl group can each be optionally substituted with methyl or —C(O)CH₃;
R² is selected from methyl, methoxy, —CN, Cl, —OCH₂CF₃, pyrazolyl, triazolyl, isoxazolyl, thiazolyl, isothiazolyl, oxazolyl, 1,2,4-thiadiazolyl, and 1,3,4-thiadiazolyl, wherein said pyrazolyl, triazolyl, isoxazolyl, thiazolyl, isothiazolyl, oxazolyl, 1,2,4-thiadiazolyl, and 1,3,4-thiadiazolyl groups can be optionally substituted with up to two R^{B} groups, which can be the same or different; and
each occurrence of R^{B} is independently selected from C₁-C₆ alkyl, F, —OH, —O—(C₁-C₆ alkyl), C₁-C₆ haloalkyl, C₁-C₆ hydroxyalkyl, C₃-C₆ cycloalkyl, 3 to 6-membered monocyclic heterocycloalkyl, —(C₁-C₆ alkylene)-N(R⁴)₂, —(C₁-C₆ alkylene)-O—(C₁-C₆ alkyl), and —(C₁-C₆ alkylene)-SO₂—(C₁-C₆ alkyl).

It is understood that the present invention includes any combination of two or more of the above embodiments.

It is also understood that the above embodiments apply to compounds of formula (I), and compounds of formula (Ia).

Other embodiments of the present invention include the following:
(a) A pharmaceutical composition comprising an effective amount of a Compound of Formula (I) or (Ia), or a pharmaceutically acceptable salt thereof, and a pharmaceutically acceptable carrier.
(b) The pharmaceutical composition of (a), further comprising a second therapeutic agent selected from the group consisting of anti-herpes agents and immunomodulators.
(c) The pharmaceutical composition of (b), wherein the anti-herpes agent is selected from the group consisting of herpesvirus polymerase inhibitors, and CMV terminase inhibitors.
(d) A pharmaceutical combination that is (i) a Compound of Formula (I) or (Ia), and (ii) a second therapeutic agent selected from the group consisting of anti-herpes agents and immunomodulators; wherein the Compound of Formula (I) or (Ia), and the second therapeutic agent are each employed in an amount that renders the combination effective for inhibiting herpesvirus replication, or for treating herpesvirus infection and/or reducing the likelihood or severity of symptoms of herpesvirus infection.
(e) The combination of (d), wherein the anti-herpes agent is selected from the group consisting of herpesvirus polymerase inhibitors, and CMV terminase inhibitors.
(f) A method of inhibiting herpesvirus replication in a subject in need thereof which comprises administering to the subject an effective amount of a Compound of Formula (I) or (Ia).
(g) A method of treating herpesvirus infection and/or reducing the likelihood or severity of symptoms of herpesvirus infection in a subject in need thereof which comprises administering to the subject an effective amount of a Compound of Formula (I) or (Ia).
(h) The method of (g), wherein the Compound of Formula (I) is administered in combination with an effective amount of at least one second therapeutic agent selected from the group consisting of anti-herpes agents and immunomodulators.
(i) The method of (h), wherein the anti-herpes agent is selected from the group consisting of herpesvirus polymerase inhibitors, and CMV terminase inhibitors.
(j) A method of inhibiting herpesvirus replication in a subject in need thereof which comprises administering to the subject the pharmaceutical composition of (a), (b) or (c) or the combination of (d) or (e).
(k) A method of treating herpesvirus infection and/or reducing the likelihood or severity of symptoms of herpesvirus infection in a subject in need thereof which comprises administering to the subject the pharmaceutical composition of (a), (b) or (c) or the combination of (d) or (e).

The present invention also includes a compound of the present invention for use (i) in, (ii) as a medicament for, or (iii) in the preparation of a medicament for: (a) medicine; (b) inhibiting herpesvirus replication or (c) treating herpesvirus infection and/or reducing the likelihood or severity of symptoms of herpesvirus infection. In these uses, the compounds of the present invention can optionally be employed in combination with one or more second therapeutic agents selected from anti-herpes agents, anti-infective agents, and immunomodulators.

Additional embodiments of the invention include the pharmaceutical compositions, combinations and methods set forth in (a)-(k) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate.

It is further to be understood that the embodiments of compositions and methods provided as (a) through (k) above are understood to include all embodiments of the compounds, including such embodiments as result from combinations of embodiments.

Non-limiting examples of the Compounds of Formula (I) include compounds 3, 4, and 6-189, as set forth in the Examples below, and pharmaceutically acceptable salts thereof.

### Methods for Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below Alternative synthetic pathways and analogous structures will be apparent to those skilled in the art of organic synthesis.

One skilled in the art of organic synthesis will recognize that the synthesis of multicyclic heterocycle cores contained in Compounds of Formula (I) may require protection of certain functional groups (i.e., derivatization for the purpose of chemical compatibility with a particular reaction condition). Suitable protecting groups for the various functional groups of these Compounds and methods for their installation and removal are well known in the art of organic chemistry. A summary of many of these methods can be found in Greene et al., Protective Groups in Organic Synthesis, Wiley-Interscience, New York, (1999).

One skilled in the art of organic synthesis will also recognize that one route for the synthesis of the multicyclic heterocycle cores of the Compounds of Formula (I) may be more desirable depending on the choice of appendage substituents.

Additionally, one skilled in the art will recognize that in some cases the order of reactions may differ from that presented herein to avoid functional group incompatibilities and thus adjust the synthetic route accordingly.

The preparation of multicyclic intermediates useful for making the multicyclic heterocycle cores of the Compounds of Formula (I) have been described in the literature and in compendia such as “Comprehensive Heterocyclic Chemistry” editions I, II and III, published by Elsevier and edited by A. R. Katritzky & R. JK Taylor. Manipulation of the required substitution patterns have also been described in the available chemical literature as summarized in compendia such as “Comprehensive Organic Chemistry” published by Elsevier and edited by DH R. Barton and W. D. Ollis; “Comprehensive Organic Functional Group Transformations” edited by edited by A. R. Katritzky & R. JK Taylor and “Comprehensive Organic Transformation” published by Wiley-CVH and edited by R. C. Larock.

The starting materials used and the intermediates prepared using the methods set forth in the Examples below may be isolated and purified if desired using conventional techniques, including but not limited to filtration, distillation, crystallization, chromatography and alike. Such materials can be characterized using conventional means, including physical constants and spectral data.

One skilled in the art will be aware of standard formulation techniques as set forth in the open literature as well as in textbooks such as Zheng, “Formulation and Analytical Development for Low-dose Oral Drug Products,” Wiley, 2009, ISBN.

### EXAMPLES

### General Methods

Solvents, reagents, and intermediates that are commercially available were used as received. Reagents and intermediates that are not commercially available were prepared in the manner as described below. ¹H NMR spectra are reported as ppm downfield from Me₄Si with number of protons, multiplicities, and coupling constants in Hertz indicated parenthetically. Where LC/MS data are presented, the retention time and observed parent ion are given. Flash column chromatography was performed using pre-packed normal phase silica or bulk silica, and using a gradient elution of hexanes/ethyl acetate, typically from 100% hexanes to 100% ethyl acetate.

Why are none of the exemplified compounds named?

### Example 1

### Preparation of Intermediate Compound Int-1

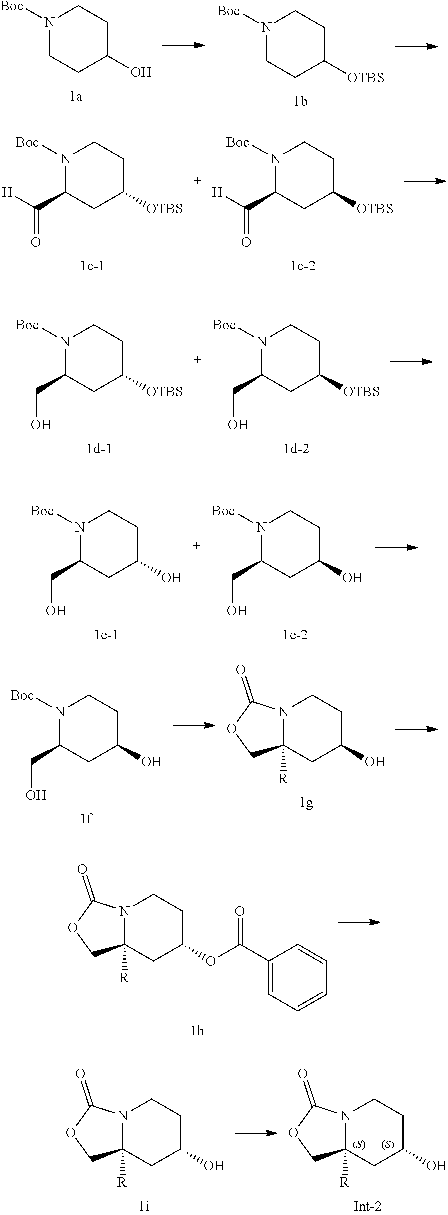

### Step A—Synthesis of Compound 1b

Into a 5-L, 3-necked round-bottom flask (purged and maintained with an inert atmosphere of nitrogen) was charged 1a (120 g, 596 mmol), imidazole (81.2 g, 1.19 mmol), and CH₂Cl₂ (2.5 L). tert-Butyldimethylsilyl chloride (116.7 g, 774.3 mmol) was added in portions. The resulting reaction was allowed to stir for 3 hours at room temperature, then cooled to 10° C. with a water/ice bath. The reaction was then quenched by the addition of saturated, aqueous NaHCO₃ (3 L). The resulting reaction was extracted with CH₂Cl₂, and the organic extract was dried over Na₂SO₄, filtered, and concentrated in vacuo. The resulting residue was purified using silica gel chromatography, eluting with 10% EtOAc in petroleum ether, to provide compound 1b as an oil.

### Step B—Synthesis of Compounds 1c-1 and 1c-2

Into a 10-L, 4-necked round-bottom flask (purged and maintained with an inert atmosphere of nitrogen) was charged 1b (170 g, 539 mmol), and TMEDA (138 g, 1188 mmol) in methyl tert-butyl ether (4.2 L). The solution was cooled to −78° C. and sec-BuLi (1.3M in cyclohexane, 748 mL, 970 mmol) was added dropwise with stirring. The resulting reaction was allowed to stir for 1 hour at −40° C. in a liquid nitrogen bath. After cooling to −78° C., DMF (395 g, 1872 mmol) was added dropwise with stirring. The resulting reaction was maintained at −40° C. for 1.5 hours, then quenched by the addition of saturated, aqueous NH₄Cl (2 L). The resulting reaction was extracted with methyl tert-butyl ether, dried over Na₂SO₄, and concentrated to provide 1c-1 and 1c-2 as an oil, which was used without further purification.

### Step C—Synthesis of Compounds 1d-1 and 1d-2

Into a 5-L, 4-necked round-bottom flask (purged and maintained with an inert atmosphere of nitrogen) was charged 1c-1 and 1c-2 (100 g, 291 mmol) in methanol (2 L). Sodium borohydride (22 g, 582 mmol) was added in portions at 0° C., and the resulting solution was allowed to stir for 2 hours at 0° C. The reaction was quenched by the addition of water (5 L). The resulting reaction was extracted with ethyl acetate, dried over Na₂SO₄, and concentrated in vacuo to provide a mixture of 1d-1 and 1d-2 as an oil, which was used without further purification.

### Step D and E—Synthesis of Compound 1f

Into a 5-L, 3-necked round-bottom flask (purged and maintained with an inert atmosphere of nitrogen) was charged a mixture of 1d-1 and 1d-2 (100 g, 289 mmol), and THE (2 L), and the solution was treated with tetra-n-butylammonium fluoride (1 M in THF, 268 mL). The resulting reaction was allowed to stir for 3 hours at room temperature. The reaction mixture was concentrated in vacuo, and the residue obtained was purified using a silica gel column, eluted with ethyl acetate/petroleum ether (10:1) to provide compound 1f as a liquid.

### Step F—Synthesis of Compound 1g

Into a 3-L, 3-necked round-bottom flask, under nitrogen atmosphere, was charged if (100 g, 432 mmol), and potassium 2-methylpropan-2-olate (29.1 g, 260 mol) in iPrOH (2 L). The resulting reaction was allowed to stir for 3 hours at 70° C., then cooled to 0° C. HCl in MeOH (4 N) was then used to adjust the resulting solution to pH 6, and the resulting mixture was concentrated in vacuo. The resulting residue was diluted with CH₂Cl₂ (1 L), filtered, and concentrated in vacuo, and the residue obtained was purified using silica gel chromatography, eluting with 90% EtOAc in petroleum ether, to provide compound 1g as a solid.

### Step G—Synthesis of Compound 1h

Into a 3-L, 4-necked round-bottom flask under an inert atmosphere of nitrogen, was placed 1 g (67 g, 426.30 mmol), benzoic acid (62.4 g, 0.51 mmol), triphenylphosphene (134.1 g, 0.51 mmol), and oxolane (1.3 L). DEAD (89.1 g, 0.51 mmol) was added dropwise at 0° C., and the resulting reaction was allowed to stir overnight at room temperature. The reaction mixture was cooled to 0° C., then the reaction was then quenched by the addition of 2 L of aqueous NaHCO₃. The resulting solution was extracted with 3×2 L of ethyl acetate, and the combined organic extracts were dried over anhydrous sodium sulfate, filtered, and concentrated in vacuo, to provide 1 h as an oil, which was used without further purification.

### Step H—Synthesis of Compound 1i

Into a 3-L, 3-necked round-bottom flask, purged and maintained under an inert atmosphere of nitrogen, was placed 1 h (118 g, 451.63 mmol), and lithium hydroxide (13 g, 0.54 mmol) in methanol (2 L). The resulting reaction was allowed to stir for 3 hours at room temperature, and was concentrated in vacuo. The resulting residue was purified using flash column chromatography on silica gel (eluting with ethyl acetate/petroleum ether, 10:1) to provide 1i as a solid.

### Step H—Chiral Resolution to Provide Intermediate Compound Int-1

Compound 1i (49 g) was purified using Prep-SFC under the following conditions: Column, Phenomenex Lux 5u Cellulose-45*25 cm, 5 um 00G-4491-V0-AX664184-1; mobile phase, Solvent A (Solvent B═CO2: 50) IPA:HEX=1:1; Detector UV 210 nm, pressure 100 bar Flow Rate (g/min£) 150 to provide Int-1 as a solid. MS (ESI, m/z): 158 [M+H]⁺. ¹H NMR (400 MHz, CDCl₃, ppm): 4.45-4.404 (1H, m), 4.31 (1H, s), 4.17-4.13 (1H, m), 3.91-3.87 (1H, m), 3.73-3.67 (1H, m), 3.36-3.31 (1H, m), 2.07 (1H, s), 1.99-1.93 (1H, d), 1.77-1.69 (2H, m), 1.60-1.52 (1H, m).

### Example 2

### Preparation of Intermediate Compound Int-2

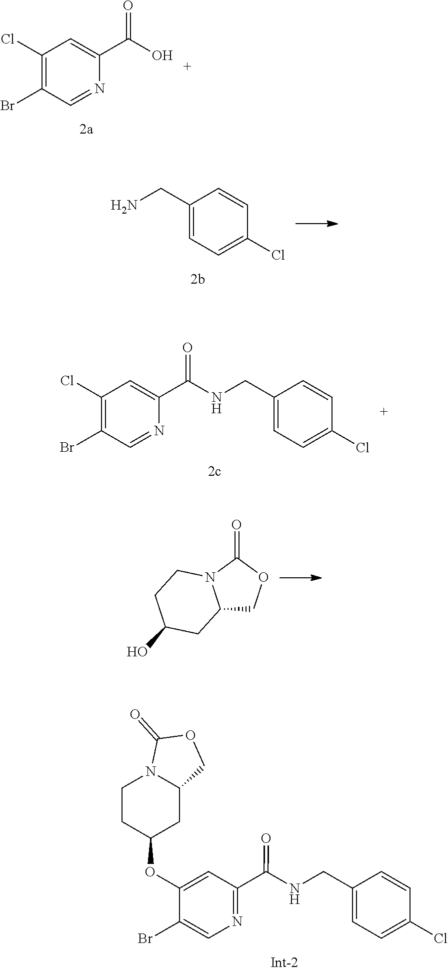

### Step A—Synthesis of Compound 2c

To a stirred solution of 2a (4.00 g, 16.9 mmol) in DMF (75.0 mL) was added 2-(3H-[1,2,3]triazolo[4,5-b]pyridin-3-yl)-1,1,3,3-tetramethylisouronium hexafluorophosphate(V) (7.40 g, 19.5 mmol), and the resulting solution was allowed to stir at room temperature for 15 minutes until homogenous. 2b (4.22 g, 23.7 mmol) was added, followed by N-ethyl-N-isopropylpropan-2-amine (6.48 mL, 37.2 mmol), and the reaction was allowed to stir at room temperature overnight. The reaction mixture was diluted with water and extracted with ethyl acetate. The organic extract was washed with brine, dried over MgSO₄, filtered, and concentrated in vacuo. The residue obtained was purified using silica gel chromatography, eluting with 50-100% chloroform in hexanes, to provide compound 2c as a solid. MS: m/z 359.0 and 361.0 and 363.0=[M+H]⁺.

### Step B—Synthesis of Compound Int-2

To a stirred solution of Int-1 (0.57 g, 3.6 mmol) in DMF (10.0 mL) was added sodium hydride (60% by weight in mineral oil, 0.19 g, 4.7 mmol), and the resulting reaction was allowed to stir at room temperature for 10 min. 2c (1.00 g, 2.8 mmol) was added, and the reaction was heated to 100° C. After stirring for an additional 1 hour, the reaction mixture was cooled to room temperature, quenched with water, and diluted with ethyl acetate. The organic extract was washed with water and brine, dried over MgSO₄, filtered, and concentrated in vacuo, and the residue obtained was purified using silica gel chromatography, eluting with 0-75% (3:1 ethyl acetate:ethanol) in hexanes, to provide compound Int-2 as a solid. MS (ESI, m/z): 480.3, 482.3 [M+H]⁺. ¹H NMR (400 MHz, methanol-d₄, ppm): δ 8.65 (s, 1H), 7.81 (s, 1H), 7.37-7.32 (m, 4H), 5.28 (brs, 1H), 4.59 (s, 2H), 4.49 (t, J=8.4 Hz, 1H), 4.24-4.16 (m, 1H), 4.01 (dd, J=6.0, 8.4 Hz, 1H), 3.77 (dd, J=5.6, 13.2 Hz, 1H), 3.40-3.33 (m, 1H), 2.32-2.28 (m, 1H), 2.11-2.07 (m, 1H), 1.95-1.80 (m, 2H).

### Example 3

### Preparation of Compound 3

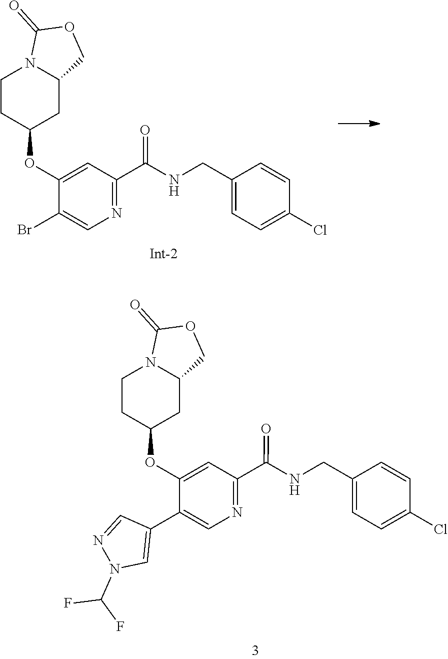 [SMILES: O=C(NCc1ccc(Cl)cc1)c1cc(O[C@H]2CCN3C(=O)OC[C@@H]3C2)c(Br)cn1 ; O=C(NCc1ccc(Cl)cc1)c1cc(O[C@H]2CCN3C(=O)OC[C@@H]3C2)c(-c2cnn(C(F)F)c2)cn1]

### Step A—Synthesis of Compound 3

A solution of Int-2 (30 mg, 0.062 mmol), 1-(difluoromethyl)-4-(4,4,5,5-tetramethyl-1,3,2-dioxaborolan-2-yl)-1H-pyrazole (15 mg, 0.062 mmol), and aqueous potassium phosphate (1 M, 0.19 mL) in dioxane (0.31 mL) was purged subsurface with nitrogen. 1,1′-Bis(di-tert-butylphosphino)ferrocene palladium dichloride (2 mg, 0.003 mmol) was added, and the reaction was purged further, then sealed and irradiated in the microwave at 130° C. for 30 minutes. The reaction was cooled to room temperature, concentrated in vacuo, and the resulting residue was purified using preparative HPLC (reverse-phase C-18), eluting with acetonitrile/water+0.1% TFA 10-95%, to provide compound 3 as a solid. MS (ESI, m/z): 518.2 [M+H]⁺. ¹H NMR (400 MHz, methanol-d₄, ppm) δ 8.75 (s, 1H), 8.54 (s, 1H), 8.23 (s, 1H), 7.84 (s, 1H), 7.58 (t, J=59.6 Hz, 1H), 7.36-7.30 (m, 4H), 5.26 (s, 1H), 4.59 (s, 2H), 4.43 (t, J=8.0 Hz, 1H), 4.41-3.95 (m, 2H), 3.78 (dd, J=4.8, 13.6 Hz, 1H), 3.24-3.16 (m, 1H), 2.35 (dd, J=2.0, 14.0 Hz, 1H), 2.14 (dd, J=1.6, 12.8 Hz, 1H), 1.93-1.79 (m, 2H).

### Example 4

### Preparation of Compound 4

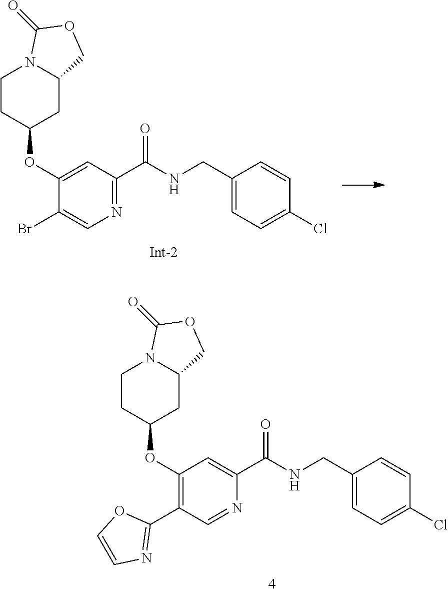 [SMILES: O=C(NCc1ccc(Cl)cc1)c1cc(O[C@H]2CCN3C(=O)OC[C@@H]3C2)c(Br)cn1 ; O=C(NCc1ccc(Cl)cc1)c1cc(O[C@H]2CCN3C(=O)OC[C@@H]3C2)c(-c2ncco2)cn1]

### Step A—Synthesis of Compound 4

A solution of Int-2 (20 mg, 0.062 2 mol), 2-(tributylstannyl)oxazole (37 mg, 0.10 mmol), and tetrakis(triphenylphosphine)palladium(0) (4.8 mg, 0.004 mmol) in dioxane (0.50 mL) was purged subsurface with nitrogen, then sealed and irradiated at 150° C. for 30 minutes. The reaction was concentrated in vacuo, and the resulting residue was purified using preparative HPLC (reverse-phase C-18), eluting with acetonitrile/water+0.1% TFA 10-95%, to provide compound 3 as a solid. MS (ESI, m/z): 469.3 [M+H]⁺. ¹H NMR (600 MHz, Chloroform-d) δ 9.02 (s, 1H), 7.89 (s, 1H), 7.85 (d, J=0.7 Hz, 1H), 7.38 (d, J=0.7 Hz, 1H), 7.31 (q, J=8.6 Hz, 4H), 5.25 (s, 1H), 4.64 (d, J=6.2 Hz, 2H), 4.45 (t, J=8.4 Hz, 1H), 4.27-4.20 (m, 1H), 3.95 (dd, J=8.7, 5.9 Hz, 1H), 3.82 (dd, J=13.4, 4.9 Hz, 1H), 3.39 (td, J=13.1, 3.2 Hz, 1H), 2.30 (d, J=11.9 Hz, 1H), 2.14-2.08 (m, 2H), 1.88 (td, J=12.6, 4.7 Hz, 1H), 1.81-1.74 (m, 1H).

### Example 5

### Preparation of Intermediate Compound Int-5

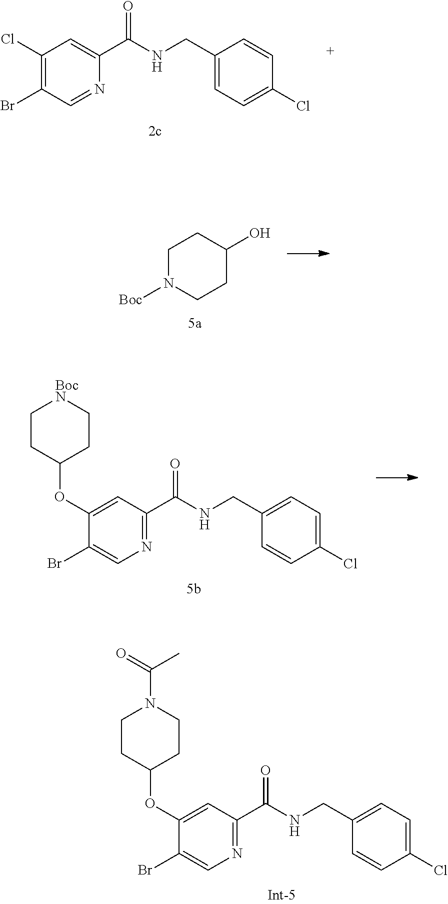 [SMILES: O=C(NCc1ccc(Cl)cc1)c1cc(Cl)c(Br)cn1 ; CC(C)(C)OC(=O)N1CCC(O)CC1 ; CC(C)(C)OC(=O)N1CCC(Oc2cc(C(=O)NCc3ccc(Cl)cc3)ncc2Br)CC1 ; CC(=O)N1CCC(Oc2cc(C(=O)NCc3ccc(Cl)cc3)ncc2Br)CC1]

### Step A—Synthesis of Compound 5b

To a solution of 5a (335 mg, 1.67 mmol) in N,N-dimethyl formamide (2.5 mL) was added sodium hydride (61 mg, 1.53 mmol) at room temperature. The resulting mixture was allowed to stir for 10 minutes, then 2c (500 mg, 1.39 mmol) was added. The reaction was heated to 85° C., and allowed to stir at this temperature for 16 hours, then quenched with water (30 mL), and extracted with ethyl acetate (2×50 mL). The combined organic extracts were washed with saturated aqueous sodium chloride (40 mL), dried over anhydrous sodium sulfate, filtered, and concentrated in vacuo. The resulting residue was purified using silica gel chromatography, eluting with a gradient of ethyl acetate:petroleum ether—0:1 to 1:1, to provide 5b. MS (ESI, m/z): 524.2, 526.2 [M+H]⁺.

### Step B—Synthesis of Intermediate Compound Int-5

To a solution of 5b (550 mg, 1.05 mmol) in dichloromethane (5 mL) was added trifluoroacetic acid (2 mL). The resulting mixture was allowed to stir for 30 minutes at room temperature, then the reaction mixture was concentrated in vacuo. The resulting residue was dissolved in dichloromethane (5 mL) and to the resulting solution was added diisopropylethylamine (0.92 mL, 5.24 mmol), followed by acetyl chloride (165 mg, 2.10 mmol). The resulting reaction was allowed to stir for 1 hour at room temperature, then was concentrated in vacuo. The resulting residue was purified using silica gel chromatography, eluting with a gradient of methanol:dichloromethane—0:10 to 1:10, to provide intermediate compound Int-5. MS (ESI, m/z): 466.0, 468.0 [M+H]⁺. ¹H NMR (500 MHz, Chloroform-d) δ 8.53 (s, 1H), 8.34 (s, 1H), 7.77 (s, 1H), 7.36-7.29 (m, 4H), 4.95 (s, 1H), 4.63 (d, J=6.2 Hz, 2H), 3.95-3.88 (m, 1H), 3.73-3.61 (m, 2H), 3.58-3.51 (m, 1H), 2.16 (s, 3H), 2.03-1.92 (m, 4H).

### Example 6

### Preparation of Compound 6

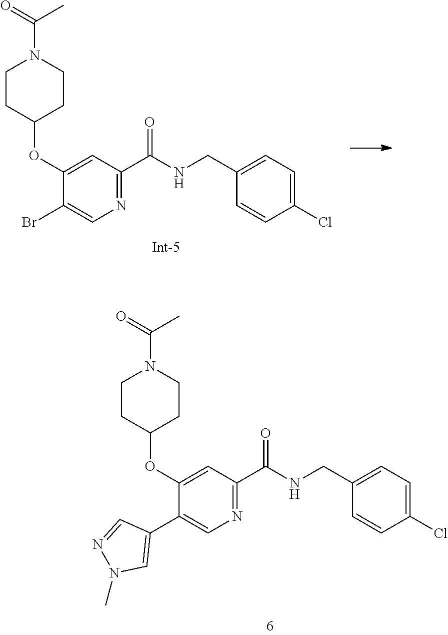 [SMILES: CC(=O)N1CCC(Oc2cc(C(=O)NCc3ccc(Cl)cc3)ncc2Br)CC1 ; CC(=O)N1CCC(Oc2cc(C(=O)NCc3ccc(Cl)cc3)ncc2-c2cnn(C)c2)CC1]

### Step A—Synthesis of Compound 6

To a mixture of Int-5 (50 mg, 0.11 mmol), 1-methyl-4-(4,4,5,5-tetramethyl-1,3,2-dioxaborolan-2-yl)-1H-pyrazole (27 mg, 0.13 mmol), potassium phosphate tribasic (68 mg, 0.32 mmol) in 1,4-dioxane (1.5 mL), and water (0.15 mL) was added tetrakis(triphenylphosphine)palladium(0) (25 mg, 0.02 mmol) at room temperature. The reaction mixture was purged with nitrogen 3 times and allowed to stir at 100° C. for 16 hours. The reaction mixture was diluted with water (20 mL), and extracted with ethyl acetate (3×30 mL). The combined organic extracts were washed with saturated aqueous sodium chloride (50 mL), dried over anhydrous sodium sulfate, filtered, and concentrated in vacuo. The resulting residue was purified using silica gel column chromatography, eluting with a gradient of methanol/dichloromethane—0:10 to 1:10, to provide compound 6. MS (ESI, m/z): 468.2 [M+H]+. ¹H NMR (400 MHz, chloroform-d, ppm): δ 8.58 (s, 1H), 7.92 (s, 1H), 7.84 (s, 1H), 7.82 (s, 1H), 7.31 (s, 4H), 4.95-4.91 (m, 1H), 4.63 (t, J=6.4 Hz, 2H), 3.98-3.91 (m, 4H), 3.73-3.68 (m, 1H), 3.52-3.40 (m, 2H), 2.15-2.05 (m, 5H), 1.92-1.83 (m, 2H).

### Example 7

### Preparation of Compound 7

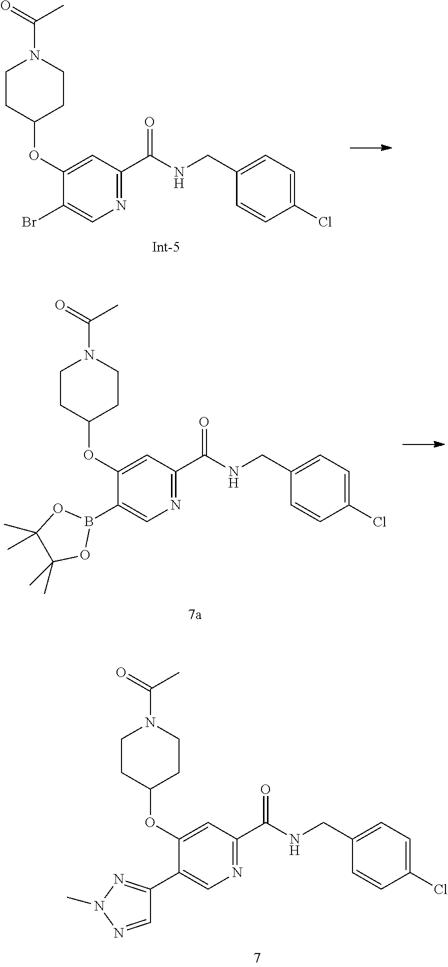 [SMILES: CC(=O)N1CCC(Oc2cc(C(=O)NCc3ccc(Cl)cc3)ncc2Br)CC1 ; CC(=O)N1CCC(Oc2cc(C(=O)NCc3ccc(Cl)cc3)ncc2B2OC(C)(C)C(C)(C)O2)CC1 ; CC(=O)N1CCC(Oc2cc(C(=O)NCc3ccc(Cl)cc3)ncc2-c2cnn(C)n2)CC1]

### Step A—Synthesis of Compound 7a

To a solution of Int-5 (1.0 g, 2.1 mmol) in 1,4-dioxane (15 mL) was added 4,4,4′,4′,5,5,5′,5′-octamethyl-2,2′-bi(1,3,2-dioxaborolane) (1.1 g, 4.3 mmol), potassium acetate (0.84 g, 8.6 mmol), and 1,1′-bis(diphenylphosphino)ferrocene-palladium(II)dichloride dichloromethane complex (0.35 g, 0.43 mmol) at room temperature, under nitrogen atmosphere. The mixture was degassed with nitrogen and allowed to stir at 100° C. for 1.5 hours. The reaction mixture was quenched by water (50 mL) and extracted with ethyl acetate (50 mL×3). The combined organic extracts were washed with brine (100 mL), dried over anhydrous sodium sulfate, and filtered. The filtrate was concentrated in vacuo to provide compound 7a as an oil that was used without further purification.

### Step B—Synthesis of Compound 7

A solution of 7a (15 mg, 0.035 mmol), 4-bromo-2-methyl-2H-1,2,3-triazole (11 mg, 0.069 mmol), and aqueous potassium phosphate (1 M, 0.087 mL) in dioxane (0.35 mL) was purged subsurface with nitrogen. G2 Xphos (2.7 mg, 0.003 mmol) was added. The reaction was further purged, then sealed, heated at 80° C., and allowed to stir at this temperature for 18 hours. The reaction mixture was cooled to room temperature, diluted with ethyl acetate, passed through an Opti Chem SPE cartridge containing 1000 mg diatomaceous earth, and concentrated in vacuo. The resulting residue was purified using preparative HPLC (reverse-phase C-18), eluting with acetonitrile/water+0.1% TFA 10-95%, to provide compound 7. MS (ESI, m/z): 469.2 [M+H]⁺. ¹H NMR (500 MHz, Chloroform-d) δ 9.03 (s, 1H), 8.46 (t, J=5.9 Hz, 1H), 8.04 (s, 1H), 7.86 (s, 1H), 7.33 (s, 4H), 4.97 (dt, J=7.3, 3.7 Hz, 1H), 4.66 (d, J=6.2 Hz, 2H), 4.29 (s, 3H), 4.22 (d, J=19.0 Hz, 1H), 4.00-3.89 (m, 1H), 3.80-3.68 (m, 1H), 3.61-3.53 (m, 1H), 3.51-3.43 (m, 1H), 2.15 (s, 4H), 1.97-1.86 (m, 2H).

### Example 8

### Preparation of Compound 8

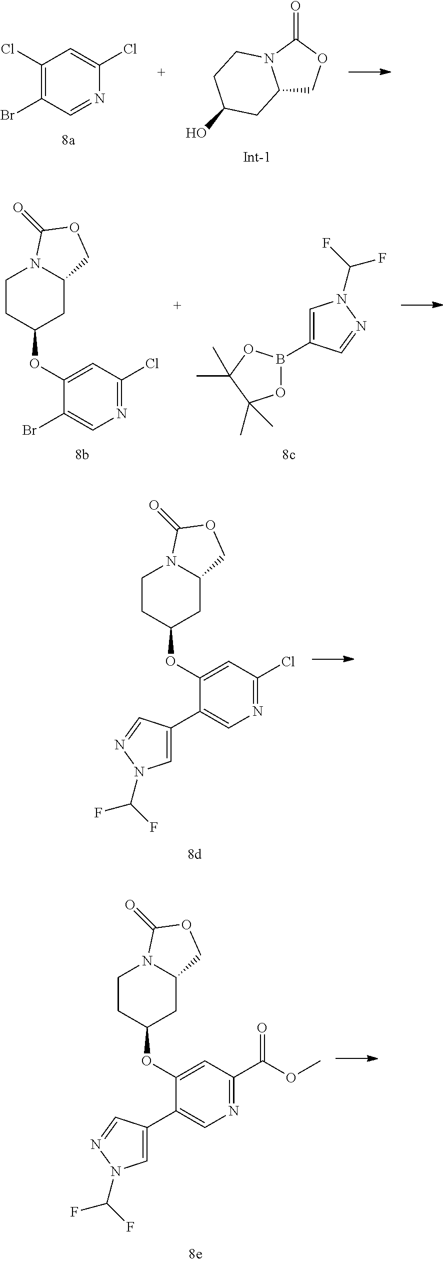 [SMILES: Clc1cc(Cl)c(Br)cn1 ; O=C1OC[C@@H]2C[C@@H](O)CCN12 ; O=C1OC[C@@H]2C[C@@H](Oc3cc(Cl)ncc3Br)CCN12 ; CC1(C)OB(c2cnn(C(F)F)c2)OC1(C)C ; O=C1OC[C@@H]2C[C@@H](Oc3cc(Cl)ncc3-c3cnn(C(F)F)c3)CCN12 ; COC(=O)c1cc(O[C@H]2CCN3C(=O)OC[C@@H]3C2)c(-c2cnn(C(F)F)c2)cn1]

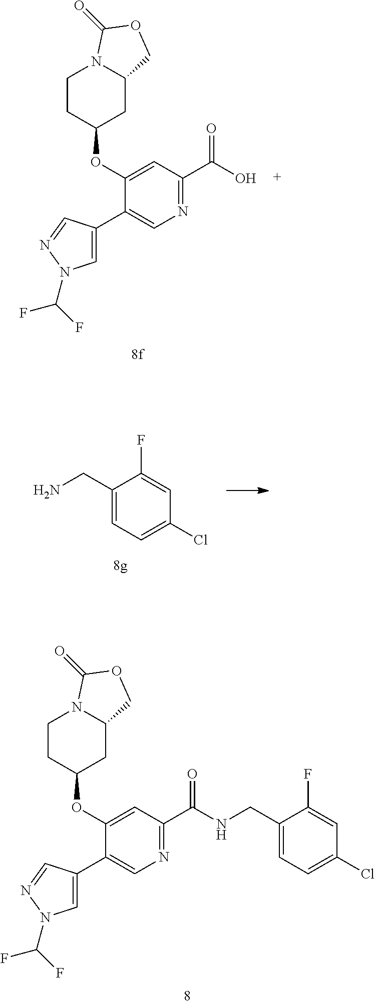 [SMILES: O=C(O)c1cc(O[C@H]2CCN3C(=O)OC[C@@H]3C2)c(-c2cnn(C(F)F)c2)cn1 ; NCc1ccc(Cl)cc1F ; O=C(NCc1ccc(Cl)cc1F)c1cc(O[C@H]2CCN3C(=O)OC[C@@H]3C2)c(-c2cnn(C(F)F)c2)cn1]

### Step A—Synthesis of Compound 8b

To a stirred solution of Int-1 (416 mg, 2.6 mmol) in DMF (12 mL) was added sodium hydride (60% by weight in mineral oil, 101 mg, 2.5 mmol), and the reaction was allowed to stir at room temperature for 20 minutes. A solution of 8a (600 mg, 2.6 mmol) in DMIF (2.0 mL) was added, and the reaction was allowed to stir at room temperature for 4 additional hours. The reaction mixture was quenched with water, and extracted with ethyl acetate. The organic extract was washed with water and brine, dried over MgSO₄, filtered, and concentrated in vacuo, and the residue obtained was purified using silica gel chromatography, eluting with 0-30%₀ ethyl acetate in hexanes, to provide compound 8b as a solid.

### Step B—Synthesis of Compound 8d

A solution of 8b (1.2 g, 3.6 mmol), 8c (0.96 g, 3.9 mmol), and sodium carbonate (1.1 g, 10.8 mmol) in dioxane (16 mL), water (4.0 mL), and THE (4.0 mL) was purged subsurface with nitrogen. 1,1′-Bis(diphenylphosphino)ferrocene palladium dichloride methylene chloride adduct (0.15 g, 0.18 mmol) was added, and the reaction was purged further, then sealed and heated at 100° C. for 18 hours. The reaction mixture was diluted with ethyl acetate, and the organic phase was washed with water and brine, and concentrated in vacuo. The residue obtained was purified using silica gel chromatography, eluting with 0-75% (3:1 ethyl acetate:ethanol) in hexanes, to provide compound 8d as a solid. MS (ESI, m/z): 385 [M+H]⁺.

### Step C—Synthesis of Compound 8e

A solution of 8d (282 mg, 0.73 mmol) in methanol (7.0 mL) was purged subsurface with nitrogen. DIEA (0.64 mL, 3.6 mmol), and 1,1′-Bis(diphenylphosphino)ferrocene palladium dichloride methylene chloride adduct (60 mg, 0.073 mmol) was added, and the reaction mixture was sealed and heated at 75° C. under 100 PSI CO for 12 hours. The reaction mixture was diluted with water, and filtered. The collected solid was taken up in ethyl acetate and concentrated in vacuo. The resulting residue was purified using silica gel chromatography, eluting with 5-75% (3:1 ethyl acetate:ethanol) in hexanes, to provide compound 8e as an oil. MS (ESI, m/z): 409.2 [M+H]⁺.

### Step D—Synthesis of Compound 8f

To a solution of 8e (282 mg, 0.69 mmol) in THE (4.0 mL) was added aqueous sodium hydroxide (1 M, 2 mL, 2.1 mmol), and the resulting reaction was allowed to stir at room temperature overnight. The reaction mixture was acidified using 1 M HCl, diluted with water and filtered. The collected solid was dried in vacuo to provide compound 8f as a solid, which was used without further purification. MS (ESI, m/z): 395.2 [M+H]⁺.

### Step E—Synthesis of Compound 8

To a stirred solution of 8f (20 mg, 0.051 mmol) in DMF (0.50 mL) was added 2-(3H-[1,2,3]triazolo[4,5-b]pyridin-3-yl)-1,1,3,3-tetramethylisouronium hexafluorophosphate(V) (39 mg, 0.10 mmol), and the resulting reaction was allowed to stir at room temperature for 15 minutes. 8 g (12 mg, 0.076 mmol) was added, followed by TEA (0.021 mL, 0.15 mmol), and the reaction was allowed to stir at room temperature for an additional 2.5 hours. The reaction mixture was then filtered and directly purified using preparative HPLC (reverse-phase C-18), eluting with acetonitrile/water+0.1% TFA 10-95%, to provide compound 8 as a solid. MS (ESI, m/z): 436.4 [M+H]⁺. ¹H NMR (600 MHz, DMSO-d₆) δ 9.38 (t, J=6.3 Hz, 1H), 8.83 (s, 1H), 8.71 (s, 1H), 8.40 (s, 1H), 7.91 (t, J=58.8 Hz, 1H), 7.71 (s, 1H), 7.48-7.11 (m, 3H), 4.50 (d, J=6.5 Hz, 2H), 4.34-4.31 (m, 1H), 3.89 (dd, J=8.8, 4.5 Hz, 2H), 3.58 (dd, J=13.2, 5.5 Hz, 2H), 3.20-2.90 (m, 1H), 2.21 (d, J=13.6 Hz, 1H), 2.13-1.88 (in, 1H), 1.88-1.60 (in, 2H).

The following compounds of the present invention were made using the methods described in Examples 2-8 above, and substituting the appropriate reactants and/or reagents:

| Compound | Structure | MS [M + H]⁺ |
|---|---|---|
| 9* | 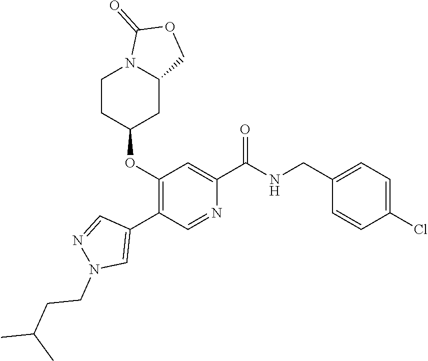 [SMILES: CC(C)CCn1cc(-c2cnc(C(=O)NCc3ccc(Cl)cc3)cc2O[C@H]2CCN3C(=O)OC[C@@H]3C2)cn1] | 538.4 |
| 10* | 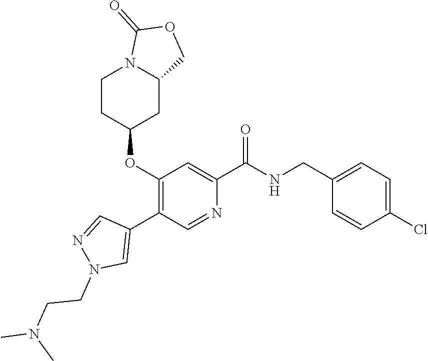 [SMILES: CN(C)CCn1cc(-c2cnc(C(=O)NCc3ccc(Cl)cc3)cc2O[C@H]2CCN3C(=O)OC[C@@H]3C2)cn1] | 539.4 |
| 11* | 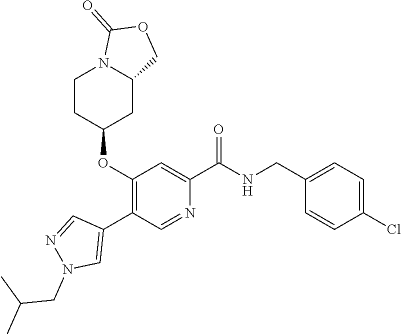 [SMILES: CC(C)Cn1cc(-c2cnc(C(=O)NCc3ccc(Cl)cc3)cc2O[C@H]2CCN3C(=O)OC[C@@H]3C2)cn1] | 524.4 |
| 12* | 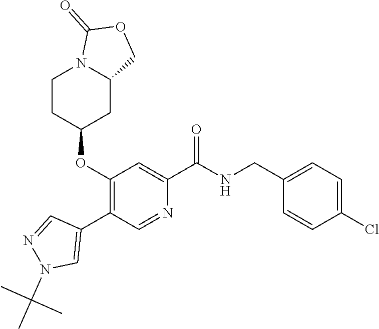 [SMILES: CC(C)(C)n1cc(-c2cnc(C(=O)NCc3ccc(Cl)cc3)cc2O[C@H]2CCN3C(=O)OC[C@@H]3C2)cn1] | 524.4 |
| 13* | 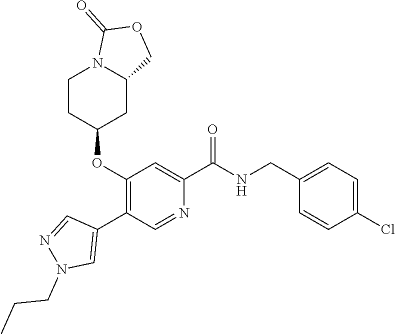 [SMILES: CCCn1cc(-c2cnc(C(=O)NCc3ccc(Cl)cc3)cc2O[C@H]2CCN3C(=O)OC[C@@H]3C2)cn1] | 510.4 |
| 14 | 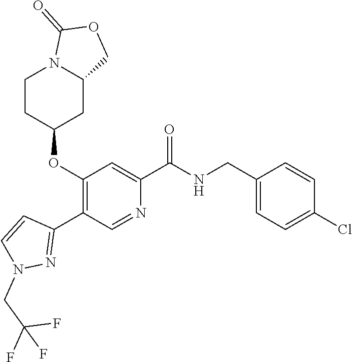 [SMILES: O=C(NCc1ccc(Cl)cc1)c1cc(O[C@H]2CCN3C(=O)OC[C@@H]3C2)c(-c2ccn(CC(F)(F)F)n2)cn1] | 550.3 |
| 15 | 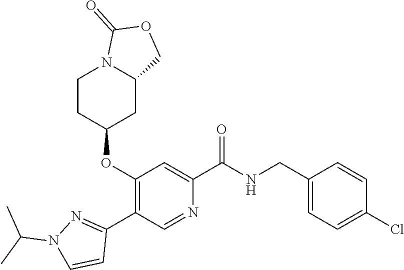 [SMILES: CC(C)n1ccc(-c2cnc(C(=O)NCc3ccc(Cl)cc3)cc2O[C@H]2CCN3C(=O)OC[C@@H]3C2)n1] | 510.4 |
| 16* | 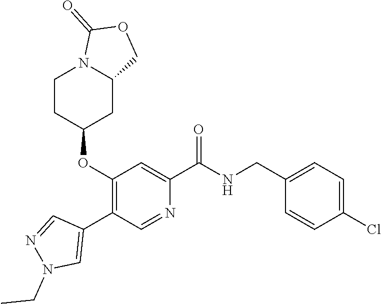 [SMILES: CCn1cc(-c2cnc(C(=O)NCc3ccc(Cl)cc3)cc2O[C@H]2CCN3C(=O)OC[C@@H]3C2)cn1] | 496.3 |
| 17 | 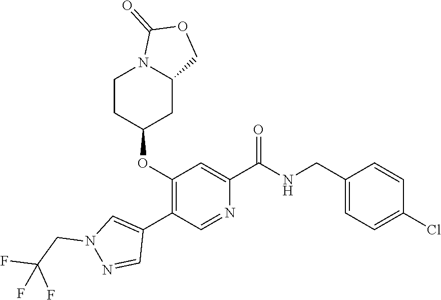 [SMILES: O=C(NCc1ccc(Cl)cc1)c1cc(O[C@H]2CCN3C(=O)OC[C@@H]3C2)c(-c2cnn(CC(F)(F)F)c2)cn1] | 550.3 |
| 18 | 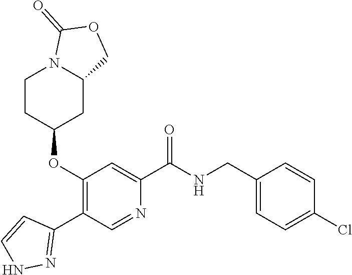 [SMILES: O=C(NCc1ccc(Cl)cc1)c1cc(O[C@H]2CCN3C(=O)OC[C@@H]3C2)c(-c2cc[nH]n2)cn1] | 468.3 |
| 19 | 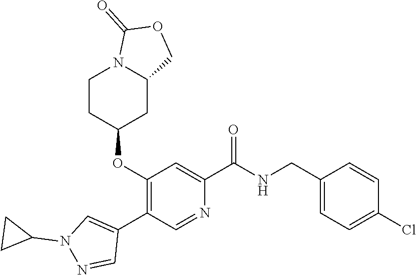 [SMILES: O=C(NCc1ccc(Cl)cc1)c1cc(O[C@H]2CCN3C(=O)OC[C@@H]3C2)c(-c2cnn(C3CC3)c2)cn1] | 508.3 |
| 20 | 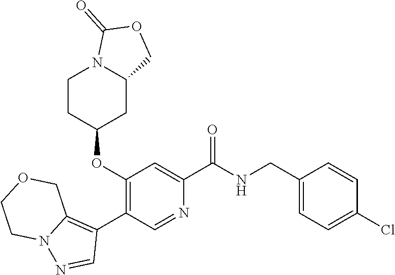 [SMILES: O=C(NCc1ccc(Cl)cc1)c1cc(O[C@H]2CCN3C(=O)OC[C@@H]3C2)c(-c2cnn3c2COCC3)cn1] | 524.2 |
| 21 | 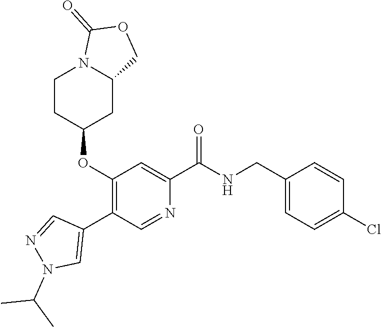 [SMILES: CC(C)n1cc(-c2cnc(C(=O)NCc3ccc(Cl)cc3)cc2O[C@H]2CCN3C(=O)OC[C@@H]3C2)cn1] | 510.4 |
| 22 | 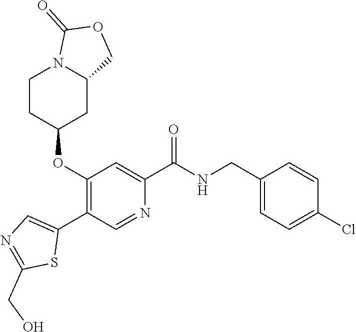 [SMILES: O=C(NCc1ccc(Cl)cc1)c1cc(O[C@H]2CCN3C(=O)OC[C@@H]3C2)c(-c2cnc(CO)s2)cn1] | 515.2 |
| 23 | 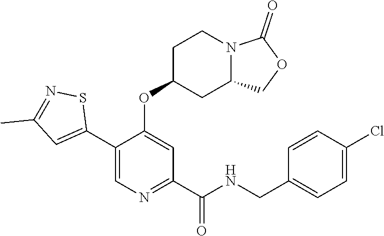 [SMILES: Cc1cc(-c2cnc(C(=O)NCc3ccc(Cl)cc3)cc2O[C@H]2CCN3C(=O)OC[C@@H]3C2)sn1] | 499.2 |
| 24 | 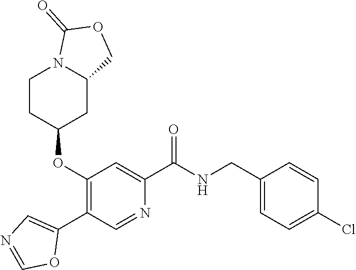 [SMILES: O=C(NCc1ccc(Cl)cc1)c1cc(O[C@H]2CCN3C(=O)OC[C@@H]3C2)c(-c2cnco2)cn1] | 469 |
| 25* | 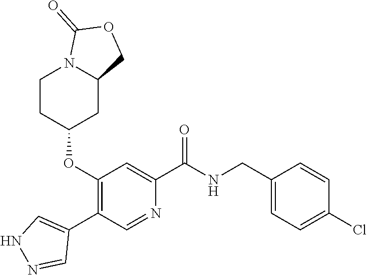 [SMILES: O=C(NCc1ccc(Cl)cc1)c1cc(O[C@@H]2CCN3C(=O)OC[C@H]3C2)c(-c2cn[nH]c2)cn1] | 468.3 |
| 26 | 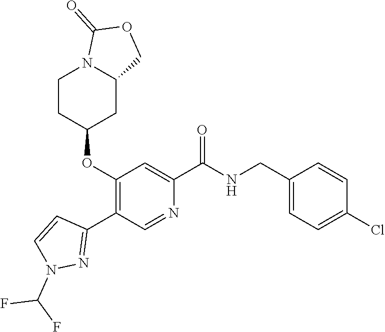 [SMILES: O=C(NCc1ccc(Cl)cc1)c1cc(O[C@H]2CCN3C(=O)OC[C@@H]3C2)c(-c2ccn(C(F)F)n2)cn1] | 518.1 |
| 27 | 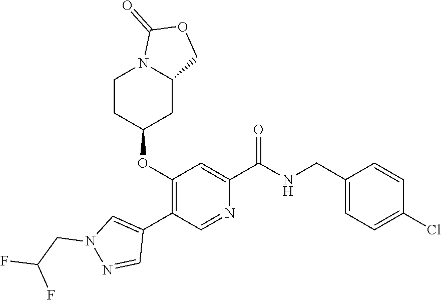 [SMILES: O=C(NCc1ccc(Cl)cc1)c1cc(O[C@H]2CCN3C(=O)OC[C@@H]3C2)c(-c2cnn(CC(F)F)c2)cn1] | 532.2 |
| 28 | 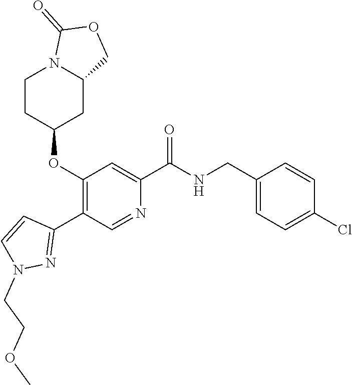 [SMILES: COCCn1ccc(-c2cnc(C(=O)NCc3ccc(Cl)cc3)cc2O[C@H]2CCN3C(=O)OC[C@@H]3C2)n1] | 526.4 |
| 29 | 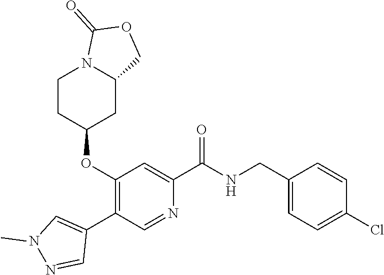 [SMILES: Cn1cc(-c2cnc(C(=O)NCc3ccc(Cl)cc3)cc2O[C@H]2CCN3C(=O)OC[C@@H]3C2)cn1] | 482.3 |
| 30 | 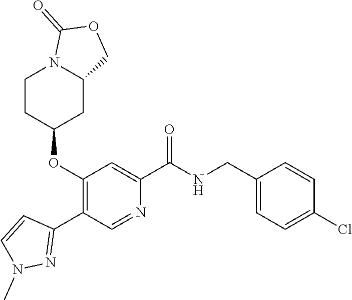 [SMILES: Cn1ccc(-c2cnc(C(=O)NCc3ccc(Cl)cc3)cc2O[C@H]2CCN3C(=O)OC[C@@H]3C2)n1] | 482.3 |
| 31 | 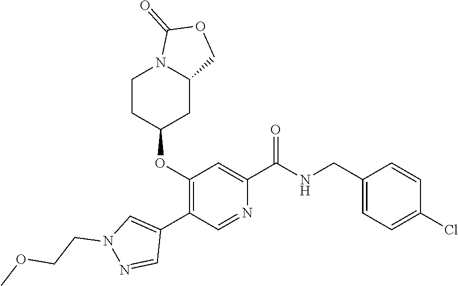 [SMILES: COCCn1cc(-c2cnc(C(=O)NCc3ccc(Cl)cc3)cc2O[C@H]2CCN3C(=O)OC[C@@H]3C2)cn1] | 526.4 |
| 32 | 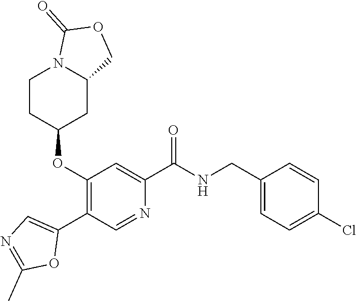 [SMILES: Cc1ncc(-c2cnc(C(=O)NCc3ccc(Cl)cc3)cc2O[C@H]2CCN3C(=O)OC[C@@H]3C2)o1] | 483.3 |
| 33 | 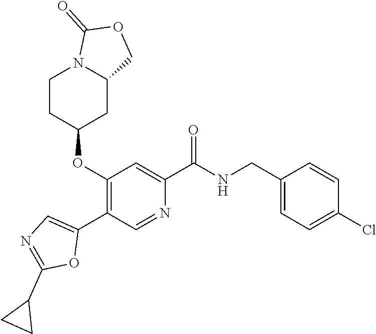 [SMILES: O=C(NCc1ccc(Cl)cc1)c1cc(O[C@H]2CCN3C(=O)OC[C@@H]3C2)c(-c2cnc(C3CC3)o2)cn1] | 509.3 |
| 34 | 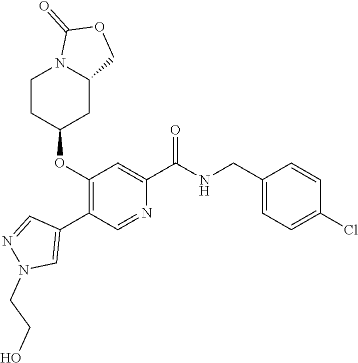 [SMILES: O=C(NCc1ccc(Cl)cc1)c1cc(O[C@H]2CCN3C(=O)OC[C@@H]3C2)c(-c2cnn(CCO)c2)cn1] | 512.3 |
| 35 | 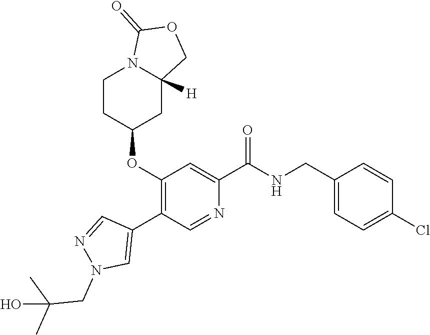 [SMILES: CC(C)(O)Cn1cc(-c2cnc(C(=O)NCc3ccc(Cl)cc3)cc2O[C@H]2CCN3C(=O)OC[C@@H]3C2)cn1] | 540.1 |
| 36 | 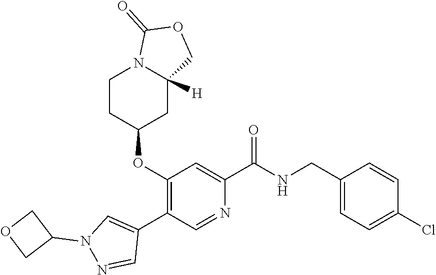 [SMILES: O=C(NCc1ccc(Cl)cc1)c1cc(O[C@H]2CCN3C(=O)OC[C@@H]3C2)c(-c2cnn(C3COC3)c2)cn1] | 524.2 |
| 37 | 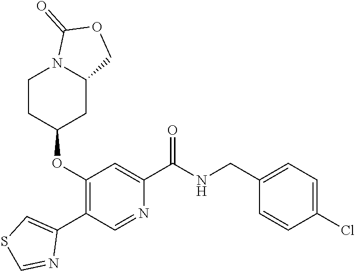 [SMILES: O=C(NCc1ccc(Cl)cc1)c1cc(O[C@H]2CCN3C(=O)OC[C@@H]3C2)c(-c2cscn2)cn1] | 485.2 |
| 38 | 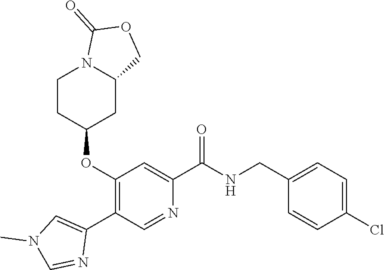 [SMILES: Cn1cnc(-c2cnc(C(=O)NCc3ccc(Cl)cc3)cc2O[C@H]2CCN3C(=O)OC[C@@H]3C2)c1] | 482.2 |
| 39 | 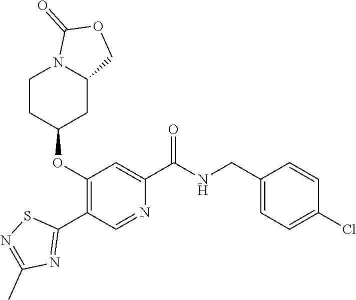 [SMILES: Cc1nsc(-c2cnc(C(=O)NCc3ccc(Cl)cc3)cc2O[C@H]2CCN3C(=O)OC[C@@H]3C2)n1] | 500.3 |
| 40 | 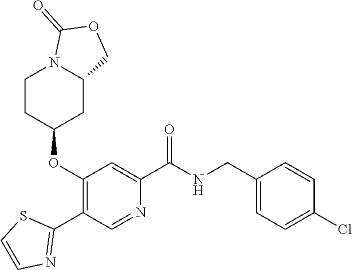 [SMILES: O=C(NCc1ccc(Cl)cc1)c1cc(O[C@H]2CCN3C(=O)OC[C@@H]3C2)c(-c2nccs2)cn1] | 485.2 |
| 41 | 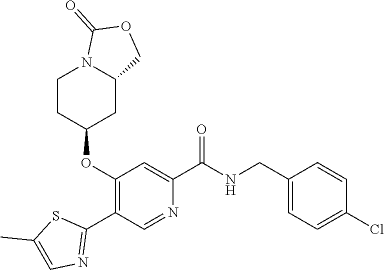 [SMILES: Cc1cnc(-c2cnc(C(=O)NCc3ccc(Cl)cc3)cc2O[C@H]2CCN3C(=O)OC[C@@H]3C2)s1] | 499.3 |
| 42 | 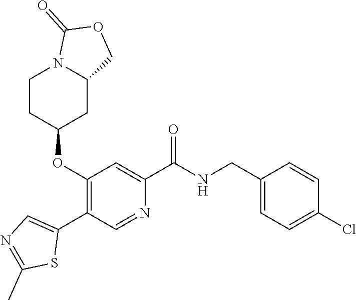 [SMILES: Cc1ncc(-c2cnc(C(=O)NCc3ccc(Cl)cc3)cc2O[C@H]2CCN3C(=O)OC[C@@H]3C2)s1] | 499.3 |
| 43 | 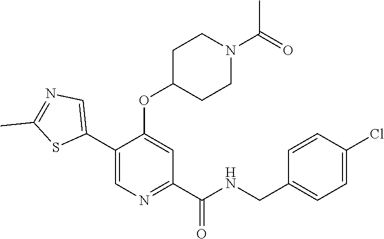 [SMILES: CC(=O)N1CCC(Oc2cc(C(=O)NCc3ccc(Cl)cc3)ncc2-c2cnc(C)s2)CC1] | 485.2 |
| 44 | 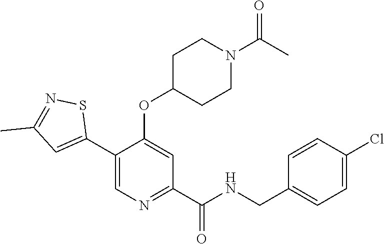 [SMILES: CC(=O)N1CCC(Oc2cc(C(=O)NCc3ccc(Cl)cc3)ncc2-c2cc(C)ns2)CC1] | 485.3 |
| 45 | 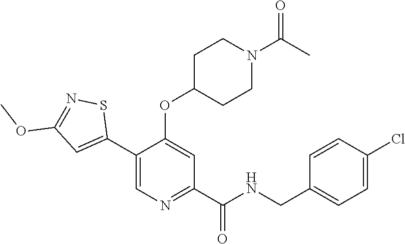 [SMILES: COc1cc(-c2cnc(C(=O)NCc3ccc(Cl)cc3)cc2OC2CCN(C(C)=O)CC2)sn1] | 501.3 |
| 46 | 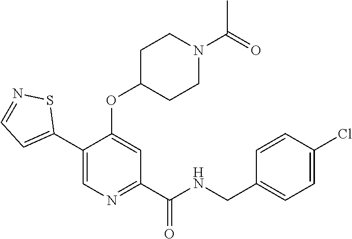 [SMILES: CC(=O)N1CCC(Oc2cc(C(=O)NCc3ccc(Cl)cc3)ncc2-c2ccns2)CC1] | 471.2 |
| 47 | 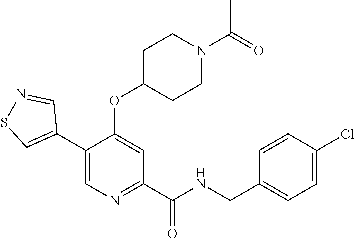 [SMILES: CC(=O)N1CCC(Oc2cc(C(=O)NCc3ccc(Cl)cc3)ncc2-c2cnsc2)CC1] | 471.2 |
| 48 | 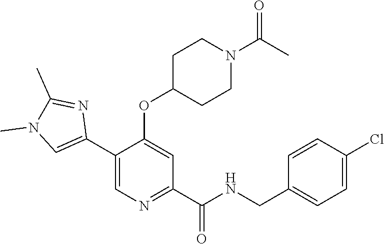 [SMILES: CC(=O)N1CCC(Oc2cc(C(=O)NCc3ccc(Cl)cc3)ncc2-c2cn(C)c(C)n2)CC1] | 482.2 |
| 49 | 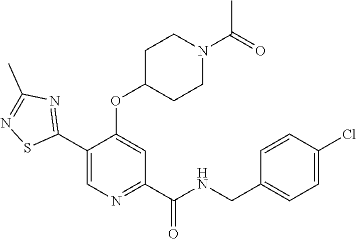 [SMILES: CC(=O)N1CCC(Oc2cc(C(=O)NCc3ccc(Cl)cc3)ncc2-c2nc(C)ns2)CC1] | 486.2 |
| 50 | 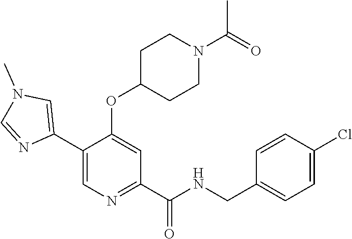 [SMILES: CC(=O)N1CCC(Oc2cc(C(=O)NCc3ccc(Cl)cc3)ncc2-c2cn(C)cn2)CC1] | 468.2 |
| 51 | 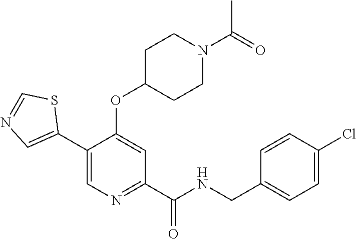 [SMILES: CC(=O)N1CCC(Oc2cc(C(=O)NCc3ccc(Cl)cc3)ncc2-c2cncs2)CC1] | 471.2 |
| 52 | 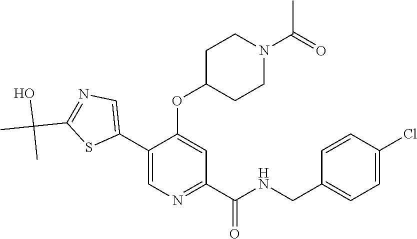 [SMILES: CC(=O)N1CCC(Oc2cc(C(=O)NCc3ccc(Cl)cc3)ncc2-c2cnc(C(C)(C)O)s2)CC1] | 528.1 |
| 53 | 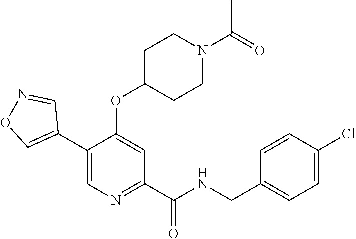 [SMILES: CC(=O)N1CCC(Oc2cc(C(=O)NCc3ccc(Cl)cc3)ncc2-c2cnoc2)CC1] | 455.2 |
| 54 | 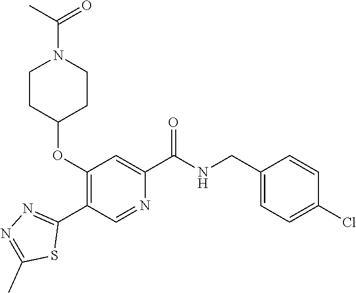 [SMILES: CC(=O)N1CCC(Oc2cc(C(=O)NCc3ccc(Cl)cc3)ncc2-c2nnc(C)s2)CC1] | 486.2 |
| 55 | 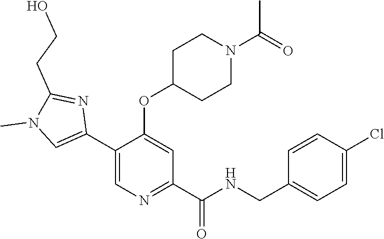 [SMILES: CC(=O)N1CCC(Oc2cc(C(=O)NCc3ccc(Cl)cc3)ncc2-c2cn(C)c(CCO)n2)CC1] | 512.3 |
| 56 | 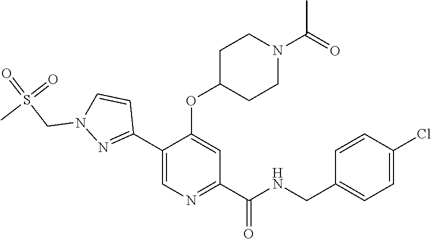 [SMILES: CC(=O)N1CCC(Oc2cc(C(=O)NCc3ccc(Cl)cc3)ncc2-c2ccn(CS(C)(=O)=O)n2)CC1] | 546.2 |
| 57 | 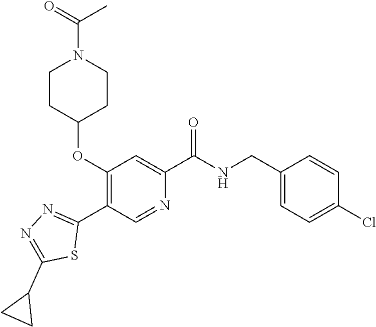 [SMILES: CC(=O)N1CCC(Oc2cc(C(=O)NCc3ccc(Cl)cc3)ncc2-c2nnc(C3CC3)s2)CC1] | 512.1 |
| 58 | 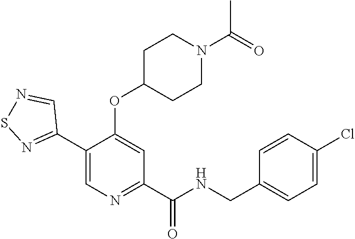 [SMILES: CC(=O)N1CCC(Oc2cc(C(=O)NCc3ccc(Cl)cc3)ncc2-c2cnsn2)CC1] | 472.2 |
| 59 | 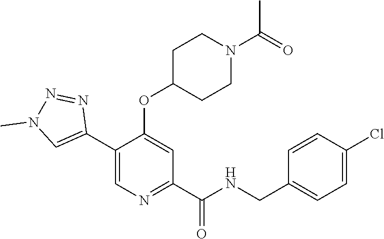 [SMILES: CC(=O)N1CCC(Oc2cc(C(=O)NCc3ccc(Cl)cc3)ncc2-c2cn(C)nn2)CC1] | 469.3 |
| 60 | 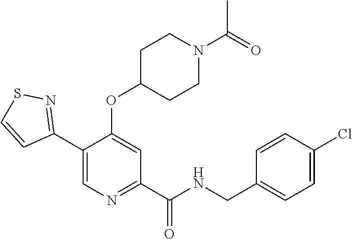 [SMILES: CC(=O)N1CCC(Oc2cc(C(=O)NCc3ccc(Cl)cc3)ncc2-c2ccsn2)CC1] | 471.2 |
| 61 | 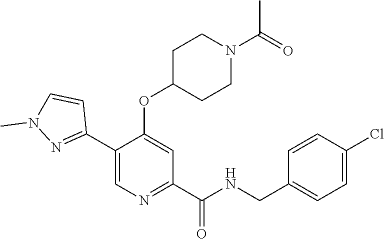 [SMILES: CC(=O)N1CCC(Oc2cc(C(=O)NCc3ccc(Cl)cc3)ncc2-c2ccn(C)n2)CC1] | 468.2 |
| 62 | 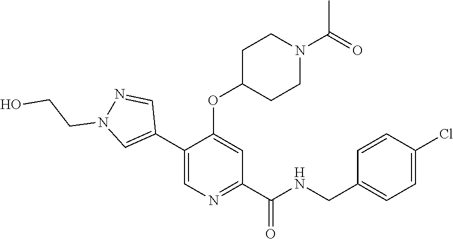 [SMILES: CC(=O)N1CCC(Oc2cc(C(=O)NCc3ccc(Cl)cc3)ncc2-c2cnn(CCO)c2)CC1] | 498.2 |
| 63 | 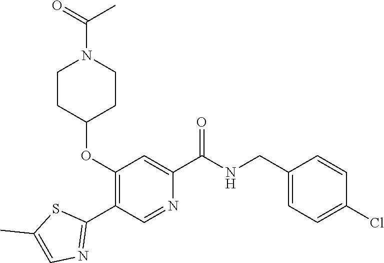 [SMILES: CC(=O)N1CCC(Oc2cc(C(=O)NCc3ccc(Cl)cc3)ncc2-c2ncc(C)s2)CC1] | 485.2 |
| 64 | 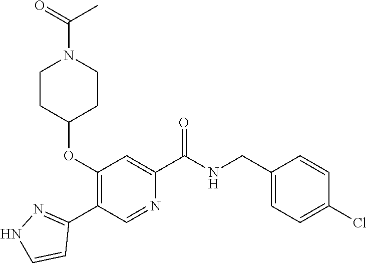 [SMILES: CC(=O)N1CCC(Oc2cc(C(=O)NCc3ccc(Cl)cc3)ncc2-c2cc[nH]n2)CC1] | 454.1 |
| 65 | 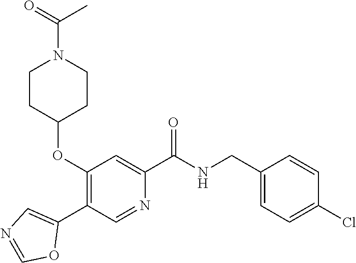 [SMILES: CC(=O)N1CCC(Oc2cc(C(=O)NCc3ccc(Cl)cc3)ncc2-c2cnco2)CC1] | 455.2 |
| 66 | 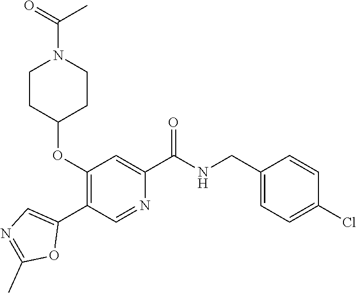 [SMILES: CC(=O)N1CCC(Oc2cc(C(=O)NCc3ccc(Cl)cc3)ncc2-c2cnc(C)o2)CC1] | 469.3 |
| 67 | 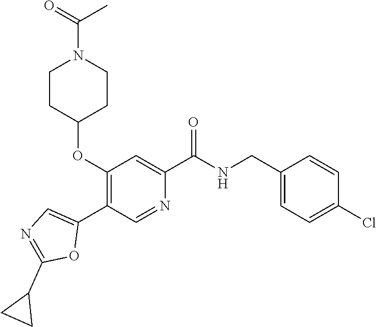 [SMILES: CC(=O)N1CCC(Oc2cc(C(=O)NCc3ccc(Cl)cc3)ncc2-c2cnc(C3CC3)o2)CC1] | 495.2 |
| 68 | 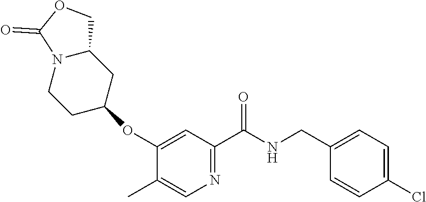 [SMILES: Cc1cnc(C(=O)NCc2ccc(Cl)cc2)cc1O[C@H]1CCN2C(=O)OC[C@@H]2C1] | 416.2 |
| 69 | 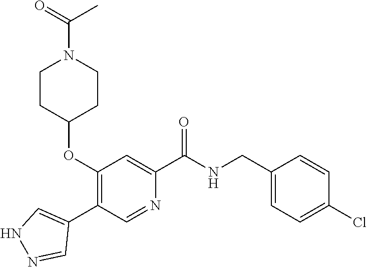 [SMILES: CC(=O)N1CCC(Oc2cc(C(=O)NCc3ccc(Cl)cc3)ncc2-c2cn[nH]c2)CC1] | 454.1 |
| 70 | 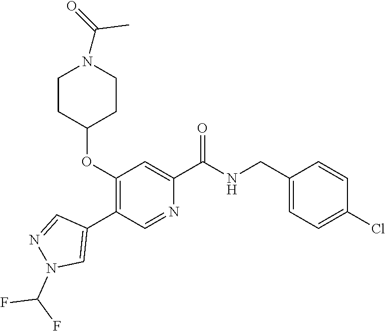 [SMILES: CC(=O)N1CCC(Oc2cc(C(=O)NCc3ccc(Cl)cc3)ncc2-c2cnn(C(F)F)c2)CC1] | 504.3 |
| 71 | 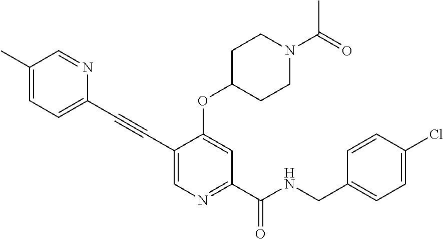 [SMILES: CC(=O)N1CCC(Oc2cc(C(=O)NCc3ccc(Cl)cc3)ncc2C#Cc2ccc(C)cn2)CC1] | 503.4 |
| 72 | 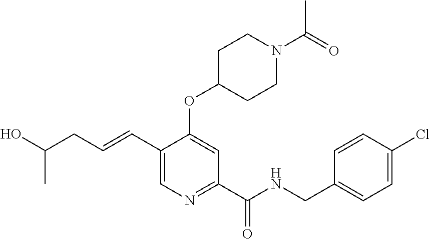 [SMILES: CC(=O)N1CCC(Oc2cc(C(=O)NCc3ccc(Cl)cc3)ncc2/C=C/CC(C)O)CC1] | 472.4 |
| 73 | 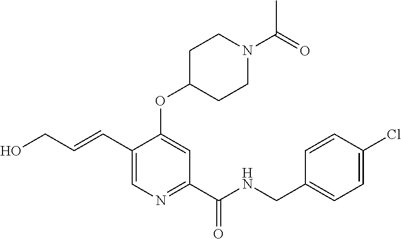 [SMILES: CC(=O)N1CCC(Oc2cc(C(=O)NCc3ccc(Cl)cc3)ncc2/C=C/CO)CC1] | 444.4 |
| 74 | 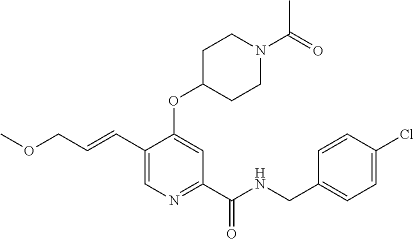 [SMILES: COC/C=C/c1cnc(C(=O)NCc2ccc(Cl)cc2)cc1OC1CCN(C(C)=O)CC1] | 458.5 |
| 75 | 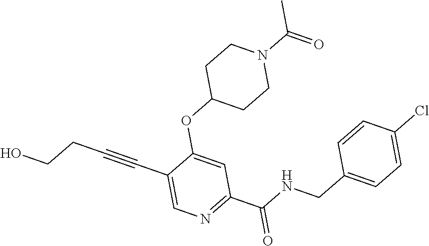 [SMILES: CC(=O)N1CCC(Oc2cc(C(=O)NCc3ccc(Cl)cc3)ncc2C#CCCO)CC1] | 455.1 |
| 76 | 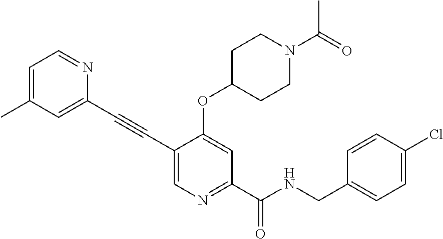 [SMILES: CC(=O)N1CCC(Oc2cc(C(=O)NCc3ccc(Cl)cc3)ncc2C#Cc2cc(C)ccn2)CC1] | 503.4 |
| 77 | 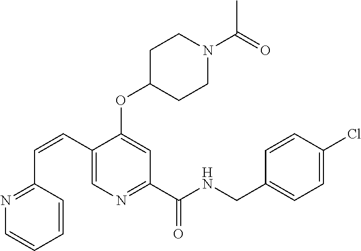 [SMILES: CC(=O)N1CCC(Oc2cc(C(=O)NCc3ccc(Cl)cc3)ncc2/C=C\c2ccccn2)CC1] | 493.4 |
| 78 | 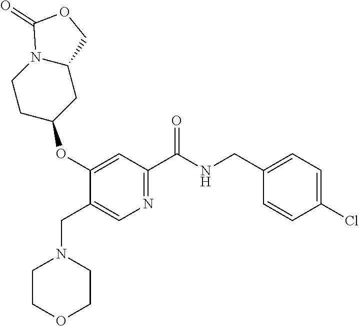 [SMILES: O=C(NCc1ccc(Cl)cc1)c1cc(O[C@H]2CCN3C(=O)OC[C@@H]3C2)c(CN2CCOCC2)cn1] | 501.3 |
| 79 | 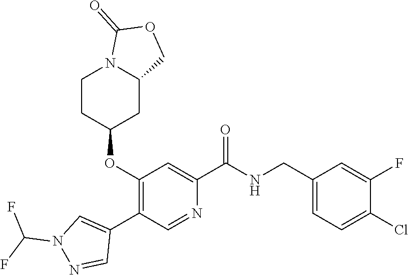 [SMILES: O=C(NCc1ccc(Cl)c(F)c1)c1cc(O[C@H]2CCN3C(=O)OC[C@@H]3C2)c(-c2cnn(C(F)F)c2)cn1] | 536.2 |
| 80 | 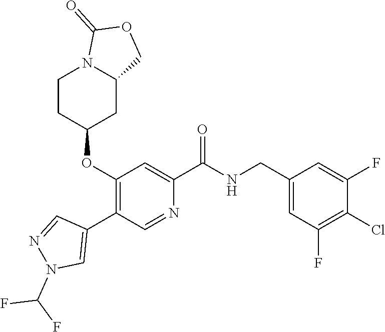 [SMILES: O=C(NCc1cc(F)c(Cl)c(F)c1)c1cc(O[C@H]2CCN3C(=O)OC[C@@H]3C2)c(-c2cnn(C(F)F)c2)cn1] | 554.5 |
| 81 | 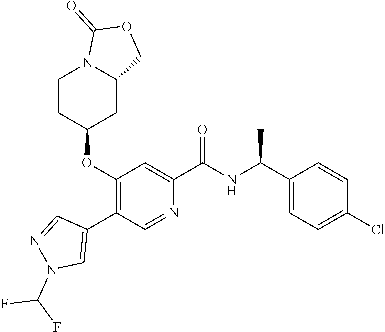 [SMILES: C[C@H](NC(=O)c1cc(O[C@H]2CCN3C(=O)OC[C@@H]3C2)c(-c2cnn(C(F)F)c2)cn1)c1ccc(Cl)cc1] | |
| 82 | 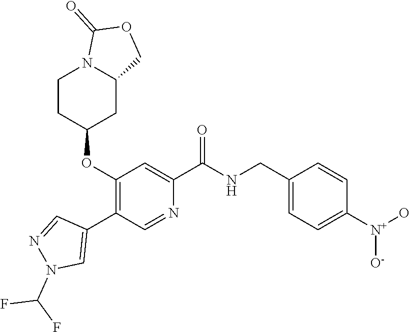 [SMILES: O=C(NCc1ccc([NH+]([O-])O)cc1)c1cc(O[C@H]2CCN3C(=O)OC[C@@H]3C2)c(-c2cnn(C(F)F)c2)cn1] | 529.3 |
| 83 | 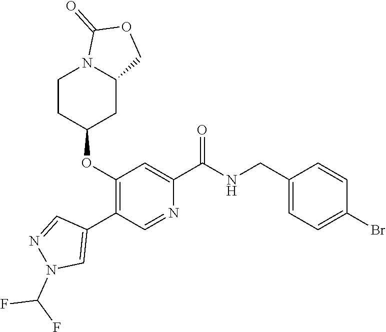 [SMILES: O=C(NCc1ccc(Br)cc1)c1cc(O[C@H]2CCN3C(=O)OC[C@@H]3C2)c(-c2cnn(C(F)F)c2)cn1] | 564.3, 562.0 |
| 84 | 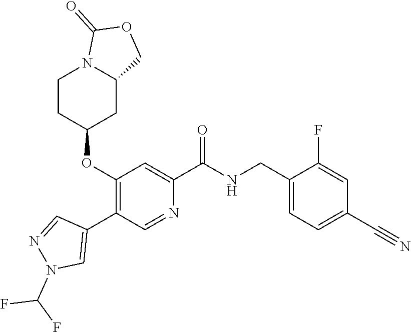 [SMILES: N#Cc1ccc(CNC(=O)c2cc(O[C@H]3CCN4C(=O)OC[C@@H]4C3)c(-c3cnn(C(F)F)c3)cn2)c(F)c1] | 527.4 |
| 85 | 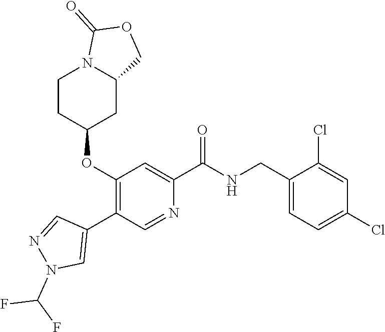 [SMILES: O=C(NCc1ccc(Cl)cc1Cl)c1cc(O[C@H]2CCN3C(=O)OC[C@@H]3C2)c(-c2cnn(C(F)F)c2)cn1] | 552.3 |
| 86 | 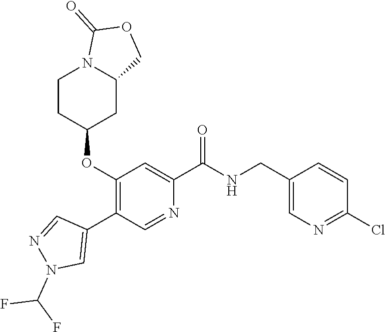 [SMILES: O=C(NCc1ccc(Cl)nc1)c1cc(O[C@H]2CCN3C(=O)OC[C@@H]3C2)c(-c2cnn(C(F)F)c2)cn1] | 519.3 |
| 87 | 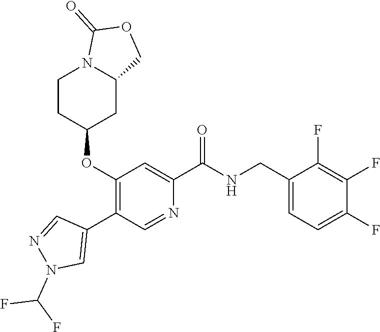 [SMILES: O=C(NCc1ccc(F)c(F)c1F)c1cc(O[C@H]2CCN3C(=O)OC[C@@H]3C2)c(-c2cnn(C(F)F)c2)cn1] | 538.4 |
| 88 | 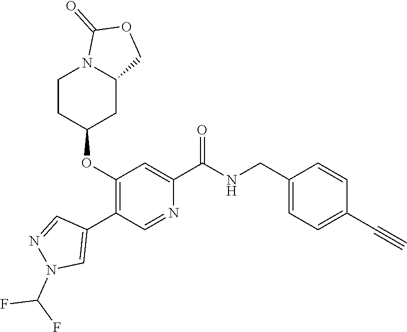 [SMILES: C#Cc1ccc(CNC(=O)c2cc(O[C@H]3CCN4C(=O)OC[C@@H]4C3)c(-c3cnn(C(F)F)c3)cn2)cc1] | 508.3 |
| 89 | 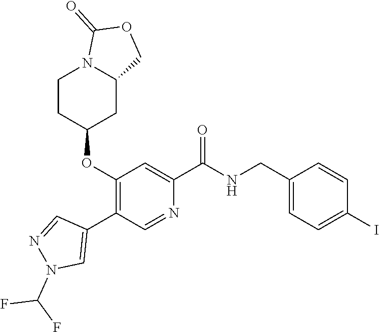 [SMILES: O=C(NCc1ccc(I)cc1)c1cc(O[C@H]2CCN3C(=O)OC[C@@H]3C2)c(-c2cnn(C(F)F)c2)cn1] | 610.3 |
| 90 | 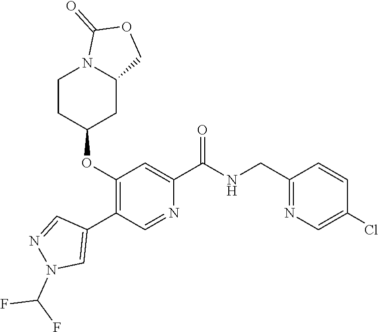 [SMILES: O=C(NCc1ccc(Cl)cn1)c1cc(O[C@H]2CCN3C(=O)OC[C@@H]3C2)c(-c2cnn(C(F)F)c2)cn1] | 519.3 |
| 91 | 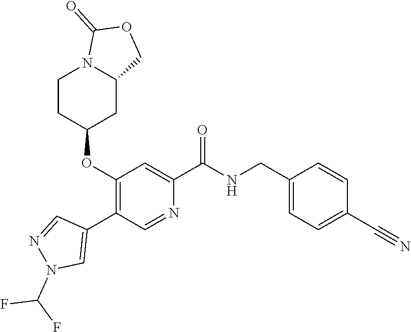 [SMILES: N#Cc1ccc(CNC(=O)c2cc(O[C@H]3CCN4C(=O)OC[C@@H]4C3)c(-c3cnn(C(F)F)c3)cn2)cc1] | 509.4 |
| 92 | 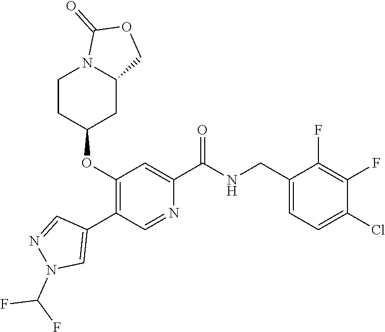 [SMILES: O=C(NCc1ccc(Cl)c(F)c1F)c1cc(O[C@H]2CCN3C(=O)OC[C@@H]3C2)c(-c2cnn(C(F)F)c2)cn1] | 554.5 |
| 93 | 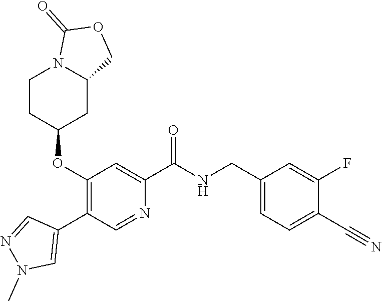 [SMILES: Cn1cc(-c2cnc(C(=O)NCc3ccc(C#N)c(F)c3)cc2O[C@H]2CCN3C(=O)OC[C@@H]3C2)cn1] | 491.2 |
| 94 | 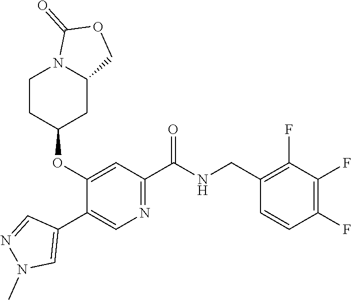 [SMILES: Cn1cc(-c2cnc(C(=O)NCc3ccc(F)c(F)c3F)cc2O[C@H]2CCN3C(=O)OC[C@@H]3C2)cn1] | 502.4 |
| 95* | 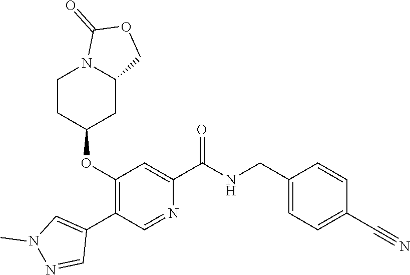 [SMILES: Cn1cc(-c2cnc(C(=O)NCc3ccc(C#N)cc3)cc2O[C@H]2CCN3C(=O)OC[C@@H]3C2)cn1] | 473.3 |
| 96 | 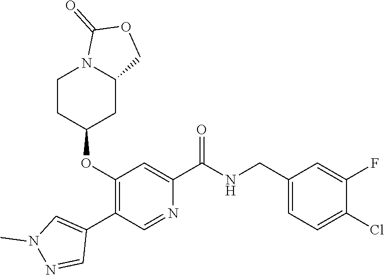 [SMILES: Cn1cc(-c2cnc(C(=O)NCc3ccc(Cl)c(F)c3)cc2O[C@H]2CCN3C(=O)OC[C@@H]3C2)cn1] | 500.3 |
| 97 | 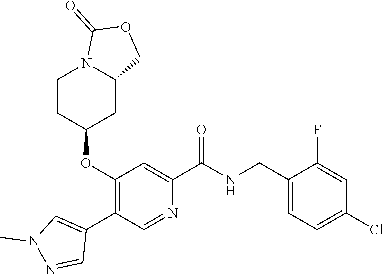 [SMILES: Cn1cc(-c2cnc(C(=O)NCc3ccc(Cl)cc3F)cc2O[C@H]2CCN3C(=O)OC[C@@H]3C2)cn1] | 500.3 |
| 98 | 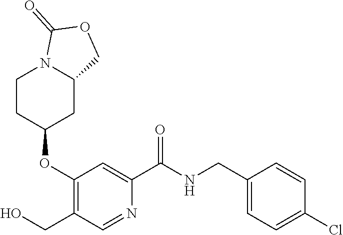 [SMILES: O=C(NCc1ccc(Cl)cc1)c1cc(O[C@H]2CCN3C(=O)OC[C@@H]3C2)c(CO)cn1] | 432.3 |
| 99 | 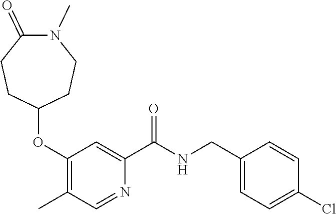 [SMILES: Cc1cnc(C(=O)NCc2ccc(Cl)cc2)cc1OC1CCC(=O)N(C)CC1] | 402.4 |
| 100 | 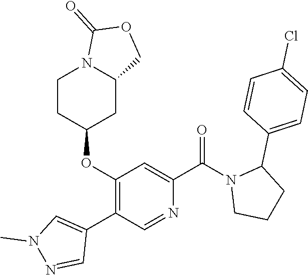 [SMILES: Cn1cc(-c2cnc(C(=O)N3CCCC3c3ccc(Cl)cc3)cc2O[C@H]2CCN3C(=O)OC[C@@H]3C2)cn1] | 522.3 |
| 101 | 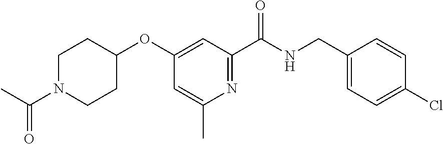 [SMILES: CC(=O)N1CCC(Oc2cc(C)nc(C(=O)NCc3ccc(Cl)cc3)c2)CC1] | 402.3 |
| 102 | 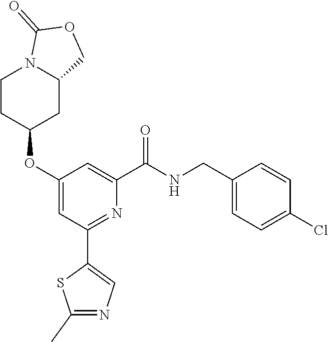 [SMILES: Cc1ncc(-c2cc(O[C@H]3CCN4C(=O)OC[C@@H]4C3)cc(C(=O)NCc3ccc(Cl)cc3)n2)s1] | 499.1 |
| 103 | 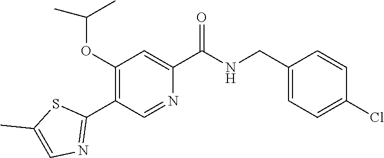 [SMILES: Cc1cnc(-c2cnc(C(=O)NCc3ccc(Cl)cc3)cc2OC(C)C)s1] | 402.2 |
| 104 | 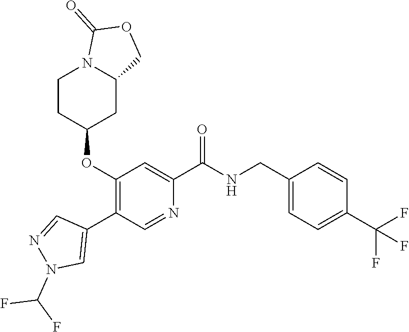 [SMILES: O=C(NCc1ccc(C(F)(F)F)cc1)c1cc(O[C@H]2CCN3C(=O)OC[C@@H]3C2)c(-c2cnn(C(F)F)c2)cn1] | 552.3 |

*denotes trans racemate

### Example 105

### Preparation of Compound 105

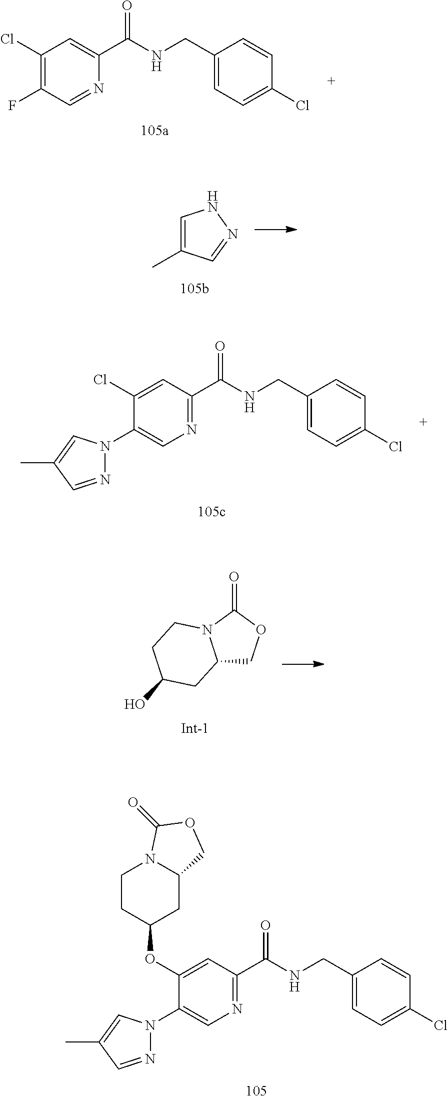

### Step A—Synthesis of Compound 105c

To a solution of 105a (75 mg, 0.25 mmol, made using the method disclosed in Example 2, Step A) in N,N-dimethylformamide (1.2 mL) was added 105b (0.042 mL, 0.50 mmol), and potassium carbonate (139 mg, 1.0 mmol), and the resulting reaction was allowed to stir at 100° C. for 1.5 hours. The reaction mixture was quenched with water (30 mL), and extracted with ethyl acetate. The organic extract was washed with water and saturated aqueous sodium chloride, dried over anhydrous magnesium sulfate, and concentrated in vacuo to provide compound 105c as a residue, which was used without further purification. MS (ESI, m/z): 361.1 [M+H]⁺.

### Step B—Synthesis of Compound 99

To a stirred solution of Int-1 (75 mg, 0.50 mmol) in DMF (1.2 mL) was added sodium hydride (60% by weight in mineral oil, 22 mg, 0.55 mmol), and the resulting reaction was allowed to stir at room temperature for 10 minutes. 105c (90 mg, 0.25 mmol) was added, and the reaction was heated to 125° C. After stirring at this temperature for 30 minutes, the reaction mixture was cooled to room temperature, and purified using preparative HPLC (reverse-phase C-18), eluting with acetonitrile/water+0.1% TFA 10-95%, to provide compound 105. MS (ESI, m/z): 482.2 [M+H]⁺. ¹H NMR (600 MHz, Chloroform-d) δ 8.74 (d, J=8.3 Hz, 1H), 8.41 (s, 2H), 7.91 (d, J=6.9 Hz, 1H), 7.66 (d, J=41.3 Hz, 2H), 7.34-7.28 (m, 3H), 5.18 (s, 1H), 4.63 (s, 2H), 4.39 (d, J=8.3 Hz, 2H), 3.87 (d, J=42.5 Hz, 4H), 3.10 (s, 1H), 2.19-2.02 (br m, 5H).

The following compound of the present invention was made using the methods described in Example 105 above, and substituting the appropriate reactants and/or reagents:

| | | MS |
|---|---|---|
| Compound | Structure | [M + H]⁺ |
| 106 | 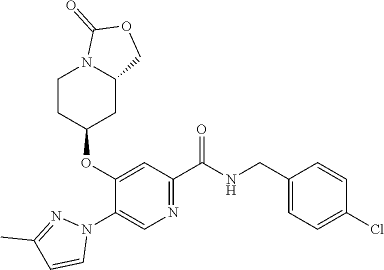 [SMILES: Cc1ccn(-c2cnc(C(=O)NCc3ccc(Cl)cc3)cc2O[C@H]2CCN3C(=O)OC[C@@H]3C2)n1] | 482.3 |

### Example 107

### Preparation of Compound 107

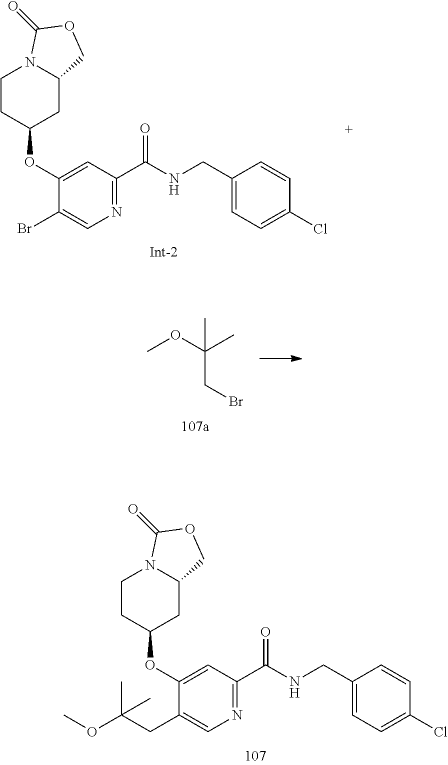

### Step A—Synthesis of Compound 107

To a solution of Int-2 (400 mg, 0.832 mmol) in ethylene glycol dimethyl ether (4 mL) was added 107a (208 mg, 1.248 mmol), nickel(II) chloride ethylene glycol dimethyl ether complex (1.8 mg, 8.32 μmol), 4,4′-di-tert-butyl-2,2′-bipyridine (2.2 mg, 8.32 μmol), Ir[dF(CF₃)ppy]₂(dtbbpy)PF₆ (19 mg, 0.017 mmol), sodium carbonate (176 mg, 1.664 mmol), and 1,1,1,3,3,3-hexamethyl-2-(trimethylsilyl)trisilane (207 mg, 0.832 mmol) at room temperature. The reaction was put under argon atmosphere, then irradiated for 2 hours with single blue LED (400 nm, 15 W), while being cooled via active cooling fan. The reaction mixture was diluted with water (30 mL) and extracted with ethyl acetate (2×30 mL). The combined organic extracts were washed with saturated aqueous sodium chloride (30 mL), dried over anhydrous sodium sulfate, filtered, and concentrated in vacuo. The resulting residue was purified using preparative HPLC, eluting with a gradient of acetonitrile:water (0.8% ammonium bicarbonate)—6:4 to 7:3, to provide compound 107. MS (ESI, m/z): 488.3 [M+H]⁺. ¹H NMR: (300 MHz, methanol-d₄, ppm): δ 8.35 (s, 1H), 7.70 (s, 1H), 7.39-7.33 (m, 4H), 5.11 (s, 1H), 4.58 (s, 2H), 4.48 (t, J=8.4 Hz, 1H), 4.17-4.12 (m, 1H), 4.02-3.96 (m, 1H), 3.79-3.73 (m, 1H), 3.31-3.27 (m, 1H), 3.23 (s, 3H), 2.99-2.89 (m, 2H), 2.32-2.27 (m, 1H), 2.11-2.04 (m, 1H), 1.92-1.77 (m, 2H), 1.21 (s, 6H).

The following compounds of the present invention were made using the methods described in Example 107 above, and substituting the appropriate reactants and/or reagents:

| | | MS |
|---|---|---|
| Compound | Structure | [M + H]⁺ |
| 108 | 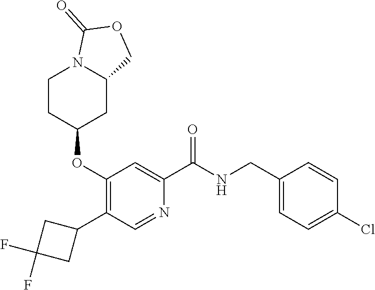 [SMILES: O=C(NCc1ccc(Cl)cc1)c1cc(O[C@H]2CCN3C(=O)OC[C@@H]3C2)c(C2CC(F)(F)C2)cn1] | 492.2 |
| 109 | 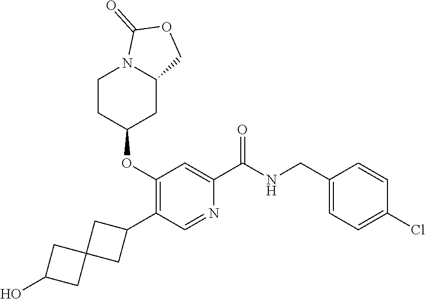 [SMILES: O=C(NCc1ccc(Cl)cc1)c1cc(O[C@H]2CCN3C(=O)OC[C@@H]3C2)c(C2CC3(CC(O)C3)C2)cn1] | 510.1 |
| 110 | 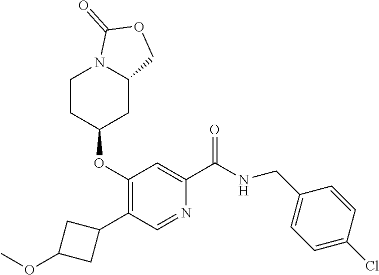 [SMILES: COC1CC(c2cnc(C(=O)NCc3ccc(Cl)cc3)cc2O[C@H]2CCN3C(=O)OC[C@@H]3C2)C1] | 486.2 |
| 111 | 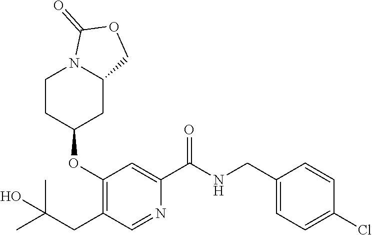 [SMILES: CC(C)(O)Cc1cnc(C(=O)NCc2ccc(Cl)cc2)cc1O[C@H]1CCN2C(=O)OC[C@@H]2C1] | 474.4 |
| 112 | 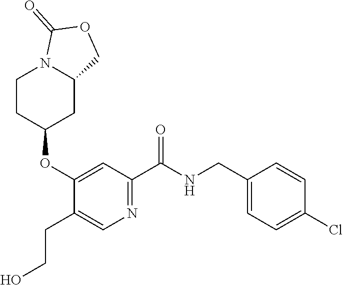 [SMILES: O=C(NCc1ccc(Cl)cc1)c1cc(O[C@H]2CCN3C(=O)OC[C@@H]3C2)c(CCO)cn1] | 446.3 |
| 113* | 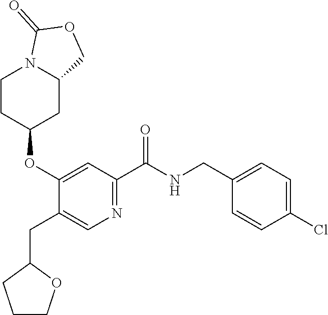 [SMILES: O=C(NCc1ccc(Cl)cc1)c1cc(O[C@H]2CCN3C(=O)OC[C@@H]3C2)c(CC2CCCO2)cn1] | 486.3 |
| 114 | 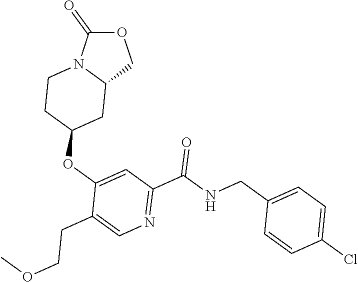 [SMILES: COCCc1cnc(C(=O)NCc2ccc(Cl)cc2)cc1O[C@H]1CCN2C(=O)OC[C@@H]2C1] | 490.3 |
| 115 | 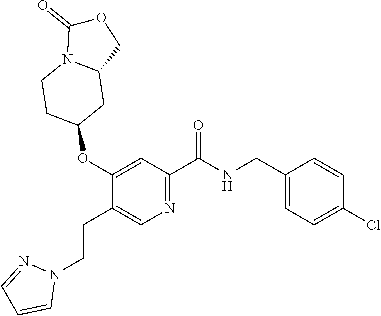 [SMILES: O=C(NCc1ccc(Cl)cc1)c1cc(O[C@H]2CCN3C(=O)OC[C@@H]3C2)c(CCn2cccn2)cn1] | 496.3 |
| 116 | 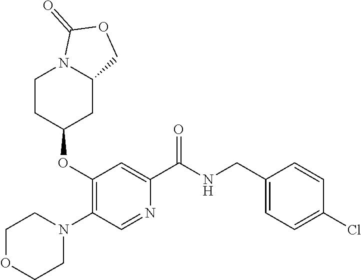 [SMILES: O=C(NCc1ccc(Cl)cc1)c1cc(O[C@H]2CCN3C(=O)OC[C@@H]3C2)c(N2CCOCC2)cn1] | 487.3 |

*denotes a mixture of 2 diastereomers

### Example 117

### Preparation of Compound 117

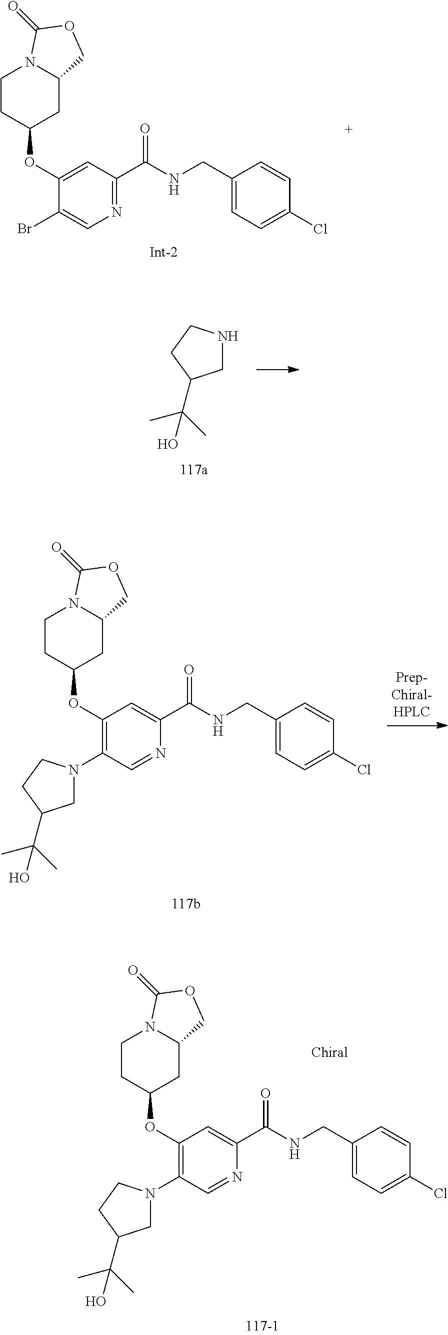

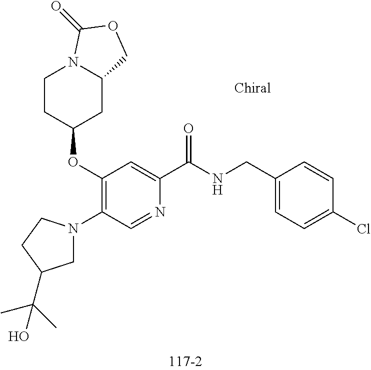 [SMILES: CC(C)(O)C1CCN(c2cnc(C(=O)NCc3ccc(Cl)cc3)cc2O[C@H]2CCN3C(=O)OC[C@@H]3C2)C1]

### Step A—Synthesis of Compound 117b

To a solution of Int-2 (80 mg, 0.166 mmol) in 1,4-dioxane (3 mL) was added 117a (32 mg, 0.250 mmol), cesium carbonate (163 mg, 0.499 mmol), Ad₂(n-Bu)P Palladacycle Gen. 2 (11 mg, 0.017 mmol), and (R)-(+)-2,2′-bis(diphenylphosphino)-1,1′-binaphthyl (10 mg, 0.017 mmol). The reaction was allowed to stir for 16 hours at 90° C. under nitrogen atmosphere, then diluted with water (20 mL). The resulting solution was extracted with ethyl acetate (3×20 mL), and the combined organic extracts were washed with saturated aqueous sodium chloride (20 mL), dried over anhydrous sodium sulfate, filtered, and concentrated in vacuo. The resulting residue was purified using silica gel chromatography, eluting with a gradient of methanol:dichloromethane—0:1 to 1:9, to provide 117b as a mixture of isomers. MS (ESI, m/z): 529.3 [M+H]⁺.

Step B—Chiral Resolution to of 117b to provide 117-1 and 117-2.

117b (90 mg, isomeric mixture) was resolved using Chiral-Prep-HPLC (column: CHIRALPAK IA), eluting with a gradient of hexane:dichloromethane:ethanol—1:1:2 to provide compound 117-1 (slow peak). MS (ESI, m/z): 529.3 [M+H]⁺. ¹H NMR (400 MHz, methanol-d₄, ppm): δ 7.87 (s, 1H), 7.60 (s, 1H), 7.34-7.29 (m, 4H), 5.07 (s, 1H), 4.55 (s, 2H), 4.46 (t, J=8.4 Hz, 1H), 4.14-4.12 (m, 1H), 3.98 (dd, J=5.6 Hz, 8.4 Hz, 1H), 3.79-3.74 (m, 1H), 3.65 (t, J=9.6 Hz, 1H), 3.58-3.47 (m, 3H), 2.37-2.26 (m, 2H), 2.15-2.11 (m, 1H), 2.04-1.91 (m, 1H), 1.88-1.86 (m, 2H), 1.81-1.74 (m, 1H), 1.36-1.29 (m, 1H), 1.25 (s, 6H). And compound 117-b (fast peak). MS (ESI, m/z): 529.3 [M+H]⁺. ¹H NMR (400 MHz, methanol-d₄, ppm): δ 7.87 (s, 1H), 7.59 (s, 1H), 7.33-7.28 (m, 4H), 5.04 (s, 1H), 4.54 (s, 2H), 4.46 (t, J=8.4 Hz, 1H), 4.13-4.11 (m, 1H), 3.98 (dd, J=6.0, 8.8 Hz, 1H), 3.74 (dd, J=5.2, 13.6 Hz, 1H), 3.63 (t, J=9.6 Hz, 1H), 3.57-3.53 (m, 2H), 3.51-3.46 (m, 1H), 3.31-3.26 (m, 1H), 2.34-2.31 (m, 2H), 2.07-2.00 (m, 2H), 1.90-1.74 (m, 3H), 1.25 (s, 6H).

The following compounds of the present invention were made using the methods described in Example 117 above, and substituting the appropriate reactants and/or reagents:

| | | MS |
|---|---|---|
| Compound | Structure | [M + H]⁺ |
| 118 | 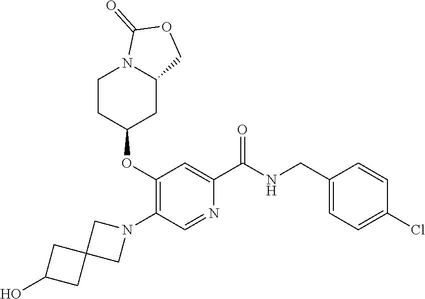 [SMILES: O=C(NCc1ccc(Cl)cc1)c1cc(O[C@H]2CCN3C(=O)OC[C@@H]3C2)c(N2CC3(CC(O)C3)C2)cn1] | 513.3 |
| 119 | 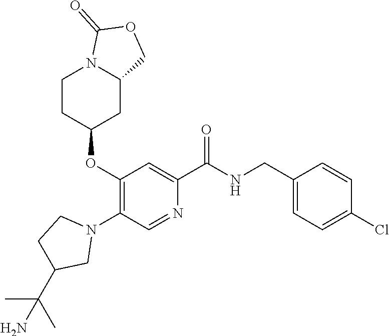 [SMILES: CC(C)(N)C1CCN(c2cnc(C(=O)NCc3ccc(Cl)cc3)cc2O[C@H]2CCN3C(=O)OC[C@@H]3C2)C1] | 528.3 |
| 120 | 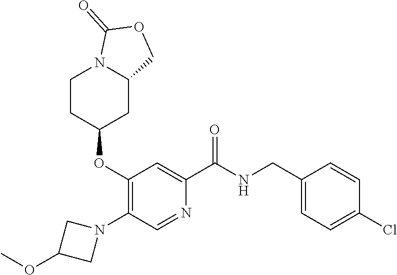 [SMILES: COC1CN(c2cnc(C(=O)NCc3ccc(Cl)cc3)cc2O[C@H]2CCN3C(=O)OC[C@@H]3C2)C1] | 487.2 |
| 121 | 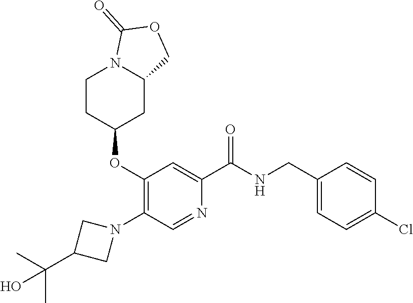 [SMILES: CC(C)(O)C1CN(c2cnc(C(=O)NCc3ccc(Cl)cc3)cc2O[C@H]2CCN3C(=O)OC[C@@H]3C2)C1] | 515.3 |
| 122 | 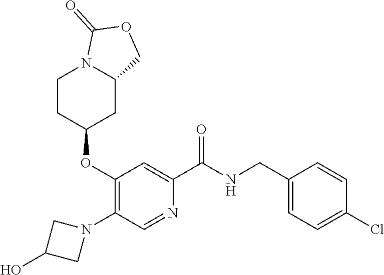 [SMILES: O=C(NCc1ccc(Cl)cc1)c1cc(O[C@H]2CCN3C(=O)OC[C@@H]3C2)c(N2CC(O)C2)cn1] | 473.4 |
| 123 | 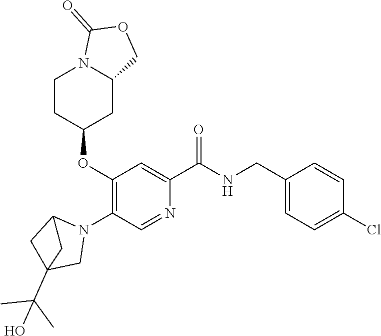 [SMILES: CC(C)(O)C12CC(C1)N(c1cnc(C(=O)NCc3ccc(Cl)cc3)cc1O[C@H]1CCN3C(=O)OC[C@@H]3C1)C2] | 541.2 |
| 124 | 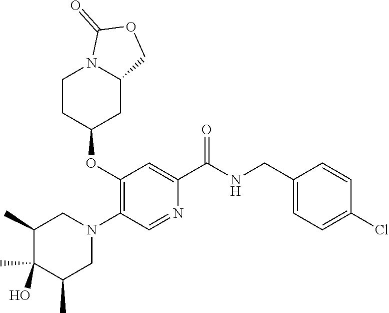 [SMILES: C[C@@H]1CN(c2cnc(C(=O)NCc3ccc(Cl)cc3)cc2O[C@H]2CCN3C(=O)OC[C@@H]3C2)C[C@H](C)[C@]1(C)O] | 543.3 |

### Example 125

### Preparation of Compound 125

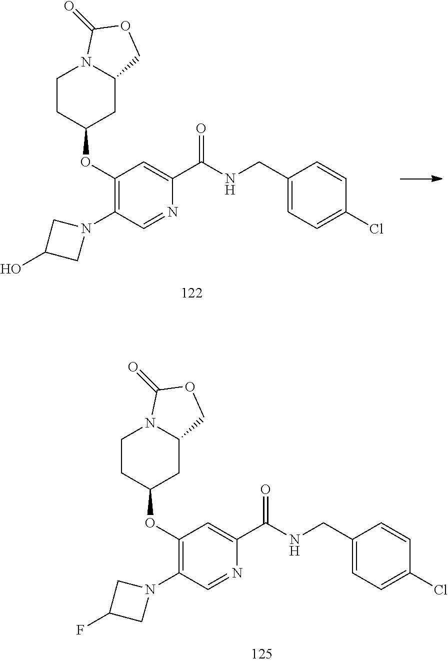 [SMILES: O=C(NCc1ccc(Cl)cc1)c1cc(O[C@H]2CCN3C(=O)OC[C@@H]3C2)c(N2CC(O)C2)cn1 ; O=C(NCc1ccc(Cl)cc1)c1cc(O[C@H]2CCN3C(=O)OC[C@@H]3C2)c(N2CC(F)C2)cn1]

### Step A—Synthesis of Compound 125

A solution of 122 (30 mg, 0.063 mmol) in dichloromethane (0.60 mL) was cooled to −20° C. and treated with DAST (0.025 mL, 0.19 mmol), then allowed to warm on its own room temperature overnight. The reaction mixture was diluted with saturated sodium bicarbonate and water, then extracted twice with dichloromethane. The combined organic extracts were washed with brine, dried over anhydrous magnesium sulfate, and concentrated in vacuo. The resulting residue was purified using silica gel column chromatography, eluting with a gradient of 0-100% (3:1 Ethyl acetate:ethanol)/hexanes, to provide compound 125 as a residue. MS (ESI, m/z): 475.2 [M+H]⁺. ¹H NMR (500 MHz, Chloroform-d) δ 8.19 (t, J=6.1 Hz, 1H), 7.65 (s, 1H), 7.59 (s, 1H), 7.31 (dd, J=8.1, 5.7 Hz, 4H), 5.54-5.38 (m, 1H), 5.05 (s, 1H), 4.61 (d, J=6.2 Hz, 2H), 4.50-4.37 (m, 3H), 4.24-4.10 (m, 1H), 4.06-3.86 (m, 3H), 3.24 (td, J=13.2, 3.3 Hz, 1H), 2.30 (d, J=13.6 Hz, 1H), 2.11 (s, 2H), 1.94-1.83 (m, 1H), 1.79-1.69 (m, 1H).

### Example 126

### Preparation of Compound 126

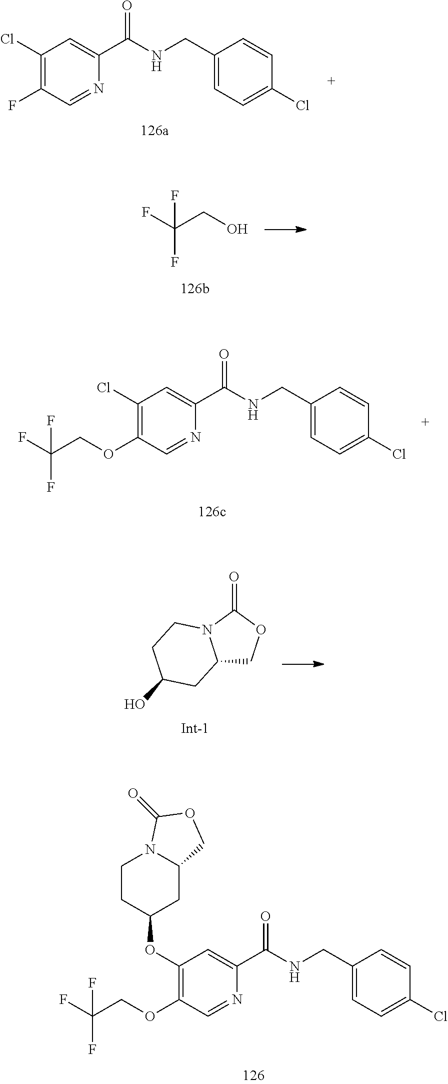 [SMILES: O=C(NCc1ccc(Cl)cc1)c1cc(Cl)c(F)cn1 ; OCC(F)(F)F ; O=C(NCc1ccc(Cl)cc1)c1cc(Cl)c(OCC(F)(F)F)cn1 ; O=C1OC[C@@H]2C[C@@H](O)CCN12 ; O=C(NCc1ccc(Cl)cc1)c1cc(O[C@H]2CCN3C(=O)OC[C@@H]3C2)c(OCC(F)(F)F)cn1]

### Step A—Synthesis of Compound 126c

To a solution of 126a (0.024 mL, 0.33 mmol, made using the methodology described in Example 2, Step A) in dimethylformamide (1.3 mL) at 25° C. was added potassium tert-butoxide (56 mg, 0.50 mmol), and the resulting suspension was allowed to stir at room temperature for 15 minutes. 126b (100 mg, 0.33 mmol) was added to the reaction mixture. The reaction was allowed to stir at room temperature for 2 hours, then was quenched with water and diluted with ethyl acetate. The organic extract was washed with water and saturated aqueous sodium chloride, dried over magnesium sulfate, and was concentrated in vacuo. The resulting residue was purified using silica gel chromatography, eluting with a gradient of ethyl acetate:hexanes—0:100 to 50:50, to provide 126c. MS: m/z=479.2 [M+H].

### Step B—Synthesis of Compound 126

To a solution of Int-1 (59 mg, 0.37 mmol) in dimethylformamide (2.0 mL) at 25° C. was added sodium hydride (20 mg, 0.49 mmol), and the resulting suspension was allowed to stir for 10 minutes. 126c (110 mg, 0.29 mmol) was added, and the reaction mixture was heated to 125° C. for 2 hours. The reaction mixture was quenched with water and diluted with ethyl acetate. The organic extracts were washed with water and saturated aqueous sodium chloride, dried over magnesium sulfate, and was concentrated in vacuo. The resulting residue was purified using reverse phase HPLC ((10-95% water (0.1% TFA)/acetonitrile (0.1%) TFA, 15 minute gradient, column—Sunfire prep C18 OBD 10 micron, 20×150 mm) to provide compound 126. MS: m/z=500.3 [M+H]⁺. ¹H NMR (600 MHz, Chloroform-d) δ 8.26 (s, 1H), 8.14 (s, 1H), 7.81 (s, 1H), 7.33-7.26 (m, 4H), 5.07 (s, 1H), 4.60 (d, J=6.2 Hz, 2H), 4.49-4.41 (m, 3H), 4.13-4.05 (m, 1H), 3.94 (dd, J=8.6, 5.5 Hz, 1H), 3.84 (dd, J=13.7, 5.2 Hz, 1H), 3.26 (td, J=13.5, 3.5 Hz, 1H), 1.87 (m, 4H).

### Example 127

### Preparation of Compound 127

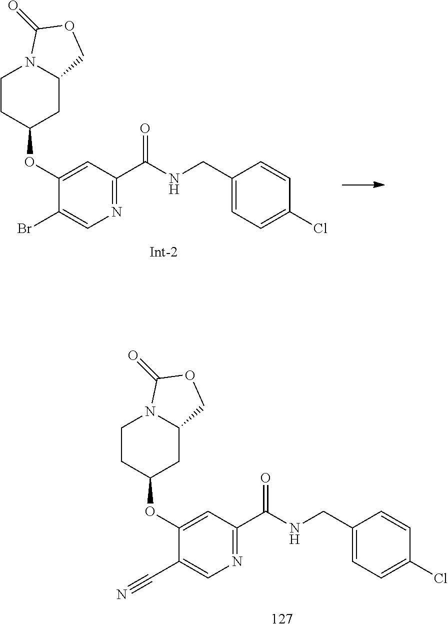 [SMILES: O=C(NCc1ccc(Cl)cc1)c1cc(O[C@H]2CCN3C(=O)OC[C@@H]3C2)c(Br)cn1 ; N#Cc1cnc(C(=O)NCc2ccc(Cl)cc2)cc1O[C@H]1CCN2C(=O)OC[C@@H]2C1]

### Step A—Synthesis of Compound 127

A solution of Int-2 (35 mg, 0.073 mmol), dicyanozinc (13 mg, 0.11 mmol), and tetrakis(triphenylphosphine)palladium(0) (17 mg, 0.015 mmol) in dimethylformamide (0.35 mL) was purged subsurface with nitrogen, sealed, and irradiated at 140° C. for 30 minutes. The reaction was filtered, and the filtrate was directly purified using preparative HPLC (reverse-phase C-18), eluting with acetonitrile/water+0.1% TFA 10-95%, to provide compound 127. MS (ESI, m/z): 427.2 [M+H]⁺. ¹H NMR (600 MHz, Chloroform-d) δ 8.63 (s, 1H), 8.33 (s, 1H), 7.85 (s, 1H), 7.32 (d, J=8.5 Hz, 2H), 7.27 (d, J=8.6 Hz, 2H), 5.21 (s, 1H), 4.62 (d, J=6.2 Hz, 2H), 4.47 (t, J=8.3 Hz, 1H), 4.23-4.16 (m, 1H), 3.96 (dd, J=8.7, 5.2 Hz, 1H), 3.87 (dd, J=14.0, 5.0 Hz, 1H), 3.31 (td, J=13.3, 3.2 Hz, 1H), 2.25 (d, J=12.0 Hz, 1H), 2.18-2.02 (m, 1H), 1.95-1.87 (m, 1H), 1.86-1.78 (m, 1H).

The following compound of the present invention was made using the methods described in Example 127 above, and substituting the appropriate reactants and/or reagents:

| Compound | Structure | MS [M + H]⁺ |
|---|---|---|
| 128 | 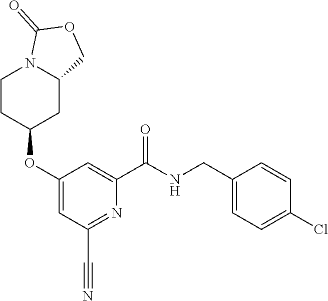 [SMILES: N#Cc1cc(O[C@H]2CCN3C(=O)OC[C@@H]3C2)cc(C(=O)NCc2ccc(Cl)cc2)n1] | 427.2 |

### Example 129

### Preparation of Compound 129

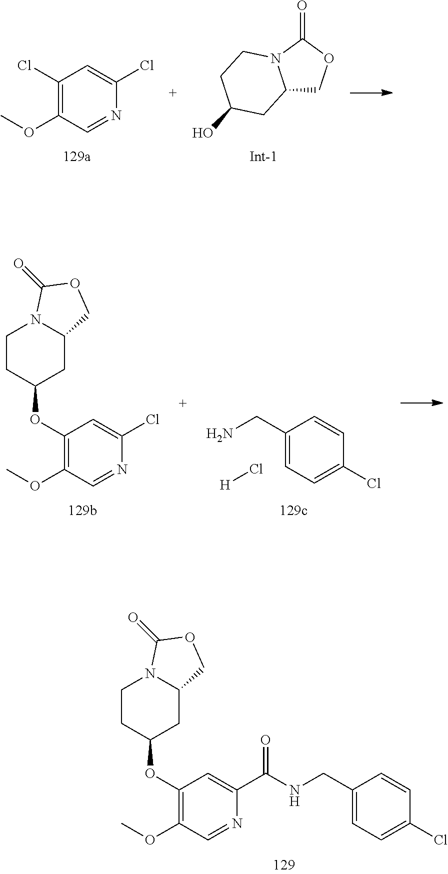

### Step A—Synthesis of Compound 129b

To a solution of Int-1 (71 mg, 0.45 mmol) in dimethylformamide (1.0 mL) at 25° C. was added sodium hydride (60% in mineral oil, 18 mg, 0.45 mmol), and the resulting suspension was allowed to stir at room temperature for 15 minutes. A solution of 129a (71 mg, 0.45 mmol) in dimethylformamide (1.5 mL) was added, and the reaction mixture was allowed to stir at room temperature for 2 hours, then the reaction mixture was then heated to 60° C. and allowed to stir at this temperature overnight. The reaction was quenched with water and diluted with ethyl acetate. The organic extract was washed with water and saturated aqueous sodium chloride, dried over magnesium sulfate, and concentrated in vacuo. The resulting residue was purified using silica gel chromatography, eluting with a gradient of ethyl acetate:hexanes—0:100 to 100:0, to provide 129b as an oil. MS: m/z=299.2 [M+H]⁺.

### Step B—Synthesis of Compound 129

To a solution of 129b (25 mg, 0.084 mmol) in dimethylformamide (0.50 mL) at 25° C. was added 129c (74 mg, 0.42 mmol), DIEA (0.073 mL, 0.42 mmol), and 1,1′-bis(diphenylphosphino)ferrocene-palladium(II)dichloride (61 mg, 0.084 mmol). The resulting reaction mixture was stirred under an atmosphere of CO₂ at 60° C. and 60 PSI for 16 hours. The reaction mixture was then filtered, and the filtrate was directly purified using reverse phase HPLC ((10-100% water (0.1% TFA)/acetonitrile (0.1%) TFA, 15 minutes gradient, column—Sunfire prep C18 OBD 10 micron, 20×150 mm) to provide 129 as a solid. MS: m/z=432.3 [M+H]⁺. ¹H NMR (600 MHz, Chloroform-d) δ 8.52 (s, 1H), 8.10 (s, 1H), 7.78 (s, 1H), 7.29 (q, J=8.4 Hz, 4H), 5.08 (s, 1H), 4.60 (d, J=6.0 Hz, 2H), 4.45 (t, J=8.4 Hz, 1H), 4.15 (s, 1H), 3.99 (s, 3H), 3.97-3.92 (m, 1H), 3.82 (dd, J=13.7, 5.3 Hz, 1H), 3.38-3.25 (m, 1H), 2.26 (d, J=14.6 Hz, 1H), 2.09 (d, J=11.2 Hz, 1H), 1.85 (d, J=8.7 Hz, 1H), 1.74 (d, J=26.1 Hz, 1H).

### Example 130

### Preparation of Compound 130

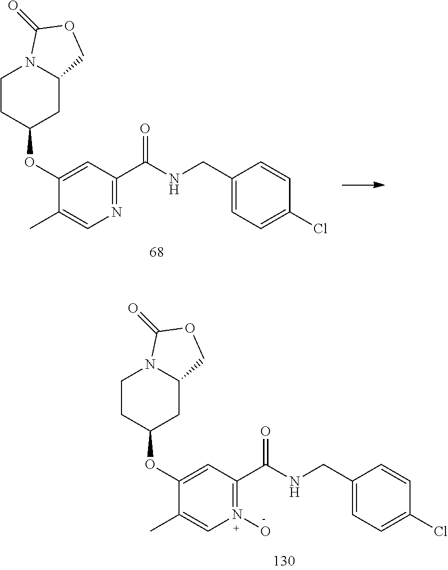

### Step A—Synthesis of Compound 130

To a solution of compound 68 (40 mg, 0.096 mmol) in dichloromethane (0.50 mL) was added m-CPBA (25 mg, 0.14 mmol), and the resulting solution was allowed to stir at room temperature for 18 hours. The reaction mixture was concentrated in vacuo, and the resulting residue was purified using preparative HPLC (reverse-phase C-18), eluting with acetonitrile/water+0.1% TFA 10-95%, to provide compound 130 as a solid. MS (ESI, m/z): 432.2 [M+H]⁺. ¹H NMR (600 MHz, Chloroform-d) δ 12.12 (s, 1H), 8.03 (s, 1H), 7.79 (s, 1H), 7.27 (s, 4H), 5.00 (s, 1H), 4.60 (d, J=5.9 Hz, 2H), 4.42 (t, J=8.3 Hz, 1H), 4.06-3.98 (m, 1H), 3.92 (dd, J=8.6, 5.8 Hz, 1H), 3.84 (dd, J=13.5, 5.1 Hz, 1H), 3.20 (td, J=13.4, 3.3 Hz, 1H), 2.22 (s, 4H), 2.06 (d, J=14.6 Hz, 1H), 1.88-1.80 (m, 1H), 1.77-1.69 (m, 1H).

The following compounds of the present invention were made using the methods described in Example 130 above, and substituting the appropriate reactants and/or reagents:

| | | MS |
|---|---|---|
| Compound | Structure | [M + H]⁺ |
| 131 | 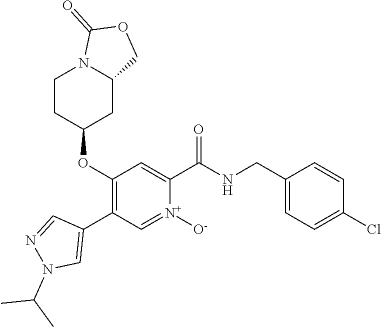 [SMILES: CC(C)n1cc(-c2c[n+]([O-])c(C(=O)NCc3ccc(Cl)cc3)cc2O[C@H]2CCN3C(=O)OC[C@@H]3C2)cn1] | 526.4 |
| 132 | 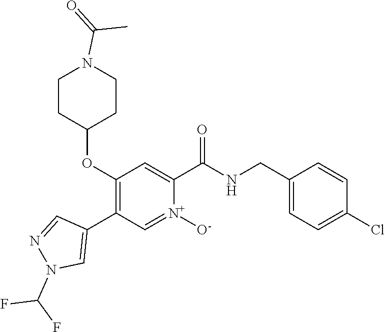 [SMILES: CC(=O)N1CCC(Oc2cc(C(=O)NCc3ccc(Cl)cc3)[n+]([O-])cc2-c2cnn(C(F)F)c2)CC1] | 520.3 |

### Example 133

### Preparation of Compound 133

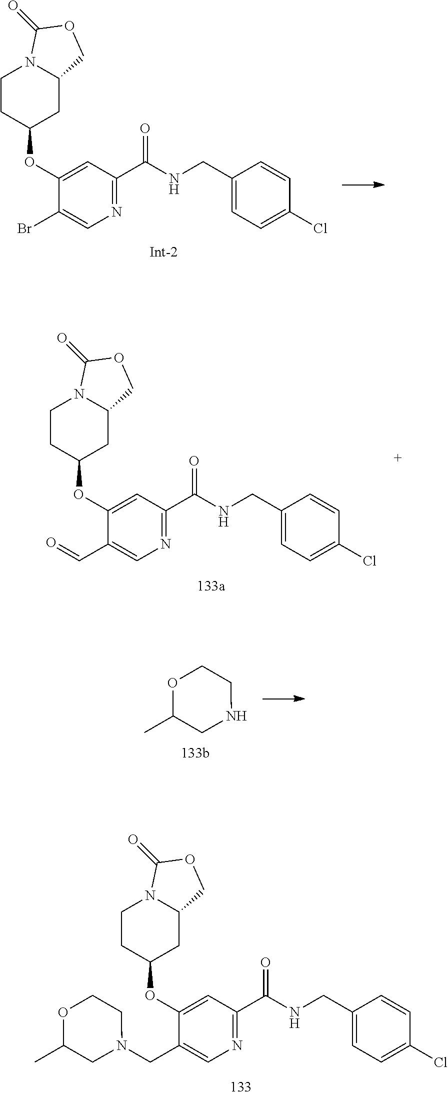

### Step A—Synthesis of Compound 133a

To a solution of Int-2 (480 mg, 1.0 mmol) in THE (5.0 mL) at −78° C. was added turbogrignard (1.3 M solution, 1.5 mL, 2.0 mmol), and the reaction mixture was warmed and held at 0° C. for 30 minutes. The reaction mixture was then re-cooled to −78° C., DMF (0.23 mL, 3.0 mmol) was added, and the reaction mixture was warmed to room temperature, and stirred at that temperature for 3 hours. The reaction was then quenched with acetic acid and poured into ethyl acetate and water. The organic phase was washed with brine, dried over sodium sulfate, filtered, and concentrated in vacuo. The resulting residue was purified using preparative HPLC (reverse-phase C-18), eluting with acetonitrile/water+0.1% TFA 10-95%, to provide compound 133a as a solid. MS (ESI, m/z): 430.3 [M+H]⁺.

### Step B—Synthesis of Compound 133

To a solution of 133a (15 mg, 0.35 mmol) in dichloroethane (0.35 mL), and AcOH (0.008 mL) was added 133b (7 mg, 0.07 mmol), and resin bound sodium cyanoborohydride. The reaction mixture was shaken at 80° C. for 3 hours, then filtered and the filtrate was directly purified using preparative HPLC (reverse-phase C-18), eluting with acetonitrile/water+0.1% TFA 10-95%, to provide compound 133. MS (ESI, m/z): 529.2 [M+H]⁺. ¹H NMR (500 MHz, DMSO-d₆) δ 9.48 (t, J=6.4 Hz, 1H), 8.61 (s, 1H), 7.71 (s, 1H), 7.46-7.26 (m, 4H), 5.22 (s, 1H), 4.47 (d, J=6.5 Hz, 2H), 4.36 (q, J=8.4 Hz, 2H), 4.14-3.89 (m, 3H), 3.61-3.48 (m, 1H), 3.41 (s, 3H), 3.31-3.13 (m, 1H), 2.54 (s, 3H), 2.15 (d, J=13.5 Hz, 1H), 1.94 (s, 1H), 1.83-1.67 (m, 2H), 1.11 (d, J=6.0 Hz, 3H).

The following compounds of the present invention were made using the methods described in Example 133 above, and substituting the appropriate reactants and/or reagents:

| | | MS |
|---|---|---|
| Compound | Structure | [M + H]⁺ |
| 134* | 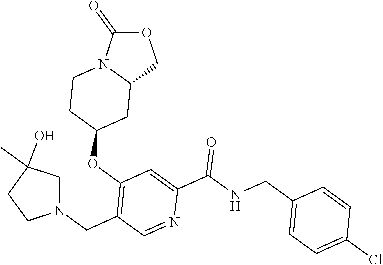 [SMILES: CC1(O)CCN(Cc2cnc(C(=O)NCc3ccc(Cl)cc3)cc2O[C@H]2CCN3C(=O)OC[C@@H]3C2)C1] | 515.3 |
| 135 | 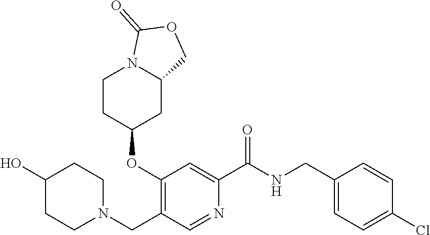 [SMILES: O=C(NCc1ccc(Cl)cc1)c1cc(O[C@H]2CCN3C(=O)OC[C@@H]3C2)c(CN2CCC(O)CC2)cn1] | 515.3 |
| 136 | 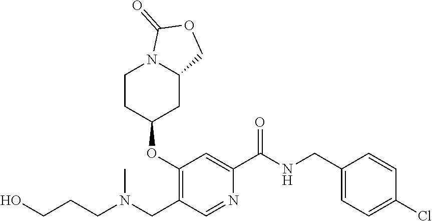 [SMILES: CN(CCCO)Cc1cnc(C(=O)NCc2ccc(Cl)cc2)cc1O[C@H]1CCN2C(=O)OC[C@@H]2C1] | 503.3 |
| 137* | 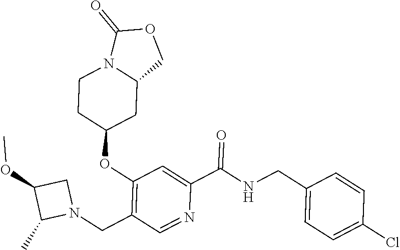 [SMILES: CO[C@H]1CN(Cc2cnc(C(=O)NCc3ccc(Cl)cc3)cc2O[C@H]2CCN3C(=O)OC[C@@H]3C2)[C@@H]1C] | 515.3 |
| 138 | 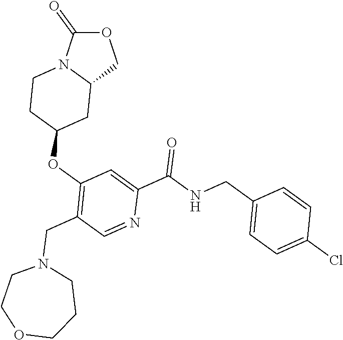 [SMILES: O=C(NCc1ccc(Cl)cc1)c1cc(O[C@H]2CCN3C(=O)OC[C@@H]3C2)c(CN2CCCOCC2)cn1] | 515.3 |
| 139 | 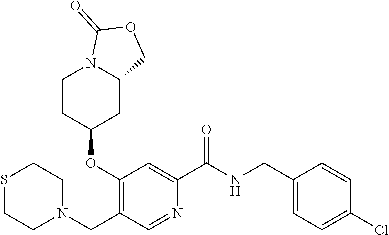 [SMILES: O=C(NCc1ccc(Cl)cc1)c1cc(O[C@H]2CCN3C(=O)OC[C@@H]3C2)c(CN2CCSCC2)cn1] | 517.1 |
| 140* | 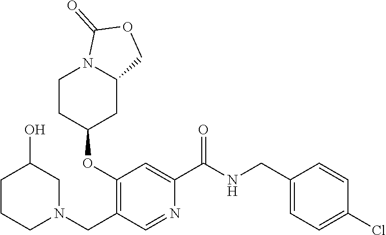 [SMILES: O=C(NCc1ccc(Cl)cc1)c1cc(O[C@H]2CCN3C(=O)OC[C@@H]3C2)c(CN2CCCC(O)C2)cn1] | 515.3 |
| 141 | 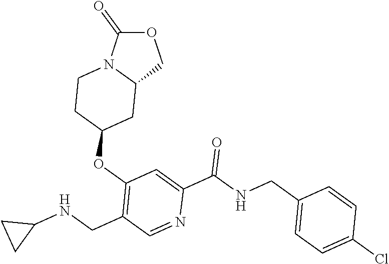 [SMILES: O=C(NCc1ccc(Cl)cc1)c1cc(O[C@H]2CCN3C(=O)OC[C@@H]3C2)c(CNC2CC2)cn1] | 471.2 |
| 142* | 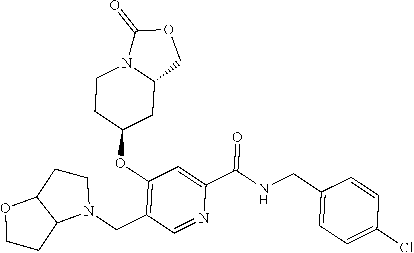 [SMILES: O=C(NCc1ccc(Cl)cc1)c1cc(O[C@H]2CCN3C(=O)OC[C@@H]3C2)c(CN2CCC3OCCC32)cn1] | 527.3 |
| 143 | 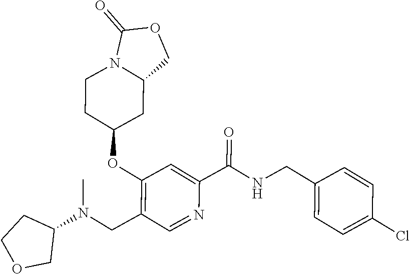 [SMILES: CN(Cc1cnc(C(=O)NCc2ccc(Cl)cc2)cc1O[C@H]1CCN2C(=O)OC[C@@H]2C1)[C@H]1CCOC1] | 515.3 |
| 144 | 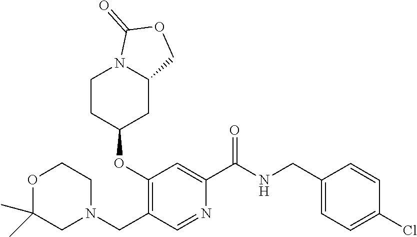 [SMILES: CC1(C)CN(Cc2cnc(C(=O)NCc3ccc(Cl)cc3)cc2O[C@H]2CCN3C(=O)OC[C@@H]3C2)CCO1] | 515.2 |
| 145 | 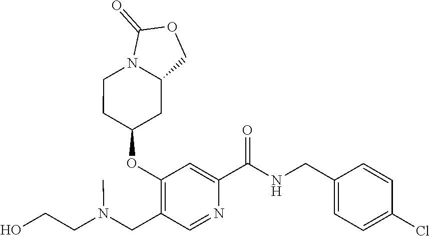 [SMILES: CN(CCO)Cc1cnc(C(=O)NCc2ccc(Cl)cc2)cc1O[C@H]1CCN2C(=O)OC[C@@H]2C1] | 531.1 |

*denotes a mixture of diastereomers

### Example 146

### Preparation of Compound 146

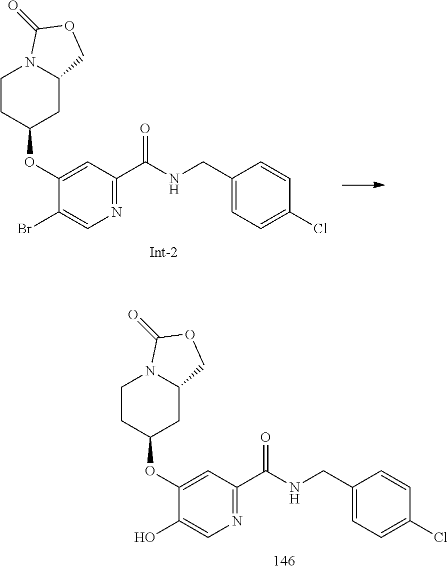 [SMILES: O=C(NCc1ccc(Cl)cc1)c1cc(O[C@H]2CCN3C(=O)OC[C@@H]3C2)c(Br)cn1 ; O=C(NCc1ccc(Cl)cc1)c1cc(O[C@H]2CCN3C(=O)OC[C@@H]3C2)c(O)cn1]

### Step A—Synthesis of Compound 146

A solution of Int-2 (20 mg, 0.042 mmol), (E)-benzaldehyde oxime (6.5 mg, 0.054 mmol), cesium carbonate (41 mg, 0.125 mmol), and ROCKPhos palladacycle G2 (1.7 mg, 0.002 mmol) in dimethylformamide (0.50 mL) was purged subsurface with nitrogen, sealed, and heated at 100° C. overnight. The reaction mixture was then filtered, and the filtrate was directly purified using preparative HPLC (reverse-phase C-18), eluting with acetonitrile/water+0.1% TFA 10-100%, to provide compound 146 as a solid. MS (ESI, m/z): 418.3 [M+H]⁺. ¹H NMR (600 MHz, Chloroform-d) δ 8.25 (s, 1H), 8.02 (s, 1H), 7.67 (s, 1H), 7.26-7.19 (m, 4H), 5.00 (s, 1H), 4.52 (d, J=5.1 Hz, 2H), 4.38 (t, J=8.4 Hz, 1H), 4.12 (d, J=6.1 Hz, 1H), 3.88 (dd, J=8.6, 6.2 Hz, 1H), 3.71 (dd, J=13.4, 5.5 Hz, 1H), 3.36-3.33 (m, 1H), 3.33-3.25 (m, 1H), 2.22 (d, J=13.8 Hz, 1H), 2.05 (d, J=14.6 Hz, 1H), 1.86-1.73 (m, 1H), 1.66 (t, J=11.9 Hz, 1H).

### Example 147

### Preparation of Compound 147

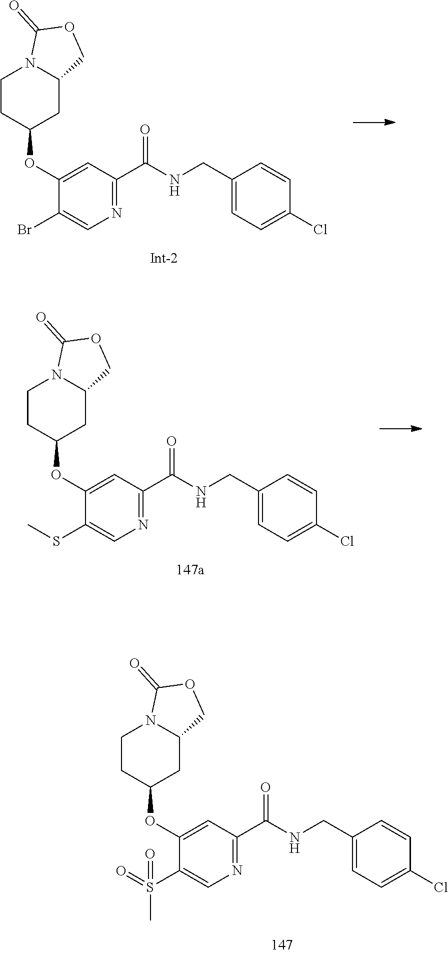 [SMILES: O=C(NCc1ccc(Cl)cc1)c1cc(O[C@H]2CCN3C(=O)OC[C@@H]3C2)c(Br)cn1 ; CSc1cnc(C(=O)NCc2ccc(Cl)cc2)cc1O[C@H]1CCN2C(=O)OC[C@@H]2C1 ; CS(=O)(=O)c1cnc(C(=O)NCc2ccc(Cl)cc2)cc1O[C@H]1CCN2C(=O)OC[C@@H]2C1]

### Step A—Synthesis of Compound 147a

To a solution of Int-2 (83 mg, 0.17 mmol) in THE (2 mL) at −78° C., was added dropwise a solution of n-butyl lithium (2.5 M, 0.14 mL, 0.35 mmol) followed by dropwise addition of 1,2-dimethyldisulfane (35 mg, 0.38 mmol). The resulting reaction mixture was allowed to stir at −78° C. for 1 hour, then was allowed to warm to room temperature while stirring overnight. The reaction was quenched with 1N HCl, then diluted with ethyl acetate and water, and extracted with ethyl acetate. The organic extract was washed with brine, concentrated in vacuo, and the resulting residue was purified using preparative HPLC (reverse-phase C-18), eluting with acetonitrile/water+0.1% TFA 10-100%, to provide compound 147a as a solid. MS (ESI, m/z): 448.3 [M+H]⁺.

### Step B—Synthesis of Compound 147

To a solution of 147a in dichloromethane (0.50 mL) at 0° C. was added m-CPBA (17 mg, 0.71 mmol), and the reaction mixture was allowed to stir for 1 hour. The reaction mixture was then directly purified using silica gel chromatography, eluting with a gradient of (3:1 ethyl acetate:ethanol):hexanes—5 to 75%, to provide 147 as a solid. MS (ESI, m/z): 480.3 [M+H]⁺. ¹H NMR (600 MHz, DMSO-d₆) δ 9.59 (s, 1H), 8.77 (s, 1H), 7.84 (s, 1H), 7.36-7.27 (m, 4H), 5.43 (s, 1H), 4.44 (d, J=6.1 Hz, 2H), 4.32 (s, 1H), 3.91 (d, J=40.1 Hz, 2H), 3.53 (s, 1H), 3.32 (s, 3H), 3.13 (s, 1H), 2.17 (s, 1H), 1.94 (s, 1H), 1.73 (d, J=14.5 Hz, 2H).

### Example 148

### Preparation of Compound 148

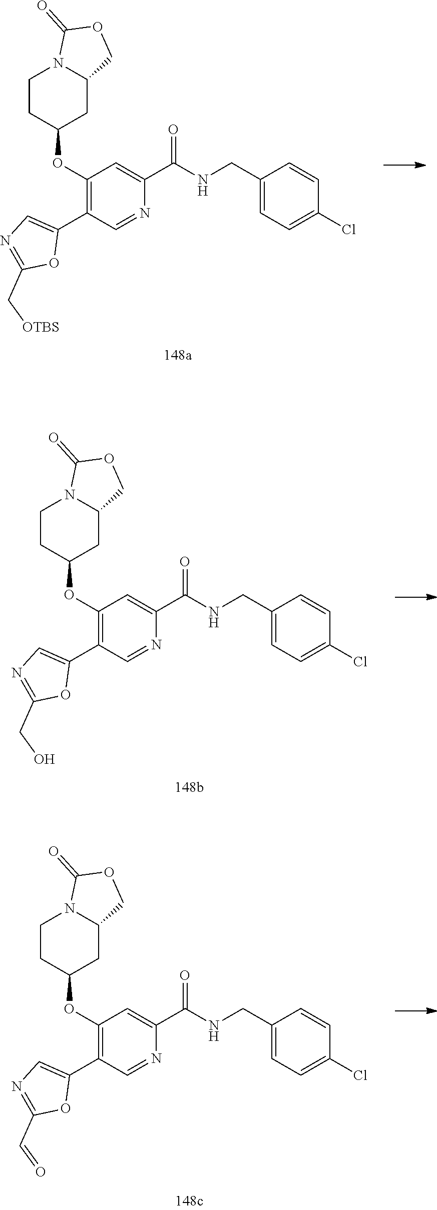 [SMILES: CC(C)(C)[Si](C)(C)OCc1ncc(-c2cnc(C(=O)NCc3ccc(Cl)cc3)cc2O[C@H]2CCN3C(=O)OC[C@@H]3C2)o1 ; O=C(NCc1ccc(Cl)cc1)c1cc(O[C@H]2CCN3C(=O)OC[C@@H]3C2)c(-c2cnc(CO)o2)cn1 ; O=Cc1ncc(-c2cnc(C(=O)NCc3ccc(Cl)cc3)cc2O[C@H]2CCN3C(=O)OC[C@@H]3C2)o1]

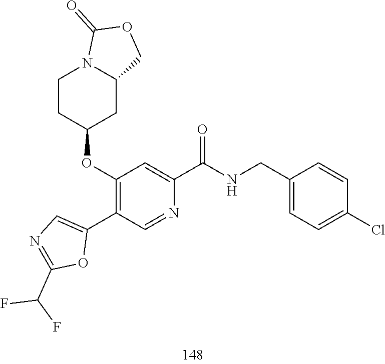 [SMILES: O=C(NCc1ccc(Cl)cc1)c1cc(O[C@H]2CCN3C(=O)OC[C@@H]3C2)c(-c2cnc(C(F)F)o2)cn1]

### Step A—Synthesis of Compound 148b

A mixture of 148a (100 mg, 0.163 mmol, made using the methodology described in Example 3, Step A) in hydrochloric acid (4 M in dioxane) (2 mL, 24.35 mmol) was allowed to stir for 1 hour at room temperature. The reaction mixture was concentrated in vacuo to provide compound 148b, which was used without further purification. MS (ESI, m/z): 499.3 [M+H]⁺. ¹H NMR (400 MHz, methanol-d₄, ppm): δ 9.01 (s, 1H), 8.21 (s, 1H), 7.79 (s, 1H), 7.44-7.36 (m, 4H), 5.52 (brs, 1H), 4.80 (s, 2H), 4.67 (s, 2H), 4.53 (t, J=8.4 Hz, 1H), 4.23-4.15 (m, 1H), 4.06-4.02 (m, 1H), 3.88-3.83 (m, 1H), 3.37-3.34 (m, 1H), 2.46-2.42 (m, 1H), 2.27-2.23 (m, 1H), 2.07-1.92 (m, 2H).

### Step B—Synthesis of Compound 148c

To a mixture of 148b (150 mg, 0.269 mmol) in dichloromethane (4 mL) at 0° C., was added Dess-Martin periodinane (228 mg, 0.538 mmol). The resulting reaction was allowed to stir at room temperature for 2 hours, then the reaction mixture was diluted with saturated aqueous sodium bicarbonate (15 mL) and extracted with ethyl acetate (3×15 mL). The combined organic extracts were washed with saturated aqueous sodium chloride (10 mL), dried over anhydrous sodium sulfate, filtered, and concentrated in vacuo to provide compound 148c, which was used without further purification. MS (ESI, m/z): 497.2 [M+H]⁺.

### Step C—Synthesis of Compound 148

To a mixture of 148c (130 mg, 0.262 mmol) in dichloromethane (4 mL) at 0° C., was added diethylaminosulfur trifluoride (0.8 mL, 6.05 mmol) dropwise under argon atmosphere. The resulting mixture was allowed to stir for 2 hours at room temperature, then the reaction mixture was quenched with saturated aqueous sodium bicarbonate (10 mL), and extracted with ethyl acetate (3×20 mL). The combined organic extracts were washed with saturated aqueous sodium chloride (10 mL), dried over anhydrous sodium sulfate, filtered, and concentrated in vacuo. The resulting residue was purified using preparative HPLC, eluting with a gradient of acetonitrile:water (0.8% ammonium bicarbonate)—63:37, to provide compound 148. MS (ESI, m/z): 519.3 [M+H]⁺. ¹H NMR (400 MHz, methanol-d₄, ppm): δ 8.96 (s, 1H), 7.94 (s, 1H), 7.81 (s, 1H), 7.45-7.34 (m, 4H), 7.03 (t, J=52.4 Hz, 1H), 5.39 (brs, 1H), 4.62 (s, 2H), 4.50 (t, J=8.4 Hz, 1H), 4.20-4.13 (m, 1H), 4.04-4.01 (m, 1H), 3.87-3.80 (m, 1H), 3.37-3.34 (m, 1H), 2.41-2.38 (m, 1H), 2.22-2.19 (m, 1H), 2.06-1.86 (m, 2H).

### Example 149

### Preparation of Compound 149

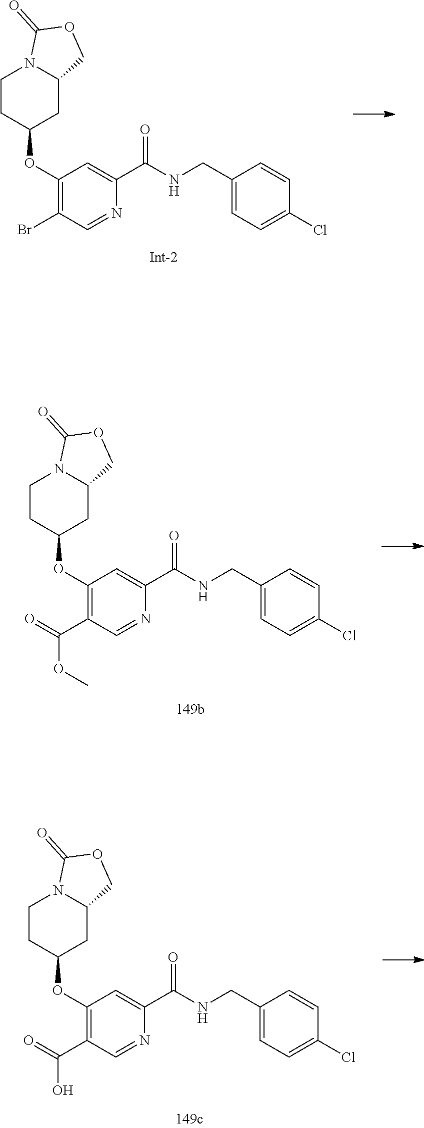 [SMILES: O=C(NCc1ccc(Cl)cc1)c1cc(O[C@H]2CCN3C(=O)OC[C@@H]3C2)c(Br)cn1 ; COC(=O)c1cnc(C(=O)NCc2ccc(Cl)cc2)cc1O[C@H]1CCN2C(=O)OC[C@@H]2C1 ; O=C(NCc1ccc(Cl)cc1)c1cc(O[C@H]2CCN3C(=O)OC[C@@H]3C2)c(C(=O)O)cn1]

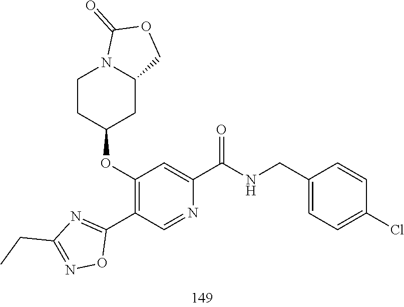 [SMILES: CCc1noc(-c2cnc(C(=O)NCc3ccc(Cl)cc3)cc2O[C@H]2CCN3C(=O)OC[C@@H]3C2)n1]

### Step A—Synthesis of Compound 149b

Compound 149b was prepared from compound Int-2, using the methodology described in Example 8, Step C. MS (ESI, m/z): 460.2 [M+H]⁺.

### Step B—Synthesis of Compound 149c

Compound 149c was prepared from 149b, using the methodology described in Example 8, Step D. MS (ESI, m/z): 446.2 [M+H]⁺.

### Step C—Synthesis of Compound 149

To a mixture of 149c (20 mg, 0.037 mmol) in DMF (0.15 mL) was added CDI (9.0 mg, 0.56 mmol) at room temperature. After 5 minutes, a 0.5 M solution of N-hydroxypropionimidamide hydrochloride (94 μl, 0.056 mmol) dissolved in pyridine (0.19 mL, 2.3 mmol), was added, and the reaction mixture was allowed to stir for 2 hours at 80° C. Trifluoroacetic acid (1 eq) and DMSO (0.1 mL) were then added, and the resulting solution was directly purified using HPLC (reverse-phase C-18), eluting with acetonitrile/water+0.1% TFA 10-95%, to provide compound 149 as a solid. MS (ESI, m/z): 498.3 [M+H]⁺. ¹H NMR (500 MHz, Chloroform-d) δ 9.16 (s, 1H), 8.47 (s, 1H), 7.94 (s, 1H), 7.33 (q, J=8.3 Hz, 4H), 4.67 (d, J=5.4 Hz, 2H), 4.48 (t, J=8.4 Hz, 1H), 4.33 (d, J=4.5 Hz, 1H), 3.98 (dd, J=8.2, 5.2 Hz, 1H), 3.87 (dd, J=13.4, 5.1 Hz, 1H), 3.44 (t, J=11.8 Hz, 1H), 2.89 (q, J=7.5 Hz, 2H), 2.50-2.03 (m, 3H), 2.03-1.71 (m, 2H), 1.42 (t, J=7.6 Hz, 3H).

### Example 150

### Preparation of Compound 150

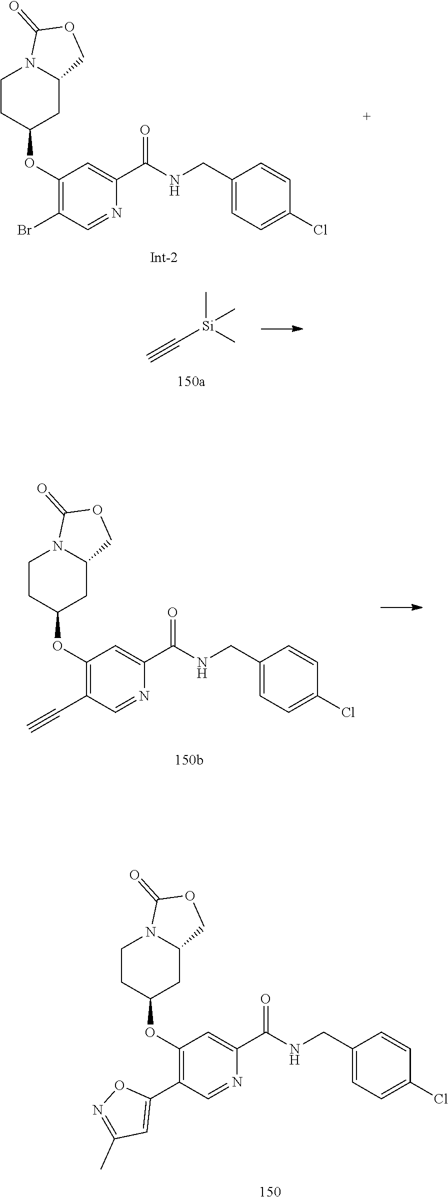

### Step A—Synthesis of Compound 150b

A solution of Int-2 (307 mg, 0.64 mmol), copper (I) iodide (6.1 mg, 0.032 mmol), bis(triphenylphosphine)palladium(II) dichloride (22.4 mg, 0.032 mmol), and triethylamine (0.22 mL, 1.60 mmol) in dioxane (3.2 mL) was purged subsurface with nitrogen. 150a (0.27 mL, 1.92 mmol) was added, and the reaction mixture was heated to 80° C., and allowed to stir at this temperature for 30 minutes. The reaction mixture was cooled to room temperature, filtered, and concentrated in vacuo to provide an oil residue, which was dissolved in MeOH. To the resulting solution was added potassium carbonate (265 mg, 1.92 mmol), and the resulting mixture was allowed to stir at room temperature for 1 hour. The reaction mixture was then filtered and concentrated in vacuo, and the resulting residue was purified using silica gel chromatography, eluting with a gradient of methanol:methylene chloride—0 to 3%, to provide 150b as an oil. MS (ESI, m/z): 426.2 [M+H]⁺.

### Step B—Synthesis of Compound 150

To a stirred solution of (E)-acetaldehyde oxime (0.035 ml, 0.59 mmol), and NCS (78 mg, 0.59 mmol) in THE (0.59 mL) at 0° C. was added a solution 150b (50 mg, 0.12 mmol) in THE (0.59 mL). TEA (0.049 ml, 0.35 mmol) was added dropwise, and the reaction mixture was warmed to room temperature overnight with stirring. The reaction mixture was then diluted with ethyl acetate and water, and the collected organic layer was washed with brine, dried over sodium sulfate, and concentrated in vacuo. The resulting residue was purified using silica gel chromatography, eluting with a gradient of (3:1 ethyl acetate:ethanol):hexanes—10 to 40%, to provide 150 as a solid. MS (ESI, m/z): 483.2 [M+H]⁺. ¹H NMR (500 MHz, Chloroform-d) δ 8.95 (s, 1H), 8.43 (t, J=6.0 Hz, 1H), 7.88 (s, 1H), 7.37-7.29 (m, 4H), 6.57 (s, 1H), 4.65 (d, J=6.3 Hz, 2H), 4.47 (t, J=8.2 Hz, 1H), 4.04 (ddq, J=15.0, 7.8, 3.6 Hz, 1H), 4.00-3.91 (m, 2H), 3.72 (s, 2H), 3.30 (td, J=13.3, 3.4 Hz, 1H), 2.43 (s, 3H), 2.38-2.31 (m, 1H), 2.18 (d, J=14.7 Hz, 1H), 2.03-1.92 (m, 1H), 1.82 (ddd, J=14.0, 11.8, 2.2 Hz, 1H).

### Example 151

### Preparation of Compound 151

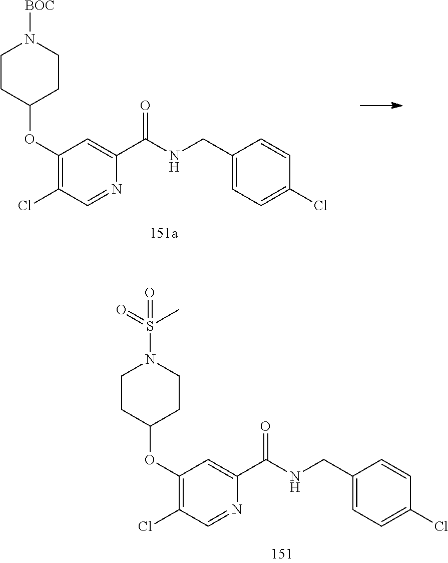 [SMILES: CC(C)(C)OC(=O)N1CCC(Oc2cc(C(=O)NCc3ccc(Cl)cc3)ncc2Cl)CC1 ; CS(=O)(=O)N1CCC(Oc2cc(C(=O)NCc3ccc(Cl)cc3)ncc2Cl)CC1]

### Step A—Synthesis of Compound 151

To a stirred suspension of 151a (15 mg, 0.031 mmol, made using the methodology described in Example 2, Step A, and Example 5, Step A) in dichloromethane (1.0 mL) was added trifluororacetic acid (1.0 mL), and the resulting reaction was allowed to stir at room temperature for 1 hour. The reaction mixture was then concentrated in vacuo, and the resulting residue was suspended in dichloromethane (1.0 mL), and DIEA (0.016 ml, 0.094 mmol). Methanesulfonyl chloride (1.22 μl, 0.016 mmol) was added and the resulting reaction was allowed to stir for an additional 1 hour. The reaction mixture was then concentrated in vacuo, and the residue obtained was purified using preparative HPLC (reverse-phase C-18), eluting with acetonitrile/water+0.1% TFA 0-100%, to provide compound 151 as a solid. MS (ESI, m/z): 458.2 [M+H]⁺.

### Example 152

### Preparation of Compound 152

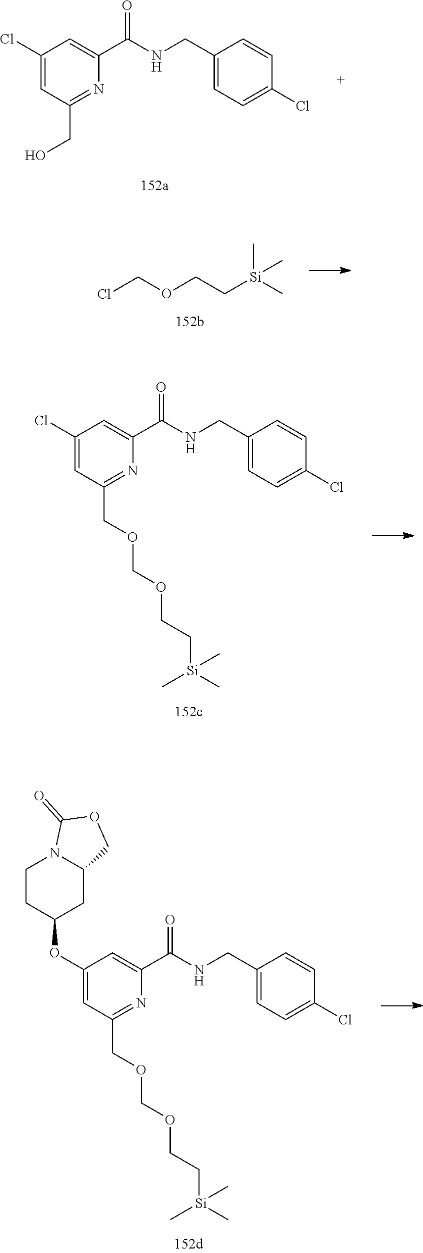

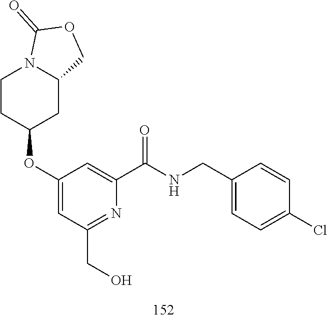 [SMILES: O=C(NCc1ccc(Cl)cc1)c1cc(O[C@H]2CCN3C(=O)OC[C@@H]3C2)cc(CO)n1]

### Step A—Synthesis of Compound 152c

To a solution of 152a (384 mg, 1.23 mmol, made using the methodology described in Example 2, Step A) in dichloromethane (10.0 mL) at 0° C. was added Hunig's Base (0.54 ml, 3.09 mmol) followed by 152b (0.29 ml, 1.61 mmol). The reaction was allowed to warm to room temperature with stirring overnight, and was then quenched with saturated aqueous sodium bicarbonate solution. The resulting solution was extracted with dichloromethane, and the organic extract was washed with brine, dried over magnesium sulfate, filtered and concentrated. The oil residue obtained was purified using silica gel chromatography, eluting with a gradient of ethyl acetate:hexanes—0 to 100%, to provide 152c as a solid. MS (ESI, m/z): 441.2 [M+H]⁺.

### Step B—Synthesis of Compound 152d

Compound 152d was prepared from compound 152c as a residue, using the methodology described in Example 2, Step B. MS (ESI, m/z): 562.3 [M+H]⁺.

### Step C—Synthesis of Compound 152

To a solution of 152d (7.0 mg, 0.012 mmol) in dichloromethane (0.50 mL) was added trifluoroacetic acid (0.009 ml, 0.125 mmol), and the reaction was allowed to stir at room temperature overnight. The reaction mixture was then quenched with MeOH and concentrated in vacuo. The resulting residue was purified using preparative HPLC (reverse-phase C-18), eluting with acetonitrile/water+0.1% TFA 0-100%, to provide compound 152 as a solid. MS (ESI, m/z): 432.2.2 [M+H]⁺. ¹H NMR (600 MHz, Methanol-d₄) δ 7.58 (d, J=2.1 Hz, 1H), 7.34-7.27 (m, 4H), 7.21 (d, J=2.1 Hz, 1H), 5.09 (s, 1H), 4.69 (s, 2H), 4.55 (s, 2H), 4.43 (t, J=8.4 Hz, 1H), 4.16-4.09 (m, 1H), 3.95 (dd, J=8.7, 5.9 Hz, 1H), 3.69 (dd, J=13.5, 4.5 Hz, 1H), 2.24 (d, J=13.9 Hz, 1H), 2.04 (d, J=14.8 Hz, 1H), 1.87-1.79 (m, 1H), 1.79-1.72 (m, 1H), 1.26 (s, 1H).

### Example 153

### Preparation of Compound 153

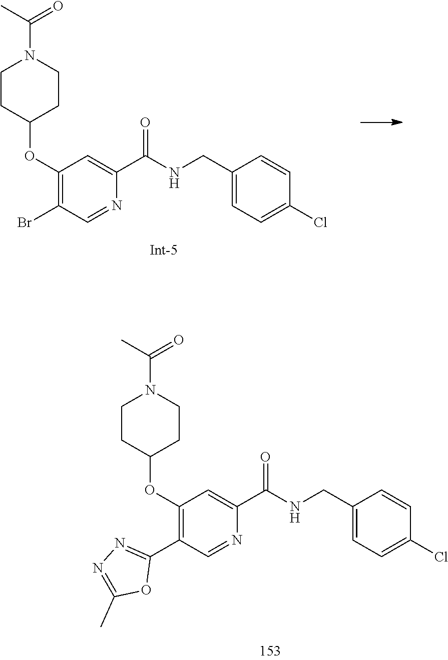 [SMILES: CC(=O)N1CCC(Oc2cc(C(=O)NCc3ccc(Cl)cc3)ncc2Br)CC1 ; CC(=O)N1CCC(Oc2cc(C(=O)NCc3ccc(Cl)cc3)ncc2-c2nnc(C)o2)CC1]

### Step A—Synthesis of Compound 153

To a solution of 2,2,6,6-tetramethylpiperidine (121 mg, 0.86 mmol) in THE (2.0 mL) at −40° C., was added dropwise 2.5 M n-butyllithium in hexane (0.34 ml, 0.86 mmol). The reaction mixture was warmed to −10° C., then allowed to stir at this temperature for 1 hour. A solution of anhydrous zinc chloride hydrate (145 mg, 0.94 mmol) in THE (2.0 mL) was added dropwise at −10° C., and the reaction was allowed to stir for 1 hour while warming to room temperature. A solution of 2-methyl-1,3,4-oxadiazole (72.1 mg, 0.86 mmol) in THE (2.0 mL) was added, and the reaction was allowed to stir for 30 minutes at room temperature. Int-5 (100 mg, 0.21 mmol) in NMP (2.0 mL), X-PHOS (10.2 mg, 0.021 mmol), and chloro(2-dicyclohexylphosphino-2′,4′,6′-triisopropyl-1,1′-biphenyl)[2-(2′-amino-1,1′-biphenyl)]palladium(II) (16.9 mg, 0.021 mmol) were added, and the reaction mixture was purged with nitrogen and irradiated with microwave radiation at 120° C. for 1 hour. The reaction was then quenched with saturated ammonium chloride and extracted with ethyl acetate. The organic extract was washed with brine, dried over sodium sulfate, filtered, and concentrated in vacuo, and the resulting residue was purified using silica gel column chromatography, eluting with a gradient of methanol:dichloromethane—0-10%, to provide compound 153 as a solid. MS (ESI, m/z): 470.2 [M+H]⁺. ¹H NMR (300 MHz, methanol-d₃, ppm): δ 9.03 (s, 1H), 7.96 (s, 1H), 7.39-7.33 (m, 4H), 5.19-5.17 (m, 1H), 4.62 (s, 2H), 3.90-3.63 (m, 4H), 2.66 (s, 3H), 2.15 (s, 3H), 2.12-1.91 (m, 4H).

### Example 154

### Preparation of Compound 154

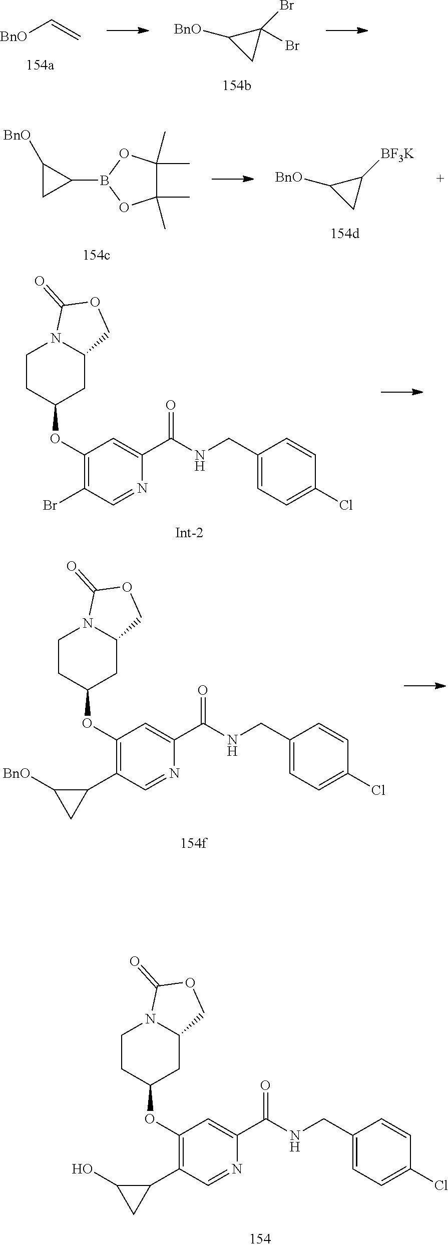

### Step A—Synthesis of Compound 154b

To a mixture of 154a (1.0 g, 7.45 mmol) in dichloromethane (40 mL) at 0° C. was added potassium hydroxide (6.02 g, 107 mmol), tetrabutylammonium hydrogensulfate (0.76 g, 2.24 mmol), and bromoform (17.33 g, 68.6 mmol). The resulting reaction was allowed to stir for 2 hours at room temperature, then the reaction mixture was filtered through a Celite pad and washed with dichloromethane (50 mL). The filtrate was diluted with water (50 mL), and the aqueous phase was extracted with ethyl acetate (3×100 mL). The combined organic extracts were dried over anhydrous sodium sulfate, filtered, and concentrated in vacuo. The resulting residue was purified using silica gel column chromatography, eluting with a gradient of diethyl ether:petroleum ether—0:1 to 1:50, to provide compound 154b. ¹H NMR (300 MHz, chloroform-d₃, ppm): δ 7.48-7.35 (m, 5H), 4.93 (d, J=8.4 Hz, 1H), 4.72 (d, J=8.4 Hz, 1H), 3.69-3.65 (m, 1H), 1.93-1.88 (m, 1H), 1.80-1.76 (m, 1H).

### Step B—Synthesis of Compound 154c

To a solution of 154b (900 mg, 2.94 mmol) in tetrahydrofuran (25 mL) at −78° C., under nitrogen atmosphere, was added n-butyllithium (2.5 M in n-hexane) (1.41 mL, 3.53 mmol) dropwise with stirring. After stirring at −78° C. for 30 minutes, a solution of 4,4,5,5-tetramethyl-1,3,2-dioxaborolane (753 mg, 5.88 mmol) in tetrahydrofuran (5 mL) was added. The resulting reaction was warmed to 50° C. and allowed to stir at this temperature for 16 hours. The reaction was quenched with saturated aqueous ammonium chloride (40 mL) and extracted with diethyl ether (3×100 mL). The combined organic extracts were washed with saturated aqueous sodium chloride (50 mL), dried over anhydrous sodium sulfate, filtered, and concentrated in vacuo. The resulting residue was purified using silica gel column chromatography, eluting with a gradient of diethyl ether:petroleum ether—1:20 to 1:10, to provide compound 154c. MS (ESI, m/z): 275.2 [M+H]⁺.

### Step C—Synthesis of Compound 154d

To a mixture of 154c (400 mg, 1.459 mmol) in methanol (8 mL) was added potassium fluoride hydrofluoride (342 mg, 4.38 mmol), and water (1.6 mL). The resulting mixture was allowed to stir for 3 hours at 15° C. The reaction mixture was concentrated in vacuo, and the resulting residue was suspended in acetonitrile (8 mL), and filtered through a pad of Celite. The filter-cake was washed with diethylether (10 mL), and the combined filtrate and washing was concentrated in vacuo to provide compound 154d, which was used without further purification. ¹H NMR (300 MHz, DMSO-d₆, ppm): δ 7.41-7.16 (m, 5H), 4.37 (s, 2H), 2.94 (s, 1H), 0.18-0.14 (m, 1H), 0.06-0.00 (m, 1H), −0.53-−0.40 (m, 1H).

### Step D—Synthesis of Compound 154e

To a mixture of Int-2 (150 mg, 0.312 mmol), 1,1′-bis(di-tert-butylphosphino)ferrocene-palladium dichloride (20 mg, 0.031 mmol), and 154e (159 mg, 0.624 mmol) in 2-methyl-2-butanol (3 mL) was added cesium carbonate (1.5 M in water) (0.624 mL, 0.936 mmol). The reaction mixture was allowed to stir for 16 hours at 100° C. under nitrogen atmosphere. The reaction was quenched with water (15 mL), and extracted with ethyl acetate (3×20 mL). The combined organic extracts were washed with saturated aqueous sodium chloride (40 mL), dried over anhydrous sodium sulfate, filtered and concentrated in vacuo. The resulting residue was purified using silica gel column chromatography, eluting with a gradient of dichloromethane:ethyl acetate—3:2 to 0:2, to provide compound 154f. MS (ESI, m/z): 548.3 [M+H]⁺.

### Step E—Synthesis of Compound 154

To a solution of 154f (130 mg, 0.237 mmol) in dichloromethane (3 mL) was added tribromoborane (1 M in dichloromethane) (1.42 mL, 1.423 mmol) at −78° C. under nitrogen atmosphere. The resulting reaction was allowed to stir for 1 hour at 0° C., then the reaction was quenched with saturated sodium bicarbonate (20 mL) and extracted with ethyl acetate (3×20 mL). The combined organic extracts were washed with saturated aqueous sodium chloride (20 mL), dried over anhydrous sodium sulfate, filtered, and concentrated in vacuo. The resulting residue was purified using silica gel column chromatography, eluting with a gradient of methanol:dichloromethane—1:20 to 1:10, to provide compound 154. MS (ESI, m/z): 458.3 [M+H]⁺. ¹H NMR (400 MHz, methanol-d₄, ppm): δ 8.31 (s, 1H), 7.69 (s, 1H), 7.36-7.30 (m, 4H), 5.12 (s, 1H), 4.58-4.54 (m, 3H), 4.47 (t, J=8.4 Hz, 1H), 4.20-4.17 (m, 1H), 3.99 (dd, J=6.4, 8.4 Hz, 1H), 3.77-3.72 (m, 1H), 3.38-3.33 (m, 1H), 2.85-2.78 (m, 1H), 2.30-2.26 (m, 1H), 2.10-2.04 (m, 1H), 1.93-1.76 (m, 4H).

### Example 155

### Preparation of Compound 155

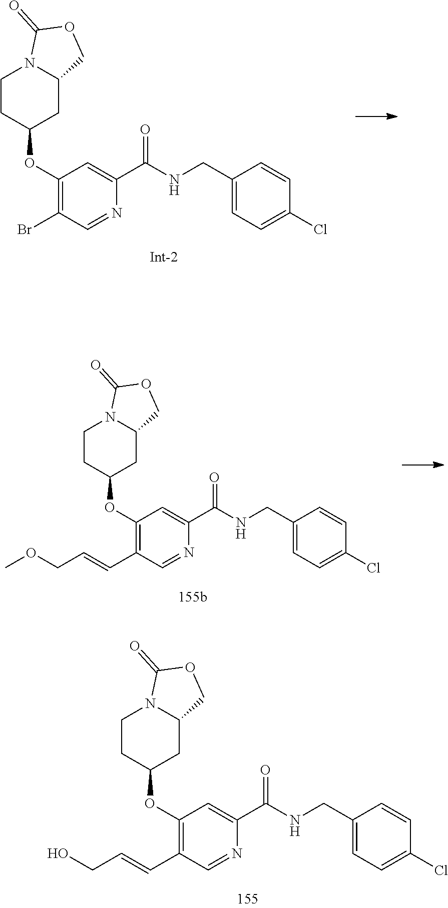 [SMILES: O=C(NCc1ccc(Cl)cc1)c1cc(O[C@H]2CCN3C(=O)OC[C@@H]3C2)c(Br)cn1 ; COC/C=C/c1cnc(C(=O)NCc2ccc(Cl)cc2)cc1O[C@H]1CCN2C(=O)OC[C@@H]2C1 ; O=C(NCc1ccc(Cl)cc1)c1cc(O[C@H]2CCN3C(=O)OC[C@@H]3C2)c(/C=C/CO)cn1]

### Step A—Synthesis of Compound 155b

Compound 155b was prepared as a solid from Int-2, using the methodology described in Example 3, Step A. MS (ESI, m/z): 472.0 [M+H]⁺. ¹H NMR (400 MHz, methanol-d₄, ppm): δ 8.67 (d, J=4.8 Hz, 1H), 7.93-7.87 (m, 1H), 7.40-7.34 (m, 4H), 6.93-6.89 (m, 1H), 6.69-6.63 (m, 1H), 5.29 (s, 1H), 4.62 (s, 2H), 4.50 (t, J=8.4 Hz, 1H), 4.20-4.12 (m, 3H), 4.01 (dd, J=6.0 Hz, 8.4 Hz, 1H), 3.79 (dd, J=5.2, 13.6 Hz, 1H), 3.44 (s, 3H), 3.34-3.27 (m, 1H), 2.34 (d, J=14.0 Hz, 1H), 2.14 (d, J=14.8 Hz, 1H), 2.05-1.82 (m, 2H).

### Step B—Synthesis of Compound 155

Compound 155 was prepared as a solid from compound 155b, using the methodology described in Example 154, Step E. MS (ESI, m/z): 458.2 [M+H]⁺. ¹H NMR (400 MHz, methanol-d₄, ppm): δ 8.59 (s, 1H), 7.72 (s, 1H), 7.35-7.30 (m, 4H), 6.89 (dd, J=1.6, 14.8 Hz, 1H), 6.66-6.59 (m, 1H), 5.18 (s, 1H), 4.57 (s, 2H), 4.46 (t, J=8.0 Hz, 1H), 4.29 (d, J=5.2 Hz, 2H), 4.16-4.14 (m, 1H), 3.99 (dd, J=6.0, 8.4 Hz, 1H), 3.74 (dd, J=14.0, 8.8 Hz, 1H), 3.34-3.27 (m, 1H), 2.31-2.26 (m, 1H), 2.10-2.06 (m, 1H), 1.89-1.77 (m, 2H).

### Example 156

### Preparation of Compound 156

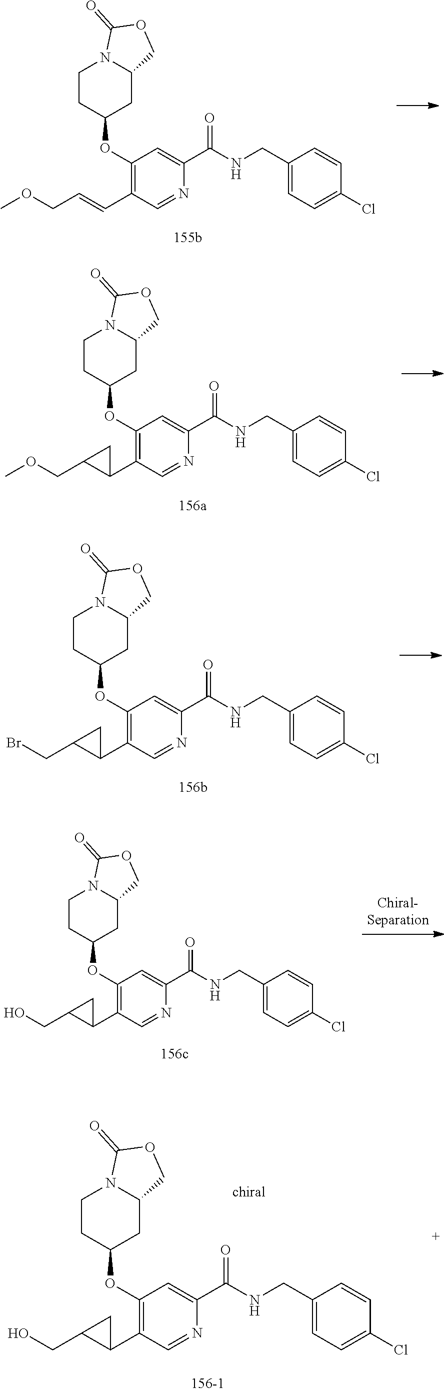 [SMILES: COC/C=C/c1cnc(C(=O)NCc2ccc(Cl)cc2)cc1O[C@H]1CCN2C(=O)OC[C@@H]2C1 ; COCC1CC1c1cnc(C(=O)NCc2ccc(Cl)cc2)cc1O[C@H]1CCN2C(=O)OC[C@@H]2C1 ; O=C(NCc1ccc(Cl)cc1)c1cc(O[C@H]2CCN3C(=O)OC[C@@H]3C2)c(C2CC2CBr)cn1 ; O=C(NCc1ccc(Cl)cc1)c1cc(O[C@H]2CCN3C(=O)OC[C@@H]3C2)c(C2CC2CO)cn1 ; O=C(NCc1ccc(Cl)cc1)c1cc(O[C@H]2CCN3C(=O)OC[C@@H]3C2)c(C2CC2CO)cn1]

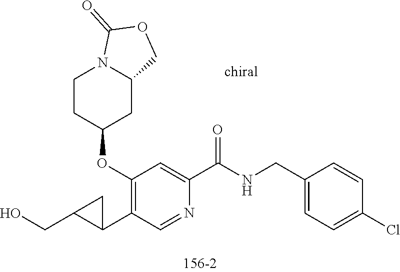 [SMILES: O=C(NCc1ccc(Cl)cc1)c1cc(O[C@H]2CCN3C(=O)OC[C@@H]3C2)c(C2CC2CO)cn1]

### Step A—Synthesis of Compound 156a

To a mixture of diethylzine (1 M in hexane) (10 mL, 10 mmol) in dichloromethane (8 mL) was added diiodomethane (1.36 g, 5.09 mmol) at −30° C. under nitrogen atmosphere. The resulting mixture was allowed to stir for 1 hour at −30° C., then 1,2-dimethoxyethane (0.3 mL, 2.54 mmol), and trichloroacetic acid (332 mg, 2.03 mmol) were added. The resulting reaction was allowed to stir for 1 hour at −15° C., then compound 155b (300 mg, 0.64 mmol) was added. The resulting reaction was allowed to stir for 16 hours at room temperature, then the reaction mixture was diluted with saturated aqueous ammonium chloride (30 mL) and extracted with ethyl acetate (3×30 mL). The combined organic extracts were washed with saturated aqueous sodium chloride (30 mL), dried over anhydrous sodium sulfate, filtered, and concentrated in vacuo. The resulting residue was purified using preparative HPLC, eluting with a gradient of acetonitrile:water (0.1% trifluoroacetic acid)—1:4 to 3:2, to provide compound 156a. MS (ESI, m/z): 486.3 [M+H]⁺.

### Step B—Synthesis of Compound 156b

To a mixture of 156a (50 mg, 0.1 mmol) in dichloromethane (2 mL) was added tribromoborane (0.2 mL, 0.2 mmol) (1 M in tetrahydrofuran) at room temperature. The reaction mixture was allowed to stir for 15 minutes, then was cooled to 0° C., and diluted with water (15 mL) and. The resulting solution was adjusted to pH 10 using saturated sodium bicarbonate at 0° C., then the mixture was extracted with ethyl acetate (3×30 mL). The combined organic extracts were washed with saturated aqueous sodium chloride (30 mL), dried over anhydrous sodium sulfate, filtered, and concentrated in vacuo. The resulting residue was purified using silica gel chromatography, eluting with a gradient of methanol:dichloromethane—0:100 to 5:95, to provide compound 156b. MS (ESI, m/z): 534.2, 536.2 [M+H]⁺.

### Step C—Synthesis of Compound 156c

To a mixture of 156b (65 mg, 0.12 mmol) in N,N-dimethylformamide (3 mL) was added sodium acetate (100 mg, 1.2 mmol). The resulting mixture was allowed to stir for 12 hours at 60° C. The reaction mixture was diluted with water (30 mL), and extracted with ethyl acetate (3×30 mL). The combined organic extracts were washed with saturated aqueous sodium chloride (30 mL), dried over anhydrous sodium sulfate, filtered, and concentrated in vacuo. The resulting residue was dissolved in tetrahydrofuran (2.5 mL), and water (2.5 mL), and to the resulting solution was added lithium hydroxide (6 mg, 0.243 mmol). The resulting reaction was allowed to stir for 1 hour at room temperature, then the reaction mixture was concentrated in vacuo. The resulting residue was purified using preparative HPLC, eluting with a gradient of acetonitrile:water (0.1% trifluoroacetic acid)—1:4 to 3:2, to provide compound 156c. MS (ESI, m/z): 472.1 [M+H]⁺.

Step D—Chiral resolution of 156c

Compound 156c (40 mg, 0.085 mmol) was resolved using Chiral-HPLC (Column: CHIRALPAK IG), eluting with a gradient of methyl tert-butyl ether:ethanol—60:40, to provide compound 156-1 (fast peak). MS (ESI, m/z): 472.1 [M+H]⁺. ¹H NMR (300 MHz, chloroform-d, ppm): δ 8.46 (brs, 1H), 8.06 (s, 1H), 7.71 (s, 1H), 7.34-7.28 (m, 4H), 5.13 (s, 1H), 4.62 (d, J=6.3 Hz, 2H), 4.46 (t, J=8.4 Hz, 1H), 4.20-4.10 (m, 1H), 3.93 (dd, J=6.0, 8.7 Hz, 1H), 3.89-3.77 (m, 2H), 3.65-3.58 (m, 1H), 3.38-3.30 (m, 1H), 2.35-2.28 (m, 1H), 2.18-2.08 (m, 1H), 2.01-1.70 (m, 3H), 1.47-1.44 (m, 1H), 1.33-1.27 (m, 1H), 1.15-1.11 (m, 1H), 1.05-1.00 (m, 1H), and compound 156-2 (slow peak). MS (ESI, m/z): 472.1 [M+H]⁺. ¹H NMR (300 MHz, chloroform-d, ppm): δ 8.46 (brs, 1H), 8.04 (s, 1H), 7.71 (s, 1H), 7.34-7.28 (m, 4H), 5.13 (s, 1H), 4.62 (d, J=6.3 Hz, 2H), 4.46 (t, J=8.4 Hz, 1H), 4.20-4.10 (m, 1H), 3.95 (dd, J=6.0, 8.7 Hz, 1H), 3.87-3.80 (m, 2H), 3.59-3.52 (m, 1H), 3.38-3.28 (m, 1H), 2.35-2.28 (m, 1H), 2.18-2.08 (m, 1H), 2.01-1.70 (m, 3H), 1.47-1.44 (m, 1H), 1.33-1.27 (m, 1H), 1.16-1.12 (m, 1H), 1.05-1.00 (m, 1H).

### Example 157

### Preparation of Compound 157

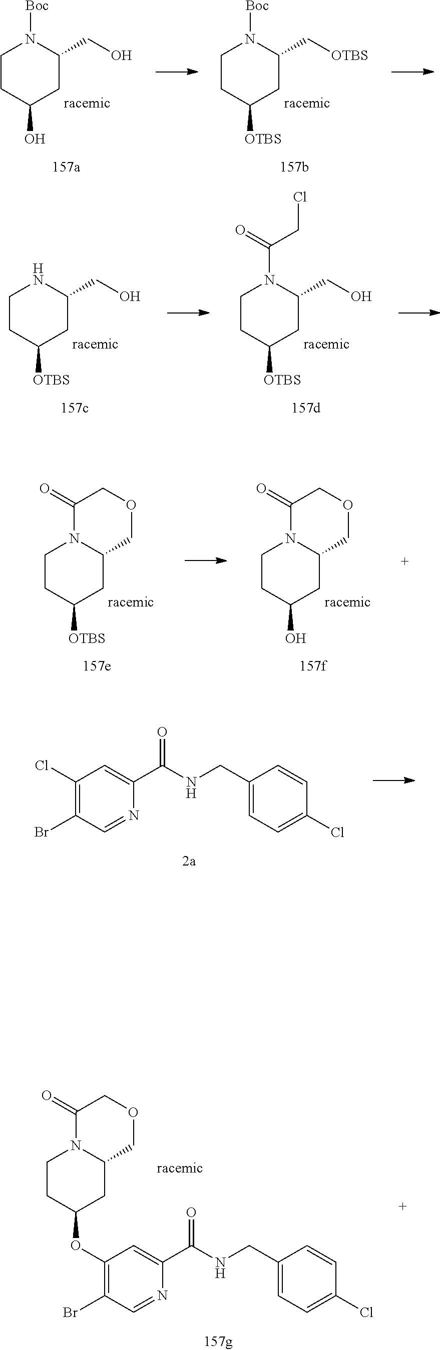

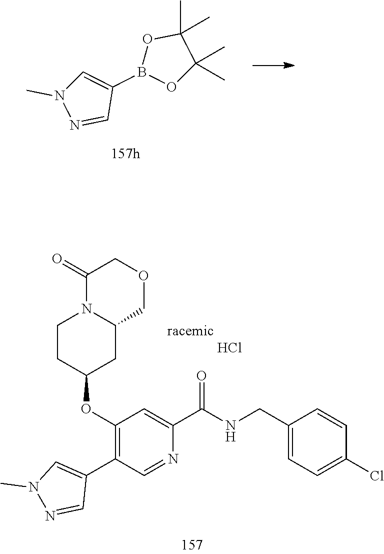

### Step A—Synthesis of Compound 157b

To a solution of 157a (330 mg, 1.427 mmol), and 1H-imidazole (291 mg, 4.28 mmol) in N,N-dimethylformamide (10 mL) was added tert-butylchlorodimethylsilane (645 mg, 4.28 mmol). The resulting mixture was allowed to stir at room temperature 16 hours. The resulting mixture was quenched with water (100 mL) and extracted with ethyl acetate (3×50 mL). The combined organic extracts were washed with saturated aqueous sodium chloride (2×50 mL), dried over anhydrous sodium sulfate, filtered, and concentrated in vacuo. The resulting residue was purified using silica gel chromatography, eluting with a gradient of ethyl acetate:petroleum ether—0:1 to 1:3, to provide compound 157b. MS (ESI, m/z): 359 [M−100]⁻.

### Step B—Synthesis of Compound 157c

To a solution of 157b (540 mg, 1.174 mmol) in dichloromethane (10 mL) was added trifluoroacetic acid (1 mL). The resulting reaction was allowed to stir for 30 minutes at room temperature, then the reaction mixture was concentrated in vacuo to provide compound 157c, which was used without further purification. MS (ESI, m/z): 246.2 [M+H]⁺.

### Step C—Synthesis of Compound 157d

To a solution of 157c (300 mg, 0.834 mmol), and triethylamine (168 mg, 1.67 mmol) in dichloromethane (10 mL) was added dropwise 2-chloroacetyl chloride (141 mg, 1.251 mmol) at room temperature. The resulting reaction was allowed to stir for 0.5 hours at room temperature, then the reaction mixture was quenched with saturated aqueous sodium bicarbonate (20 mL). The mixture was extracted with ethyl acetate (3×50 mL). The combined organic extracts were washed with saturated aqueous sodium chloride (50 mL), dried over anhydrous sodium sulfate, filtered, and concentrated in vacuo. The resulting residue was purified using silica gel chromatography, eluting with a gradient of methanol:dichloromethane—0:10 to 1:10 to provide compound 157d. MS (ESI, m/z): 322 [M+H]⁺.

### Step D—Synthesis of Compound 157e

To a solution of 157d (200 mg, 0.621 mmol) in N,N-dimethylformamide (10 mL) was added sodium hydride (37.3 mg, 0.932 mmol). The resulting reaction was allowed to stir for 2 hours at room temperature, then was quenched with water (20 mL). The mixture was extracted with ethyl acetate (3×50 mL). The combined organic extracts were washed with saturated aqueous sodium chloride (50 mL), dried over anhydrous sodium sulfate, filtered, and concentrated in vacuo. The resulting residue was purified using silica gel chromatography, eluting with a gradient of ethyl acetate:petroleum ether—1:10 to 3:4, to provide compound 157e. MS (ESI, m/z): 286 [M+H]⁺.

### Step E—Synthesis of Compound 157f

To a solution of 157e (110 mg, 0.385 mmol) in tetrahydrofuran (5 mL) was added dropwise tetrabutylammonium fluoride (1 M in tetrahydrofuran, 504 mg, 1.927 mmol). The resulting mixture was allowed to stir for 1 hour at room temperature. The reaction mixture was quenched with water (20 mL). The mixture was extracted with ethyl acetate (3×50 mL). The combined organic extracts were washed with saturated aqueous sodium chloride (50 mL), dried over anhydrous sodium sulfate, filtered, and concentrated in vacuo. The resulting residue was purified using silica gel chromatography, eluting with a gradient of methanol:dichloromethane—0:10 to 1:10 to provide compound 157f. MS (ESI, m/z): 172 [M+H]⁺.

### Step F—Synthesis of Compound 157g

To a mixture of sodium hydride (21 mg, 0.526 mmol) in N,N-dimethylformamide (1.5 mL) was added a solution of 157f (60 mg, 0.350 mmol) in N,N-dimethylformamide (1 mL) under argon atmosphere. The resulting reaction was allowed to stir for 0.5 hours at room temperature, then 2a (139 mg, 0.386 mmol) was added, and the reaction was allowed to stir for 16 hours at 80° C. The reaction was quenched with water (50 mL), and the resulting solution was extracted with dichloromethane (2×50 mL). The combined organic extracts were washed with saturated aqueous sodium chloride (50 mL), dried over anhydrous sodium sulfate, filtered, and concentrated in vacuo. The resulting residue was purified using silica gel chromatography, eluting with a gradient of methanol:dichloromethane—0:10 to 1:10, to provide compound 157g. MS (ESI, m/z): 494.1, 496.1 [M+H]⁺.

Step G—synthesis of compound 157

To a solution of potassium carbonate (28 mg, 0.20 mmol), 157h (25 mg, 0.121 mmol), 157g (50 mg, 0.101 mmol) in 1,4-dioxane (1 mL), and water (0.1 mL) was added tetrakis(triphenylphosphine)palladium(0) (23.36 mg, 0.020 mmol). The reaction mixture was allowed to stir for 16 hours at 85° C. under nitrogen atmosphere. The resulting mixture was diluted with water (50 mL) and extracted with ethyl acetate (3×50 mL). The combined organic extracts were washed with saturated aqueous sodium chloride (50 mL), dried over anhydrous sodium sulfate, filtered, and concentrated in vacuo. The resulting residue was purified using silica gel chromatography, eluting with a gradient of methanol:dichloromethane—0:10 to 1:10 to provide compound 157 MS (ESI, m/z): 496.2 [M−Cl]⁺. ¹H NMR (300 MHz, methanol-d₄, ppm): δ 8.73 (s, 1H), 8.16 (s, 1H), 8.02 (s, 1H), 7.87 (s, 1H), 7.38-7.31 (m, 4H), 5.25 (s, 1H), 4.60-4.52 (m, 3H), 4.12 (s, 2H), 4.02-3.96 (m, 4H), 3.78-3.76 (m, 1H), 3.63-3.56 (m, 1H), 2.99-2.90 (m, 1H), 2.21-2.14 (m, 2H), 1.97-1.88 (m, 2H).

### Example 158

### Preparation of Compound 158

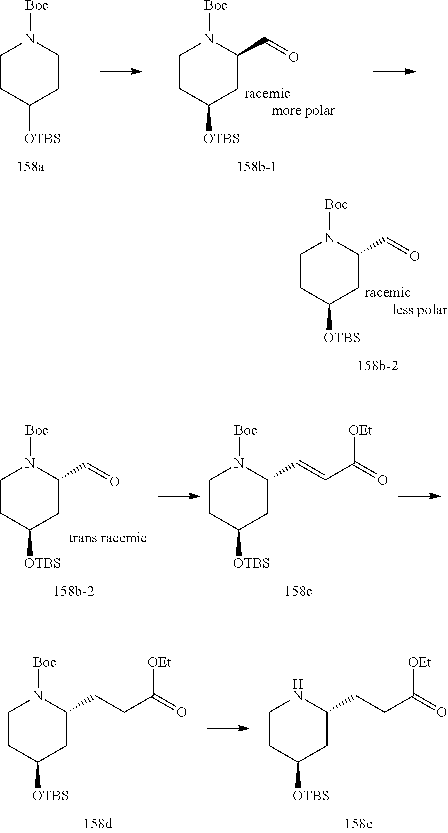

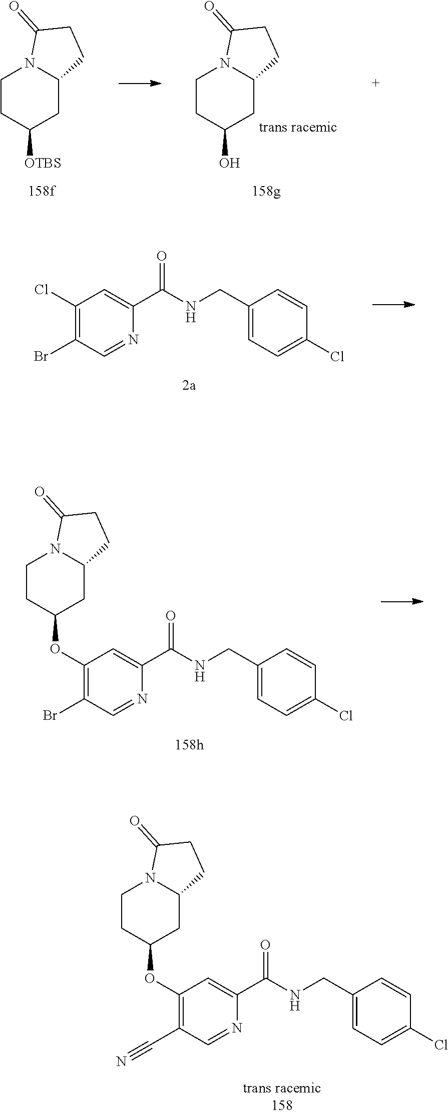 [SMILES: CC(C)(C)[Si](C)(C)O[C@H]1CCN2C(=O)CC[C@@H]2C1 ; O=C1CC[C@@H]2C[C@@H](O)CCN12 ; O=C(NCc1ccc(Cl)cc1)c1cc(Cl)c(Br)cn1 ; O=C(NCc1ccc(Cl)cc1)c1cc(O[C@H]2CCN3C(=O)CC[C@@H]3C2)c(Br)cn1 ; N#Cc1cnc(C(=O)NCc2ccc(Cl)cc2)cc1O[C@H]1CCN2C(=O)CC[C@@H]2C1]

### Step A—Synthesis of Compound 158b-1 and 158b-2

To a mixture of 158a (10 g, 31.7 mmol), N¹,N¹,N²,N²-tetramethylethane-1,2-diamine (11.05 g, 95 mmol), and ethoxyethane (50 mL) was added sec-butyllithium (48.8 mL, 63.4 mmol) dropwise at −78° C. under nitrogen atmosphere. The resulting mixture was allowed to stir for 2 h at −78° C. To this mixture was added N,N-dimethylformamide (11.58 g, 158 mmol) at −78° C. The resulting mixture was allowed to stir at −78° C. for 1 hour. The reaction mixture was diluted with saturated aqueous ammonium chloride (100 mL), then extracted with ethyl acetate (3×150 mL). The combined organic extracts were washed with saturated aqueous sodium chloride (2×50 mL), dried over anhydrous sodium sulfate, filtered, and concentrated in vacuo. The resulting residue was purified using silica gel chromatography, eluting with a gradient of ethyl acetate:petroleum ether—0:25 to 3:25 to provide 158b-2 (less polar), and 158b-1 (less polar). MS (ESI, m/z): 344.2 [M+H]⁺.

### Step B—Synthesis of Compound 158c

To a mixture of ethyl 2-(diethoxyphosphoryl)acetate (1.96 g, 8.73 mmol), and sodium hydride (0.35 g, 8.73 mmol) in tetrahydrofuran (15 mL) was added 158b-2 (2.0 g, 5.82 mmol). The resulting mixture was allowed to stir for 2 hours at room temperature. The reaction mixture was diluted with saturated aqueous ammonium chloride (50 mL), then extracted with ethyl acetate (3×50 mL). The combined organic extracts were washed with saturated aqueous sodium chloride (2×50 mL), dried over anhydrous sodium sulfate, filtered, and concentrated in vacuo. The resulting residue was purified using silica gel chromatography, eluting with a gradient of ethyl acetate:petroleum ether—0:1 to 3:25 to provide to provide compound 158c. (ESI, m/z): 414.2 [M+H]⁺.

### Step C—Synthesis of Compound 158d

To a solution of 158c (2.0 g, 4.84 mmol) in methanol (10 mL) was added palladium on carbon (10%, 200 mg, 1.69 mmol). The mixture was evacuated and flushed three times with nitrogen, followed by flushing with hydrogen. The mixture was allowed to stir at room temperature for 1 hour under an atmosphere of hydrogen (2 atm). The reaction mixture was filtered, and the filtrate was concentrated in vacuo to provide compound 158d, which was used without further purification. (ESI, m/z): 416.2[M+H]⁺.

### Step D—Synthesis of Compound 158e

To a solution of 158d (1.9 g, 4.57 mmol) in dichloromethane (10 mL) was added trifluoroacetic acid (3.0 mL). The mixture was allowed to stir for 1 hour at room temperature. The mixture was concentrated in vacuo to provide compound 158e. (ESI, m/z): 316.2 [M+H]⁺.

### Step E—Synthesis of Compound 158f

To a mixture of 158e (1.5 g, 4.75 mmol), water (5 mL), and tetrahydrofuran (5 mL) was added lithium hydroxide hydrate (0.6 g, 14.26 mmol) at room temperature. The resulting mixture was allowed to stir for 16 hours at room temperature. The reaction mixture was diluted with saturated aqueous ammonium chloride (20 mL), then extracted with ethyl acetate (3×50 mL). The combined organic extracts were washed with saturated aqueous sodium chloride (2×50 mL), dried over anhydrous sodium sulfate, filtered, and concentrated in vacuo. The resulting residue was purified using silica gel chromatography eluting with a gradient of methanol:dichloromethane—0:20 to 1:20 to provide compound 158f. (ESI, m/z): 270.1 [M+H]⁺.

### Step F—Synthesis of Compound 158g

To a mixture of 158f (350 mg, 1.299 mmol) in tetrahydrofuran (5 mL) was added tetrabutylammonium fluoride (1.3 mL, 1.3 mmol) at room temperature. The resulting mixture was allowed to stir for 16 hours at room temperature. The reaction was diluted with methanol (10 mL) and concentrated in vacuo. The resulting residue was purified using silica gel chromatography, eluting with a gradient of methanol:dichloromethane—1:50 to 2:25 to provide compound 158g. (ESI, m/z): 156[M+H]⁺.

### Step G—Synthesis of Compound 158h

To a solution of 158 g (100 mg, 0.644 mmol) in N,N-dimethylformamide (10 mL) was added sodium hydride (39 mg, 0.967 mmol) at room temperature. The resulting mixture was allowed to stir for 1 hour at room temperature. To this was added 2c (302 mg, 0.838 mmol) at room temperature. The resulting mixture was warmed to 80° C. and stirred for 2 hours at 80° C. The reaction mixture was diluted with water (50 mL), then extracted with ethyl acetate (3×50 mL). The combined organic extracts were washed with saturated aqueous sodium chloride (2×50 mL), dried over anhydrous sodium sulfate, filtered, and concentrated in vacuo. The resulting residue was purified using silica gel chromatography eluting with a gradient of methanol:dichloromethane—1:50 to 1:10 to provide compound 158h. (ESI, m/z): 478[M+H]⁺.

### Step H—Synthesis of Compound 158h

To a mixture of tetrakis(triphenylphosphine)palladium(0) (36.2 mg, 0.031 mmol), 158 h (30 mg, 0.063 mmol) in N,N-dimethylformamide (1 mL) was added dicyanozinc (11 mg, 0.094 mmol) at room temperature. The reaction mixture was irradiated with microwave radiation for 2 hours at 140° C. The reaction mixture was quenched with water (50 mL) and extracted with ethyl acetate (3×10 mL). The combined organic extracts were washed with saturated aqueous sodium chloride (40 mL), dried over anhydrous sodium sulfate, filtered, and concentrated in vacuo. The resulting residue was purified using silica gel chromatography, eluting with a gradient of methanol:dichloromethane—1:50 to 1:9 to provide compound 158. MS (ESI, m/z): 425.2 [M+H]⁺. ¹H NMR: (400 MHz, methanol-d₄, ppm): δ 8.77 (s, 1H), 7.90 (s, 1H), 7.35-7.30 (m, 4H), 5.29 (s, 1H), 4.58 (s, 2H), 4.02-3.91 (m, 2H), 3.14-3.07 (m, 1H), 2.44-2.25 (m, 4H), 2.10 (d, J=14.8 Hz, 1H), 1.87-1.80 (m, 1H), 1.79-1.64 (m, 2H).

### Example 159

### Preparation of Compound 159

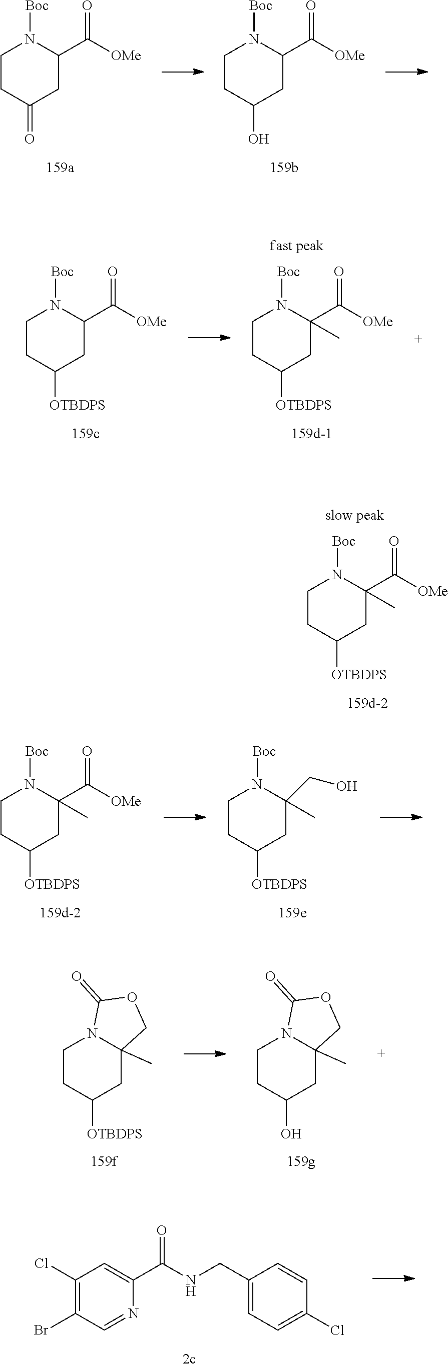

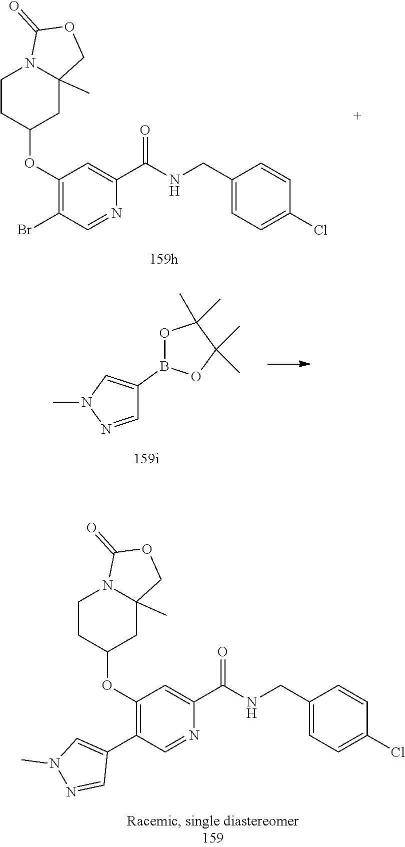

### Step A—Synthesis of Compound 159b

To a solution of 159a (2 g, 7.77 mmol) in tetrahydrofuran (20 mL) was added sodium borohydride (0.324 g, 8.55 mmol) at 0° C. The resulting mixture was allowed to stir for 1 hour at 0° C., then quenched with saturated ammonium chloride aqueous (20 mL), and extracted with ethyl acetate (3×50 mL). The combined organic extracts were washed with saturated aqueous sodium chloride (80 mL), dried over anhydrous sodium sulfate, filtered, and concentrated in vacuo. The resulting residue was purified using silica gel chromatography, eluting with a gradient of ethyl acetate:petroleum ether—1:5 to 7:1 to provide compound 159b. MS (ESI, m/z): 260.2 [M+H]⁺.

### Step B—Synthesis of Compound 159c

To a mixture of imidazole (1.418 g, 20.83 mmol), and 159b (1.8 g, 6.94 mmol) in dimethyl formamide (5 mL) was added tert-butylchlorodiphenylsilane (2.140 mL, 8.33 mmol) at room temperature. The solution was allowed to stir for 16 hours at room temperature. The resulting reaction was quenched with water (50 mL), and extracted with ethyl acetate (2×50 mL). The combined organic extracts were washed with saturated aqueous sodium chloride (50 mL), dried over anhydrous sodium sulfate, filtered, and concentrated in vacuo. The resulting residue was purified using silica gel chromatography, eluting with a gradient of ethyl acetate:petroleum ether—0:1 to 1:5 to provide compound 159c. MS (ESI, m/z): 498.1 [M+H]⁺.

### Step C—Synthesis of Compound 159d-1 and 159d-2

To the solution of 159c (2.9 g, 5.83 mmol) in dry toluene (6 mL) was added potassium bis(trimethylsilyl)amide (12.49 mL, 8.74 mmol) at −78° C. under nitrogen atmosphere. The mixture stirred for 30 minutes at −78° C. To this was added iodomethane (1.093 mL, 17.48 mmol). The temperature was allowed to rise to room temperature, and the mixture was allowed to stir for 16 hours. The reaction mixture was quenched with saturated ammonium chloride aqueous (50 mL), and extracted with ethyl acetate (3×50 mL). The combined organic extracts were washed with saturated aqueous sodium chloride (100 mL), dried over anhydrous sodium sulfate, filtered, and concentrated in vacuo. The resulting residue was purified using preparative HPLC eluting with a gradient of acetonitrile:water (20 mmol/L ammonium bicarbonate)—91:9 to 93:7 to provide compounds 159d-1 (fast peak), and 159d-2 (slow peak). MS (ESI, m/z): 512.1 [M+H]⁺.

### Step D—Synthesis of Compound 159e

To a solution of 159d-2 (760 mg, 1.485 mmol) in dry tetrahydrofuran (8 mL) was added lithium aluminum hydride (2.97 mL, 2.97 mmol) at 0° C. under nitrogen atmosphere. The mixture was allowed to stir for 1 hour at 0° C. The resulting reaction was quenched with water (115 μL), 15% sodium hydroxide aqueous (115 μL), and water (350 μL) at 0° C., then filtered and washed with ethyl acetate (200 mL). The filtrate was washed with water (50 mL), and saturated aqueous sodium chloride (50 mL). The combined organic extracts were dried over anhydrous sodium sulfate, filtered, and concentrated in vacuo. The resulting residue was purified using silica gel chromatography, eluting with a gradient of methanol:dichloromethane—0:10 to 1:20 to provide compound 159e. MS (ESI, m/z): 484.4 [M+H]⁺.

### Step E—Synthesis of Compound 159f

To a solution of 159e (500 mg, 1.034 mmol) in 2-propanol (15 mL) was added potassium tert-butoxide (116 mg, 1.034 mmol). The resulting mixture was heated to 70° C. and stirred for 2 hours. After cooling to 0° C., the pH value of the reaction mixture was adjusted to 6 with 1 M hydrochloric acid. The resulting mixture was concentrated in vacuo. The resulting residue was purified using silica gel chromatography, eluting with a gradient of methanol:dichloromethane—0:20 to 1:20 to provide compound 159f. MS (ESI, m/z): 410.2 [M+H]⁺.

### Step F—Synthesis of Compound 159g

To a solution of 159f (300 mg, 0.732 mmol) in tetrahydrofuran (1.5 mL) was added tetrabutylammoniumfluoride (0.732 mL, 0.732 mmol), and the mixture was allowed to stir for 16 hours at room temperature. The resulting reaction was diluted with methanol (5 mL), and concentrated in vacuo. The resulting residue was purified using silica gel chromatography, eluting with a gradient of methanol:dichloromethane—0:10 to 1:10 to provide compound 159g. MS (ESI, m/z): 172.2 [M+H]⁺.

### Step G—Synthesis of Compound 159h

To a solution of 159 g (51 mg, 0.300 mmol) in N,N-dimethyl formamide (2 mL) was added sodium hydride (11 mg, 0.275 mmol), and the resulting mixture was allowed to stir for 10 minutes at room temperature. To this mixture was added 2c (90 mg, 0.250 mmol), and the mixture was heated to 85° C. and stirred for 16 hours. The reaction was quenched with water (30 mL), and extracted with ethyl acetate (3×30 mL). The organic extracts were washed with saturated aqueous sodium chloride (80 mL), dried over anhydrous sodium sulfate, filtered, and concentrated in vacuo. The resulting residue was purified using silica gel chromatography, eluting with a gradient of methanol:dichloromethane—0:10 to 1:10 to provide compound 159h. MS (ESI, m/z): 494.2, 496.2 [M+1]⁺.

### Step H—Synthesis of Compound 159

To a mixture of 159 h (70 mg, 0.14 mmol), 159i (58.9 mg, 0.283 mmol), potassium phosphate tribasic (90 mg, 0.424 mmol) in 1,4-dioxane (1 mL), and water (0.1 mL) was added tetrakis(triphenylphosphine)palladium(0) (32.7 mg, 0.028 mmol). The reaction mixture was allowed to stir for 16 hours at 80° C. under a nitrogen atmosphere. The resulting mixture was cooled to room temperature, diluted with water (20 mL), and extracted with ethyl acetate (3×30 mL). The combined organic extracts were washed with saturated aqueous sodium chloride (5×30 mL), dried over anhydrous sodium sulfate, filtered, and concentrated in vacuo. The resulting residue was purified using preparative HPLC eluting with a gradient of acetonitrile:water (20 mmol/L ammonium bicarbonate)—4:6 to 6:4. This provide compound 159. MS (ESI, m/z): 496.2 [M+H]⁺. ¹H NMR: (400 MHz, methanol-d₄, ppm): δ 8.71 (d, J=4.4 Hz, 1H), 8.15 (s, 1H), 8.00 (s, 1H), 7.78 (d, J=3.6 Hz, 1H), 7.38-7.33 (m, 4H), 5.21 (m, 1H), 4.61 (s, 2H), 4.08 (d, J=8.8 Hz, 1H), 4.00 (s, 3H), 3.96 (d, J=8.8 Hz, 1H), 3.81-3.76 (m, 1H), 3.43-3.33 (m, 1H), 2.31 (d, J=14.4 Hz, 1H), 2.20 (d, J=15.2 Hz, 1H), 2.06-1.94 (m, 2H), 1.21 (s, 3H).

### Example 160

### Preparation of Compound 160

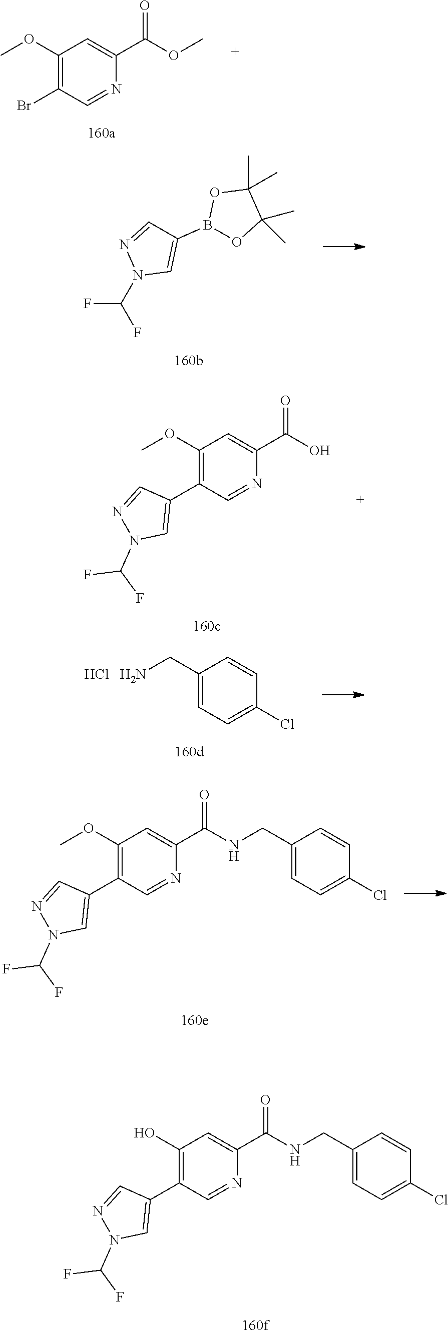

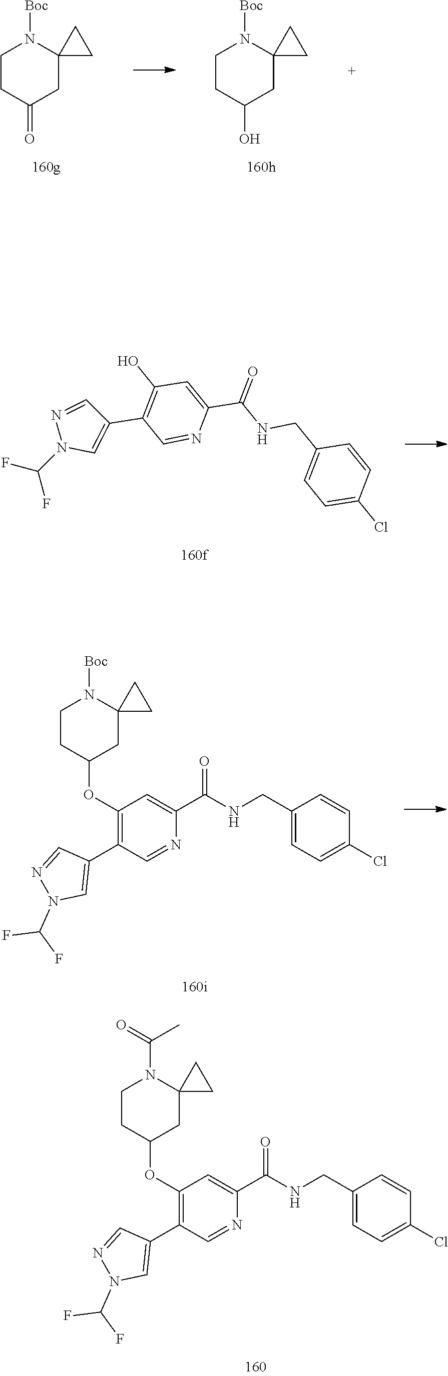 [SMILES: CC(C)(C)OC(=O)N1CCC(=O)CC12CC2 ; CC(C)(C)OC(=O)N1CCC(O)CC12CC2 ; O=C(NCc1ccc(Cl)cc1)c1cc(O)c(-c2cnn(C(F)F)c2)cn1 ; CC(C)(C)OC(=O)N1CCC(Oc2cc(C(=O)NCc3ccc(Cl)cc3)ncc2-c2cnn(C(F)F)c2)CC12CC2 ; CC(=O)N1CCC(Oc2cc(C(=O)NCc3ccc(Cl)cc3)ncc2-c2cnn(C(F)F)c2)CC12CC2]

### Step A—Synthesis of Compound 160c

A mixture of 160a (13 g, 52.8 mmol), 160b (15 g, 63.4 mmol), sodium carbonate (11 g, 106 mmol), and 1,1′-bis(diphenylphosphino)ferrocene-palladium(II)dichloride dichloromethane complex (8.6 g, 10.57 mmol) in dioxane (100 mL), and water (10 mL) was placed under nitrogen atmosphere. The reaction was heated to 90° C. and was allowed to stir at this temperature for 16 hours. The reaction mixture was filtered, and the collected solid was and dried in vacuo to provide compound 160c, which was used without further purification. MS (ESI, m/z): 270.2 [M+H]⁺.

### Step B—Synthesis of Compound 160e

A mixture of 160c (2.37 g, 8.82 mmol), and 1-(bis(dimethylamino)methylene)-1H-[1,2,3]triazolo[4,5-b]pyridine-1-ium 3-oxide hexafluorophosphate(V) (4.36 g, 11.47 mmol) in N,N-dimethylformamide (20 mL) was allowed to stir for 30 minutes at room temperature. 160d (2.042 g, 11.47 mmol), and N-ethyl-N-isopropylpropan-2-amine (3.99 g, 30.9 mmol) were added, and the resulting reaction was allowed to stir for 2 hours at room temperature. The reaction mixture was diluted with water (100 mL), extracted with ethyl acetate (3×60 mL), and the combined organic extracts were washed with water (60 mL), and brine (50 mL), dried over sodium sulfate, filtered, and concentrated in vacuo. The resulting residue was purified using silica gel chromatography, eluting with a gradient of ethyl acetate:petroleum ether—1:20 to 1:1, to provide compound 160e. MS (ESI, m/z): 393.0 [M+H]⁺.

### Step C—Synthesis of Compound 160f

To a mixture of 160e (2 g, 5.09 mmol) in acetonitrile (15 mL) was added iodotrimethylsilane (30.6 g, 153 mmol) at room temperature. The resulting reaction was heated to 80° C., and allowed to stir for 16 hours. The reaction mixture was then concentrated in vacuo, and the resulting residue was diluted with saturated aqueous sodium bicarbonate (50 mL). The resulting solution was extracted with ethyl acetate (5×60 mL), and the combined organic extracts were dried over sodium sulfate, filtered, and concentrated in vacuo. The resulting residue was purified using silica gel chromatography, eluting with a gradient of ethyl acetate:petroleum ether—1:9 to 8:2, to provide compound 160f. MS (ESI, m/z): 379.2 [M+H]⁺.

### Step D—Synthesis of Compound 160h

To a 0° C. mixture of 160 g (50 mg, 0.222 mmol) in methanol (1 mL) was added sodium tetrahydroborate (4 mg, 0.111 mmol), and the resulting reaction was allowed to stir for 1 hour at 0° C. The reaction was quenched with water (10 mL) and extracted with ethyl acetate (3×20 mL). The combined organic extracts were washed with saturated aqueous sodium chloride (50 mL), dried over anhydrous sodium sulfate, filtered, and concentrated in vacuo. The resulting residue was purified using silica gel column chromatography, eluting with a gradient of methanol:dichloromethane—0:1 to 1:20, to provide compound 160h. MS (ESI, m/z): 228.2 [M+H]⁺.

### Step E—Synthesis of Compound 160i

A solution of triphenylphosphine (69 mg, 0.264 mmol), and di-tert-butyl diazene-1,2-dicarboxylate (61 mg, 0.264 mmol) in tetrahydrofuran (1 mL) was cooled to 0° C. under nitrogen atmosphere. 160 h (20 mg, 0.088 mmol), and 160f (40 mg, 0.106 mmol) were added, and the resulting reaction was warmed to room temperature and allowed to stir at this temperature for 2 hours. The reaction was quenched with saturated aqueous ammonium chloride (10 mL) and extracted with ethyl acetate (3×30 mL). The combined organic extracts were washed with saturated aqueous sodium chloride (50 mL), dried over anhydrous sodium sulfate, filtered, and concentrated in vacuo. The resulting residue was purified using silica gel column chromatography, eluting with a gradient of methanol:dichloromethane—0:1 to 1:20, to provide compound 160i. MS (ESI, m/z): 588.2 [M+H]⁺.

### Step F—Synthesis of Compound 160

To a mixture of 160i (43 mg, 0.073 mmol) in dichloromethane (1 mL) was added trifluoroacetic acid (167 mg, 1.462 mmol), and the resulting reaction was allowed to stir for 30 minutes at room temperature. The reaction mixture was concentrated in vacuo, and the resulting residue was dissolved in dichloromethane (1 mL). The resulting solution was cooled to 0° C., and N-ethyl-N-isopropylpropan-2-amine (95 mg, 0.731 mmol), and acetyl chloride (29 mg, 0.366 mmol) were added. The cooling bath was removed, and the resulting reaction was allowed to stir for 30 minutes at room temperature. The reaction was quenched with water (10 mL), extracted with ethyl acetate (3×30 mL), and the combined organic extracts were washed with saturated aqueous sodium chloride (30 mL), dried over anhydrous sodium sulfate, filtered, and concentrated in vacuo. The resulting residue was purified using silica gel column chromatography, eluting with a gradient of methanol:dichloromethane—0:1 to 1:20, to provide compound 160. MS (ESI, m/z): 530.2 [M+H]⁺. ¹H NMR (300 MHz, DMSO-d₆, 353 K, ppm): δ 9.03 (brs, 1H), 8.79 (s, 1H), 8.62 (s, 1H), 8.32 (s, 1H), 8.00 (t, J=59.4 Hz, 1H), 7.74 (s, 1H), 7.35 (s, 4H), 5.09 (brs, 1H), 4.50 (d, J=6.0 Hz, 2H), 4.05-3.92 (m, 1H), 3.35-3.25 (m, 1H), 2.15-2.06 (m, 4H), 1.94-1.78 (m, 2H), 1.69-1.66 (m, 1H), 1.06 (s, 1H), 0.81-0.76 (m, 3H).

### Example 161

### Preparation of Compound 161

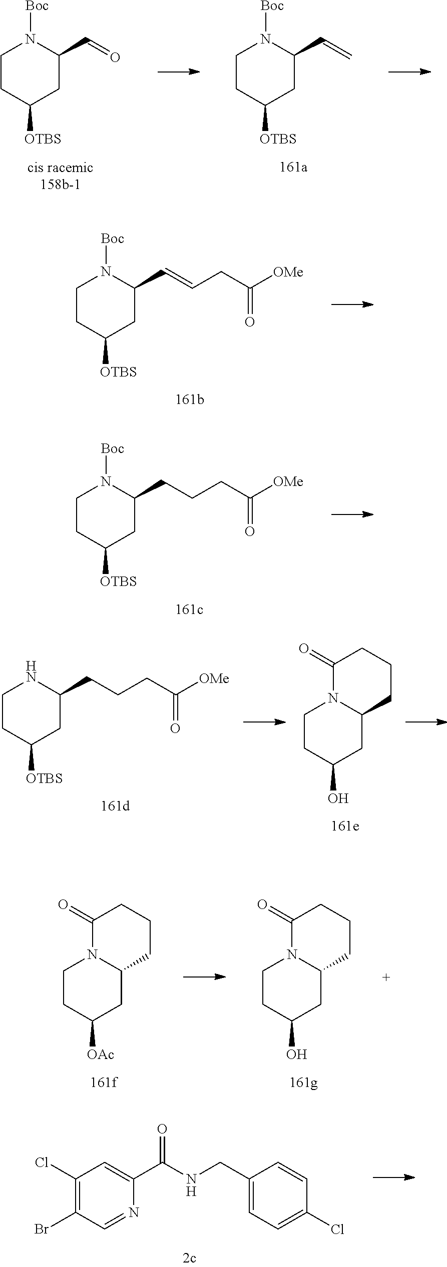

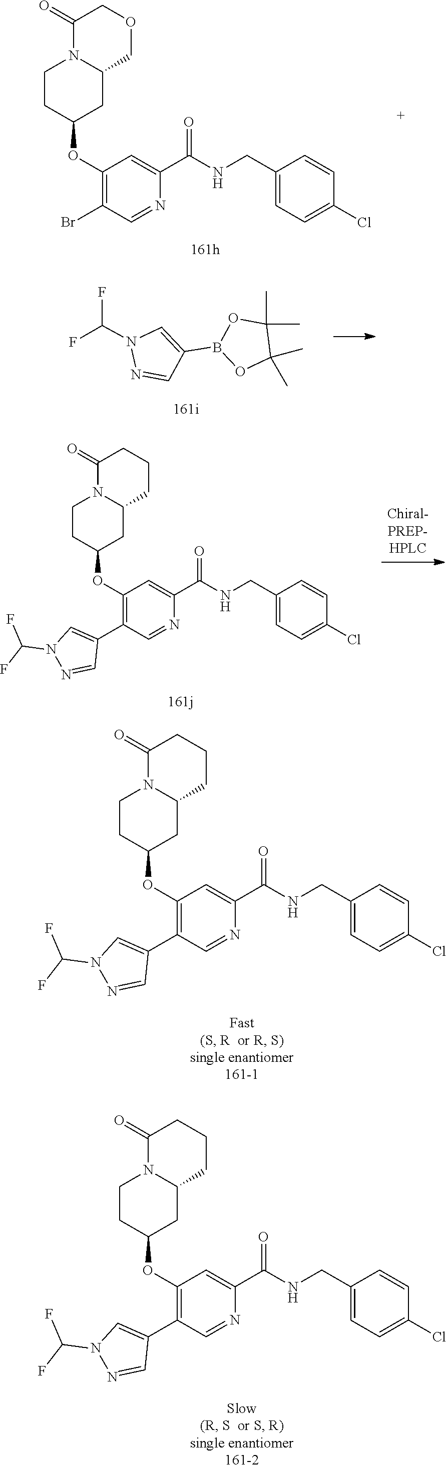 [SMILES: O=C(NCc1ccc(Cl)cc1)c1cc(O[C@H]2CCN3C(=O)COC[C@@H]3C2)c(Br)cn1 ; CC1(C)OB(c2cnn(C(F)F)c2)OC1(C)C ; O=C(NCc1ccc(Cl)cc1)c1cc(O[C@H]2CCN3C(=O)CCC[C@@H]3C2)c(-c2cnn(C(F)F)c2)cn1 ; O=C(NCc1ccc(Cl)cc1)c1cc(O[C@H]2CCN3C(=O)CCC[C@@H]3C2)c(-c2cnn(C(F)F)c2)cn1 ; O=C(NCc1ccc(Cl)cc1)c1cc(O[C@H]2CCN3C(=O)CCC[C@@H]3C2)c(-c2cnn(C(F)F)c2)cn1]

### Step A Synthesis of Compound 161a

To a solution of potassium 2-methylpropan-2-olate (196 mg, 1.747 mmol) in diethyl ether (20 mL) was added methyltriphenylphosphonium bromide (624 mg, 1.75 mmol) at room temperature. The resulting mixture was heated to 50° C. and allowed to stir for 1 hour at this temperature. The reaction was warmed to room temperature, and 158b-1 (300 mg, 0.873 mmol) was added. The reaction was allowed stir at room temperature for 1 hour, then was quenched with water (50 mL), and extracted with ethyl acetate (3×50 mL). The combined organic extracts were washed with saturated aqueous sodium chloride (40 mL), dried over anhydrous sodium sulfate, filtered, and concentrated in vacuo. The resulting residue was purified using silica gel chromatography, eluting with a gradient of ethyl acetate:petroleum ether—0:1 to 1:4, to provide compound 161a. MS (ESI, m/z): 342.2 [M+H]⁺.

### Step B—Synthesis of Compound 161b

To a mixture of 161a (240 mg, 0.703 mmol), and methyl but-3-enoate (106 mg, 1.054 mmol) in dichloromethane (7 mL) was added Grubbs catalyst 2nd generation (6 mg, 7.03 μmol). The reaction mixture was allowed to stir at 50° C. for 4 hours under argon atmosphere. The reaction mixture was quenched with water (50 mL), and extracted with ethyl acetate (3×50 mL). The combined organic extracts were washed with saturated aqueous sodium chloride (50 mL), dried over anhydrous sodium sulfate, filtered, and concentrated in vacuo. The resulting residue was purified using silica gel chromatography eluting with ethyl acetate/petroleum—1:19 to 1:1, to provide compound 161b. MS (ESI, m/z): 414.3 [M+H]⁺.

### Step C—Synthesis of Compound 161c

To a solution of 161b (120 mg, 0.290 mmol) in methanol (5 mL) was added palladium on carbon (10%, 31 mg, 0.290 mmol). The reaction vessel was evacuated and flushed three times with nitrogen, then put under hydrogen atmosphere (2 atm). The reaction was allowed to stir for 10 minutes at room temperature, then the reaction mixture was filtered, and the filtrate was concentrated in vacuo to provide compound 161c, which was used without further purification. MS (ESI, m/z): 416.3 [M+H]⁺.

### Step D—Synthesis of Compound 161d

To a solution of 161c (110 mg, 0.265 mmol) in dichloromethane (10 mL) was added trifluoroacetic acid (1 mL) at room temperature. The resulting reaction was allowed to stir for 0.5 hours at room temperature, then the reaction mixture was concentrated in vacuo to provide compound 161d, which was used without further purification. MS (ESI, m/z): 316.2 [M+H]⁺.

### Step E—Synthesis of Compound 161e

To a mixture of 161d (150 mg, 0.475 mmol), and tetrahydrofuran (5 mL) in water (5 mL) was added lithium hydroxide (34 mg, 1.426 mmol), and the resulting reaction was allowed to stir for 16 hours at room temperature. The reaction mixture was concentrated in vacuo, and the resulting residue was purified using silica gel chromatography, eluting with a gradient of methanol:dichloromethane—3:97 to 3:47, to provide compound 161e. MS (ESI, m/z): 170.1 [M+H]⁺.

### Step F—Synthesis of Compound 161f

To a mixture of triphenylphosphine (697 mg, 2.66 mmol) in tetrahydrofuran was added (E)-diisopropyl diazene-1,2-dicarboxylate (538 mg, 2.66 mmol) dropwise at 0° C. under nitrogen atmosphere. The resulting mixture was allowed to stir for 30 minutes at 0° C., then acetic acid (80 mg, 1.33 mmol), and 161e (150 mg, 0.89 mmol) were added, and the reaction mixture was warmed to room temperature and allowed to stir at this temperature for 2 hours. The reaction was quenched with water (100 mL), and the resulting solution was extracted with ethyl acetate (3×50 mL). The combined organic extracts were washed with saturated aqueous sodium chloride (50 mL), dried over anhydrous sodium sulfate, filtered, and concentrated in vacuo. The resulting residue was purified using silica gel chromatography, eluting with a gradient of ethyl acetate:petroleum ether—0:10 to 1:10, to provide compound 161f. MS (ESI, m/z): 212.3 [M+H]⁺.

### Step G—Synthesis of Compound 161g

To a mixture of 161f (100 mg, 0.47 mmol) in tetrahydrofuran (2 mL) and water (2 mL) was added lithium hydroxide hydrate (99 mg, 2.37 mmol), and the reaction was allowed to stir for 16 hours at room temperature. The reaction mixture was concentrated in vacuo, and the resulting residue was purified using silica gel chromatography, eluting with a gradient of methanol:dichloromethane—0:10 to 1:10, to provide compound 161g. MS (ESI, m/z): 169.9 [M+H]⁺.

### Step H—Synthesis of Compound 161h

To a solution of 161 g in N,N-dimethylformamide (5 mL) was added sodium hydride (25 mg, 0.62 mmol), and the resulting mixture was allowed to stir for 1 hour at room temperature. 2c (194 mg, 0.54 mmol) was added, and the reaction was heated to 80° C. and allowed to stir for 16 hours. The reaction mixture was quenched with water (50 mL), extracted with ethyl acetate (3×50 mL), and the combined extracts were washed with saturated aqueous sodium chloride (50 mL), dried over anhydrous sodium sulfate, filtered, and concentrated in vacuo. The resulting residue was purified using silica gel chromatography, eluting with a gradient of methanol:dichloromethane—0:10 to 1:10, to provide compound 161h. MS (ESI, m/z): 492.1, 494.1 [M+H]⁺.

### Step I—Synthesis of Compound 161i

To a mixture of tetrakis(triphenylphosphine)palladium(0) (23 mg, 0.02 mmol), 161 h (100 mg, 0.2 mmol), and potassium carbonate (84 mg, 0.61 mmol) in 1,4-dioxane (5 mL), and water (1 mL) was added 161i (99 mg, 0.41 mmol) at room temperature. The reaction was heated to 90° C. under nitrogen atmosphere, and allowed to stir at this temperature for 16 hours. The reaction was quenched with water (50 mL), extracted with ethyl acetate (3×50 mL), and the combined extracts were washed with saturated aqueous sodium chloride (50 mL), dried over anhydrous sodium sulfate, filtered, and concentrated in vacuo. The resulting residue was purified using silica gel chromatography, eluting with a gradient of methanol:dichloromethane—0:10 to 1:10, to provide compound 161j. MS (ESI, m/z): 530.5 [M+H]⁺.

Step J—Chiral Resolution of 161j to provide 161-1

80 mg of 161j was resolved using Chiral-Prep-HPLC (Column: CHIRALPAK IA) eluting with a gradient of hexane:ethanol—1:1 to provide compound 161-1 (fast peak). MS (ESI, m/z): 530.5 [M+H]⁺. ¹H NMR: (300 MHz, methanol-d₄, ppm): δ 8.76 (s, 1H), 8.56 (s, 1H), 8.24 (s, 1H), 7.82 (s, 1H), 7.57 (t, J=59.7 Hz, 1H), 7.37-7.30 (m, 4H), 5.16 (s, 1H), 4.72-4.51 (m, 3H), 3.78-3.58 (m, 1H), 2.87-2.78 (m, 1H), 2.37-2.33 (m, 2H), 2.29-2.11 (m, 2H), 2.08-1.98 (m, 1H), 1.91-1.82 (m, 3H), 1.75-1.64 (m, 1H), 1.60-1.50 (m, 1H).

### Example 162

### Preparation of Compound 162

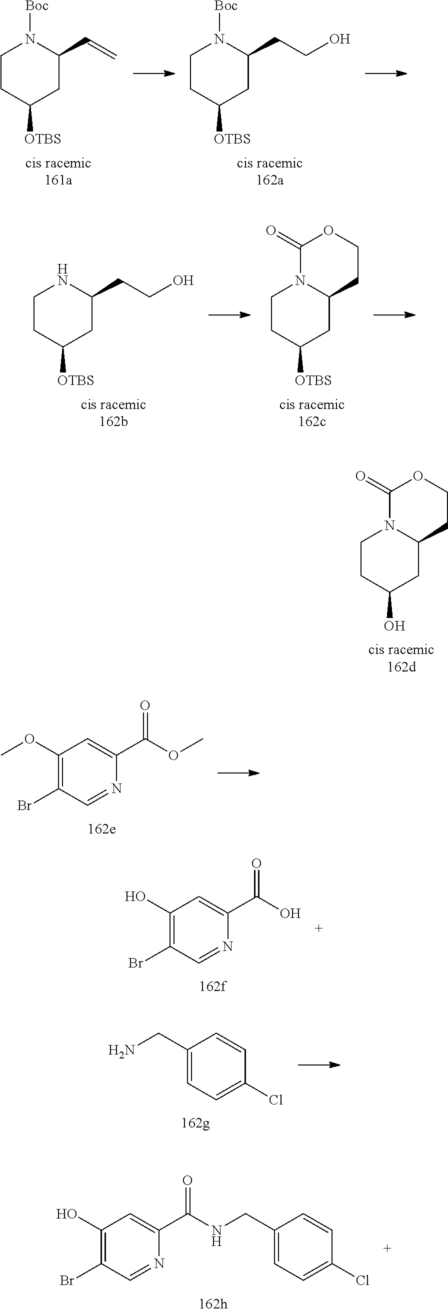

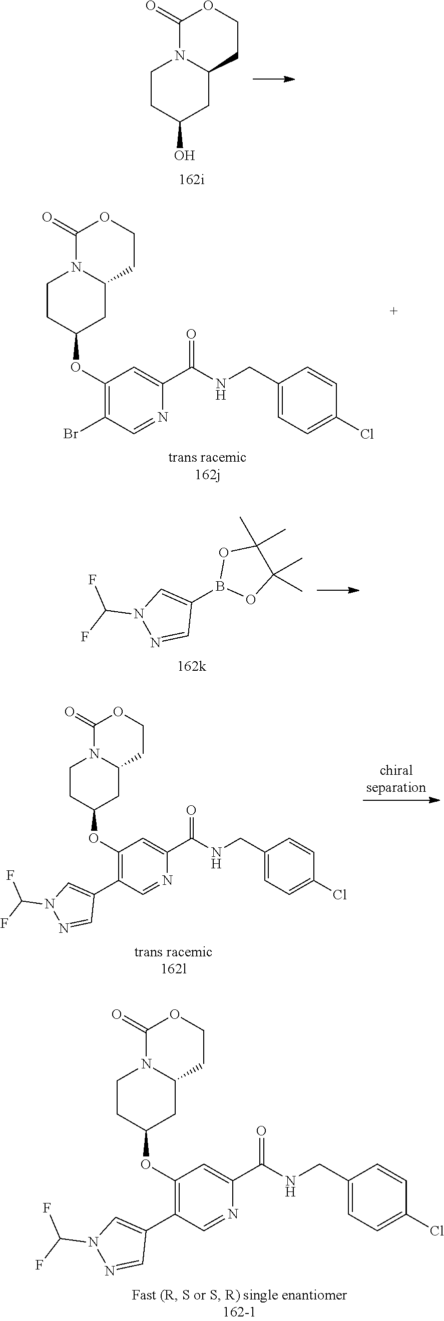 [SMILES: O=C1OCC[C@H]2C[C@@H](O)CCN12 ; O=C(NCc1ccc(Cl)cc1)c1cc(O[C@H]2CCN3C(=O)OCC[C@@H]3C2)c(Br)cn1 ; CC1(C)OB(c2cnn(C(F)F)c2)OC1(C)C ; O=C(NCc1ccc(Cl)cc1)c1cc(O[C@H]2CCN3C(=O)OCC[C@@H]3C2)c(-c2cnn(C(F)F)c2)cn1 ; O=C(NCc1ccc(Cl)cc1)c1cc(O[C@H]2CCN3C(=O)OCC[C@@H]3C2)c(-c2cnn(C(F)F)c2)cn1]

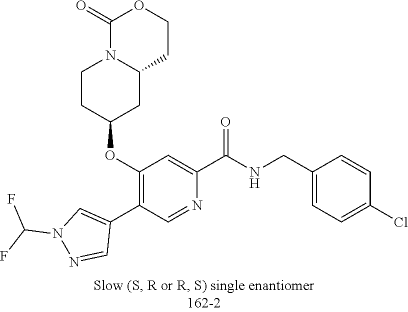 [SMILES: O=C(NCc1ccc(Cl)cc1)c1cc(O[C@H]2CCN3C(=O)OCC[C@@H]3C2)c(-c2cnn(C(F)F)c2)cn1]

### Step A—Synthesis of Compound 162a

To a mixture of 161a (2 g, 5.86 mmol), and tetrahydrofuran (20 mL) at 0° C., was added borane-tetrahydrofuran complex (17.6 mL, 17.57 mmol) and the resulting reaction was heated to 50° C., and allowed to stir for 2 hours. Water (0.13 mL), sodium hydroxide (0.5 mL, 3 N), and hydrogen peroxide (1.0 g, 8.78 mmol) were added, and the resulting mixture was allowed to stir for 2 hours at room temperature. The reaction mixture was diluted with saturated aqueous sodium chloride (50 mL) and extracted with ethyl acetate (3×50 mL). The combined organic extracts were washed with saturated aqueous sodium chloride (2×50 mL), dried over anhydrous sodium sulfate, filtered, and concentrated in vacuo. The resulting residue was purified using silica gel chromatography, eluting with a gradient of ethyl acetate:petroleum ether—1:9 to 3:7, to provide compound 162a. MS (ESI, m/z): 360.3 [M+H]⁺.

### Step B—Synthesis of Compound 162b

A mixture of 162a (500 mg, 1.391 mmol), trifluoroacetic acid (1 mL), and dichloromethane (5 mL) was allowed to stir for 1 hour at room temperature. The reaction mixture was concentrated in vacuo, and diluted with sodium hydroxide (20 mL, 3 N). The mixture was extracted with ethyl acetate (3×30 mL), and the combined organic extracts were washed with saturated aqueous sodium chloride (2×30 mL), dried over anhydrous sodium sulfate, filtered, and concentrated in vacuo to provide compound 162b. MS (ESI, m/z): 260.3 [M+H]⁺.

Step C—Synthesis of Compound 162c A mixture of sodium hydride (162 mg, 4.05 mmol), and methanol (5 mL) was allowed to stir for 1 hour at room temperature. To this mixture were added 162b (350 mg, 1.349 mmol), and dimethyl carbonate (608 mg, 6.74 mmol). The resulting mixture was allowed to stir for 2 hours at 80° C., then the reaction mixture was diluted with saturated aqueous ammonium chloride (10 mL) and was extracted with ethyl acetate (3×20 mL). The combined organic extracts were washed with saturated aqueous sodium chloride (2×20 mL), dried over anhydrous sodium sulfate, and the concentrated in vacuo. The resulting residue was purified using silica gel chromatography, eluting with a gradient of methanol:dichloromethane—3:97 to 10:90, to provide compound 162c. MS (ESI, m/z): 286.2 [M+H]⁺.

### Step D—Synthesis of Compound 162d

A mixture of 162c tetrabutylammonium fluoride trihydrate (3.5 mL, 3.50 mmol), and tetrahydrofuran (4 mL) was allowed to stir for 16 hours at room temperature. The reaction mixture was diluted with methanol (10 mL), and concentrated in vacuo. The resulting residue was purified using silica gel chromatography, eluting with a gradient of methanol:dichloromethane—4:96 to 10:90 to provide compound 162d. MS (ESI, m/z): 172.2 [M+H]⁺.

### Step E—Synthesis of Compound 162f

A mixture of 162e (20 g, 81 mmol) in hydrobromic acid (150 mL) was allowed to stir for 2 days at 90° C. The reaction mixture was concentrated under vacuum, to provide compound 162f, which was used without further purification. MS (ESI, m/z): 217.8, 219.8 [M+H]⁺.

Step F—synthesis of compound 162h

To a solution of 162f (6.0 g, 27.5 mmol) in dichloromethane (40 mL) were added 1-propanephosphonic anhydride (42.0 g, 66.1 mmol), and 162 g (7.79 g, 55.0 mmol) at room temperature. The reaction mixture was allowed to stir for 2 hours at 50° C., then was diluted with water (40 mL). The resulting solution was filtered, and the collected solid was washed with water (3×50 mL) and dried in vacuo to provide compound 162h, which was used without further purification. MS (ESI, m/z): 342.9 [M+H]⁺.

### Step G—Synthesis of Compound 162i

To a solution of triphenyl phosphine (598 mg, 2.278 mmol) in tetrahydrofuran (8 mL) at 0° C. under nitrogen atmosphere, was added di-tert-butyl azodicarboxylate (525 mg, 2.278 mmol). The resulting reaction was allowed to stir for 15 minutes at 0° C., then 162 h and 162i (389 mg, 1.139 mmol) were added, and the reaction was allowed to stir for 1 hour at 25° C. The reaction mixture was quenched with water (20 mL), extracted with ethyl acetate (3×30 mL), and the combined organic extracts were washed with saturated aqueous sodium chloride (20 mL), dried over anhydrous sodium sulfate, filtered, and concentrated in vacuo. The resulting residue was purified using silica gel chromatography, eluting with a gradient of ethyl acetate:petroleum ether—40:60 to 100:0, to provide compound 162i. MS (ESI, m/z): 496.1 [M+H]⁺.

### Step H—Synthesis of Compound 162L

To a solution of 162i (140 mg, 0.283 mmol) in 1,4-dioxane (5 mL), and water (0.5 mL) was added 162k (207 mg, 0.849 mmol), tetrakis(triphenylphosphine)-palladium(0) (49.0 mg, 0.042 mmol), and potassium phosphate tribasic (180 mg, 0.849 mmol). The reaction mixture was allowed to stir for 8 hours at 90° C. under nitrogen atmosphere. The resulting mixture was diluted with water (25 mL) and extracted with ethyl acetate (3×30 mL). The combined organic extracts were washed with saturated aqueous sodium chloride (25 mL), dried over anhydrous sodium sulfate, filtered, and concentrated in vacuo. The resulting residue was purified using preparative HPLC, eluting with a gradient of acetonitrile:water (0.05% trifluoroacetic acid)—40:60 to 70:30, to provide compound 162L, as a mixture of diastereomers. MS (ESI, m/z): 532.2 [M+H]⁺.

Step I—Chiral Resolution of compound 162L

162 L (106 mg, 0.169 mmol, diasteromeric mixture) was resolved using the Prep-Chiral-HPLC (Column: CHIRALPAK IA), eluting with a gradient of hexane:dichloromethane:ethanol—5:1:6 to provide compound 162-1 (fast peak). MS (ESI, m/z): 532.3 [M+H]⁺. ¹H NMR (400 MHz, methanol-d₄, ppm): δ 8.76 (s, 1H), 8.56 (s, 1H), 8.25 (s, 1H), 7.82 (s, 1H), 7.58 (t, J=60.0 Hz, 1H), 7.40-7.30 (m, 4H), 5.19 (s, 1H), 4.59 (s, 2H), 4.29-4.16 (m, 3H), 3.74-3.70 (m, 1H), 3.12-3.06 (m, 1H), 2.26-2.13 (m, 3H), 2.03-1.77 (m, 3H).

### Example 163

### Preparation of Compound 163

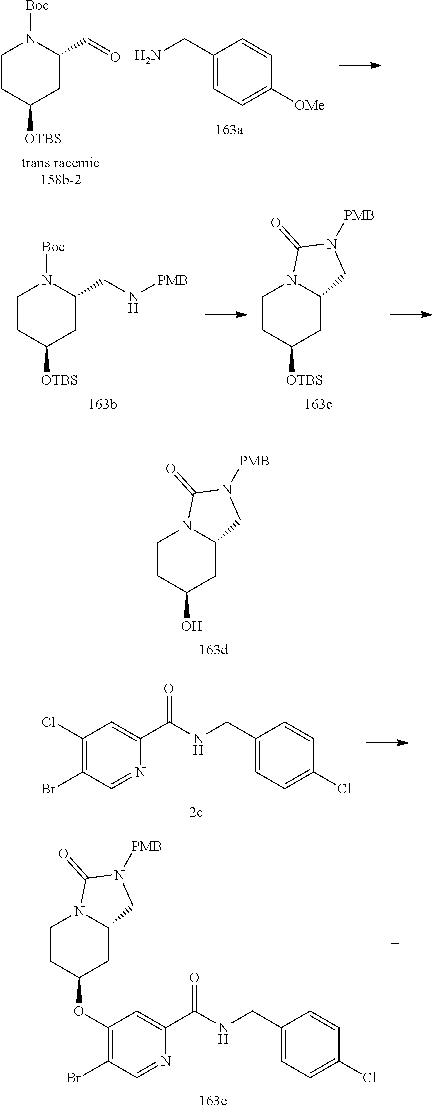

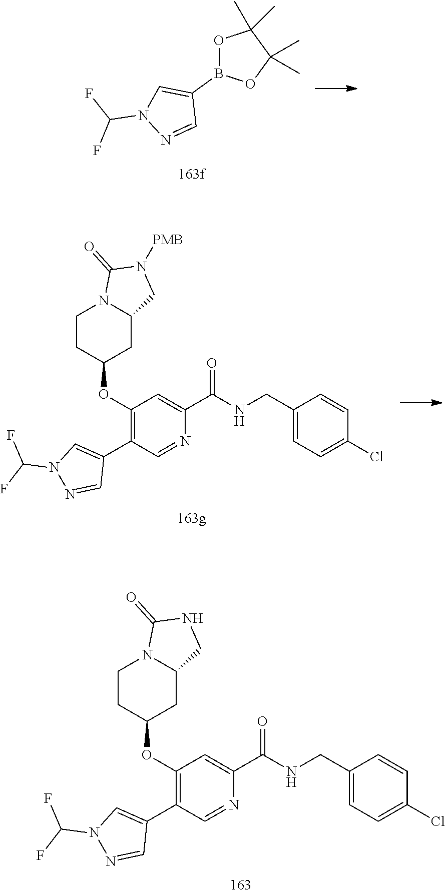

### Step A—Synthesis of Compound 163b

To a mixture of 158b-2 (500 mg, 1.455 mmol) in 1,2-dichloroethane (15 mL) was added 163a (399 mg, 2.91 mmol), and the resulting reaction was allowed to stir for 1 hour at room temperature. Sodium cyanotrihydroborate (274 mg, 4.37 mmol) was added, and the resulting mixture was allowed to stir for an additional 2 hours at room temperature. The resulting reaction was diluted with water (50 mL) and extracted with ethyl acetate (3×50 mL). The combined organic extracts were washed with saturated aqueous sodium chloride (50 mL), dried over anhydrous sodium sulfate, filtered, and concentrated in vacuo. The resulting residue was purified using silica gel chromatography, eluting with a gradient of ethyl acetate:petroleum ether—0:10 to 1:5, to provide compound 163b. MS (ESI, m/z): 465.0 [M+H]⁺.

### Step B—Synthesis of Compound 163c

To a mixture of 163b (370 mg, 0.796 mmol) in tetrahydrofuran (15 mL) was added potassium 2-methylpropan-2-olate (357 mg, 3.18 mmol) at room temperature. The resulting mixture was heated to 80° C. and allowed to stir at this temperature for 16 hours. The reaction mixture was diluted with water (10 mL), extracted with ethyl acetate (3×50 mL), and the combined organic extracts were washed with saturated aqueous sodium chloride (50 mL), dried over anhydrous sodium sulfate, filtered, and concentrated in vacuo. The resulting residue was purified using silica gel chromatography, eluting with a gradient of ethyl acetate:petroleum ether—2:5 to 5:2, to provide compound 163c. MS (ESI, m/z): 391.2 [M+H]⁺.

### Step C—Synthesis of Compound 163d

To a mixture of 163c (300 mg, 0.768 mmol) in tetrahydrofuran (2 mL) was added tetrabutylammonium fluoride (1M in tetrahydrofuran, 1.5 mL, 1.5 mmol), and the reaction was allowed to stir for 16 hours at room temperature. The reaction mixture was diluted with water (20 mL) and extracted with ethyl acetate (3×20 mL). The combined organic extracts were washed with saturated aqueous sodium chloride (20 mL), dried over anhydrous sodium sulfate, filtered, and concentrated in vacuo. The resulting residue was purified using silica gel chromatography, eluting with a gradient of methanol:dichloromethane—2:98 to 8:92, to provide compound 163d. MS (ESI, m/z): 277.1 [M+H]⁺.

### Step D—Synthesis of Compound 163e

To a solution of 163d (100 mg, 0.362 mmol) in N,N-dimethylformamide (2 mL) was added sodium hydride (22 mg, 0.543 mmol), and the resulting mixture was allowed to stir for 10 minutes at room temperature. To this mixture was added 2c (156 mg, 0.434 mmol), and the resulting reaction was heated to 85° C. and allowed to stir at this temperature for 8 hours. The reaction mixture was diluted with water (40 mL), extracted with ethyl acetate (3×50 mL), and the combined organic extracts were washed with saturated aqueous sodium chloride (80 mL), dried over anhydrous sodium sulfate, filtered, and concentrated in vacuo. The resulting residue was purified using silica gel chromatography, eluting with a gradient of methanol:dichloromethane—1:99 to 10:90, to provide compound 163e. MS (ESI, m/z): 599.4, 601.1 [M+H]⁺.

### Step E—Synthesis of Compound 163g

A mixture of potassium phosphate tribasic (170 mg, 0.800 mmol), 163f (98 mg, 0.400 mmol), and 163e (160 mg, 0.267 mmol) in 1,4-dioxane (2 mL), and water (0.2 mL) was added tetrakis(triphenylphosphine)palladium(0) (62 mg, 0.053 mmol) was put under nitrogen atmosphere. The reaction was heated to 90° C. and allowed to stir at this temperature for 16 hours. The reaction mixture was diluted with water (20 mL), extracted with ethyl acetate (3×30 mL), and the combined organic extracts were washed with saturated aqueous sodium chloride (80 mL), dried over anhydrous sodium sulfate, filtered, and concentrated in vacuo. The resulting residue was purified using silica gel chromatography, eluting with a gradient of methanol:dichloromethane—1:99 to 10:90, to provide compound 163g. MS (ESI, m/z): 637.6 [M+H]⁺.

### Step F—Synthesis of Compound 163

To a mixture of 163 g (35 mg, 0.055 mmol) in trifluoroacetic acid (1.8 mL) was added anisole (0.6 mL), and the reaction was heated to 80° C., and allowed to stir at this temperature for 16 hours. The reaction mixture was concentrated in vacuo, and the residue obtained was purified using preparative HPLC, eluting with a gradient of acetonitrile:water (20 mmol/L ammonium bicarbonate)—1:4 to 3:1, to provide compound 163. MS (ESI, m/z): 517.2 [M+H]⁺. ¹H NMR (400 MHz, methanol-d₄, ppm): δ 8.75 (s, 1H), 8.55 (s, 1H), 8.24 (s, 1H), 7.82 (s, 1H), 7.59 (t, J=60.0 Hz, 1H), 7.36-7.30 (m, 4H), 5.24 (s, 1H), 4.59 (s, 2H), 3.88-3.82 (m, 1H), 3.77-3.72 (m, 1H), 3.51 (t, J=8.8 Hz, 1H), 3.08-3.00 (m, 2H), 2.23 (d, J=14.0 Hz, 1H), 2.08 (t, J=14.4 Hz, 1H), 1.89-1.83 (m, 2H).

The following compounds of the present invention were made using the methods described in Examples 157-163 above, and substituting the appropriate reactants and/or reagents:

| | | MS |
|---|---|---|
| Compound | Structure | [M + H]⁺ |
| 164 | 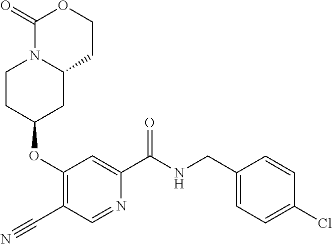 [SMILES: N#Cc1cnc(C(=O)NCc2ccc(Cl)cc2)cc1O[C@H]1CCN2C(=O)OCC[C@@H]2C1] | 441.2 |
| 165 | 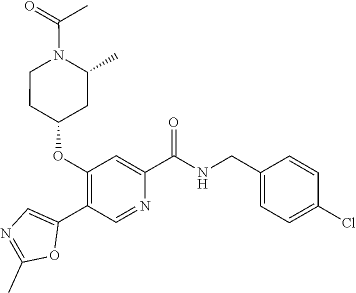 [SMILES: CC(=O)N1CC[C@@H](Oc2cc(C(=O)NCc3ccc(Cl)cc3)ncc2-c2cnc(C)o2)C[C@H]1C] | 483.4 |
| 166 | 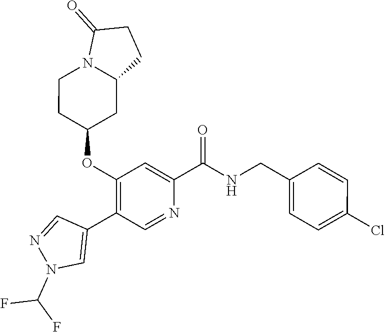 [SMILES: O=C(NCc1ccc(Cl)cc1)c1cc(O[C@H]2CCN3C(=O)CC[C@@H]3C2)c(-c2cnn(C(F)F)c2)cn1] | 516.3 |
| 167 | 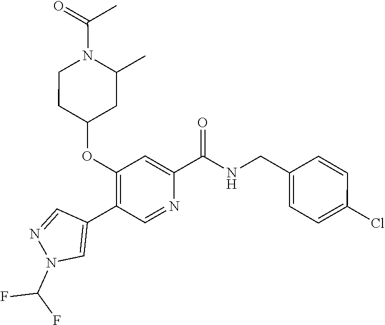 [SMILES: CC(=O)N1CCC(Oc2cc(C(=O)NCc3ccc(Cl)cc3)ncc2-c2cnn(C(F)F)c2)CC1C] | 518.3 |
| 168 | 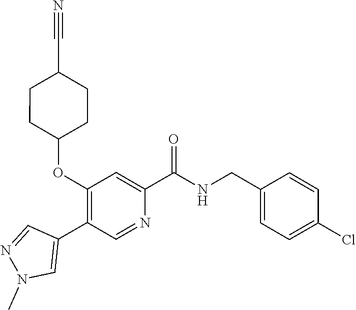 [SMILES: Cn1cc(-c2cnc(C(=O)NCc3ccc(Cl)cc3)cc2OC2CCC(C#N)CC2)cn1] | 450.3 |
| 169 | 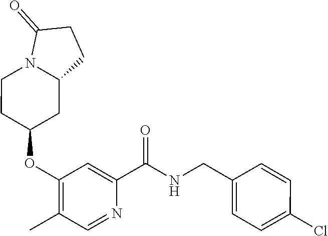 [SMILES: Cc1cnc(C(=O)NCc2ccc(Cl)cc2)cc1O[C@H]1CCN2C(=O)CC[C@@H]2C1] | 414.1 |
| 170* | 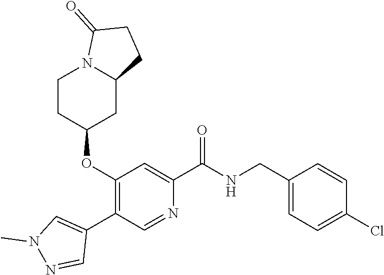 [SMILES: Cn1cc(-c2cnc(C(=O)NCc3ccc(Cl)cc3)cc2O[C@H]2CCN3C(=O)CC[C@H]3C2)cn1] | 480.4 |
| 171* | 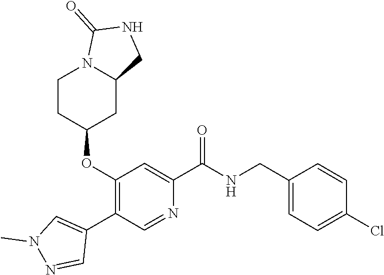 [SMILES: Cn1cc(-c2cnc(C(=O)NCc3ccc(Cl)cc3)cc2O[C@H]2CCN3C(=O)NC[C@H]3C2)cn1] | 481.1 |

*denotes mixture of cis isomers

### Example 172

### Preparation of Compound 172

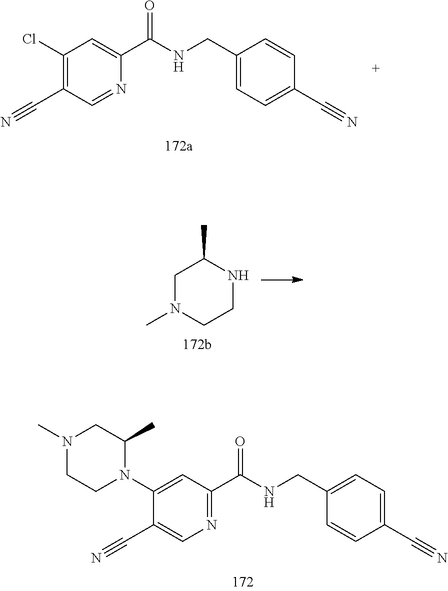

### Step A—Synthesis of Compound 172

To a solution of 172a (180 mg, 0.61 mmol, prepared using the methodology described in Example 2, Step A) in DMSO (2.5 mL) was added 172b (0.49 mL, 3.6 mmol), and triethylamine (0.25 mL, 1.8 mmol), and the resulting reaction mixture was heated to 60° C. and allowed to stir at this temperature for 48 hours. The reaction was mixture was cooled to room temperature, filtered, and purified using preparative HPLC (reverse-phase C-18), eluting with acetonitrile/water+0.1% TFA 5-75%, to provide compound 172. MS (ESI, m/z): 375.2 [M+H]⁺. ¹H NMR (600 MHz, Chloroform-d) δ 8.46-8.41 (m, 2H), 7.67-7.60 (m, 3H), 7.43 (d, J=8.3 Hz, 2H), 4.68 (d, J=6.4 Hz, 2H), 4.53 (s, 1H), 3.84 (d, J=13.0 Hz, 1H), 3.54 (td, J=12.7, 3.2 Hz, 1H), 2.90 (d, J=10.8 Hz, 1H), 2.72 (d, J=11.4 Hz, 1H), 2.33 (dd, J=11.2, 3.3 Hz, 1H), 2.29 (s, 3H), 2.20-2.15 (m, 1H), 1.38 (d, J=6.7 Hz, 3H).

The following compounds of the present invention were made using the methods described in Example 172 above, and substituting the appropriate reactants and/or reagents:

| | | MS |
|---|---|---|
| Compound | Structure | [M + H]⁺ |
| 173 | 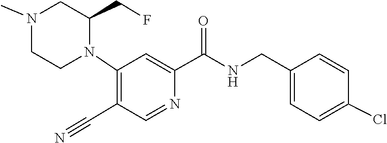 [SMILES: CN1CCN(c2cc(C(=O)NCc3ccc(Cl)cc3)ncc2C#N)[C@H](CF)C1] | 402.1 |
| 174 | 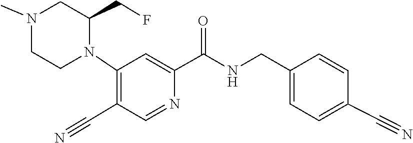 [SMILES: CN1CCN(c2cc(C(=O)NCc3ccc(C#N)cc3)ncc2C#N)[C@H](CF)C1] | 393.2 |
| 175 | 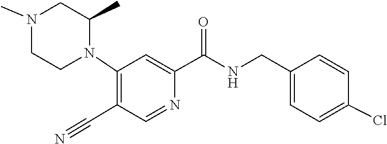 [SMILES: C[C@@H]1CN(C)CCN1c1cc(C(=O)NCc2ccc(Cl)cc2)ncc1C#N] | 384 |

### Example 176

### Preparation of Compound 176

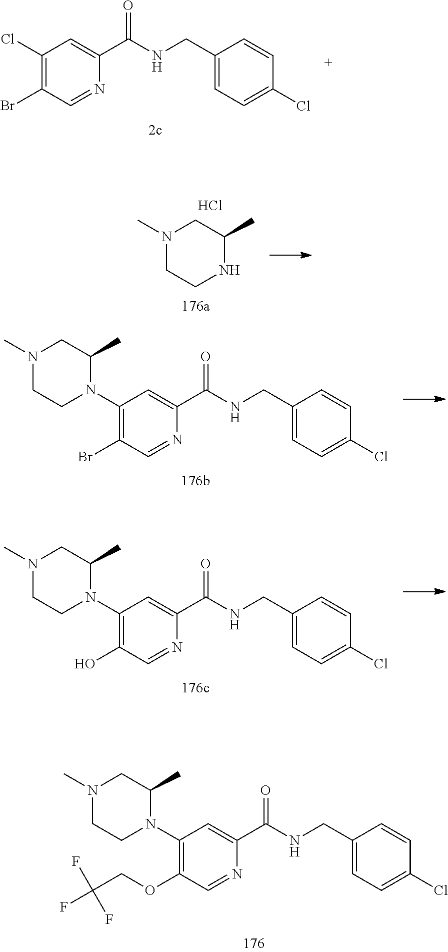

### Step A—Synthesis of Compound 176b

A mixture of 2c (600 mg, 1.67 mmol), 176a (468 mg, 2.50 mmol), and N-ethyl-N-isopropylpropan-2-amine (646 mg, 5.00 mmol) in nitrobenzene (4 mL) was allowed to stir for 16 hours at 200° C. under nitrogen atmosphere. The reaction mixture was concentrated in vacuo. The resulting residue was purified using silica gel chromatography, eluting with a gradient of methanol:dichloromethane—1:10 to 1:30 to provide compound 176b. MS (ESI, m/z): 437.1, 439.1 [M+H]⁺.

### Step B—Synthesis of Compound 176c

A mixture of 176b (40 mg, 0.091 mmol), benzaldoxime (33.2 mg, 0.274 mmol), cesium carbonate (119 mg, 0.365 mmol), and [(2-di-tert-butylphosphino-3-methoxy-6-methyl-2′,4′,6′-triisopropyl-1,1′-biphenyl)-2-(2-aminobiphenyl)]palladium(II) methanesulfonate (7.66 mg, 9.14 μmol) in N,N-dimethylformamide (1.5 mL). The reaction mixture was irradiated with microwave radiation for 2 hours at 110° C. The reaction mixture was then quenched with water (10 mL), extracted with ethyl acetate (3×10 mL). The combined organic extracts were washed with saturated aqueous sodium chloride (10 mL), dried over anhydrous sodium sulfate, filtered, and concentrated in vacuo. The resulting residue was purified using silica gel chromatography, eluting with a gradient of methanol:dichloromethane—1:99 to 5:95 to provide compound 176c. MS (ESI, m/z): 375.2 [M+H]⁺.

### Step C—Synthesis of Compound 176

To a solution of 176c (50 mg, 0.133 mmol), and cesium carbonate (130 mg, 0.400 mmol) in N,N-dimethylformamide (2 mL) was added 2,2,2-trifluoroethyl trifluoromethanesulfonate (46 mg, 0.200 mmol). The resulting mixture was allowed to stir for 1.5 hours at room temperature. The reaction mixture was diluted with water (10 mL) and extracted with ethyl acetate (2×10 mL). The combined organic extracts were washed with saturated aqueous sodium chloride (10 mL), dried over anhydrous sodium sulfate, filtered, and concentrated in vacuo. The resulting residue was purified using preparative HPLC, eluting with a gradient of acetonitrile:water (20 mmol/L ammonium bicarbonate)—62:38 to 66:34 to provide compound 176. MS (ESI, m/z): 457.3 [M+H]⁺. ¹H NMR (400 MHz, methanol-d₄, ppm): δ 8.16 (s, 1H), 7.65 (s, 1H), 7.33-7.28 (m, 4H), 4.75-4.64 (m, 2H), 4.56 (s, 2H), 4.12-4.09 (m, 1H), 3.48-3.42 (m, 1H). 3.19-3.15 (m, 1H), 2.79-2.75 (m, 1H), 2.60-2.49 (m, 2H), 2.38-2.30 (m, 1H), 2.29 (s, 3H), 1.14 (d, J=6.8 Hz, 3H).

### Example 177

### Preparation of Compound 177

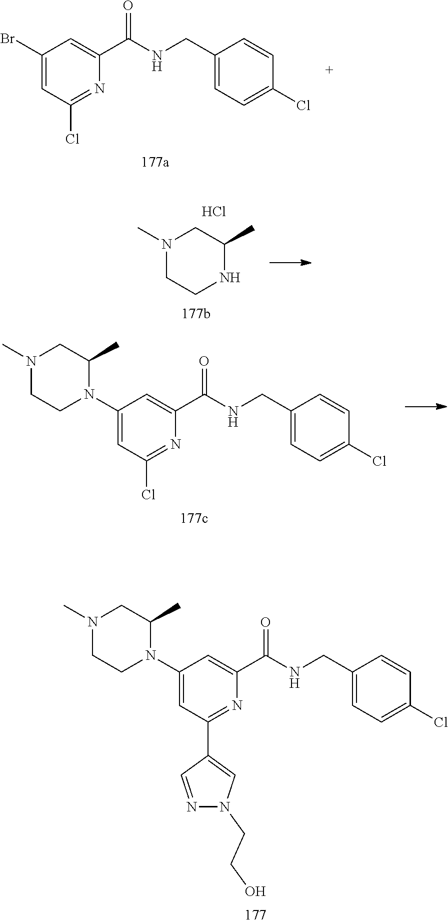

### Step A—Synthesis of Compound 177c

A mixture of 177a (200 mg, 0.56 mmol, made using the methodology described in Example 2, Step A), 177b (312 mg, 1.67 mmol), 2-((2,6-dimethoxyphenyl)amino)-2-oxoacetic acid (50 mg, 0.22 mmol), potassium phosphate tribasic (700 mg, 3.33 mmol), and copper(I) iodide (10.6 mg, 0.056 mmol) in DMSO (5 mL) was allowed to stir for 48 hours at 100° C. under nitrogen atmosphere. The reaction mixture was diluted with ethyl acetate, washed with water and brine, dried of magnesium sulfate, filtered and concentrate in vacuo to provide an oil. The oil was purified using silica gel chromatography, eluting with a gradient of 0-75% (3:1 ethyl acetate:ethanol)/hexanes to provide compound 177c. MS (ESI, m/z): 494.2 [M+H]⁺.

### Step B—Synthesis of Compound 177

Compound 177 was prepared from 177c, using the methodology described in Example 3, Step A to provide compound 177. MS (ESI, m/z): 469.2 [M+H]⁺. ¹H NMR (600 MHz, Chloroform-d) δ 8.47 (d, J=5.7 Hz, 1H), 7.91 (s, 2H), 7.49 (d, J=2.1 Hz, 1H), 7.29 (s, 4H), 6.82 (d, J=2.0 Hz, 1H), 4.62 (d, J=6.3 Hz, 2H), 4.28-4.24 (m, 2H), 4.21 (s, 1H), 4.03-3.99 (m, 2H), 3.69-3.14 (m, 2H), 2.92 (s, 1H), 2.77 (d, J=10.2 Hz, 1H), 2.30 (s, 3H), 2.29-1.94 (m, 3H), 1.25 (d, J=6.7 Hz, 3H).

### Example 178

### Preparation of Compound 178

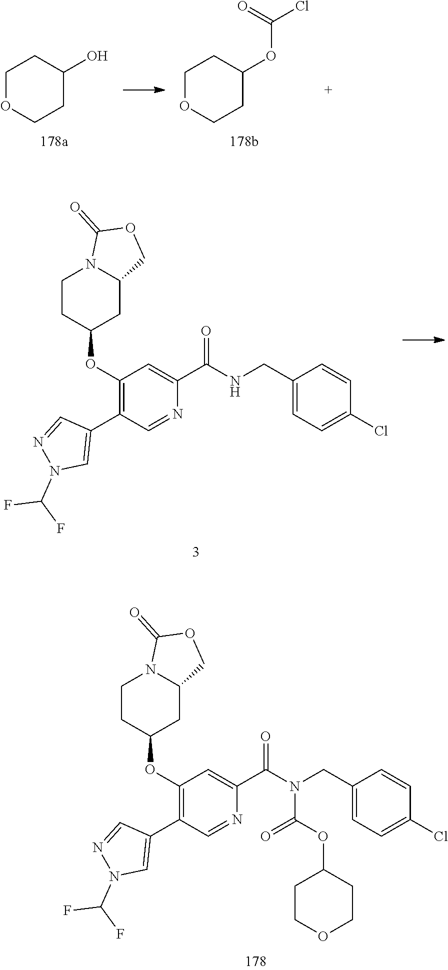 [SMILES: OC1CCOCC1 ; O=C(Cl)OC1CCOCC1 ; O=C(NCc1ccc(Cl)cc1)c1cc(O[C@H]2CCN3C(=O)OC[C@@H]3C2)c(-c2cnn(C(F)F)c2)cn1 ; O=C(OC1CCOCC1)N(Cc1ccc(Cl)cc1)C(=O)c1cc(O[C@H]2CCN3C(=O)OC[C@@H]3C2)c(-c2cnn(C(F)F)c2)cn1]

### Step A—Synthesis of Compound 178b

To a mixture of 178a (1.8 g, 17.33 mmol), and pyridine (1.4 mL, 17.33 mmol) in dichloromethane (30 mL) was added bis(trichloromethyl) carbonate (2.6 g, 8.67 mmol) in dichloromethane (10 mL) dropwise at 0° C. The resulting mixture was allowed to stir for 2 hours at room temperature, and concentrated in vacuo. The resulting residue was dissolved in ethyl acetate (50 mL), and stirred for 30 minutes at room temperature. The resulting mixture was filtered, and the filtrate was concentrated in vacuo to provide compound 178b. ¹H NMR (400 MHz, chloroform-d, ppm): δ 5.07-5.01 (m, 1H), 3.98-3.92 (m, 2H), 3.59-3.53 (m, 2H), 2.08-2.02 (m, 2H), 1.94-1.75 (m, 2H).

### Step B—Synthesis of Compound 178

To a mixture of compound 3 (500 mg, 0.97 mmol) in N,N-dimethylformamide (5.0 mL) was added potassium bis(trimethylsilyl)amide (1 M in tetrahydrofuran, 1.5 mL, 1.448 mmol) dropwise at 0° C. under nitrogen atmosphere. The resulting mixture was allowed to stir for 10 minutes at 0° C. To this was added 178b (191 mg, 1.16 mmol) at 0° C. The resulting mixture was allowed to stir for 16 hours at room temperature. The reaction mixture was quenched with saturated aqueous ammonium chloride (15 mL) and extracted with ethyl acetate (3×20 mL). The combined organic extracts were washed with saturated aqueous sodium chloride (10 mL), dried over anhydrous sodium sulfate, filtered, and concentrated in vacuo. The resulting residue was purified using preparative HPLC, eluting with a gradient of water (0.8% ammonium bicarbonate):acetonitrile—35:65 to provide compound 178. MS (ESI, m/z): 646.5 [M+H]⁺. ¹H NMR (400 MHz, methanol-d₄, ppm): δ 8.68 (s, 1H), 8.55 (s, 1H), 8.23 (s, 1H), 7.58 (t, J=59.6 Hz, 1H), 7.54 (s, 1H), 7.45-7.43 (m, 2H), 7.35-7.34 (m, 2H), 5.27 (s, 1H), 5.02 (s, 2H), 4.80-4.77 (m, 1H), 4.47-4.40 (m, 1H), 4.00-3.96 (m, 2H), 3.78-3.74 (m, 1H), 3.56-3.51 (m, 2H), 3.41-3.31 (m, 2H), 3.22-3.18 (m, 1H), 2.35-2.31 (m, 1H), 2.12-2.03 (m, 1H), 1.96-1.78 (m, 2H), 1.72-1.68 (m, 2H), 1.30-1.25 (m, 2H).

The following compound of the present invention was made using the methods described in Example 178 above, and substituting the appropriate reactants and/or reagents:

| | | MS |
|---|---|---|
| Compound | Structure | [M + H]⁺ |
| 179 | 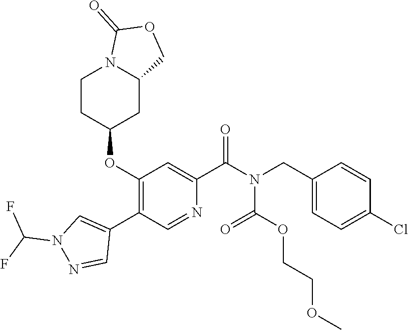 [SMILES: COCCOC(=O)N(Cc1ccc(Cl)cc1)C(=O)c1cc(O[C@H]2CCN3C(=O)OC[C@@H]3C2)c(-c2cnn(C(F)F)c2)cn1] | 620.5 |

### Example 180

### Preparation of Compound 180

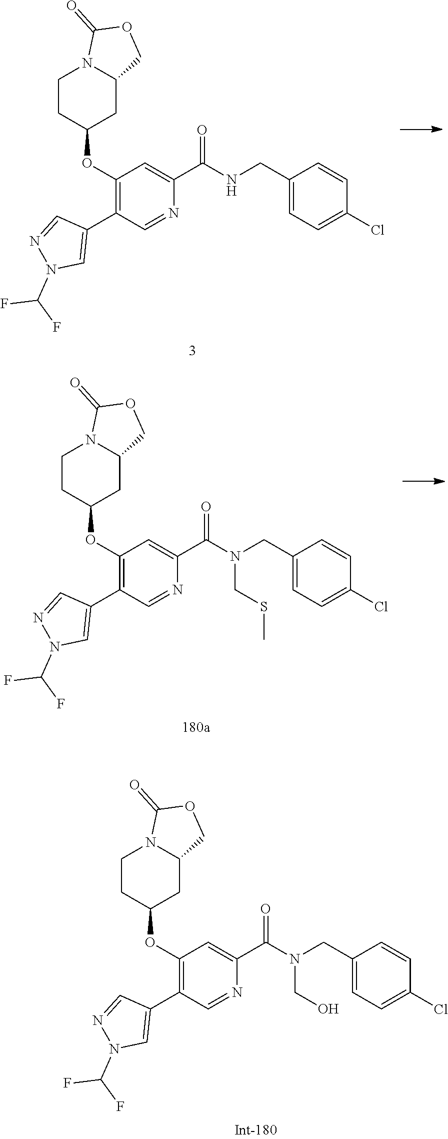 [SMILES: O=C(NCc1ccc(Cl)cc1)c1cc(O[C@H]2CCN3C(=O)OC[C@@H]3C2)c(-c2cnn(C(F)F)c2)cn1 ; CSCN(Cc1ccc(Cl)cc1)C(=O)c1cc(O[C@H]2CCN3C(=O)OC[C@@H]3C2)c(-c2cnn(C(F)F)c2)cn1 ; O=C(c1cc(O[C@H]2CCN3C(=O)OC[C@@H]3C2)c(-c2cnn(C(F)F)c2)cn1)N(CO)Cc1ccc(Cl)cc1]

### Step A—Synthesis of Compound 180b

To a mixture of compound 3 (600 mg, 1.16 mmol) in N,N-dimethylformamide (10.0 mL) at 0° C. under nitrogen atmosphere, was added sodium iodide (208 mg, 1.39 mmol), and potassium hexamethyldisilazane (1 M in tetrahydrofuran) (1.9 mL, 1.85 mmol). To the resulting mixture was added chloromethyl methyl sulfane (280 mg, 2.90 mmol), and the resulting reaction was allowed to stir for 5 minutes at 0° C., then for 8 hours at room temperature. The reaction mixture was then concentrated in vacuo, and the resulting residue was purified using preparative HPLC eluting with a gradient of acetonitrile:water (0.1% trifluoroacetic acid)—1:9 to 6:4, to provide 180a. MS (ESI, m/z): 578.3 [M+H]⁺.

### Step B—Synthesis of Intermediate Compound Int-180

To a mixture of 180a (70 mg, 0.12 mmol) in tetrahydrofuran (5.0 mL) was added 1-iodopyrrolidine-2,5-dione (40 mg, 0.18 mmol). The resulting reaction was allowed to stir at room temperature for 5 minutes, then water (0.5 mL) was added, and the reaction was allowed to stir for an additional 20 minutes. The reaction mixture was diluted with water (20 mL), and extracted with ethyl acetate (3×20 mL). The combined organic extracts were washed with saturated aqueous sodium chloride (20 mL), dried over anhydrous sodium sulfate, filtered, and concentrated in vacuo. The resulting residue was purified using silica gel chromatography, eluting with a gradient of methanol:dichloromethane—0:100 to 5:95 to provide intermediate compound 180. MS (ESI, m/z): 548.2 [M+H]⁺.

### Example 177

### Preparation of Compound 177

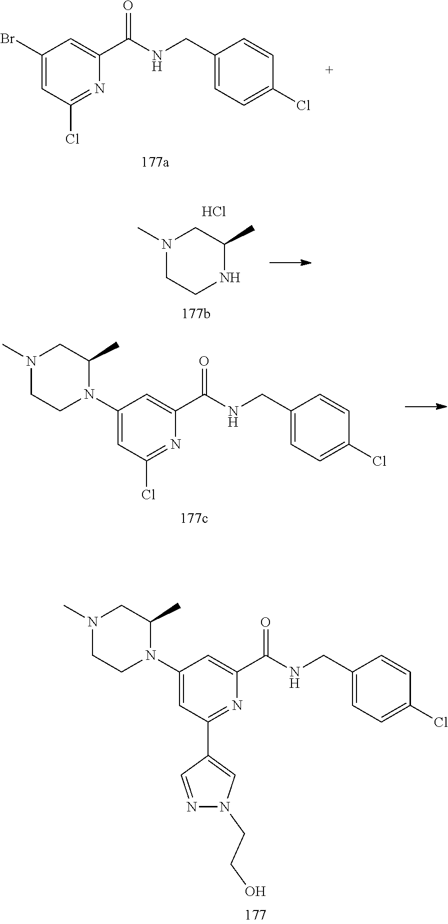

### Step A—Synthesis of Compound 177c

A mixture of 177a (200 mg, 0.56 mmol, made using the methodology described in Example 2, Step A), 177b (312 mg, 1.67 mmol), 2-((2,6-dimethoxyphenyl)amino)-2-oxoacetic acid (50 mg, 0.22 mmol), potassium phosphate tribasic (700 mg, 3.33 mmol), and copper(I) iodide (10.6 mg, 0.056 mmol) in DMSO (5 mL) was allowed to stir for 48 hours at 100° C. under nitrogen atmosphere. The reaction mixture was diluted with ethyl acetate, washed with water and brine, dried of magnesium sulfate, filtered and concentrate in vacuo to provide an oil. The oil was purified using silica gel chromatography, eluting with a gradient of 0-75% (3:1 ethyl acetate:ethanol)/hexanes to provide compound 177c. MS (ESI, m/z): 494.2 [M+H]⁺.

### Step B—Synthesis of Compound 177

Compound 177 was prepared from 177c, using the methodology described in Example 3, Step A to provide compound 177. MS (ESI, m/z): 469.2 [M+H]⁺. ¹H NMR (600 MHz, Chloroform-d) δ 8.47 (d, J=5.7 Hz, 1H), 7.91 (s, 2H), 7.49 (d, J=2.1 Hz, 1H), 7.29 (s, 4H), 6.82 (d, J=2.0 Hz, 1H), 4.62 (d, J=6.3 Hz, 2H), 4.28-4.24 (m, 2H), 4.21 (s, 1H), 4.03-3.99 (m, 2H), 3.69-3.14 (m, 2H), 2.92 (s, 1H), 2.77 (d, J=10.2 Hz, 1H), 2.30 (s, 3H), 2.29-1.94 (m, 3H), 1.25 (d, J=6.7 Hz, 3H).

### Example 178

### Preparation of Compound 178

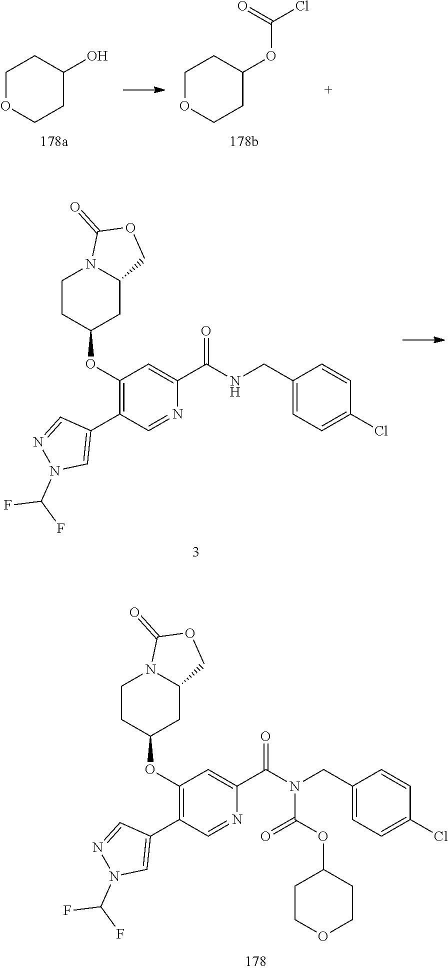 [SMILES: OC1CCOCC1 ; O=C(Cl)OC1CCOCC1 ; O=C(NCc1ccc(Cl)cc1)c1cc(O[C@H]2CCN3C(=O)OC[C@@H]3C2)c(-c2cnn(C(F)F)c2)cn1 ; O=C(OC1CCOCC1)N(Cc1ccc(Cl)cc1)C(=O)c1cc(O[C@H]2CCN3C(=O)OC[C@@H]3C2)c(-c2cnn(C(F)F)c2)cn1]

### Step A—Synthesis of Compound 178b

To a mixture of 178a (1.8 g, 17.33 mmol), and pyridine (1.4 mL, 17.33 mmol) in dichloromethane (30 mL) was added bis(trichloromethyl) carbonate (2.6 g, 8.67 mmol) in dichloromethane (10 mL) dropwise at 0° C. The resulting mixture was allowed to stir for 2 hours at room temperature, and concentrated in vacuo. The resulting residue was dissolved in ethyl acetate (50 mL), and stirred for 30 minutes at room temperature. The resulting mixture was filtered, and the filtrate was concentrated in vacuo to provide compound 178b. ¹H NMR (400 MHz, chloroform-d, ppm): δ 5.07-5.01 (m, 1H), 3.98-3.92 (m, 2H), 3.59-3.53 (m, 2H), 2.08-2.02 (m, 2H), 1.94-1.75 (m, 2H).

### Step B—Synthesis of Compound 178

To a mixture of compound 3 (500 mg, 0.97 mmol) in N,N-dimethylformamide (5.0 mL) was added potassium bis(trimethylsilyl)amide (1 M in tetrahydrofuran, 1.5 mL, 1.448 mmol) dropwise at 0° C. under nitrogen atmosphere. The resulting mixture was allowed to stir for 10 minutes at 0° C. To this was added 178b (191 mg, 1.16 mmol) at 0° C. The resulting mixture was allowed to stir for 16 hours at room temperature. The reaction mixture was quenched with saturated aqueous ammonium chloride (15 mL) and extracted with ethyl acetate (3×20 mL). The combined organic extracts were washed with saturated aqueous sodium chloride (10 mL), dried over anhydrous sodium sulfate, filtered, and concentrated in vacuo. The resulting residue was purified using preparative HPLC, eluting with a gradient of water (0.8% ammonium bicarbonate):acetonitrile—35:65 to provide compound 178. MS (ESI, m/z): 646.5 [M+H]⁺. ¹H NMR (400 MHz, methanol-d₄, ppm): δ 8.68 (s, 1H), 8.55 (s, 1H), 8.23 (s, 1H), 7.58 (t, J=59.6 Hz, 1H), 7.54 (s, 1H), 7.45-7.43 (m, 2H), 7.35-7.34 (m, 2H), 5.27 (s, 1H), 5.02 (s, 2H), 4.80-4.77 (m, 1H), 4.47-4.40 (m, 1H), 4.00-3.96 (m, 2H), 3.78-3.74 (m, 1H), 3.56-3.51 (m, 2H), 3.41-3.31 (m, 2H), 3.22-3.18 (m, 1H), 2.35-2.31 (m, 1H), 2.12-2.03 (m, 1H), 1.96-1.78 (m, 2H), 1.72-1.68 (m, 2H), 1.30-1.25 (m, 2H).

The following compounds of the present invention were made using the methods described in Example 181 above, and substituting the appropriate reactants and/or reagents:

| | | MS |
|---|---|---|
| Compound | Structure | [M + H]⁺ |
| 182 | 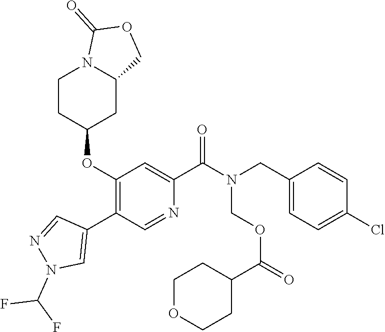 [SMILES: O=C(OCN(Cc1ccc(Cl)cc1)C(=O)c1cc(O[C@H]2CCN3C(=O)OC[C@@H]3C2)c(-c2cnn(C(F)F)c2)cn1)C1CCOCC1] | 660.6 |
| 183 | 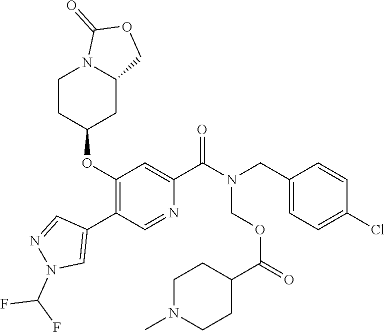 [SMILES: CN1CCC(C(=O)OCN(Cc2ccc(Cl)cc2)C(=O)c2cc(O[C@H]3CCN4C(=O)OC[C@@H]4C3)c(-c3cnn(C(F)F)c3)cn2)CC1] | 673.7 |
| 184 | 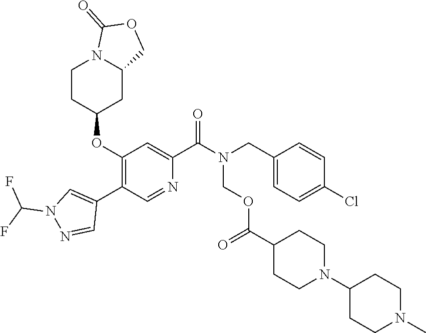 [SMILES: CN1CCC(N2CCC(C(=O)OCN(Cc3ccc(Cl)cc3)C(=O)c3cc(O[C@H]4CCN5C(=O)OC[C@@H]5C4)c(-c4cnn(C(F)F)c4)cn3)CC2)CC1] | 756.7 |
| 185 | 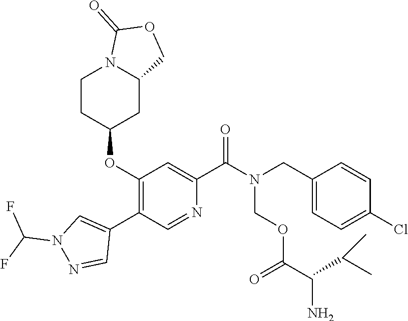 | 669 [M + Na] |

### Example 186

### Preparation of Compound 186

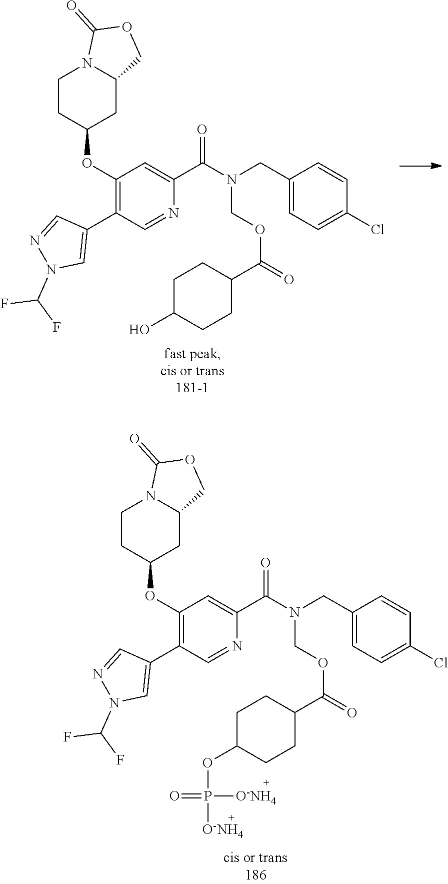

### Synthesis of Compound 186

To a mixture of 181-1 (cis or trans) (130 mg, 0.19 mmol) in tetrahydrofuran (2.5 mL) was added dropwise a mixture of pyrophosphoryl chloride (121 mg, 0.48 mmol) in tetrahydrofuran (0.2 mL) at −10° C. under argon atmosphere. The reaction mixture was allowed to stir at −10° C. for 30 minutes, then warmed to 0° C., and allowed to stir at this temperature for another 30 minutes. The reaction was quenched with saturated aqueous sodium bicarbonate (0.1 mL), and the resulting solution was allowed to stir for 20 minutes at room temperature. The reaction mixture was then directly purified using preparative HPLC, eluting with a gradient of acetonitrile:water (10 mmol/L ammonium bicarbonate)—18:82 to 50:50, to provide compound 186. MS (ESI, m/z): 754.2 [M−2NH₃+H]⁺. ¹H NMR (400 MHz, methanol-d₄, ppm): δ 8.69 (s, 1H), 8.55 (s, 1H), 8.23 (s, 1H), 7.73-7.28 (m, 6H), 5.60-5.57 (m, 2H), 5.26 (s, 1H), 4.82 (s, 2H), 4.45 (t, J=7.6 Hz, 1H), 4.01-3.96 (m, 3H), 3.78-3.73 (m, 1H), 3.20-3.17 (m, 1H), 2.39-2.26 (m, 1H), 2.18-2.03 (m, 4H), 1.95-1.81 (m, 4H), 1.39-1.29 (m, 4H).

### Example 187

### Preparation of Compound 187

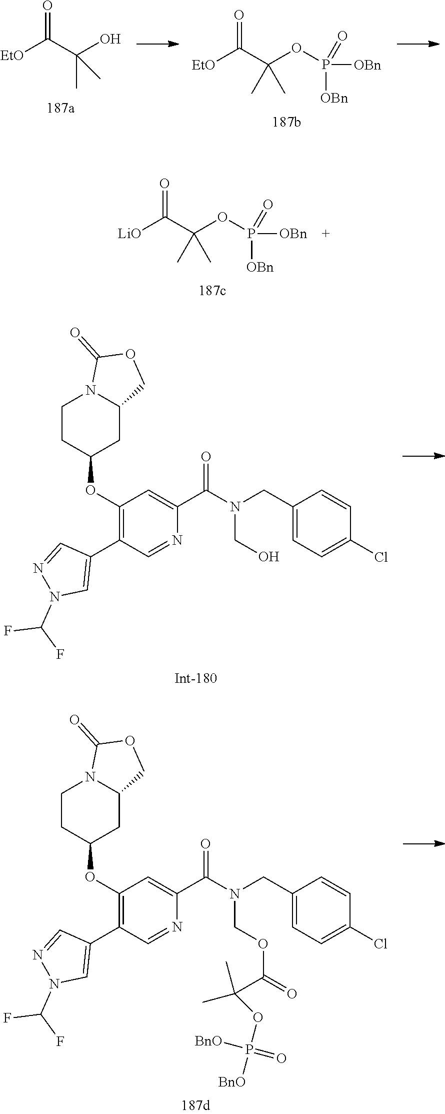

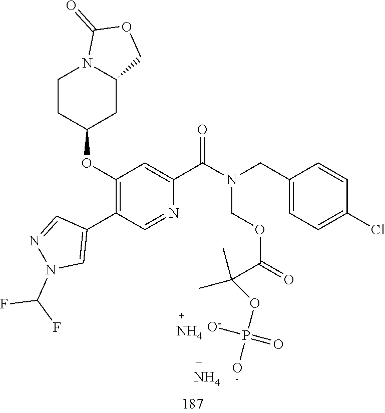

### Step A—Synthesis of Compound 187b

To a mixture of 187a (1.0 g, 7.57 mmol), and 1H-tetrazole (1.0 g, 15.1 mmol) in dry dichloromethane (20 mL) was added dibenzyl diisopropylphosphoramidite (4.7 g, 13.6 mmol) under argon atmosphere. The resulting reaction was allowed to stir for 2 hours at room temperature, then the reaction mixture was cooled to 0° C. and m-CPBA (2.8 g, 13.62 mmol) in dichloromethane (5 mL) was added. The reaction was then warmed to room temperature and allowed to stir at this temperature for 1 hour. The reaction mixture was then diluted with dichloromethane (50 mL), and the organic phase was washed with saturated aqueous sodium metabisulfite (20 mL), and saturated aqueous sodium bicarbonate (20 mL), dried over anhydrous sodium sulfate, filtered, and concentrated in vacuo. The resulting residue was purified using silica gel chromatography, eluting with a gradient of ethyl acetate:petroleum ether—0:1 to 1:1, to provide compound 187b. MS (ESI, m/z): 393.1 [M+H]⁺.

### Step B—Synthesis of Compound 187c

To a mixture of 187b (500 mg, 1.27 mmol) in methanol (1.0 mL), water (1.0 mL), and tetrahydrofuran (3.0 mL) was added lithium hydroxide (61 mg, 2.55 mmol). The resulting reaction was allowed to stir for 3 hours at room temperature. The reaction mixture was then concentrated in vacuo to provide compound 187c, which was used without further purification. MS (ESI, m/z): 365.0 [M+2H−Li]⁺.

### Step C—Synthesis of Compound 187d

To a mixture of Int-180 (300 mg, 0.55 mmol) in N,N-dimethyl formamide (2.0 mL), and 2-(7-azabenzotriazol-1-yl)-N,N,N′,N′-tetramethyluronium hexafluorophosphate (833 mg, 2.19 mmol) was added 187c (385 mg, 1.04 mmol), and N,N-diisopropylethylamine (0.5 mL, 2.74 mmol). The resulting reaction was allowed to stir at room temperature for 16 hours, then the reaction mixture was diluted with water (10 mL) and extracted with ethyl acetate (3×20 mL). The combined organic extracts were washed with saturated aqueous sodium chloride (20 mL), dried over anhydrous sodium sulfate, filtered, and concentrated in vacuo. The resulting residue was purified using silica gel chromatography, eluting with a gradient of ethyl acetate:petroleum ether—0:1 to 1:1, to provide compound 187d. MS (ESI, m/z): 894.1 [M+H]⁺.

### Step D—Synthesis of Compound 187

To a mixture of 187d (40 mg, 0.045 mmol) in methanol (2.0 mL), and ethyl acetate (20 mL) was added palladium on carbon (10%, wet) (45 mg, 0.038 mmol) under nitrogen atmosphere. The resulting mixture was allowed to stir for 30 minutes at room temperature under hydrogen (1.5 atm). The reaction mixture was filtered through Celite diatomaceous earth, and the filtrate was concentrated in vacuo. The resulting residue was purified using preparative HPLC, eluting with a gradient of acetonitrile:water (0.8% ammonium bicarbonate)—20:80, to 60:40 to provide compound 187. MS (ESI, m/z): 714.1 [M+H−2NH₃]⁺. ¹H NMR (400 MHz, methanol-d₄, ppm): δ 8.74 (s, 1H), 8.58 (s, 1H), 8.26 (s, 1H), 7.75-7.33 (m, 6H), 5.64-5.49 (m, 2H), 5.33 (s, 1H), 4.91-4.84 (m, 2H), 4.49-4.45 (m, 1H), 4.03-3.99 (m, 2H), 3.81-3.77 (m, 1H), 3.33-3.15 (m, 1H), 2.44-2.41 (m, 1H), 2.21-2.18 (m, 1H), 1.94-1.75 (m, 2H), 1.64-1.53 (m, 6H).

### Example 188

### Preparation of Compound 188

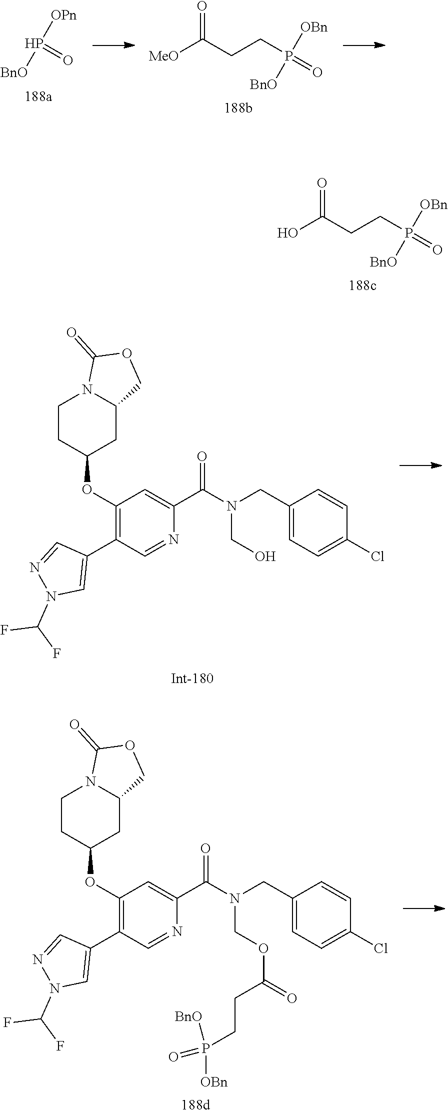

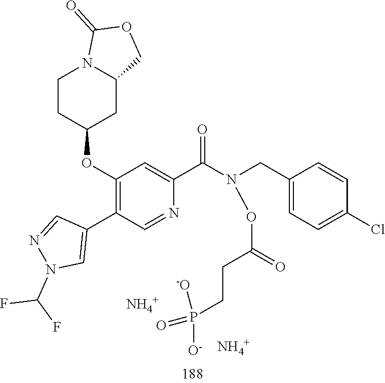

### Step A—Synthesis of Compound 188b

To a mixture of 188a (5 g, 19.1 mmol) in tetrahydrofuran (50.0 mL) was added sodium hydride (1.14 g, 28.6 mmol) at 0° C. The reaction was allowed to stir for 10 minutes at 0° C., then methyl 3-bromopropanoate (3.18 g, 19.1 mmol) was added, and the reaction mixture was allowed to stir at room temperature for 48 hours. The reaction was quenched with saturated aqueous ammonium chloride (50 mL), extracted with ethyl acetate (100 mL), and the organic extract was dried over anhydrous sodium sulfate, filtered, and concentrated in vacuo. The resulting residue was purified using silica gel chromatography, eluting with a gradient of ethyl acetate:petroleum ether—1:3, to provide compound 188b. MS: (ESI, m/z) 349.1 [M+H]⁺.

### Step B—Synthesis of Compound 188c

To a mixture of 188b (1.0 g, 2.75 mmol) in tetrahydrofuran (6.0 mL), methanol (2.0 mL), and water (2.0 mL) was added lithium hydroxide hydrate (69 mg, 3.30 mmol). The resulting reaction was allowed to stir for 5 minutes at room temperature, then the reaction mixture was neutralized with hydrochloric acid (3 M, 2.0 mL) at 0° C. The resulting mixture was diluted with water (10 mL) and extracted with chloroform/isopropanol (3:1, 3×50 mL). The combined organic extracts were washed with saturated aqueous sodium chloride (50 mL), dried over anhydrous sodium sulfate, filtered, and concentrated in vacuo to provide compound 188c, which was used without further purification. MS: (ESI, m/z) 335.0 [M+H]⁺.

### Step C and D—Synthesis of Compound 188

Compound 188 was synthesized from compound 188c, using the methodology described in Example 187, Steps C and D. MS: (ESI, m/z) 684.2 [M−2NH₃+H]⁺. ¹H NMR (400 MHz, methanol-d₄, ppm): δ 8.73 (s, 1H), 8.58 (s, 1H), 8.27 (s, 1H), 7.61 (t, J=59.6 Hz, 1H), 7.54-7.31 (m, 5H), 5.63-5.52 (m, 2H), 5.29 (s, 1H), 4.93-4.85 (m, 2H), 4.47-4.45 (m, 1H), 4.03-4.00 (m, 2H), 3.82-3.77 (m, 1H), 3.38-3.19 (m, 1H), 2.70-2.39 (m, 3H), 2.20 (d, J=14.0 Hz, 1H), 1.99-1.78 (m, 4H).

### Example 189

### Preparation of Compound 189

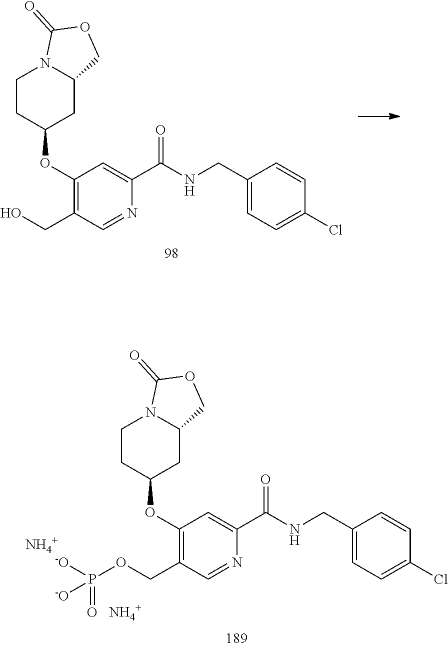

### Synthesis of Compound 189

To compound 98 in THE (0.5 mL) at −15° C. was added pyrophosphoryl chloride (0.024 mL, 0.18 mmol), and the reaction was allowed to stir for 30 minutes, then was allowed to warm to 0° C., and stir at this temperature for 30 minutes. The reaction was quenched with aqueous saturated sodium bicarbonate solution, and the reaction mixture was directly purified using preparative HPLC, eluting with a gradient of acetonitrile:water (NH₄OH)—5-95%, to provide compound 189. MS (ESI, m/z): 512.3 [M+H]⁺. ¹H NMR (600 MHz, DMSO-d₆) δ 9.36 (s, 1H), 8.49 (s, 1H), 7.56 (s, 1H), 7.35-7.27 (m, 4H), 5.11 (s, 1H), 4.79 (d, J=6.3 Hz, 2H), 4.41 (d, J=6.4 Hz, 2H), 4.31 (t, J=8.0 Hz, 2H), 3.92 (d, J=98.5 Hz, 2H), 3.56 (s, 1H), 3.48 (s, 1H), 3.13 (d, J=13.7 Hz, 1H), 2.03 (s, 1H), 1.72 (s, 1H), 1.65 (d, J=12.9 Hz, 1H), 1.47 (s, 1H).

### Example 190

### HCMV and VZV Polymerase Assays

Human cytomegalovirus and varicella zoster virus DNA polymerases were expressed via baculovirus vector in SF21 cells and purified. Heterodimeric nucleic acid substrate used in the herpesvirus polymerase reactions was generated by annealing a 59-mer template to a 17-mer digoxigenin-labeled primer. Polymerase (HCMV final concentration of 0.2 nM; VZV final concentration of 0.4 nM) was combined with an inhibitor compound or DMSO in assay buffer (10 mM HEPES, pH 7.5, 25 mM KCl, 25 mM NaCl, 5 mM MgCl₂, 5% glycerol, 0.67 mg/ml bovine serum albuminutes, and 1 mM tris(2-carboxyethyl)phosphine), and this mixture was pre-incubated for 30 minutes at room temperature in 384-well microtiter plates. The polymerization reaction was initiated by the addition of template/primer substrate (final concentration: 1.6 nM), and dNTPs (final concentration: 24 nM dCTP, 24 nMdGTP, 16 nM dATP, 16 nM dTTP, and 0.8 nM biotin-dUTP). After 60 minutes incubation at 37° C., reactions were terminated with quench buffer (25 mM HEPES pH 7.5, 100 mM NaCl, 0.25% Tween-20, 12 mM EDTA, and 1 mg/ml bovine serum albumin). Incorporation of biotinylated UTP was detected with 2.5-5 ug/ml anti-DIG AlphaLISA acceptor beads and 5-10 ug/ml streptavidin AlphaLISA donor beads (Perkin Elmer). Compound effects were normalized to the window defined by the controls (DMSO only and pre-quenched wells), and were fit using a 4-parameter algorithm to report an IC₅₀.

### Example 191

### Viral qPCR Assays

Viral qPCR Assays (V0/V1)

MRC5 cells, Vero cells, and MeWo cells were obtained from ATCC and were maintained at 37° C./5% CO2/90% relative humidity in Minimal Essential Medium with 10% fetal bovine serum, 2.0 nM L-glutamine, 100 units/ml penicillin and 100 ug/ml streptomycin. Assay plates were prepared by dispensing compounds dissolved in DMSO into wells of 384 well collagen-coated plates using an ECHO acoustic dispenser. Each compound was tested in a 10-point, serial 3-fold dilution. Controls included uninfected cells and infected cells treated only with DMSO. Assays were initiated by mixing selected cells, in suspension, with virus, and dispensing 50 μl/well infected cells to pre-plated compounds. Plates were incubated at 37° C./5% CO2/90% relative humidity for ˜72 hrs to permit genomic replication, and infected cells were lysed by the addition of an equal volume of lysis buffer (10 mM Tris-HCl, pH8, 50 mM KCl, 2 mM MgCl₂, 0.45% NP-40, 0.45% Tween-20, and 100 μg/ml proteinase K). An aliquot of the lysate was transferred to a 384-well PCR plate and incubated at 56° C. for 1 hour and then at 95° C. for 10 min. Levels of a viral gene and of the cellular control, PPIA (Thermo Fisher Assay ID=Hs04194521_s1), were measured in separate 10 μL qPCR assays using TaqMan® Gene Expression Master Mix (Applied Biosystems), and an 7900HT Fast Real-Time PCR System with 384-Well Block Module. 7-point, serial 10-fold dilutions of a plasmid standard were run on each plate to generate a standard curve, and genome copies numbers were calculated by plotting experimental Ct onto linear regression of the standard curve. Viral genome copy numbers were normalized by cellular control copy number, and compound effects were normalized to the window defined by the controls. Calculated % effects were fit using a 4-parameter algorithm, and EC₅₀ was reported.

Viral qPCR Assays (V2/V3)

MRC5 cells, Vero cells, and MeWo cells were obtained from ATCC and were maintained at 37° C./5% CO2/90% relative humidity in Minimal Essential Medium with 10% fetal bovine serum, 2.0 nM L-glutamine, 100 units/ml penicillin and 100 ug/ml streptomycin. Assay plates were prepared by dispensing compounds dissolved in DMSO into wells of 384 well collagen-coated plates using an ECHO acoustic dispenser. Each compound was tested in a 10-point, serial 3-fold dilution. Controls included uninfected cells and infected cells treated only with DMSO. Assays were initiated by mixing selected cells, in suspension, with virus, and dispensing 50 μl/well infected cells to pre-plated compounds. Plates were incubated at 37° C./5% CO2/90% relative humidity for ˜72 hrs to permit genomic replication, and infected cells were lysed by the addition of an equal volume of lysis buffer (10 mM Tris-HCl, pH8, 50 mM KCl, 2 mM MgCl₂, 0.45% NP-40, 0.45% Tween-20, and 100 μg/ml proteinase K). An aliquot of the lysate was transferred to a 384-well PCR plate and incubated at 56° C. for 1 hour and then at 95° C. for 10 min. Levels of a viral gene were measured in 10 μL qPCR assays using TaqMan® Gene Expression Master Mix (Applied Biosystems), and an 7900HT Fast Real-Time PCR System with 384-Well Block Module. 7-point, serial 10-fold dilutions of a plasmid standard were run on each plate to generate a standard curve, and genome copies numbers were calculated by plotting experimental Ct onto linear regression of the standard curve. Compound effects on viral genome copy number were normalized to the window defined by the controls. Calculated % effects were fit using a 4-parameter algorithm, and EC₅₀ was reported.
HCMV: Strain AD169 Street was assayed in MRC-5 cells and was used at 0.05-0.1 pfu/cell. The assays were performed in either growth media or in the same media with 50% fetal bovine serum. Primer-probe set was Thermo Fisher Assay ID=AIFATFK.
VZV: Strain Street was maintained as an infected cell stock in MRC-5 cells. It was assayed in MeWo or MRC5 cells and was used at 0.04-0.1 pfu/cell. The assays were performed in growth medium. Primer-probe set was Thermo Fisher Assay ID=AIPAEXQ.
HSV-1: Strain F Street was assayed in Vero or MRC5 cells and was used at 0.0005-0.004 pfu/cell in growth medium. Primer-probe set was Thermo Fisher Assay ID=AIBJZIB

Illustrative compounds of the present invention were tested in one or more of the above assays and results are provided in the table below:

| | | | | VZV Cell^{b} | VZV Cell^{b} | HSV1 Cell^{b} | HSV1 Cell^{b} |
|---|---|---|---|---|---|---|---|
| | | | | (EC₅₀) | (EC₅₀) | (EC₅₀) | (EC₅₀) |
| | HCMVª | VZV^{a} | HCMV Cell^{b} | MEWO | MRC5 | VERO | MRC5 |
| Compound | (IC₅₀) | (IC₅₀) | (EC₅₀) | 10% | 10% | 10% | 10% |
| 3 | 10.27 | 7.391 | 180.5 | 112.1 | 169.9 | 70.93 | 126.9 |
| 4 | 59.07 | 20.32 | 291.6 | N/A | N/A | 209.7 | N/A |
| 6 | 79 | 30.75 | 341.7 | 84.52 | N/A | 143.7 | N/A |
| 7 | 121.9 | N/A | 551.8 | N/A | 706.7 | N/A | 530.3 |
| 8 | 26.17 | 33.28 | 440.1 | 368.2 | N/A | 258.7 | N/A |
| 9 | 146.5 | 113.6 | 581.9 | 858.9 | N/A | 1126 | N/A |
| 10 | 80.1 | 41.75 | 178.4 | 375 | N/A | 1365 | N/A |
| 11 | 70.5 | 51.17 | 684.9 | 712.5 | N/A | 812.9 | N/A |
| 12 | 70.53 | 69.65 | 1160 | 977 | N/A | 881.5 | N/A |
| 13 | 50.89 | 38.01 | 363 | 600.7 | N/A | 388.5 | N/A |
| 14 | 43.94 | 40.86 | 563 | 233.9 | N/A | 319.2 | N/A |
| 15 | 29.24 | 19.88 | 176.3 | 159.4 | N/A | 160.4 | N/A |
| 16 | 20.79 | 11.25 | 187.8 | 165.4 | N/A | 200.4 | N/A |
| 17 | 23.05 | 20.99 | 380.1 | 368.5 | N/A | 426.1 | N/A |
| 18 | 15.65 | 10.49 | 120.4 | 392.4 | 126.4 | 835.8 | 194 |
| 19 | 17.42 | 9.125 | 213.9 | 105.4 | N/A | 315.8 | N/A |
| 20 | 16.42 | 12.83 | 132.2 | N/A | 342.8 | N/A | 235.4 |
| 21 | 15.56 | 13.94 | 282 | 205.3 | N/A | 206.4 | N/A |
| 22 | 0.222 | 1.125 | 68.78 | N/A | 108.9 | 224.2 | 149.6 |
| 23 | 1.159 | 623.8 | 102.1 | N/A | 59.61 | N/A | 25.74 |
| 24 | 1.598 | 0.444 | 98.68 | N/A | 121.2 | N/A | 56.43 |
| 25 | 13.89 | 4.44 | 259.4 | N/A | N/A | 1672 | N/A |
| 26 | 13.93 | 13.89 | 280.2 | N/A | 4591 | 119.4 | 80200 |
| 27 | 14.07 | 12.49 | 137.8 | 248.5 | N/A | 300.2 | N/A |
| 28 | 14.73 | 12.7 | 325.3 | 421.5 | N/A | 326.8 | N/A |
| 29 | 9.236 | 7.854 | 107.4 | 51.62 | 109.4 | 183.8 | 102 |
| 30 | 9.86 | 16.32 | 116.1 | 82.02 | 121.1 | 132 | 78.18 |
| 31 | 8.59 | 6.536 | 116.5 | 111.1 | 259.5 | 309.3 | 224.1 |
| 32 | 2.728 | 0.550 | 76.75 | N/A | 106 | N/A | 74.96 |
| 33 | 6.238 | 2.717 | 121.2 | N/A | 194.4 | N/A | 124.8 |
| 34 | 6.901 | 4.766 | 71.3 | 190.1 | N/A | 1242 | 412.1 |
| 35 | 8.151 | 7.503 | 396.2 | N/A | 439.8 | 1003 | 205.1 |
| 36 | 4.44 | 2.468 | 171.7 | N/A | 174.9 | 431.6 | 145.8 |
| 37 | 15.92 | 6.028 | 115.2 | 20.83 | 129.7 | 35.6 | 100.5 |
| 38 | 11.61 | 6.53 | 72.83 | 53.22 | N/A | 193.8 | 80200 |
| 39 | 10.31 | 1.247 | 86.67 | N/A | 98.04 | N/A | 60.13 |
| 40 | 10.43 | 9.39 | 55.67 | 16.74 | N/A | 30.82 | N/A |
| 41 | 4.995 | 3.881 | 89.91 | 70.8 | 97.43 | 12.19 | 39.04 |
| 42 | 5.301 | 3.76 | 58.9 | 48.55 | 79.87 | 40.78 | 51.71 |
| 43 | 24.87 | 14.56 | 179.1 | N/A | 139.5 | N/A | 106 |
| 44 | 26.2 | 1.981 | 181.5 | N/A | 119.8 | N/A | 154.5 |
| 45 | 28.94 | 3.367 | 455.1 | N/A | 127.7 | N/A | 135.1 |
| 46 | 20.81 | 3.983 | 147.7 | N/A | 113.4 | N/A | 92.08 |
| 47 | 136.4 | N/A | 1156 | N/A | N/A | N/A | N/A |
| 48 | 31.86 | 29.41 | 373.3 | N/A | 294.4 | N/A | 190.3 |
| 49 | 36.32 | 17.62 | 154.7 | N/A | 793.8 | N/A | 509.6 |
| 50 | 37.25 | N/A | 461 | N/A | 243 | N/A | 180.6 |
| 51 | 48.5 | N/A | 368.8 | N/A | 170.3 | N/A | 164 |
| 52 | 51.83 | N/A | 934.6 | N/A | N/A | N/A | N/A |
| 53 | 56.47 | N/A | 1836 | N/A | N/A | N/A | N/A |
| 54 | 61.57 | 14.95 | 768.5 | N/A | 181 | N/A | 240.6 |
| 55 | 65.37 | N/A | 780.9 | N/A | N/A | N/A | N/A |
| 56 | 68.8 | N/A | 687.2 | N/A | 1431 | N/A | 847 |
| 57 | 86.86 | 12.69 | 132.6 | N/A | 203.2 | N/A | 110.2 |
| 58 | 155.5 | N/A | 1808 | N/A | N/A | N/A | N/A |
| 59 | 150.9 | N/A | 975.7 | N/A | N/A | N/A | N/A |
| 60 | 197.3 | N/A | 1494 | N/A | N/A | N/A | N/A |
| 61 | 238.3 | 181 | 484.5 | 81.58 | N/A | 107.7 | N/A |
| 62 | 221.7 | 67.53 | 1580 | 162.4 | N/A | 4879 | N/A |
| 63 | 13.47 | 11.12 | 246.5 | N/A | 215.4 | 34.18 | 310.5 |
| 64 | 163.6 | 65.3 | 1022 | 440.3 | N/A | 1169 | N/A |
| 65 | 35.46 | 11.83 | 768.8 | N/A | 721.4 | N/A | 287.7 |
| 66 | 47.4 | 18.59 | 249.6 | N/A | 288.1 | N/A | 241.5 |
| 67 | 50.67 | 21.73 | 612.5 | N/A | 706.4 | N/A | 659.7 |
| 68 | 51.42 | 38.69 | 602.8 | 126.3 | 1084 | 270.2 | 689.1 |
| 69 | 109.3 | 32.51 | 677.5 | 351 | N/A | 1111 | N/A |
| 70 | 116.6 | 27.85 | 1218 | 298.2 | N/A | 430.2 | N/A |
| 71 | 171.7 | 119.2 | 1508 | N/A | N/A | 354.3 | N/A |
| 72 | 68.85 | 51.84 | 854.6 | N/A | N/A | 1113 | N/A |
| 73 | 93.16 | 45.03 | 1781 | N/A | N/A | 842.2 | N/A |
| 74 | 83.04 | 35.02 | 1438 | N/A | N/A | 280 | N/A |
| 75 | 188.8 | 153.6 | 1393 | N/A | N/A | 1597 | N/A |
| 76 | 209.3 | 164.7 | 1231 | N/A | N/A | 624.2 | N/A |
| 77 | 102.9 | 37.2 | 825.3 | 230.1 | N/A | 122 | N/A |
| 78 | 83.21 | 31.73 | 446.1 | 641.9 | N/A | 560.3 | N/A |
| 79 | 6.896 | 5.185 | 102.9 | N/A | 208.7 | 195.6 | 219.2 |
| 80 | 8.759 | N/A | 247.7 | N/A | 401.3 | N/A | 246.8 |
| 81 | 14.29 | 11.93 | 312.3 | N/A | 306.8 | N/A | 153.3 |
| 82 | 15.98 | 9.022 | 90.46 | N/A | 243.3 | N/A | 125.4 |
| 83 | 12.01 | N/A | 549.2 | N/A | 146.6 | N/A | 273.7 |
| 84 | 25.94 | 26.7 | 349.5 | 66.75 | 344.9 | 520.2 | 462.8 |
| 85 | 65.28 | N/A | 888.4 | N/A | 4714 | N/A | 1644 |
| 86 | 188.8 | 20.55 | 754.4 | 824.8 | N/A | 1448 | N/A |
| 87 | 86.33 | 38.78 | 625.6 | 440.6 | N/A | 378.5 | N/A |
| 88 | 183.7 | N/A | 615.7 | N/A | 1179 | N/A | 785.2 |
| 89 | 186.6 | N/A | 1522 | N/A | N/A | N/A | N/A |
| 90 | 188.6 | 104.9 | 1044 | 737.1 | 5986 | 853.4 | 2546 |
| 91 | 32.45 | 17.35 | 239.2 | 264.9 | N/A | 239.1 | N/A |
| 92 | 10.3 | N/A | 227 | N/A | 338.8 | N/A | 200.2 |
| 93 | 1.421 | 1.994 | 66.72 | N/A | 118.1 | 240.8 | 123.6 |
| 94 | 31.65 | 21.4 | 111.1 | 155 | 426.7 | 368.2 | 412.3 |
| 95 | 38.78 | 23.88 | 181 | 241.3 | N/A | 1925 | N/A |
| 96 | 5.456 | 3.364 | 86.35 | 53.61 | 132.3 | 118.5 | 71 |
| 97 | 13.95 | 15.65 | 101.4 | 95.53 | N/A | 191.2 | N/A |
| 98 | 45.57 | 27.9 | 508.9 | N/A | N/A | 1520 | N/A |
| 99 | 407.3 | N/A | 4012 | N/A | N/A | N/A | N/A |
| 100 | 982.6 | 425.7 | N/A | N/A | N/A | N/A | N/A |
| 101 | 2024 | 1300 | N/A | N/A | N/A | N/A | N/A |
| 102 | 198.6 | 117.4 | 8398 | N/A | N/A | 427.3 | N/A |
| 103 | 2310 | 956.2 | 19420 | N/A | N/A | 2999 | N/A |
| 104 | 272.8 | N/A | 5976 | N/A | N/A | N/A | N/A |
| 105 | 223.1 | 139.2 | 1304 | 869.7 | N/A | 655.1 | N/A |
| 106 | 223.8 | 196.9 | 922.6 | 287.8 | N/A | 574.6 | N/A |
| 107 | 24 | 3.072 | 548.9 | N/A | 582.3 | N/A | 473.2 |
| 108 | 45.11 | 26.43 | 843.5 | N/A | 1623 | N/A | 645.7 |
| 109 | 8.502 | N/A | 132.8 | N/A | 742.9 | N/A | 253.1 |
| 110 | 21.96 | 4.841 | 354.4 | N/A | 1524 | N/A | 997.9 |
| 111 | 13.57 | 13.77 | 396.2 | N/A | 1157 | 719 | 1116 |
| 112 | 54.36 | 31.1 | 635.1 | 481.7 | N/A | 3255 | N/A |
| 113 | 56.62 | 18.41 | 357.4 | N/A | N/A | 227.3 | N/A |
| 114 | 57.01 | 26.53 | 656 | N/A | N/A | 332.4 | N/A |
| 115 | 172.9 | 131.9 | 1716 | 899.6 | N/A | 1277 | N/A |
| 116 | 84.78 | N/A | 791.3 | N/A | 3601 | N/A | 2422 |
| 117-1 | 22.72 | 10.48 | 360.4 | N/A | 844.1 | N/A | 566.4 |
| 117-2 | 12.13 | 3.048 | 211 | N/A | 9103 | N/A | 4782 |
| 118 | 38.85 | N/A | 578.8 | N/A | N/A | N/A | N/A |
| 119 | 65.24 | N/A | 747.1 | N/A | N/A | N/A | N/A |
| 120 | 16.24 | 2.596 | 210.5 | N/A | 441.2 | N/A | 433.6 |
| 121 | 36.53 | N/A | 257.4 | N/A | N/A | N/A | N/A |
| 122 | 19.16 | 9.04 | 121.3 | N/A | 581.8 | N/A | 519.8 |
| 123 | 160 | N/A | 1310 | N/A | N/A | N/A | N/A |
| 124 | 171.7 | N/A | 1271 | N/A | 6049 | N/A | 2851 |
| 125 | 37.66 | 20.04 | 197.8 | N/A | 719.7 | N/A | 531.1 |
| 126 | 25.76 | 16.55 | 572.4 | 487.8 | 1461 | 154.3 | 1965 |
| 127 | 30.02 | 28.31 | 349.2 | 239.3 | 1112 | 234 | 799.6 |
| 128 | 169 | N/A | 1785 | N/A | 5336 | N/A | 8332 |
| 129 | 53.99 | 18.03 | 545.9 | 278 | N/A | 338.8 | N/A |
| 130 | 89.05 | 43.37 | 557.4 | 251.7 | N/A | 495.7 | N/A |
| 131 | 11.81 | 11.03 | 98.54 | 158.1 | N/A | 267.5 | N/A |
| 132 | 160.6 | 40.32 | 1317 | 510.1 | N/A | 400.6 | N/A |
| 133 | 21.25 | 17.7 | 566.8 | N/A | 1139 | 199.8 | N/A |
| 134 | 123.9 | 95.42 | 1933 | N/A | 3972 | 5850 | N/A |
| 135 | 90.01 | 41.12 | 634 | 343.6 | N/A | 9211 | N/A |
| 136 | 213.9 | 152.3 | 989.4 | 386.2 | N/A | 9263 | N/A |
| 137 | 34.95 | 36.72 | 574.8 | N/A | 1415 | 601.9 | N/A |
| 138 | 65.78 | 13.7 | 295.6 | 197.5 | N/A | 419.5 | N/A |
| 139 | 32.97 | 13.31 | 416.7 | N/A | 1176 | 545.3 | N/A |
| 140 | 61.95 | 54.59 | 855.3 | N/A | 2563 | 4316 | N/A |
| 141 | 77.52 | 44.84 | 553.6 | N/A | 2209 | 287.3 | N/A |
| 142 | 17.77 | 12.86 | 242.9 | N/A | 490.2 | 452.7 | N/A |
| 143 | 14.78 | 13.35 | 252.6 | N/A | 1268 | 471 | N/A |
| 144 | 20.59 | 9.225 | 626.1 | N/A | 690.9 | 172.9 | N/A |
| 145 | 176.9 | 159.8 | 756.5 | N/A | 3542 | 8235 | N/A |
| 146 | 20.1 | 8.039 | 1145 | N/A | N/A | 6219 | N/A |
| 147a | 19.03 | 23.35 | 245.2 | N/A | 622.5 | N/A | 413.8 |
| 147 | 804.3 | N/A | 12680 | N/A | N/A | N/A | N/A |
| 148b | 1.889 | N/A | 102 | N/A | 220.4 | N/A | 115.7 |
| 148 | 2.533 | 4.47 | 163.1 | N/A | 228.7 | N/A | 129.1 |
| 149 | 16.43 | 6.308 | 123.8 | N/A | 269.3 | N/A | 124.8 |
| 150 | 7.758 | 0.444 | 109.5 | N/A | 143.5 | N/A | 78.16 |
| 151 | 237.9 | 232 | 5342 | 1414 | N/A | 1031 | N/A |
| 152 | 201.2 | 133.8 | 1891 | N/A | N/A | 10670 | N/A |
| 153 | 66.23 | N/A | 477.7 | N/A | 913 | N/A | 635.7 |
| 154 | 36.71 | 11.41 | 255.3 | N/A | 896.3 | N/A | 661.6 |
| 155b | 8.498 | 9.559 | 380 | N/A | 394.7 | 209.3 | 262.6 |
| 155 | 7.368 | 4.715 | 204 | N/A | 250.7 | 418.6 | 243.3 |
| 156-1 | 40.31 | 24.88 | 1282 | N/A | 2630 | 3497 | N/A |
| 156-2 | 8.027 | 4.965 | 599.6 | N/A | 1181 | 737.9 | N/A |
| 157 | 150 | 70.77 | 901.4 | 851.5 | N/A | 222.2 | N/A |
| 158 | 67.88 | 40.72 | 1040 | 573.6 | N/A | 473 | N/A |
| 159 | 59.17 | 19.34 | 440.7 | 276.8 | N/A | 329.2 | N/A |
| 160 | 135.1 | N/A | 1400 | N/A | N/A | N/A | N/A |
| 161-1 | 130.3 | 47.27 | 1333 | 210.2 | N/A | 460.7 | N/A |
| 162-1 | 3.076 | 2.425 | 88.64 | 61.08 | 446.8 | 77.01 | 106.7 |
| 163 | 203 | 104.8 | 2728 | 588.4 | N/A | 762.3 | N/A |
| 164 | 18.26 | 2.371 | 436.4 | N/A | 684 | N/A | 469.2 |
| 165 | 5.279 | 6.248 | 141.4 | N/A | 138.2 | N/A | 121.7 |
| 166 | 9.495 | 8.577 | 565.1 | N/A | 240.6 | N/A | 369.6 |
| 167 | 28.64 | 8.551 | 198.5 | N/A | 678.6 | N/A | 422.6 |
| 168 | 103.5 | 67.61 | 1112 | 590.2 | N/A | 528.9 | N/A |
| 169 | 46.73 | 5.454 | 1490 | N/A | N/A | 594.3 | N/A |
| 170 | 178.2 | 87.4 | 735.5 | 599.3 | N/A | 266.3 | N/A |
| 171 | 219.4 | 87.67 | 1093 | 883.2 | N/A | 1949 | N/A |
| 172 | 128.4 | N/A | 418.8 | N/A | 4062 | N/A | 4194 |
| 173 | 12.28 | N/A | 278.9 | N/A | 682.4 | N/A | 832.9 |
| 174 | 36.54 | N/A | 471.8 | N/A | 1190 | N/A | 965.6 |
| 175 | 48.2 | 76.87 | 1222 | N/A | 3323 | N/A | 3391 |
| 176 | 217.8 | 115.3 | 2479 | 1237 | N/A | 2183 | N/A |
| 177 | 601.2 | N/A | 1412 | N/A | N/A | N/A | N/A |
| 178 | N/A | N/A | N/A | N/A | N/A | N/A | N/A |
| 179 | 106.6 | N/A | 1390 | N/A | N/A | N/A | N/A |
| 180 | 29.2 | N/A | 183.2 | N/A | 264.9 | N/A | 175.6 |
| 181-1 | N/A | N/A | N/A | N/A | N/A | N/A | N/A |
| 181-2 | N/A | N/A | N/A | N/A | N/A | N/A | N/A |
| 182 | 226.7 | N/A | 147.8 | N/A | 212.8 | N/A | 105.4 |
| 183 | 201.1 | N/A | 149.6 | N/A | N/A | N/A | N/A |
| 184 | 893.9 | N/A | 518.7 | N/A | 314 | N/A | 173 |
| 185 | 25.22 | N/A | 126.4 | N/A | 263.3 | N/A | 152.5 |
| 186 | N/A | N/A | N/A | N/A | N/A | N/A | N/A |
| 187 | N/A | N/A | N/A | N/A | N/A | N/A | N/A |
| 188 | N/A | N/A | N/A | N/A | N/A | N/A | N/A |
| 189 | N/A | N/A | N/A | N/A | N/A | N/A | N/A |

N/A = not available
^{a}data generated using the assay described in Example 190
^{b}data generated using the assay described in Example 191

### Uses of the Amido-Substituted Pyridyl Compounds

The Amido-Substituted Pyridyl Compounds are useful in human and veterinary medicine for treating or preventing a viral infection in a patient. In one embodiment, the Amido-Substituted Pyridyl Compounds can be inhibitors of viral replication. In another embodiment, the Amido-Substituted Pyridyl Compounds can be inhibitors of herpesvirus replication. Accordingly, the Amido-Substituted Pyridyl Compounds are useful for treating viral infections, such as herpesvirus. In accordance with the invention, the Amido-Substituted Pyridyl Compounds can be administered to a patient in need of treatment or prevention of a viral infection.

Accordingly, in one embodiment, the invention provides methods for treating or preventing a viral infection in a patient comprising administering to the patient an effective amount of at least one Amido-Substituted Pyridyl Compound or a pharmaceutically acceptable salt thereof.

### Treatment or Prevention of Herpesvirus Infection

The Amido-Substituted Pyridyl Compounds are useful in the inhibition of herpesvirus replication, the treatment of herpesvirus infection and/or reduction of the likelihood or severity of symptoms of herpesvirus infection and the inhibition of herpesvirus viral replication and/or herpesvirus viral production in a cell-based system. For example, the Amido-Substituted Pyridyl Compounds are useful in treating infection by herpesvirus after suspected past exposure to herpesvirus by such means as blood transfusion, exchange of body fluids, bites, accidental needle stick, or exposure to patient blood during surgery or other medical procedures. Accordingly, in one embodiment, the invention provides a method for treating herpesvirus infection in a patient, the method comprising administering to the patient an effective amount of at least one Amido-Substituted Pyridyl Compound or a pharmaceutically acceptable salt thereof.

In one embodiment, the herpesvirus being treated or prevented is of the family α-herpesviridae. Herpesviruses of the family α-herpesviridae include, but are not limited to, herpes simplex virus 1 (HSV-1), herpes simplex 2 (HSV-2), and varicella zoster virus (VZV).

In another embodiment, the herpesvirus being treated or prevented is of the family (3-herpesviridae. Herpesviruses of the family β-herpesviridae include, but are not limited to, human cytomegalovirus (CMV), human herpesvirus 6 (HHV6), and human herpesvirus 7 (HHV7).

In another embodiment, the herpesvirus being treated or prevented is of the family γ-herpesviridae. Herpesviruses of the family γ-herpesviridae include, but are not limited to, Epstein-Barr virus (EBV), human herpesvirus 4 (HHV4), and Kaposi's sarcoma-associated herpesvirus (KHSV), also known as human herpesvirus 8 (HHV8).

In one embodiment, the herpesvirus being treated or prevented is HSV-1.

In another embodiment, the herpesvirus being treated or prevented is HSV-2.

In another embodiment, the herpesvirus being treated or prevented is VZV.

In still another embodiment, the herpesvirus being treated or prevented is CMV.

In another embodiment, the herpesvirus being treated or prevented is HHV6.

In yet another embodiment, the herpesvirus being treated or prevented is HHV7.

In another embodiment, the herpesvirus being treated or prevented is EBV.

In a further embodiment, the herpesvirus being treated or prevented is HHV4.

In another embodiment, the herpesvirus being treated or prevented is KSHV.

In a specific embodiment, the amount administered is effective to treat or prevent infection by herpesvirus in the patient. In another specific embodiment, the amount administered is effective to inhibit herpesvirus viral replication and/or viral production in the patient.

The Amido-Substituted Pyridyl Compounds are also useful in the preparation and execution of screening assays for antiviral compounds. Furthermore, the Amido-Substituted Pyridyl Compounds are useful in establishing or determining the binding site of other antivirals to the herpesvirus polymerase.

The compositions and combinations of the present invention can be useful for treating a patient suffering from infection related to any herpesvirus infection. Herpesvirus types may differ in their antigenicity, level of viremia, severity of disease produced, and response to therapy. See Poole et al., Clinical Therapeutics, 40:8 (2018), 1282-1298.

### Combination Therapy

In another embodiment, the present methods for treating or preventing herpesvirus infection can further comprise the administration of one or more additional therapeutic agents which are not Amido-Substituted Pyridyl Compounds.

In one embodiment, the additional therapeutic agent is an antiviral agent. In another embodiment, the additional therapeutic agent is an anti-herpes agent.

Anti-herpes agents useful in the present compositions and methods include, but are not limited to, nucleoside polymerase inhibitors, such as acyclovir, valaciclovir, famciclovir, penciclovir, cidofovir, brincidofovir (CMX-001), valmanciclovir, ganciclovir, valganciclovir, and N-methanocarbathymidine (N-MCT); pyrophosphate polymerase inhibitors, such as foscarnet; CMV terminase inhibitors, such as letermovir; viral kinase inhibitors, such as maribavir; and helicase-primase inhibitors, such as pritelivir (AIC-316), and amenamevir (ASP-2151).

In another embodiment, the additional therapeutic agent is an immunomodulatory agent, such as an immunosuppressive agent. Immunosuppressant agents useful in the present compositions and methods include, but are not limited to, cytotoxic agents, such as cyclophosphamide and cyclosporin A; corticosteroids, such as hydrocortisone and dexamethasone, and non-steroidal anti-inflammatory agents (NSAID).

Accordingly, in one embodiment, the present invention provides methods for treating a herpesvirus infection in a patient, the method comprising administering to the patient: (i) at least one Amido-Substituted Pyridyl Compound, or a pharmaceutically acceptable salt thereof, and (ii) at least one additional therapeutic agent that is other than an Amido-Substituted Pyridyl Compound, wherein the amounts administered are together effective to treat or prevent the herpesvirus infection.

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, an Amido-Substituted Pyridyl Compound and an additional therapeutic agent may be present in fixed amounts (dosage amounts) in a single dosage unit (e.g., a capsule, a tablet and the like).

In one embodiment, the at least one Amido-Substituted Pyridyl Compound is administered during a time when the additional therapeutic agent(s) exert their prophylactic or therapeutic effect, or vice versa.

In another embodiment, the at least one Amido-Substituted Pyridyl Compound and the additional therapeutic agent(s) are administered in doses commonly employed when such agents are used as monotherapy for treating a herpesvirus infection.

In another embodiment, the at least one Amido-Substituted Pyridyl Compound and the additional therapeutic agent(s) are administered in doses lower than the doses commonly employed when such agents are used as monotherapy for treating a herpesvirus infection.

In still another embodiment, the at least one Amido-Substituted Pyridyl Compound and the additional therapeutic agent(s) act synergistically and are administered in doses lower than the doses commonly employed when such agents are used as monotherapy for treating a herpesvirus infection.

In one embodiment, the at least one Amido-Substituted Pyridyl Compound and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

The at least one Amido-Substituted Pyridyl Compound and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one Amido-Substituted Pyridyl Compound and the additional therapeutic agent(s) may inhibit the resistance of a herpesvirus infection to these agents.

The doses and dosage regimen of the other agents used in the combination therapies of the present invention for the treatment or prevention of herpesvirus infection can be determined by the attending clinician, taking into consideration the approved doses and dosage regimen in the package insert; the age, sex and general health of the patient; and the type and severity of the viral infection or related disease or disorder. When administered in combination, the Amido-Substituted Pyridyl Compound(s), and the other agent(s) can be administered simultaneously (i.e., in the same composition or in separate compositions one right after the other) or sequentially. This particularly useful when the components of the combination are given on different dosing schedules, e.g., one component is administered once daily and another component is administered every six hours, or when the preferred pharmaceutical compositions are different, e.g., one is a tablet and one is a capsule. A kit comprising the separate dosage forms is therefore advantageous.

In one embodiment, one or more compounds of the present invention are administered with one or more additional therapeutic agents selected from: an immunomodulator, an anti-herpes agent, a viral replication inhibitor, an antisense agent, a therapeutic vaccine, a virion production inhibitor, a viral entry inhibitor, a viral assembly inhibitor, an antibody therapy (monoclonal or polyclonal), and any agent useful for treating any type of herpesvirus infection.

### Compositions and Administration

Due to their activity, the Amido-Substituted Pyridyl Compounds are useful in veterinary and human medicine. As described above, the Amido-Substituted Pyridyl Compounds are useful for treating or preventing herpesvirus infection in a patient in need thereof.

Accordingly, in one embodiment, the present invention provides pharmaceutical compositions comprising an effective amount of a compound of formula(I), or a pharmaceutically acceptable salt thereof, and a pharmaceutically acceptable carrier.

In another embodiment, the present invention provides pharmaceutical compositions comprising (i) an effective amount of a compound of formula(I), or a pharmaceutically acceptable salt thereof, and a pharmaceutically acceptable carrier; and (ii) one or more additional therapeutic agents, wherein said additional therapeutic agents are selected from anti-herpes agents and immunomodulators.

When administered to a patient, the Amido-Substituted Pyridyl Compounds can be administered as a component of a composition that comprises a pharmaceutically acceptable carrier or vehicle. The present invention provides pharmaceutical compositions comprising an effective amount of at least one Amido-Substituted Pyridyl Compound and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms), and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral or intravenous injection.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Additionally, the compositions of the present invention may be formulated in sustained release form to provide the rate-controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more Amido-Substituted Pyridyl Compounds are administered orally.

In another embodiment, the one or more Amido-Substituted Pyridyl Compounds are administered intravenously.

In still another embodiment, the one or more Amido-Substituted Pyridyl Compounds are administered sublingually.

In one embodiment, a pharmaceutical preparation comprising at least one Amido-Substituted Pyridyl Compound is in unit dosage form. In such form, the preparation is subdivided into unit doses containing effective amounts of the active components.

Compositions can be prepared according to conventional mixing, granulating or coating methods, respectively, and the present compositions can contain, in one embodiment, from about 0.1%, to about 99% of the Amido-Substituted Pyridyl Compound(s) by weight or volume. In various embodiments, the present compositions can contain, in one embodiment, from about 1%, to about 70% or from about 5%, to about 60% of the Amido-Substituted Pyridyl Compound(s) by weight or volume.

The amount and frequency of administration of the Amido-Substituted Pyridyl Compounds will be regulated according to the judgment of the attending clinician considering such factors as age, condition and size of the patient as well as severity of the symptoms being treated. Generally, a total daily dosage of the at least one Amido-Substituted Pyridyl Compound(s) alone, or when administered as combination therapy, can range from about 1 to about 2500 mg per day, although variations will necessarily occur depending on the target of therapy, the patient and the route of administration. In one embodiment, the dosage is from about 10 to about 1000 mg/day, administered in a single dose or in 2-4 divided doses. In another embodiment, the dosage is from about 1 to about 500 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In another embodiment, the dosage is from about 500 to about 1500 mg/day, administered in a single dose or in 2-4 divided doses. In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

The compositions of the invention can further comprise one or more additional therapeutic agents, selected from those listed above herein. Accordingly, in one embodiment, the present invention provides compositions comprising: (i) at least one Amido-Substituted Pyridyl Compound or a pharmaceutically acceptable salt thereof; (ii) one or more additional therapeutic agents that are not an Amido-Substituted Pyridyl Compound; and (iii) a pharmaceutically acceptable carrier, wherein the amounts in the composition are together effective to treat herpesvirus infection.

In one embodiment, the present invention provides compositions comprising a Compound of Formula (I), and a pharmaceutically acceptable carrier.

In another embodiment, the present invention provides compositions comprising a Compound of Formula (I), a pharmaceutically acceptable carrier, and a second therapeutic agent selected from the group consisting of anti-herpes agents and immunomodulators.

In another embodiment, the present invention provides compositions comprising a Compound of Formula (I), a pharmaceutically acceptable carrier, and two additional therapeutic agents, each of which are independently selected from the group consisting of anti-herpes agents and immunomodulators.

### Kits

In one aspect, the present invention provides a kit comprising a therapeutically effective amount of at least one Amido-Substituted Pyridyl Compound, or a pharmaceutically acceptable salt, solvate, ester or prodrug of said compound and a pharmaceutically acceptable carrier, vehicle or diluent.

In another aspect the present invention provides a kit comprising an amount of at least one Amido-Substituted Pyridyl Compound, or a pharmaceutically acceptable salt, solvate, ester or prodrug of said compound and an amount of at least one additional therapeutic agent listed above, wherein the amounts of the two or more active ingredients result in a desired therapeutic effect. In one embodiment, the one or more Amido-Substituted Pyridyl Compounds and the one or more additional therapeutic agents are provided in the same container. In one embodiment, the one or more Amido-Substituted Pyridyl Compounds and the one or more additional therapeutic agents are provided in separate containers.

1. A compound of formula (I): 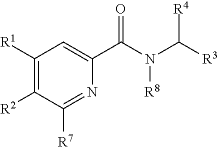
or a pharmaceutically acceptable salt thereof,
wherein:
R¹ is selected from —O—(C₁-C₆ alkyl), 5 to 7-membered monocyclic heterocycloalkyl, —O-(5 to 7-membered monocyclic heterocycloalkyl), 6 to 10-membered bicyclic heterocycloalkyl —O-(6 to 10-membered bicyclic heterocycloalkyl), wherein said 5 to 7-membered monocyclic heterocycloalkyl group, and said 6 to 10-membered bicyclic heterocycloalkyl group, may each be optionally substituted with one or more R^{A} groups, which can be the same or different;
R² is selected from H, —(C₁-C₆ alkylene)ₘ-(8-10 membered bicyclic heteroaryl), C₁-C₆ alkyl, —OH, halo, —O—(C₁-C₆ alkyl), —(C₁-C₆ alkylene)—O—P(O)(OH)₂, —P(O)(OH)₂, —SO₂—(C₁-C₆ alkyl), C₁-C₆ hydroxyalkyl, C₁-C₆ hydroxyalkenyl, —(C₁-C₆ alkenylene)-R⁶, —(C₁-C₆ alkynylene)-R⁶, —CN, —O—(C₁-C₆ haloalkyl), —(C₁-C₆ alkylene)-N(R⁵)₂, —(C₁-C₆ alkylene)—O—(C₁-C₆ alkyl), C₃-C₆ cycloalkyl, 6 to 10-membered bicyclic heterocycloalkyl, —(C₁-C₆ alkylene)ₘ-(3 to 7-membered monocyclic heterocycloalkyl), —(C₁-C₆ alkylene)ₘ-(5 or 6-membered monocyclic heteroaryl), and 9 or 10-membered bicyclic heteroaryl, wherein said C₃-C₆ cycloalkyl group, said 6 to 10-membered bicyclic heterocycloalkyl group, said 3 to 7-membered monocyclic heterocycloalkyl group, said 5 or 6-membered monocyclic heteroaryl group, and said 9 or 10-membered bicyclic heteroaryl group, may each be optionally substituted with one or more R^{B} groups, which can be the same or different;
R³ is phenyl or 5 or 6-membered monocyclic heteroaryl, each of which may each be optionally substituted with one or more R^{C} groups, which can be the same or different, or R³ and R⁸, together with the common nitrogen atom to which they are attached, can combine to form a pyrrolidine ring, which can be optionally substituted with C₁-C₆ alkyl, phenyl or 5 or 6-membered monocyclic heteroaryl, wherein said phenyl group or said 5 or 6-membered monocyclic heteroaryl group, may each be optionally substituted with one or more R^{C} groups, which can be the same or different;
R⁴ is H or C₁-C₆ alkyl;
each occurrence of R⁵ is independently selected from H, C₁-C₆ alkyl, C₃-C₆ cycloalkyl, 3 to 6-membered monocyclic heterocycloalkyl, and C₁-C₆ hydroxyalkyl;
R⁶ is selected from H, 5 or 6-membered monocyclic heteroaryl, C₁-C₆ hydroxyalkyl, and —(C₁-C₆ alkylene)—O—(C₁-C₆ alkyl), wherein said 5 or 6-membered monocyclic heteroaryl group may be optionally substituted with one or more R^{C} groups, which can be the same or different;
R⁷ is selected from H, halo, C₁-C₆ alkyl, C₁-C₆ hydroxyalkyl, —CN, and 5 or 6-membered monocyclic heteroaryl, wherein said 5 or 6-membered monocyclic heteroaryl group may be optionally substituted with one or more R^{C} groups, which can be the same or different;
R⁸ is selected from H, —C(O)O-(5 or 6-membered monocyclic heterocycloalkyl), —C(O)O—(C₁-C₆ alkylene)—O—(C₁-C₆ alkyl), C₁-C₆ hydroxyalkyl, and —(C₁-C₆ alkylene)ₘ-OC(O)—R⁹;
R⁹ is selected from C₃-C₆ cycloalkyl, 5 or 6-membered monocyclic heterocycloalkyl, —(C₁-C₆ alkylene)-OC(═O)—(C₁-C₆ alkyl), C₁-C₆ aminoalkyl, and —(C₁-C₆ alkylene)—O—P(O)(OH)₂, —(C₁-C₆ alkylene)-P(O)(OH)₂, wherein said C₃-C₆ cycloalkyl group, and said 5 or 6-membered monocyclic heterocycloalkyl group may be optionally substituted with one substituent, selected from C₁-C₆ alkyl, —OH, 5 or 6-membered monocyclic heterocycloalkyl, and —O—P(O)(OH)₂, and wherein said 5 or 6-membered monocyclic heterocycloalkyl substituent group may be further optionally substituted with C₁-C₆ alkyl;
each occurrence of R^{A} is independently selected from C₁-C₆ alkyl, —C(O)—C₁-C₆ alkyl, C₁-C₆ haloalkyl, —S(O)₂—(C₁-C₆ alkyl), oxo, and —CN;
each occurrence of R^{B} is independently selected from —OH, halo C₁-C₆ alkyl, —O—(C₁-C₆ alkyl), C₁-C₆ haloalkyl, C₁-C₆ hydroxyalkyl, C₃-C₆ cycloalkyl, 3 to 6-membered monocyclic heterocycloalkyl, —(C₁-C₆ alkylene)-N(R⁴)₂, —(C₁-C₆ alkylene)—O—(C₁-C₆ alkyl), and —(C₁-C₆ alkylene)—SO₂—(C₁-C₆ alkyl);
each occurrence of R^{C} is independently selected from C₁-C₆ alkyl, C₁-C₆ haloalkyl, C₁-C₆ hydroxyalkyl, halo, —CN, C₁-C₆ alkynyl, and NO₂; and
each occurrence of m is independently 0 or 1;
wherein the pyridyl ring nitrogen atom present in formula(I) may be in the form of its N-oxide derivative.

2. The compound of claim 1, or a pharmaceutically acceptable salt thereof, wherein R¹ is selected from:

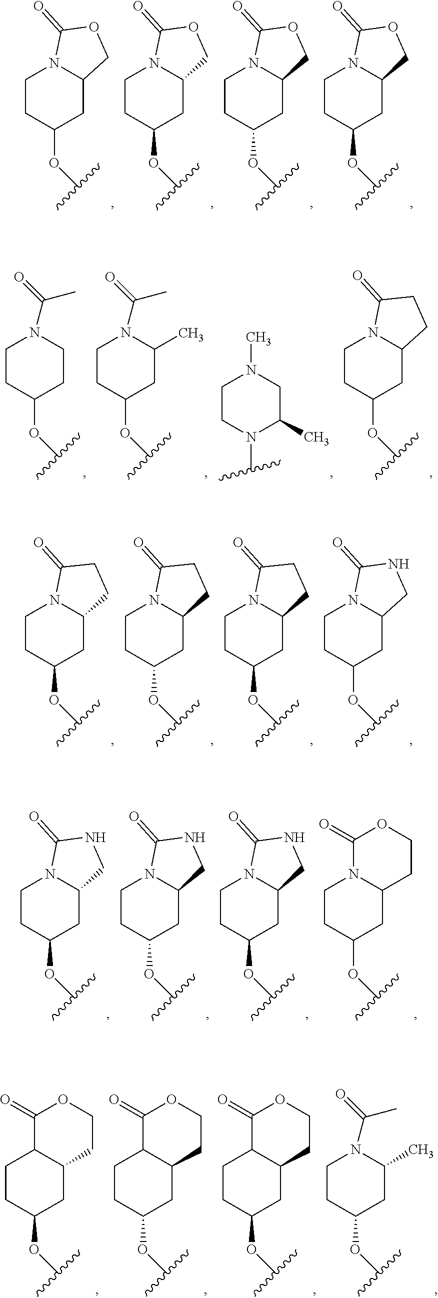

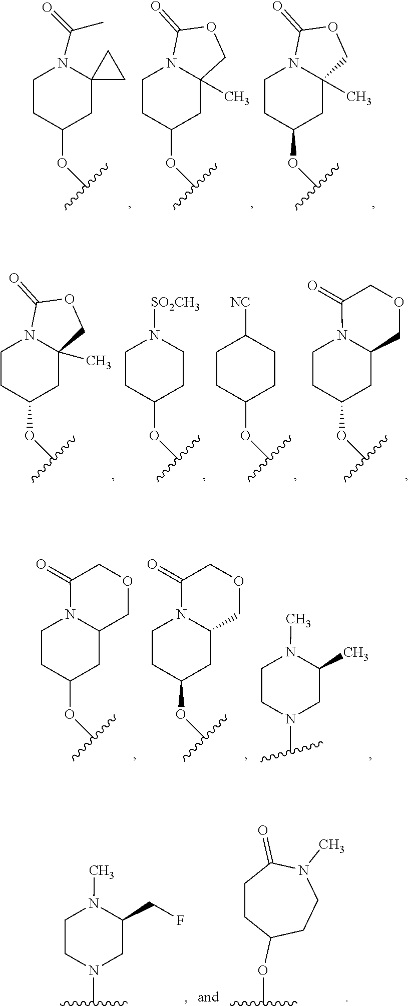

3. The compound of claim 2, or a pharmaceutically acceptable salt thereof wherein R¹ is selected from:

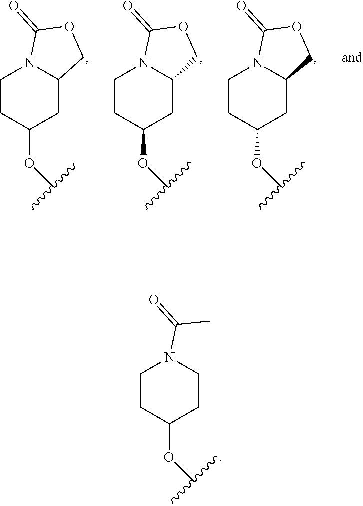

4. The compound of claim 1, or a pharmaceutically acceptable salt thereof, wherein R³ is phenyl or pyridyl, which can be optionally substituted with up to three R^{C} groups, which can be the same or different, and are each individually selected from F, Cl, Br, I, —CN, —C≡CH, and NO₂.

5. The compound of claim 1, or a pharmaceutically acceptable salt thereof, wherein R³ is selected from:

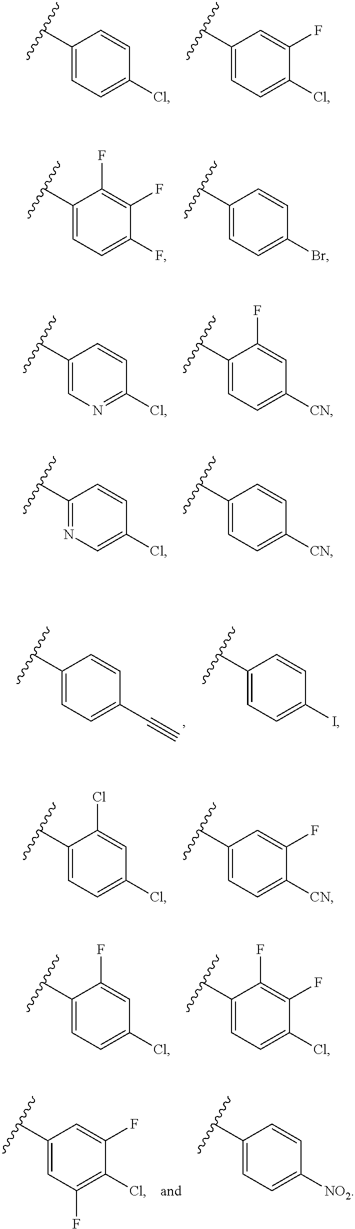

6. The compound of claim 5, or a pharmaceutically acceptable salt thereof, wherein R³ is:

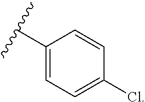 [SMILES: *c1ccc(Cl)cc1]

7. The compound of claim 1, or a pharmaceutically acceptable salt thereof, wherein R⁴ is H or methyl.

8. The compound of claim 1, or a pharmaceutically acceptable salt thereof, wherein R² is selected from H, C₁-C₆ alkyl, halo, —CN, —O—(C₁-C₆ alkyl), —CN, —O—(C₁-C₆ haloalkyl), —(C₁-C₆ alkylene)—O—P(O)(OH)₂, and —(C₁-C₆ alkylene)ₘ-(5 or 6-membered monocyclic heteroaryl), wherein said 5 or 6-membered monocyclic heteroaryl group can be optionally substituted with one or more R^{B} groups, which can be the same or different.

9. The compound of claim 1 or a pharmaceutically acceptable salt thereof, wherein R² is selected from methyl, methoxy, Cl—CN, —CH₂OH, —OH, —SO₂CH₃, —CH₂CH₂OH, —CH₂C(CH₃)₂—OH, —CH═CH—CH₂—OH, —CH₂C(CH₃)₂—OCH₃, —CH₂N(CH₃)—CH₂CH₂OH, —CH₂NH-cyclopropyl, —CH₂N(CH₃)—CH₂CH₂CH₂OH, cyclopropyl, cyclobutyl, —CH₂—O—P(O)(OH)₂, —P(O)(O⁻NH₄⁺)₂, —P(O)(OH)₂, —OCH₂CF₃, tetrahydroypyranyl, —CH₂-tetrahydropyranyl, piperadinyl, —CH₂-piperadinyl, morpholinyl, —CH₂-morpholinyl, azetidinyl, —CH-azetidinyl, oxetanyl, spiro[3,3]heptanyl, 2λ²-azaspiro[3,3]heptanyl, pyrazolyl, triazolyl, isoxazolyl, thiazolyl, isothiazolyl, oxazolyl, 1,2,4-oxadiazolyl, 1,3,4-oxadiazolyl, 1,2,4-thiadiazolyl, and 1,3,4-thiadiazolyl, wherein said cyclopropyl, cyclobutyl, tetrahydropyranyl, piperadinyl, morpholinyl, azetidinyl, oxetanyl, spiro[3,3]heptanyl, 2λ²-azaspiro[3,3]heptanyl, pyrazolyl, triazolyl, isoxazolyl, thiazolyl, isothiazolyl, oxazolyl, 1,3,4-oxadiazolyl, 1,2,4-thiadiazolyl, and 1,3,4-thiadiazolyl groups can be optionally substituted with one or more groups, which can be the same or different, and are selected from —CHF₂, F, —OH, methyl, ethyl, cyclopropyl, n-propyl, isopropyl, t-butyl, isobutyl, —CH₂CF₃, —CH₂C(OH)(CH₃)₂, —CH₂N(CH₃)₂, —CH₂CH₂N(CH₃)₂, —CH₂OH, —CH₂CH₂OH, —CH₂CH₂OCH₃, —CH₂CHF₂, —CH₂CH₂CH(CH₃)₂, oxetane, methoxy, —C(OH)(CH₃)₂, and —CH₂S(O)₂CH₃.

10. The compound of claim 9, or a pharmaceutically acceptable salt thereof, wherein R² is selected from:

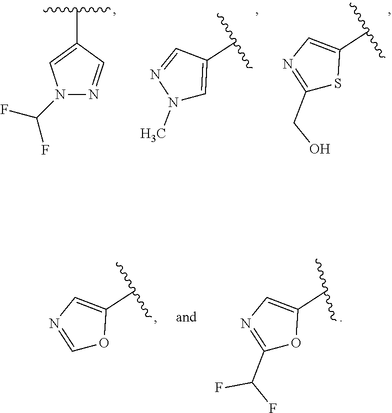

11. The compound of claim 1, or a pharmaceutically acceptable salt thereof, wherein R⁷ is selected from H, methyl, Cl, —CH₂OH, CN,

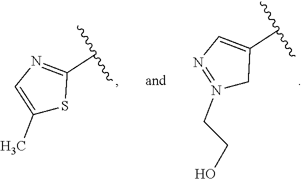

12. The compound of claim 1, or a pharmaceutically acceptable salt thereof, wherein R⁸ is selected from H, —CH₂OH, —CH₂SCH₃, —C(O)CH₂CH₂OCH₃,

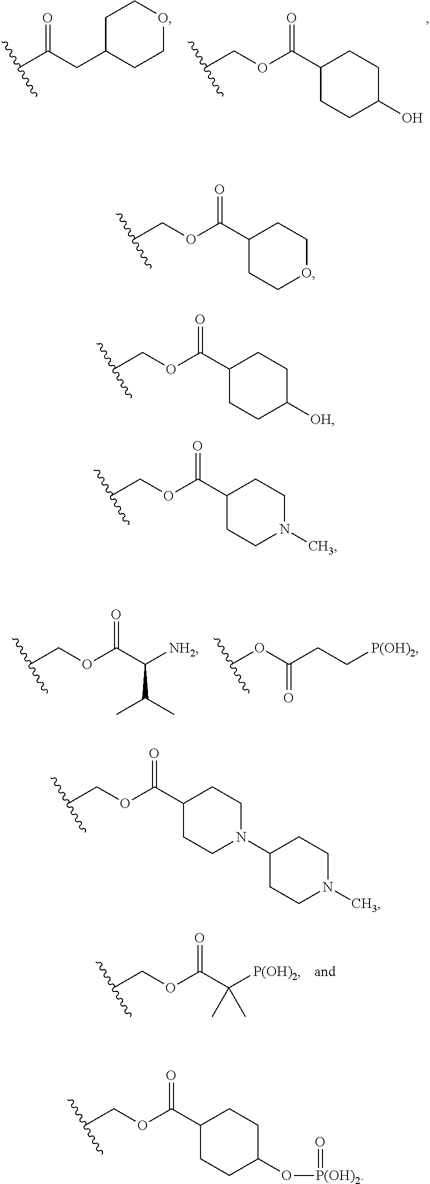

13. (canceled)

14. The compound of claim 1, of formula (Ia): 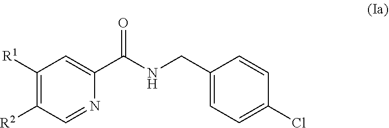
or a pharmaceutically acceptable salt thereof,
wherein:
R¹ is —O-(5 or 6-membered monocyclic heterocycloalkyl) or —O-(9 or 10-membered bicyclic heterocycloalkyl), wherein said 5 or 6-membered monocyclic heteroaryl group, and said 9 or 10-membered bicyclic heterocycloalkyl group can each be optionally substituted with methyl or —C(O)CH₃;
R² is selected from methyl, methoxy, —CN, Cl, —OCH₂CF₃, pyrazolyl, triazolyl, isoxazolyl, thiazolyl, isothiazolyl, oxazolyl, 1,2,4-thiadiazolyl, and 1,3,4-thiadiazolyl, wherein said pyrazolyl, triazolyl, isoxazolyl, thiazolyl, isothiazolyl, oxazolyl, 1,2,4-thiadiazolyl, and 1,3,4-thiadiazolyl groups can be optionally substituted with up to two R^{B} groups, which can be the same or different; and
each occurrence of R^{B} is independently selected from C₁-C₆ alkyl, F, —OH, —O—(C₁-C₆ alkyl), C₁-C₆ haloalkyl, C₁-C₆ hydroxyalkyl, C₃-C₆ cycloalkyl, 3 to 6-membered monocyclic heterocycloalkyl, —(C₁-C₆ alkylene)-N(R⁴)₂, —(C₁-C₆ alkylene)—O—(C₁-C₆ alkyl), and —(C₁-C₆ alkylene)—SO₂—(C₁-C₆ alkyl).

15. A compound being one of the compounds numbered 3, 4, and 6-189, in the above specification, or a pharmaceutically acceptable salt thereof.

16. A pharmaceutical composition comprising an effective amount of the compound of claim 1, or a pharmaceutically acceptable salt thereof, and a pharmaceutically acceptable carrier.

17. The pharmaceutical composition of claim 16, further comprising one or more additional therapeutic agents, wherein said additional therapeutic agents are selected from anti-herpes agents, and immunomodulators.

18. A method of treating a patient infected with a herpesvirus, comprising the step of administering an amount of the compound of claim 1, or a pharmaceutically acceptable salt thereof, effective to treat infection by said herpesvirus in said patient.

19. The method of claim 18, further comprising administering one or more additional therapeutic agents, wherein said additional therapeutic agents are selected from anti-herpes agents, and immunomodulators.

20. The pharmaceutical composition of claim 17, wherein said additional therapeutic agents comprise letermovir.

21. The method of claim 19, wherein said additional therapeutic agents comprise letermovir.
